(19) United States
(12) Patent Application Publication
(10) Pub. No.: US 2026/0337579 A1
(43) Pub. Date: Oct. 29, 2026

(54) MATERIALS AND PROCESSES FOR BIOENGINEERING CELLULAR HYPOIMMUNOGENICITY

(71) Applicant: JANSSEN BIOTECH, INC, Horsham, PA (US)

(72) Inventors: Heather MOWER, Raritan, NJ (US); Jennifer McCaffrey, Raritan, NJ (US); Michael Ports, Raritan, NJ (US); Michael Kalos, Raritan, NJ (US); Michael Allegrezza, Raritan, NJ (US)

(21) Appl. No.: 19/107,748

(22) PCT Filed: Aug. 30, 2023
(86) PCT No.: PCT/IB2023/058590

§ 371 Date: Feb. 28, 2025

Related U.S. Application Data
(60) Provisional application No. 63/403,608, filed on Sep. 2, 2022.
(60) Provisional application No. 63/403,612, filed on Sep. 2, 2022.
(60) Provisional application No. 63/403,617, filed on Sep. 2, 2022.
(60) Provisional application No. 63/431,410, filed on Dec. 9, 2022.
(60) Provisional application No. 63/450,714, filed on Mar. 8, 2023.

(51) Int. Cl. C12N 5/074; A61K 40/11; A61K 40/32; C12N 5/0783; C12N 9/22; C12N 15/11; C12N 15/90

(52) U.S. Cl. CPC C12N 5/0696; A61K 40/11; A61K 40/32; C12N 5/0636; C12N 9/226; C12N 15/11; C12N 15/907; C12N 2310/20; C12N 2510/00

(57) ABSTRACT

Provided herein are methods of hypoimmunogenicity, such as bioengineering methodologies and materials, including hypoimmunogenicity (such as engineering hypoimmunogenicity) methodologies and materials useful in, for example, genetically modifying and/or otherwise altering at least one target gene or gene product, processes for producing hypoimmunogenic cells (such as engineered hypoimmunogenic cells), manufacturing of hypoimmunogenic cellular compositions (such as engineered hypoimmunogenic cellular compositions), hypoimmunogenic cell systems (such as engineered hypoimmunogenic cell systems) and uses thereof.

### 1. CROSS-REFERENCE TO RELATED APPLICATIONS

This application claims the benefit of each of U.S. Ser. No. 63/403,608, filed on Sep. 2, 2023; 63/403,612, filed on Sep. 2, 2023; 63/403,617, filed on Sep. 2, 2023; 63/431,410, filed on Dec. 9, 2022; and 63/450,714, filed on Mar. 8, 2023, the disclosures of each of which is incorporated by reference herein in its entirety.

### 2. SEQUENCE LISTING

The instant application contains a Sequence Listing which has been submitted electronically in XML file format and is hereby incorporated by reference in its entirety. Said XML copy, created on Aug. 29, 2023, is named 253505_000361_SL.xml and is 352,323 bytes in size.

### 3. FIELD

Provided herein are, inter alia, methods of hypoimmunogenicity, such as bioengineering methodologies and materials, including hypoimmunogenicity (such as engineering hypoimmunogenicity) methodologies and materials useful in, for example, genetically modifying and/or otherwise altering at least one target gene or gene product, processes for producing hypoimmunogenic cells (such as engineered hypoimmunogenic cells), manufacturing of hypoimmunogenic cellular compositions (such as engineered hypoimmunogenic cellular compositions), hypoimmunogenic cell systems (such as engineered hypoimmunogenic cell systems), and uses thereof.

### 4. BACKGROUND

Cell therapy approaches are emerging and evolving, and in some instances include efforts to effectively target and neutralize complex diseases, such as varying types of neoplasia, cancers and tumors, in their various forms and locations within hosts. See, “Studies Test CAR T-Cell Therapies Designed to Overcome Key Limitations”, National Cancer Institute, available online (www dot cancer dot gov/news-events/cancer-currents-blog/2023/car-t-cell-therapies-overcoming-limitations), Feb. 8, 2023, by Sharon Reynolds. Challenges have also been reported to stem from, for example, chemical and molecular interference with immune cells, cell to cell interference, competition for nutrients, cellular exhaustion, apoptosis, manufacturing methodologies, etc. Even so, there is a dearth in cell therapy approvals. See, News & Analysis, 2022 FDA Approvals, Asher Mullard, Nature Reviews Drug Discovery, Volume 22, February 2023, pages 83-88.

### 5. SUMMARY

The inventors provide herein, inter alia, methods of hypoimmunogenicity, such as bioengineering methodologies and materials, including hypoimmunogenicity (such as engineering hypoimmunogenicity) methodologies and materials useful in, for example, genetically modifying and/or otherwise altering at least one target gene or gene product, processes for producing hypoimmunogenic cells (such as engineered hypoimmunogenic cells), manufacturing of hypoimmunogenic cellular compositions (such as engineered hypoimmunogenic cellular compositions), hypoimmunogenic cell systems (such as engineered hypoimmunogenic cell systems), and uses thereof. In one aspect, provided herein is a method of hypoimmunogenicity (such as engineering hypoimmunogenicity), comprising: a) genetically modifying a regulatory factor X (RFX) gene of at least one immunogenic human cell, wherein genetically modifying the RFX gene reduces expression of the RFX protein in the immunogenic human cell; b) forming at least one embryoid body or multicellular body from the cell of a) to produce at least one hypoimmunogenic cell (such as an engineered hypoimmunogenic cell); c) subjecting the hypoimmunogenic cell (such as an engineered hypoimmunogenic cell) to an immune system; and d) determining immunogenicity of the hypoimmunogenic cell (such as an engineered hypoimmunogenic cell), wherein the immunogenicity is altered as compared to an immunogenic human cell where the RFX gene is not genetically modified, optionally wherein step a) further comprises genetically modifying one or more of a class II major histocompatibility complex transactivator (CIITA) gene, a beta-2-microglobulin (B2M) gene, and a CD58 gene of the immunogenic human cell.

In one aspect, provided herein is a method of hypoimmunogenicity (such as engineering hypoimmunogenicity), comprising: a) reprogramming an immunogenic human cell to produce an induced pluripotent stem (iPS) human cell, wherein the immunogenic human cell comprises a heterodimeric T-cell receptor comprising a γ chain and a δ chain; b) genetically modifying a regulatory factor X (RFX) gene of the iPS human cell, wherein genetically modifying the RFX gene reduces expression of the RFX protein by the iPS human cell; c) forming at least one embryoid body from the cell of step b) to produce at least one hypoimmunogenic cell (such as an engineered hypoimmunogenic cell); d) subjecting the hypoimmunogenic cell (such as an engineered hypoimmunogenic cell) to an immune system; and e) determining immunogenicity of the hypoimmunogenic cell (such as an engineered hypoimmunogenic cell), wherein the immunogenicity is altered as compared to an iPS human cell where the RFX gene is not genetically modified, optionally wherein step b) further comprises genetically modifying one or more of a class II major histocompatibility complex transactivator (CIITA) gene, a beta-2-microglobulin (B2M) gene, and a CD58 gene of the iPS human cell.

In one aspect, provided herein is a method of hypoimmunogenicity (such as engineering hypoimmunogenicity), comprising: a) genetically modifying a regulatory factor X (RFX) gene of an immunogenic human cell to produce a hypoimmunogenic cell (such as an engineered hypoimmunogenic cell), wherein genetically modifying the RFX gene reduces expression of the RFX protein by the immunogenic human cell; b) subjecting the hypoimmunogenic cell (such as an engineered hypoimmunogenic cell) to an immune system; and c) determining immunogenicity of the hypoimmunogenic cell (such as an immunogenic engineered hypoimmunogenic cell), wherein the immunogenicity is altered as compared to an immunogenic human cell where the RFX gene is not genetically modified, optionally wherein step a) further comprises genetically modifying one or more of a class II major histocompatibility complex transactivator (CIITA) gene, a beta-2-microglobulin (B2M) gene, and a CD58 gene of the immunogenic human cell.

In one aspect, provided herein is a method of producing a hypoimmunogenic cell (such as an engineered hypoimmunogenic cell) from an immunogenic cell, comprising: (i) genetically modifying a regulatory factor X (RFX) gene in the immunogenic cell, wherein genetically modifying the RFX gene reduces expression of the RFX protein in said cell, and (ii) optionally further genetically modifying one or more genes selected from a class II major histocompatibility complex transactivator (CIITA) gene, a beta-2-microglobulin (B2M) gene, and a CD58 gene in said immunogenic cell, wherein genetically modifying the one or more genes reduces expression of the corresponding one or more proteins in said immunogenic cell, wherein said method results in production of the hypoimmunogenic cell (such as the engineered hypoimmunogenic cell), which has one or more of the following properties: a) having a reduced immunogenicity upon the hypoimmunogenic cell's (such as the engineered hypoimmunogenic cell's) presence in an allogeneic or non-MHC matched subject as compared to a corresponding immunogenic cell, but without the genetic modification(s) of (i) and (ii); b) causing a reduced immune response to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii); and c) causing a reduced alloreactive T cell cytotoxicity to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii).

In one aspect, provided herein is a method of producing a hypoimmunogenic cell (such as an engineered hypoimmunogenic cell) from an immunogenic cell, comprising: a) reprogramming the immunogenic cell to produce an induced pluripotent stem (iPS) cell; b) (i) genetically modifying a regulatory factor X (RFX) gene in the iPS cell produced in step (a), wherein genetically modifying the RFX gene reduces expression of the RFX protein in said iPS cell, and (ii) optionally further genetically modifying one or more genes selected from a class II major histocompatibility complex transactivator (CIITA) gene, a beta-2-microglobulin (B2M) gene, and a CD58 gene in said iPS cell, wherein genetically modifying the one or more genes reduces expression of the corresponding one or more proteins in said iPS cell; and c) optionally, differentiating the cell produced in step (b); wherein said method results in production of the hypoimmunogenic cell (such as the engineered hypoimmunogenic cell) which has one or more of the following properties: 1) having a reduced immunogenicity upon the hypoimmunogenic cell's, such as the engineered hypoimmunogenic cell's, presence in an allogeneic or non-MHC matched subject, as compared to a corresponding iPS cell, or a cell corresponding to the cell produced in step (c), but without the genetic modification(s) of step (b); 2) causing a reduced immune response to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b); 3) causing a reduced alloreactive T cell cytotoxicity to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b).

In some embodiments, the hypoimmunogenic cell (such as the engineered hypoimmunogenic cell) comprises a T-cell receptor (TCR) comprising a γ chain and a δ chain.

In some embodiments, the immunogenic cell or the human immunogenic cell is an immune cell, optionally selected from T cells, natural killer (NK) cells, B cells, and hematopoietic stem cells (HSCs).

In some embodiments, the reduced immunogenicity of the hypoimmunogenic cell (such as the engineered hypoimmunogenic cell) comprises one or more of the following: i) a reduced or ablated myeloid cell response to the hypoimmunogenic cell (such as an engineered hypoimmunogenic cell) upon the cell's presence in an allogeneic or non-MHC matched subject, as compared to a cell corresponding to the cell that was modified but without said genetic modification(s); ii) a reduced or ablated T cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iii) a reduced or ablated natural killer (NK) cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iv) a reduced or ablated neutralizing antibody response to the hypoimmunogenic cell (such as the engineered hypoimmunogenic cell) upon the cell's presence in an allogeneic or non-MHC matched subject, as compared to a cell corresponding to the cell that was modified but without said genetic modification(s); v) a reduced or ablated MHC class II mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); vi) a reduced or ablated neutralizing MHC class I mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); and vii) a reduced or ablated allogeneic host versus graft rejection of to the hypoimmunogenic cell (such as the engineered hypoimmunogenic cell) upon the cell's presence in an allogeneic subject, as compared to a cell corresponding to the cell that was modified but without said genetic modification(s).

In some embodiments, the immunogenic cell is a human cell.

In some embodiments, in the hypoimmunogenic cell (such as the engineered hypoimmunogenic cell): i) expression of HLA class II molecules is reduced or ablated; ii) expression of HLA-A, HLA-B, and/or HLA-C is reduced; and iii) expression of HLA-E is reduced but remains detectable.

In some embodiments, the method comprises forming at least one embryoid body or multicellular body from the genetically modified cell to produce the hypoimmunogenic cell (such as an engineered hypoimmunogenic cell).

In some embodiments, the method further comprises determining immunogenicity of the hypoimmunogenic cell (such as the engineered hypoimmunogenic cell).

In some embodiments, the method further comprises administering the hypoimmunogenic cell (such as the engineered hypoimmunogenic cell) to an allogeneic or non-MHC matched subject.

In some embodiments, the immunogenicity of the hypoimmunogenic cell (such as the engineered hypoimmunogenic cell) is altered as compared to an immunogenic cell or an immunogenic human cell or an iPS human cell or iPS cell where the only difference between the hypoimmunogenic cell (such as an engineered hypoimmunogenic cell) and the immunogenic cell or the immunogenic human cell or the iPS human cell or iPS cell is that the REX gene and optionally one or more of the CIITA gene, the B2M gene, and the CD58 gene is not genetically modified in the immunogenic cell or the immunogenic human cell or the iPSC human cell or iPS cell.

In some embodiments, the immunogenic human cell or the immunogenic cell is allogeneic or non-HLA matched or non-MHC matched to cells, receptors, or polypeptides of the immune system of a recipient subject.

In some embodiments, altering the immunogenicity comprises balancing, reducing, or neutralizing the immunogenicity, such as reducing or neutralizing the immunogenicity. In some embodiments, altering the immunogenicity comprises reducing or neutralizing a myeloid cell response to the hypoimmunogenic cells (such as engineered hypoimmunogenic cells). In some embodiments, altering the immunogenicity comprises reducing or neutralizing a T cell response to the hypoimmunogenic cells (such as engineered hypoimmunogenic cells). In some embodiments, altering the immunogenicity comprises reducing or neutralizing a natural killer cell response to the hypoimmunogenic cells (such as engineered hypoimmunogenic cells). In some embodiments, altering the immunogenicity comprises reducing or neutralizing an antibody response to the hypoimmunogenic (such as engineered hypoimmunogenic cells). In some embodiments, altering the immunogenicity comprises reducing or neutralizing an allogeneic host versus graft rejection.

In some embodiments, altering the immunogenicity comprises one or more of the following in the hypoimmunogenic cell (such as the engineered hypoimmunogenic cell): a) expression of HLA class II molecules are reduced or ablated; b) expression of HLA-A, HLA-B, and/or HLA-C are reduced; and c) expression of HLA-E is reduced but remains detectable.

In some embodiments, altering the immunogenicity comprises reducing or ablating MHC class II mediated response to the hypoimmunogenic cell (such as the engineered hypoimmunogenic cell). In some embodiments, altering the immunogenicity comprises reducing or neutralizing MHC class I mediated response to the hypoimmunogenic cell (such as the engineered hypoimmunogenic cell).

In some embodiments, the RFX gene is RFX5, RFXANK or RFXAP. In some embodiments, two or more of RFX5, RFXANK or RFXAP are genetically modified. In some embodiments, each of RFX5, RFXANK, and RFXAP are genetically modified.

In some embodiments, methods disclosed herein further comprises genetically modifying a CD58 gene, wherein genetically modifying the CD58 gene eliminates or reduces the CD58 protein expression. In some embodiments, genetically modifying the CD58 gene reduces or ablates a costimulatory immune cell response, and/or impairs the formation of an immune synapse.

In some embodiments, methods disclosed herein further comprises genetically modifying a B2M gene, wherein genetically modifying the B2M gene results in reducing or ablating expression of HLA class I molecules on the hypoimmunogenic cell (such as the engineered hypoimmunogenic cell), optionally the HLA class I molecules are selected from the group consisting of HLA-A, HLA-B, HLA-C, HLA-E, and combinations thereof.

In some embodiments, methods disclosed herein further comprises genetically modifying a CIITA gene, wherein genetically modifying the CIITA gene results in reducing or ablating expression of HLA class II molecules on the hypoimmunogenic cell (such as the engineered hypoimmunogenic cell).

In some embodiments, genetically modifying the RFX gene comprises: (i) modifying the DNA sequence of the RFX gene, optionally through a CRISPR-Cas system; (ii) repressing transcription or translation of the RFX mRNA through a RNAi system, optionally the RNAi system comprises shRNA, siRNA, miR-adapted shRNA, or a combination thereof; or (iii) reducing or ablating transcription of the RFX gene, optionally through recruiting or directing transcriptional repressors to the RFX gene.

In some embodiments, genetically modifying the CIITA gene and/or the B2M gene and/or the CD58 gene comprises: (i) modifying the DNA sequence of the CIITA gene and/or the B2M gene and/or the CD58 gene, optionally through a CRISPR-Cas system; (ii) repressing transcription or translation of the CIITA gene and/or the B2M gene and/or the CD58 gene through a RNAi system, optionally wherein the RNAi system comprises shRNA, siRNA, miR-adapted shRNA, or a combination thereof; or (iii) reducing or ablating transcription of the CIITA gene and/or the B2M gene and/or the CD58 gene, optionally through recruiting or directing transcriptional repressors to the CIITA gene and/or the B2M gene and/or the CD58 gene.

In some embodiments, the method further comprises genetically modifying at least one of a TNFRSF14 gene, a TNFRSF1A gene, a TNFRSF1B gene, an ICAM1 gene, and a herpesvirus entry mediator (HVEM) gene.

In one aspect, provided herein is a non-naturally occurring hypoimmunogenic human cell produced by the method disclosed herein.

In one aspect, provided herein is a non-naturally occurring hypoimmunogenic human cell, comprising a genetically modified regulatory factor X (RFX) gene, wherein the genetically modified RFX gene reduces expression of the RFX protein, and the hypoimmunogenic human cell is produced from an embryoid body; optionally the hypoimmunogenic human cell further comprises one or more of a genetically modified class II major histocompatibility complex transactivator (CIITA) gene, a genetically modified beta-2-microglobulin (B2M) gene, and a genetically modified CD58 gene.

In one aspect, provided herein is a composition comprising the hypoimmunogenic human cell disclosed herein.

In one aspect, provided herein is a γδT cell-derived induced pluripotent stem (iPS) human cell, comprising a genetically modified regulatory factor X (RFX) gene, wherein the genetically modified RFX gene reduces expression of the RFX protein; optionally the iPS human cell further comprises one or more of a genetically modified class II major histocompatibility complex transactivator (CIITA) gene, a genetically modified beta-2-microglobulin (B2M) gene, and a genetically modified CD58 gene.

In one aspect, provided herein is a composition comprising the iPS human cell disclosed herein.

In one aspect, provided herein is a method of hypoimmunogenicity (such as engineering hypoimmunogenicity), comprising: a) a step for performing a function of genetically modifying a regulatory factor X (RFX) gene of at least one immunogenic cell (such as an immunogenic human cell), wherein genetically modifying the RFX gene reduces expression of the RFX protein in the immunogenic cell (such as an immunogenic human cell); b) a step for performing a function of forming at least one embryoid body or multicellular body from the cell of a) to produce at least one hypoimmunogenic cell (such as an engineered hypoimmunogenic cell); c) a step for performing a function of subjecting the hypoimmunogenic cell (such as the engineered hypoimmunogenic cell) to an immune system; and d) a step for performing a function of determining immunogenicity of the hypoimmunogenic cell (such as the engineered hypoimmunogenic cell), wherein the immunogenicity is altered as compared to an immunogenic cell (such as an immunogenic human cell) where the RFX gene is not genetically modified, optionally wherein step a) further comprises a step for performing a function of genetically modifying a class II major histocompatibility complex transactivator (CIITA) gene, a beta-2-microglobulin (B2M) gene, and/or a CD58 gene of the immunogenic human cell.

In one aspect, provided herein is a method of hypoimmunogenicity (such as engineering hypoimmunogenicity), comprising: a) a step for performing a function of reprogramming an immunogenic human cell to produce an induced pluripotent stem (iPS) human cell, wherein the immunogenic human cell comprises a heterodimeric T-cell receptor comprising a γ chain and a δ chain; b) a step for performing a function of genetically modifying a regulatory factor X (RFX) gene of the iPS human cell, wherein genetically modifying the RFX gene reduces expression of the RFX protein by the iPS human cell; c) a step for performing a function of forming at least one embryoid body from the cell of step b) to produce at least one hypoimmunogenic cell (such as an engineered hypoimmunogenic cell); d) a step for performing a function of subjecting the hypoimmunogenic cell (such as the engineered hypoimmunogenic cell) to an immune system; and e) a step for performing a function of determining immunogenicity of the hypoimmunogenic cell (such as the engineered hypoimmunogenic cell), wherein the immunogenicity is altered as compared to an iPS human cell where the RFX gene is not genetically modified, optionally wherein step b) further comprises a step for performing a function of genetically modifying a class II major histocompatibility complex transactivator (CIITA) gene, a beta-2-microglobulin (B2M) gene, and/or a CD58 gene of the iPS human cell.

In one aspect, provided herein is a method of hypoimmunogenicity (such as engineering hypoimmunogenicity), comprising: a) a step for performing a function of genetically modifying a regulatory factor X (RFX) gene of an immunogenic human cell to produce a hypoimmunogenic cell (such as an engineered hypoimmunogenic cell), wherein genetically modifying the RFX gene reduces expression of the RFX protein by the immunogenic human cell; b) a step for performing a function of subjecting the hypoimmunogenic cell (such as the engineered hypoimmunogenic cell) to an immune system; and c) a step for performing a function of determining immunogenicity of the hypoimmunogenic cell (such as the engineered hypoimmunogenic cell), wherein the immunogenicity is altered as compared to an immunogenic human cell where the RFX gene is not genetically modified, optionally wherein step a) further comprises a step for performing a function of genetically modifying a class II major histocompatibility complex transactivator (CIITA) gene, a beta-2-microglobulin (B2M) gene, and/or a CD58 gene of the immunogenic human cell.

In one aspect, provided herein is a non-naturally occurring hypoimmunogenic human cell (such as an engineered hypoimmunogenic cell), comprising a means for reducing expression of an RFX protein through a genetically modified RFX gene, and/or a means for altering immunogenicity of an immune system to the hypoimmunogenic human cell (such as the engineered hypoimmunogenic cell) as compared to an immunogenic human cell where the RFX gene is not genetically modified; optionally wherein the hypoimmunogenic human cell (such as the engineered hypoimmunogenic cell) further comprises a means for reducing expression of a CIITA protein, a B2M protein, and/or a CD58 protein through a genetically modified CIITA gene, a genetically modified B2M gene, and/or a genetically modified CD58 gene.

In one aspect, provided herein is a γδT cell-derived induced pluripotent stem (iPS) human cell, comprising a means for reducing expression of an RFX protein through a genetically modified RFX gene, and/or a means for altering immunogenicity of an immune system to the iPS human cell as compared to an iPS human cell where the RFX gene is not genetically modified; optionally wherein the iPS human cell further comprises a means for reducing expression of a CIITA protein, a B2M protein, and/or a CD58 protein through a genetically modified CIITA gene, a genetically modified B2M gene, and/or a genetically modified CD58 gene.

In one aspect, provided herein is a method of hypoimmunogenicity (such as engineering hypoimmunogenicity), comprising: a) reprogramming an immunogenic human cell to produce an induced pluripotent (iPS) human cell, wherein the immunogenic human cell comprises a heterodimeric T-cell receptor comprising a γ chain and a δ chain; b) genetically modifying a beta-2-microglobulin (B2M) gene of the iPS human cell, wherein genetically modifying the B2M gene reduces expression of the B2M protein by the iPS human cell; c) forming at least one embryoid body or multicellular body from the cell of step b) to produce at least one hypoimmunogenic cell (such as an engineered hypoimmunogenic cell); d) subjecting the hypoimmunogenic cell (such as the engineered hypoimmunogenic cell) to an immune system; and e) determining immunogenicity of the hypoimmunogenic cell (such as the engineered hypoimmunogenic cell), wherein the immunogenicity is altered as compared to an iPS human cell where the B2M gene is not genetically modified, optionally wherein step b) further comprises genetically modifying a class II major histocompatibility complex transactivator (CIITA) gene, a regulatory factor X (RFX) gene, and/or a CD58 gene of the iPS human cell.

In one aspect, provided herein is a method of hypoimmunogenicity (such as engineering hypoimmunogenicity), comprising: a) genetically modifying a beta-2-microglobulin (B2M) gene of at least one immunogenic human cell, wherein genetically modifying the B2M gene reduces expression of the B2M by the immunogenic human cell; b) forming at least one embryoid body or multicellular body from the cell of a) to produce at least one hypoimmunogenic cell (such as an engineered hypoimmunogenic cell); c) subjecting the hypoimmunogenic cell (such as the engineered hypoimmunogenic cell) to an immune system; and d) determining immunogenicity of the hypoimmunogenic cell (such as the engineered hypoimmunogenic cell), wherein the immunogenicity is altered as compared to an immunogenic human cell where the B2M gene is not genetically modified, optionally wherein step a) further comprises genetically modifying one or more of a class II major histocompatibility complex transactivator (CIITA) gene, a regulatory factor X (RFX) gene, and a CD58 gene of the immunogenic human cell.

In one aspect, provided herein is a method of hypoimmunogenicity (such as engineering hypoimmunogenicity), comprising: a) genetically modifying a beta-2-microglobulin (B2M) gene of an immunogenic human cell to produce a hypoimmunogenic cell (such as an engineered hypoimmunogenic cell), wherein genetically modifying the B2M gene reduces expression of the B2M protein by the immunogenic human cell; b) subjecting the hypoimmunogenic cell (such as the engineered hypoimmunogenic cell) to an immune system; and c) determining immunogenicity of the hypoimmunogenic cell (such as the engineered hypoimmunogenic cell), wherein the immunogenicity is altered as compared to an immunogenic human cell where the B2M gene is not genetically modified, optionally wherein step a) further comprises genetically modifying one or more of a class II major histocompatibility complex transactivator (CIITA) gene, a regulatory factor X (RFX) gene, and a CD58 gene of the immunogenic human cell.

In one aspect, provided herein is a method of producing a hypoimmunogenic cell (such as an engineered hypoimmunogenic cell) from an immunogenic cell, comprising: (i) genetically modifying a beta-2-microglobulin (B2M) gene in the immunogenic cell, wherein genetically modifying the B2M gene reduces expression of the B2M protein in said cell, and (ii) optionally further genetically modifying one or more genes selected from a class II major histocompatibility complex transactivator (CIITA) gene, a regulatory factor X (RFX) gene, and a CD58 gene in said immunogenic cell, wherein genetically modifying the one or more genes reduces expression of the corresponding one or more proteins in said immunogenic cell, wherein said method results in production of the hypoimmunogenic cell (such as the engineered hypoimmunogenic cell), which has one or more of the following properties: a) having a reduced immunogenicity upon the hypoimmunogenic cell's (such as the engineered hypoimmunogenic cell's) presence in an allogeneic or non-MHC matched subject as compared to a corresponding immunogenic cell, but without the genetic modification(s) of (i) and (ii); b) causing a reduced immune response to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii); and c) causing a reduced alloreactive T cell cytotoxicity to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii).

In one aspect, provided herein is a method of producing a hypoimmunogenic cell (such as an engineered hypoimmunogenic cell) from an immunogenic cell, comprising: a) reprogramming the immunogenic cell to produce an induced pluripotent stem (iPS) cell; b) (i) genetically modifying a beta-2-microglobulin (B2M) gene in the iPS cell, wherein genetically modifying the B2M gene reduces expression of the B2M protein in said iPS cell, and (ii) optionally further genetically modifying one or more genes selected from a class II major histocompatibility complex transactivator (CIITA) gene, a regulatory factor X (RFX) gene, and a CD58 gene in said iPS cell, wherein genetically modifying the one or more genes reduces expression of the corresponding one or more proteins in said iPS cell; and c) optionally, differentiating the cell produced in step (b); wherein said method results in production of the hypoimmunogenic cell (such as the engineered hypoimmunogenic cell), which has one or more of the following properties: 1) having a reduced immunogenicity upon the hypoimmunogenic cell's, such as the engineered hypoimmunogenic cell's, presence in an allogeneic or non-MHC matched subject, as compared to a corresponding iPS cell, or a cell corresponding to the cell produced in step (c), but without the genetic modification(s) of step (b); 2) causing a reduced immune response to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b); and 3) causing a reduced alloreactive T cell cytotoxicity to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b).

In some embodiments, the hypoimmunogenic cell (such as the engineered hypoimmunogenic cell) comprises a T-cell receptor (TCR) comprising a γ chain and a δ chain.

In some embodiments, the immunogenic cell or the human immunogenic cell is an immune cell, optionally selected from T cells, natural killer (NK) cells, B cells, and hematopoietic stem cells (HSCs).

In some embodiments, the reduced immunogenicity of the hypoimmunogenic cell (such as the engineered hypoimmunogenic cell) comprises one or more of the following: i) a reduced or ablated myeloid cell response to the hypoimmunogenic cell (such as an engineered hypoimmunogenic cell) upon the cell's presence in an allogeneic or non-MHC matched subject, as compared to a cell corresponding to the cell that was modified but without said genetic modification(s); ii) a reduced or ablated T cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iii) a reduced or ablated natural killer (NK) cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iv) a reduced or ablated neutralizing antibody response to the hypoimmunogenic cell (such as the engineered hypoimmunogenic cell) upon the cell's presence in an allogeneic or non-MHC matched subject, as compared to a cell corresponding to the cell that was modified but without said genetic modification(s); v) a reduced or ablated MHC class II mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); vi) a reduced or ablated neutralizing MHC class I mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); and vii) a reduced or ablated allogeneic host versus graft rejection of to the hypoimmunogenic cell (such as the engineered hypoimmunogenic cell) upon the cell's presence in an allogeneic subject, as compared to a cell corresponding to the cell that was modified but without said genetic modification(s).

In some embodiments, the immunogenic cell is a human cell.

In some embodiments, in the hypoimmunogenic cell (such as the engineered hypoimmunogenic cell): i) expression of HLA class II molecules is reduced or ablated; ii) expression of HLA-A, HLA-B, and/or HLA-C is reduced; and iii) expression of HLA-E is reduced but remains detectable.

In some embodiments, the method comprises forming at least one embryoid body or multicellular body from the genetically modified cell to produce the hypoimmunogenic cell (such as the engineered hypoimmunogenic cell).

In some embodiments, the method further comprises determining immunogenicity of the hypoimmunogenic cell (such as the engineered hypoimmunogenic cell).

In some embodiments, the method further comprises administering the hypoimmunogenic cell (such as the engineered hypoimmunogenic cell) to an allogeneic or non-MHC matched subject.

In some embodiments, the immunogenicity of the hypoimmunogenic cell (such as the engineered hypoimmunogenic cell) is altered as compared to an immunogenic cell or an immunogenic human cell or an iPS human cell or an iPS cell where the only difference between the hypoimmunogenic cell (such as the engineered hypoimmunogenic cell) and the immunogenic cell or the immunogenic human cell or the iPS human cell or the iPS cell is that the B2M gene and optionally one or more of the RFX gene, the CIITA gene, and the CD58 gene is not genetically modified in the immunogenic cell or the immunogenic human cell or the iPS human cell or the iPS cell.

In some embodiments, the immunogenic human cell or immunogenic cell is allogeneic or non-HLA matched or non-MHC matched to cells, receptors, or polypeptides of the immune system of a recipient subject.

In some embodiments, altering the immunogenicity comprises balancing, reducing, or neutralizing the immunogenicity, such as reducing or neutralizing the immunogenicity.

In some embodiments, altering the immunogenicity comprises reducing or neutralizing a myeloid cell response to the hypoimmunogenic cells (such as the engineered hypoimmunogenic cell).

In some embodiments, altering the immunogenicity comprises reducing or neutralizing a T cell response to the hypoimmunogenic cells (such as the engineered hypoimmunogenic cell).

In some embodiments, altering the immunogenicity comprises reducing or neutralizing a natural killer cell response to the hypoimmunogenic cells (such as the engineered hypoimmunogenic cell).

In some embodiments, altering the immunogenicity comprises reducing or neutralizing an antibody response to the hypoimmunogenic cells (such as the engineered hypoimmunogenic cell).

In some embodiments, altering the immunogenicity comprises reducing or neutralizing an allogeneic host versus graft rejection.

In some embodiments, altering the immunogenicity comprises reducing or ablating expression of HLA class I molecules on the hypoimmunogenic cell (such as the engineered hypoimmunogenic cell).

In some embodiments, the method disclosed herein further comprises genetically modifying a RFX gene, wherein the RFX gene is RFX5, RFXANK or RFXAP. In some embodiments, two or more of RFX5, RFXANK or RFXAP are genetically modified. In some embodiments, each of RFX5, RFXANK, and RFXAP are genetically modified.

In some embodiments, genetically modifying the RFX gene results in one or more of the following in the hypoimmunogenic cell (such as the engineered hypoimmunogenic cell): a) expression of HLA class II molecules are reduced or ablated; and/or b) expression of HLA-A, HLA-B, and/or HLA-C are reduced.

In some embodiments, genetically modifying the RFX gene results in reducing or ablating MHC class II mediated response to the hypoimmunogenic cell (such as the engineered hypoimmunogenic cell).

In some embodiments, genetically modifying the RFX gene results in reducing or neutralizing MHC class I mediated response to the hypoimmunogenic cell (such as the engineered hypoimmunogenic cell).

In some embodiments, the method disclosed herein further comprises genetically modifying a CIITA gene, wherein genetically modifying the CIITA gene results in reducing or ablating expression of HLA class II molecules on the hypoimmunogenic cell (such as the engineered hypoimmunogenic cell).

In some embodiments, the method disclosed herein further comprises genetically modifying a CD58 gene, wherein genetically modifying the CD58 gene eliminates or reduces the CD58 expression.

In some embodiments, genetically modifying the CD58 gene reduces or ablates a co-stimulatory immune cell response, and/or impairs the formation of an immune synapse.

In some embodiments, genetically modifying the B2M gene comprises: (i) modifying the DNA sequence of the B2M gene, optionally through a CRISPR-Cas system; (ii) repressing transcription or translation of the B2M mRNA through RNAi system, optionally the RNAi system comprises shRNA, siRNA, or miR-adapted shRNA; or (iii) reducing or ablating transcription of the B2M gene, optionally through recruiting or directing transcriptional repressors to the B2M gene.

In some embodiments, genetically modifying the CIITA gene and/or the RFX gene and/or the CD58 gene comprises: (i) modifying the DNA sequence of the CIITA gene and/or the RFX gene and/or the CD58 gene, optionally through a CRISPR-Cas system; (ii) repressing transcription or translation of the CIITA gene and/or the RFX gene and/or the CD58 gene through a RNAi system, optionally wherein the RNAi system comprises shRNA, siRNA, miR-adapted shRNA, or a combination thereof; or (iii) reducing or ablating transcription of the CIITA gene and/or the RFX gene and/or the CD58 gene, optionally through recruiting or directing transcriptional repressors to the CIITA gene and/or the RFX gene and/or the CD58 gene.

In some embodiments, the method disclosed herein further comprises genetically modifying at least one of a TNFRSF14 gene, a TNFRSF1A gene, a TNFRSF1B gene, an ICAM1 gene, and a herpesvirus entry mediator (HVEM) gene.

In one aspect, provided herein is a non-naturally occurring hypoimmunogenic human cell (such as an engineered hypoimmunogenic human cell), produced by the method disclosed herein.

In one aspect, provided herein is a non-naturally occurring hypoimmunogenic human cell (such as an engineered hypoimmunogenic human cell), comprising a genetically modified B2M gene, wherein the genetically modified B2M gene reduces expression of the B2M protein, and the hypoimmunogenic human cell (such as the engineered hypoimmunogenic human cell) is produced from an embryoid body; optionally the hypoimmunogenic human cell (such as the engineered hypoimmunogenic human cell) further comprises one or more of a genetically modified CIITA gene, a genetically modified RFX gene, and a genetically modified CD58 gene.

In one aspect, provided herein is a composition comprising the hypoimmunogenic human cell (such as the engineered hypoimmunogenic human cell) disclosed herein.

In one aspect, provided herein is a γδT cell-derived induced pluripotent stem (iPS) human cell, comprising a genetically modified B2M gene, wherein the genetically modified B2M gene reduces expression of the B2M protein; optionally the iPS human cell further comprises one or more of a genetically modified CIITA gene, a genetically modified RFX gene, and a genetically modified CD58 gene.

In one aspect, provided herein is a composition comprising the iPS human cell disclosed herein.

In one aspect, provided herein is a method of hypoimmunogenicity (such as engineering hypoimmunogenicity), comprising: a) a step for performing a function of genetically modifying a B2M gene of at least one immunogenic human cell, wherein genetically modifying the B2M gene reduces expression of the B2M protein in the immunogenic human cell; b) a step for performing a function of forming at least one embryoid body or multicellular body from the cell of a) to produce at least one hypoimmunogenic cell (such as an engineered hypoimmunogenic human cell); c) a step for performing a function of subjecting the hypoimmunogenic cell (such as the engineered hypoimmunogenic human cell) to an immune system; and d) a step for performing a function of determining immunogenicity of the hypoimmunogenic cell (such as the engineered hypoimmunogenic cell), wherein the immunogenicity is altered as compared to an immunogenic human cell where the B2M gene is not genetically modified, optionally wherein step a) further comprises a step for performing a function of genetically modifying a RFX gene, a CIITA gene, and/or a CD58 gene of the immunogenic human cell.

In one aspect, provided herein is a method of hypoimmunogenicity (such as engineering hypoimmunogenicity), comprising: a) a step for performing a function of reprogramming an immunogenic human cell to produce an induced pluripotent stem (iPS) human cell, wherein the immunogenic human cell comprises a heterodimeric T-cell receptor comprising a γ chain and a δ chain; b) a step for performing a function of genetically modifying a B2M gene of the iPS human cell, wherein genetically modifying the B2M gene reduces expression of the B2M protein by the iPS human cell; c) a step for performing a function of forming at least one embryoid body from the cell of step b) to produce at least one hypoimmunogenic cell (such as an engineered hypoimmunogenic cell); d) a step for performing a function of subjecting the hypoimmunogenic cell (such as the engineered hypoimmunogenic cell) to an immune system; and e) a step for performing a function of determining immunogenicity of the hypoimmunogenic cell (such as the engineered hypoimmunogenic cell), wherein the immunogenicity is altered as compared to an iPS human cell where the B2M gene is not genetically modified, optionally wherein step b) further comprises a step for performing a function of genetically modifying a RFX gene, a CIITA gene, and/or a CD58 gene of the iPS human cell.

In one aspect, provided herein is a method of hypoimmunogenicity (such as engineering hypoimmunogenicity), comprising: a) a step for performing a function of genetically modifying a B2M gene of an immunogenic human cell to produce a hypoimmunogenic cell (such as an engineered hypoimmunogenic cell), wherein genetically modifying the B2M gene reduces expression of the B2M protein by the immunogenic human cell; b) a step for performing a function of subjecting the hypoimmunogenic cell (such as the engineered hypoimmunogenic cell) to an immune system; and c) a step for performing a function of determining immunogenicity of the hypoimmunogenic cell (such as the engineered hypoimmunogenic cell), wherein the immunogenicity is altered as compared to an immunogenic human cell where the B2M gene is not genetically modified, optionally wherein step a) further comprises a step for performing a function of genetically modifying a RFX gene, a CIITA gene, and/or a CD58 gene of the immunogenic human cell.

In one aspect, provided herein is a non-naturally occurring hypoimmunogenic human cell (such as an engineered hypoimmunogenic human cell), comprising a means for reducing expression of a B2M protein through a genetically modified B2M gene, and/or a means for altering immunogenicity of an immune system to the hypoimmunogenic human cell (such as the engineered hypoimmunogenic human cell) as compared to an immunogenic human cell where the B2M gene is not genetically modified; optionally wherein the hypoimmunogenic human cell (such as the engineered hypoimmunogenic human cell) further comprises a means for reducing expression of a RFX protein, a CD58 protein, and/or a CIITA protein through a genetically modified RFX gene, a genetically modified CD58 gene, and/or a genetically modified CIITA gene.

In one aspect, provided herein is a γδT cell-derived induced pluripotent stem (iPS) human cell, comprising a means for reducing expression of a B2M protein through a genetically modified B2M gene, and/or a means for altering immunogenicity of an immune system to the iPS human cell as compared to an iPS human cell where the B2M gene is not genetically modified; optionally wherein the iPS human cell further comprises a means for reducing expression of a RFX protein, a CD58 protein, and/or a CIITA protein through a genetically modified RFX gene, a genetically modified CD58 gene, and/or a genetically modified CIITA gene.

In one aspect, provided herein is a method of hypoimmunogenicity (such as engineering hypoimmunogenicity), comprising: a) genetically modifying a CD58 gene of at least one immunogenic human cell, wherein genetically modifying the CD58 gene reduces expression of the CD58 protein by the immunogenic human cell; b) forming at least one embryoid body or multicellular body from the cell of a) to produce at least one hypoimmunogenic cell (such as an engineered hypoimmunogenic cell); c) subjecting the hypoimmunogenic cell (such as the engineered hypoimmunogenic cell) to an immune system; and d) determining immunogenicity of the hypoimmunogenic cell (such as the engineered hypoimmunogenic cell), wherein the immunogenicity is altered as compared to an immunogenic human cell where the CD58 gene is not genetically modified, optionally wherein step a) further comprises genetically modifying one or more of a class II major histocompatibility complex transactivator (CIITA) gene, a regulatory factor X (RFX) gene, and a beta-2-microglobulin (B2M) gene of the immunogenic human cell.

In one aspect, provided herein is a method of hypoimmunogenicity (such as engineering hypoimmunogenicity), comprising: a) reprogramming an immunogenic human cell to produce an induced pluripotent (iPS) human cell, wherein the immunogenic human cell comprises a heterodimeric T-cell receptor comprising a γ chain and a δ chain; b) genetically modifying a CD58 gene of the iPS human cell, wherein genetically modifying the CD58 gene reduces expression of the CD58 protein by the iPS human cell; c) forming at least one embryoid body from the cell of step b) to produce at least one hypoimmunogenic cell (such as an engineered hypoimmunogenic cell); d) subjecting the hypoimmunogenic cell (such as the engineered hypoimmunogenic cell) to an immune system; and e) determining immunogenicity of the hypoimmunogenic cell (such as the engineered hypoimmunogenic cell), wherein the immunogenicity is altered as compared to an iPS human cell where the CD58 gene is not genetically modified, optionally wherein step b) further comprises genetically modifying one or more of a class II major histocompatibility complex transactivator (CIITA) gene, a regulatory factor X (RFX) gene, and a beta-2-microglobulin (B2M) gene of the iPS human cell.

In one aspect, provided herein is a method of hypoimmunogenicity (such as engineering hypoimmunogenicity), comprising: a) genetically modifying a CD58 gene of an immunogenic human cell to produce a hypoimmunogenic cell (such as an engineered hypoimmunogenic cell), wherein genetically modifying the CD58 gene reduces expression of the CD58 protein by the immunogenic human cell; b) subjecting the hypoimmunogenic cell (such as the engineered hypoimmunogenic cell) to an immune system; and c) determining immunogenicity of the hypoimmunogenic cell (such as the engineered hypoimmunogenic cell), wherein the immunogenicity is altered as compared to an immunogenic human cell where the CD58 gene is not genetically modified, optionally wherein step a) further comprises genetically modifying one or more of a class II major histocompatibility complex transactivator (CIITA) gene, a regulatory factor X (RFX) gene, and a beta-2-microglobulin (B2M) gene of the immunogenic human cell.

In one aspect, provided herein is a method of producing a hypoimmunogenic cell (such as an engineered hypoimmunogenic cell) from an immunogenic cell, comprising: (i) genetically modifying a CD58 gene in the immunogenic cell, wherein genetically modifying the CD58 gene reduces expression of the CD58 protein in said cell, and (ii) optionally further genetically modifying one or more genes selected from a class II major histocompatibility complex transactivator (CIITA) gene, a regulatory factor X (RFX) gene, and a beta-2-microglobulin (B2M) gene in said immunogenic cell, wherein genetically modifying the one or more genes reduces expression of the corresponding one or more proteins in said immunogenic cell, wherein said method results in production of the hypoimmunogenic cell (such as the engineered hypoimmunogenic cell), which has one or more of the following properties: a) having a reduced immunogenicity upon the hypoimmunogenic cell's (such as the engineered hypoimmunogenic cell's) presence in an allogeneic or non-MHC matched subject as compared to a corresponding immunogenic cell, but without the genetic modification(s) of (i) and (ii); b) causing a reduced immune response to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii); and c) causing a reduced alloreactive T cell cytotoxicity to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii).

In one aspect, provided herein is a method of producing a hypoimmunogenic cell (such as an engineered hypoimmunogenic cell) from an immunogenic, comprising: a) reprogramming the immunogenic cell to produce an induced pluripotent stem (iPS) cell; b) (i) genetically modifying a CD58 gene in the iPS cell, wherein genetically modifying the CD58 gene reduces expression of the CD58 protein in said iPS cell, and (ii) optionally further genetically modifying one or more genes selected from a class II major histocompatibility complex transactivator (CIITA) gene, a regulatory factor X (RFX) gene, and a beta-2-microglobulin (B2M) gene in said iPS cell, wherein genetically modifying the gene reduces expression of the corresponding protein in said iPS cell; and c) optionally, differentiating the cell produced in step (b); wherein said method results in production of the hypoimmunogenic cell (such as the engineered hypoimmunogenic cell), which has one or more of the following properties: 1) having a reduced immunogenicity upon the hypoimmunogenic cell's, such as the engineered hypoimmunogenic cell's, presence in an allogeneic or non-MHC matched subject, as compared to a corresponding iPS cell, or a cell corresponding to the cell produced in step (c), but without the genetic modification(s) of step (b); 2) causing a reduced immune response to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b); and 3) causing a reduced alloreactive T cell cytotoxicity to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b).

In some embodiments, the hypoimmunogenic cell (such as the engineered hypoimmunogenic cell) comprises a T-cell receptor (TCR) comprising a γ chain and a δ chain.

In some embodiments, the immunogenic cell or the human immunogenic cell is an immune cell, optionally selected from T cells, natural killer (NK) cells, B cells, and hematopoietic stem cells (HSCs).

In some embodiments, the reduced immunogenicity of the hypoimmunogenic cell (such as the engineered hypoimmunogenic cell) comprises one or more of the following: i) a reduced or ablated myeloid cell response to the hypoimmunogenic cell (such as an engineered hypoimmunogenic cell) upon the cell's presence in an allogeneic or non-MHC matched subject, as compared to a cell corresponding to the cell that was modified but without said genetic modification(s); ii) a reduced or ablated T cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iii) a reduced or ablated natural killer (NK) cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iv) a reduced or ablated neutralizing antibody response to the hypoimmunogenic cell (such as the engineered hypoimmunogenic cell) upon the cell's presence in an allogeneic or non-MHC matched subject, as compared to a cell corresponding to the cell that was modified but without said genetic modification(s); v) a reduced or ablated MHC class II mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); vi) a reduced or ablated neutralizing MHC class I mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); and vii) a reduced or ablated allogeneic host versus graft rejection of to the hypoimmunogenic cell (such as the engineered hypoimmunogenic cell) upon the cell's presence in an allogeneic subject, as compared to a cell corresponding to the cell that was modified but without said genetic modification(s).

In some embodiments, the immunogenic cell is a human cell.

In some embodiments, in the hypoimmunogenic cell (such as the engineered hypoimmunogenic cell): i) expression of HLA class II molecules is reduced or ablated; ii) expression of HLA-A, HLA-B, and/or HLA-C is reduced; and iii) expression of HLA-E is reduced but remains detectable.

In some embodiments, the method comprises forming at least one embryoid body or multicellular body from the genetically modified cell to produce the hypoimmunogenic cell (such as the engineered hypoimmunogenic cell).

In some embodiments, the method further comprises determining immunogenicity of the hypoimmunogenic cell (such as the engineered hypoimmunogenic cell).

In some embodiments, the method further comprises administering the hypoimmunogenic cell (such as the engineered hypoimmunogenic cell) to an allogeneic or non-MHC matched subject.

In some embodiments, the immunogenicity of the hypoimmunogenic cell (such as the engineered hypoimmunogenic cell) is altered as compared to an immunogenic cell or an immunogenic human cell or an iPS human cell or an iPS cell where the only difference between the hypoimmunogenic cell (such as the engineered hypoimmunogenic cell) and the immunogenic cell or the immunogenic human cell or the iPS human cell or the iPS cell is that the CD58 gene and optionally one or more of the RFX gene, the CIITA gene, and the B2M gene is not genetically modified in the immunogenic cell or the immunogenic human cell or the iPS human cell or the iPS cell.

In some embodiments, the immunogenic human cell or immunogenic cell is allogeneic or non-HLA matched or non-MHC matched to cells, receptors, or polypeptides of the immune system of a recipient subject.

In some embodiments, altering the immunogenicity comprises balancing, reducing, or neutralizing the immunogenicity, such as reducing or neutralizing the immunogenicity.

In some embodiments, altering the immunogenicity comprises reducing or neutralizing a myeloid cell response to the hypoimmunogenic cells (such as the engineered hypoimmunogenic cell).

In some embodiments, altering the immunogenicity comprises reducing or neutralizing a T cell response to the hypoimmunogenic cells (such as the engineered hypoimmunogenic cell).

In some embodiments, altering the immunogenicity comprises reducing or neutralizing a natural killer cell response to the hypoimmunogenic cells (such as the engineered hypoimmunogenic cell).

In some embodiments, altering the immunogenicity comprises reducing or neutralizing an allogeneic host versus graft rejection.

In some embodiments, altering the immunogenicity comprises reducing or ablating a co-stimulatory immune cell response, and/or impairing the formation of an immune synapse.

In some embodiments, the method disclosed herein further comprises genetically modifying a RFX gene, wherein the RFX gene is RFX5, RFXANK, or RFXAP. In some embodiments, two or more of RFX5, RFXANK or RFXAP are genetically modified. In some embodiments, each of RFX5, RFXANK, and RFXAP are genetically modified.

In some embodiments, genetically modifying the RFX gene results in one or more of the following in the hypoimmunogenic cell (such as the engineered hypoimmunogenic cell): a) expression of HLA class II molecules are reduced or ablated; b) expression of HLA-A, HLA-B, and/or HLA-C are reduced; and c) expression of HLA-E is reduced but remains detectable.

In some embodiments, genetically modifying the RFX gene results in reducing or ablating MHC class II mediated response to the hypoimmunogenic cell (such as the engineered hypoimmunogenic cell). In some embodiments, genetically modifying the RFX gene results in reducing or neutralizing MHC class I mediated response to the hypoimmunogenic cell (such as the engineered hypoimmunogenic cell).

In some embodiments, the method disclosed herein further comprises genetically modifying a B2M gene, wherein genetically modifying the B2M gene results in reducing or ablating expression of HLA class I molecules.

In some embodiments, the method disclosed herein further comprises genetically modifying a CIITA gene, wherein genetically modifying the CIITA gene results in reducing or ablating expression of HLA class II molecules.

In some embodiments, genetically modifying the CD58 gene comprises: (i) modifying the DNA sequence of the CD58 gene, optionally through a CRISPR-Cas system; (ii) repressing transcription or translation of the CD58 mRNA through RNAi system, optionally the RNAi system comprises shRNA, siRNA, or miR-adapted shRNA; or (iii) reducing or ablating transcription of the CD58 gene, optionally through recruiting or directing transcriptional repressors to the CD58 gene.

In some embodiments, genetically modifying the CIITA gene and/or the B2M gene and/or the RFX gene comprises: (i) modifying the DNA sequence of the CIITA gene and/or the B2M gene and/or the RFX gene, optionally through a CRISPR-Cas system; (ii) repressing transcription or translation of the CIITA gene and/or the B2M gene and/or the RFX gene through a RNAi system, optionally wherein the RNAi system comprises shRNA, siRNA, miR-adapted shRNA, or a combination thereof; or (iii) reducing or ablating transcription of the CIITA gene and/or the B2M gene and/or the RFX gene, optionally through recruiting or directing transcriptional repressors to the CIITA gene and/or the B2M gene and/or the RFX gene.

In some embodiments, the method disclosed herein further comprises genetically modifying at least one of a TNFRSF14 gene, a TNFRSF1A gene, a TNFRSF1B gene, an ICAM1 gene, and a herpesvirus entry mediator (HVEM) gene.

In one aspect, provided herein is a non-naturally occurring hypoimmunogenic human cell (such as an engineered hypoimmunogenic human cell) produced by the method disclosed herein.

In one aspect, provided herein is a non-naturally occurring hypoimmunogenic human cell (such as an engineered hypoimmunogenic human cell, comprising a genetically modified CD58 gene, wherein the genetically modified CD58 gene reduces expression of the CD58 protein, and the hypoimmunogenic human cell (such as the engineered hypoimmunogenic human cell) is produced from an embryoid body; optionally the hypoimmunogenic human cell (such as the engineered hypoimmunogenic human cell) further comprises one or more of a genetically modified CIITA gene, a genetically modified RFX gene, and a genetically modified B2M gene.

In one aspect, provided herein is a composition comprising the hypoimmunogenic human cell (such as the engineered hypoimmunogenic human cell) disclosed herein.

In one aspect, provided herein is a γδT cell-derived induced pluripotent stem (iPS) human cell, comprising a genetically modified CD58 gene, wherein the genetically modified CD58 gene reduces expression of the CD58 protein; optionally the iPS human cell further comprises one or more of a genetically modified CIITA gene, a genetically modified RFX gene, and a genetically modified B2M gene.

In one aspect, provided herein is a composition comprising the iPS human cell disclosed herein.

In one aspect, provided herein is a method of hypoimmunogenicity (such as engineering hypoimmunogenicity), comprising: a) a step for performing a function of genetically modifying a CD58 gene of at least one immunogenic human cell, wherein genetically modifying the CD58 gene reduces expression of the CD58 protein in the immunogenic human cell; b) a step for performing a function of forming at least one embryoid body or multicellular body from the cell of a) to produce at least one hypoimmunogenic cell (such as an engineered hypoimmunogenic cell); c) a step for performing a function of subjecting the hypoimmunogenic cell (such as the engineered hypoimmunogenic cell) to an immune system; and d) a step for performing a function of determining immunogenicity of the hypoimmunogenic cell (such as the engineered hypoimmunogenic cell), wherein the immunogenicity is altered as compared to an immunogenic human cell where the CD58 gene is not genetically modified, optionally wherein step a) further comprises a step for performing a function of genetically modifying a RFX gene, a CIITA gene, and/or a B2M gene of the immunogenic human cell.

In one aspect, provided herein is a method of hypoimmunogenicity (such as engineering hypoimmunogenicity), comprising: a) a step for performing a function of reprogramming an immunogenic human cell to produce an induced pluripotent stem (iPS) human cell, wherein the immunogenic human cell comprises a heterodimeric T-cell receptor comprising a γ chain and a δ chain; b) a step for performing a function of genetically modifying a CD58 gene of the iPS human cell, wherein genetically modifying the CD58 gene reduces expression of the CD58 protein by the iPS human cell; c) a step for performing a function of forming at least one embryoid body from the cell of step b) to produce at least one hypoimmunogenic cell (such as an engineered hypoimmunogenic cell); d) a step for performing a function of subjecting the hypoimmunogenic cell (such as the engineered hypoimmunogenic cell) to an immune system; and e) a step for performing a function of determining immunogenicity of the hypoimmunogenic cell (such as the engineered hypoimmunogenic cell), wherein the immunogenicity is altered as compared to an iPS human cell where the B2M gene is not genetically modified, optionally wherein step b) further comprises a step for performing a function of genetically modifying a RFX gene, a CIITA gene, and/or a B2M gene of the iPS human cell.

In one aspect, provided herein is a method of hypoimmunogenicity (such as engineering hypoimmunogenicity), comprising: a) a step for performing a function of genetically modifying a CD58 gene of an immunogenic human cell to produce a hypoimmunogenic cell (such as an engineered hypoimmunogenic cell), wherein genetically modifying the CD58 gene reduces expression of the CD58 protein by the immunogenic human cell; b) a step for performing a function of subjecting the hypoimmunogenic cell (such as the engineered hypoimmunogenic cell) to an immune system; and c) a step for performing a function of determining immunogenicity of the hypoimmunogenic cell (such as the engineered hypoimmunogenic cell), wherein the immunogenicity is altered as compared to an immunogenic human cell where the CD58 gene is not genetically modified, optionally wherein step a) further comprises a step for performing a function of genetically modifying a RFX gene, a CIITA gene, and/or a B2M gene of the immunogenic human cell.

In one aspect, provided herein is a non-naturally occurring hypoimmunogenic human cell (such as an engineered hypoimmunogenic human cell), comprising a means for reducing expression of a CD58 protein through a genetically modified CD58 gene, and/or a means for altering immunogenicity of an immune system to the hypoimmunogenic human cell (such as the engineered hypoimmunogenic human cell) as compared to an immunogenic human cell where the CD58 gene is not genetically modified; optionally wherein the hypoimmunogenic human cell (such as the engineered hypoimmunogenic human cell) further comprises a means for reducing expression of a CIITA protein, a B2M protein, and/or an RFX protein through a genetically modified CIITA gene, a genetically modified B2M gene, and/or a genetically modified RFX gene.

In one aspect, provided herein is a γδT cell-derived induced pluripotent stem (iPS) human cell, comprising a means for reducing expression of a CD58 protein through a genetically modified CD58 gene, and/or a means for altering immunogenicity of an immune system to the iPS human cell as compared to an iPS human cell where the CD58 gene is not genetically modified; optionally wherein the iPS human cell further comprises a means for reducing expression of a CIITA protein, a B2M protein, and/or an RFX protein through a genetically modified CIITA gene, a genetically modified B2M gene, and/or a genetically modified RFX gene.

### 6. BRIEF DESCRIPTION OF THE FIGURES

FIG. 1 depicts genetic knockout strategies to prevent HLA surface expression. The top panel shows a summarization of the process for generating the HLA-altered T cells. The lower panel shows results in human donor D149399 for HLA class I and HLA class II expression measured by flow cytometry on CD4⁺ T cells with the indicated gene knockouts by CRISPR/Cas9 editing. Knockout of B2M resulted in cells lacking HLA class I expression with unaltered HLA class II expression. Knockout of CIITA resulted in cells lacking HLA class II expression with unaltered HLA class I expression. Knockout of RFX5, RFXANK, or RFXAP individually resulted in cells that lack HLA class II surface expression and have reduced, but not absent, HLA class I expression. Combined knockout of B2M and RFX5, B2M and RFXANK, B2M and RFXAP, or B2M and CIITA resulted in cells completely absent of HLA class I and II expression. Primary T cells were analyzed 14-15 days after CRISPR editing and CD3/CD28 activation. Similar results were obtained with CD8⁺ T cells. The lower right-hand panel shows expression of HLA-E on unedited, B2M deficient, and RFX5 deficient pan T cells from human donor D149399. Similar results for HLA-E expression were obtained with RFXANK and RFXAP edited T cells.

FIG. 2 depicts that RFX knockout T cells from additional human donors also had down-regulation of HLA class I and II molecules. The left-hand and right-hand panels show results for HLA class I and HLA class II expression measured by flow cytometry on CD4⁺ and CD8⁺ T cells, respectively, with the indicated gene knockouts by CRISPR/Cas9 editing. Results from two human donors shown (D151100, top row, and D144786, bottom row). NTC=unedited T cells.

FIG. 3 depicts that RFX5 knockout T cells using CRISPR/Cas12a had down-regulation of HLA class I and II molecules. The experimental scheme is shown in FIG. 1, top panel. As shown are the results for HLA class I and HLA class II expression measured by flow cytometry on D147297 pan T cells (combination of CD4⁺ and CD8⁺) with the indicated gene knockouts by CRISPR/Cas12a editing. NTC=unedited.

FIG. 4 depicts stability of reduced HLA surface expression in CD4⁺ T cells after stimulation. 14 days after the generation of HLA class I and II altered T cells from two human donors (D151100 and D144786), the cells were cryopreserved, thawed, and then stimulated with IFN-gamma or CD3/CD28 stimulation (TransAct) as indicated. 24 hours later the cells were analyzed for surface expression of pan HLA class I (top) and class II (bottom) on CD4⁺ T cells. The top left-hand panel shows HLA Class I Expression on D151100—CD4⁺ T cells. The top right-hand panel shows HLA Class I Expression on D144786—CD4⁺ T cells. The bottom left-hand panel shows HLA Class II Expression on D151100—CD4⁺ T cells. The bottom right-hand panel shows HLA Class II Expression on D144786—CD4⁺ T cells.

FIG. 5 depicts stability of reduced HLA surface expression in CD8⁺ T cells after stimulation. 14 days after the generation of HLA class I and II altered T cells from two human donors (D151100 and D144786), the cells were cryopreserved, thawed, and then stimulated with IFN-gamma or CD3/CD28 stimulation (TransAct) as indicated. 24 hours later the cells were analyzed for surface expression of pan HLA class I (top panels) and class II (bottom panels) on CD8⁺ T cells. The top left-hand panel shows HLA Class I Expression on D151100—CD8⁺ T cells. The top right-hand panel shows HLA Class I Expression on D144786—CD8⁺ T cells. The bottom left-hand panel shows HLA Class II Expression on D151100—CD8⁺ T cells. The bottom right-hand panel shows HLA Class II Expression on D144786—CD8⁺ T cells.

FIG. 6 depicts that HLA-altered T cells avoided allogeneic effector T cell responses. The top panel depicts the methodology to generate allogeneic effector T cells. The bottom panel shows the degranulation (CD107a^{High}) of allogeneic effector CD8⁺ and CD4⁺ T cells in response to a 4-hour stimulation by pan T cells with the indicated genetic modifications. The positive control was CD3/CD28 stimulation.

FIG. 7 depicts that RFX knockout T cells had intermediate protection from both allogeneic T cells and NK cells. The survival of pan T cells with the indicated genetic modifications after co-culture with allogeneic effector T cells (top panel) or resting primary NK cells (bottom panel) are shown. As compared to unedited (NTC) T cells, HLA-altered T cells (D151100) show enhanced ability to survive challenge with allogeneic effector T cells. Of the HLA-altered T cells, RFX knockout T cells showed the most ability to survive challenge with primary NK cells.

FIG. 8 depicts expansion of allo-primed effector cells against human donor 147297 (donor 297). FIG. 6 depicts the methodology to generate allogeneic effector T cells and profiling of these cells from two human donors (500 and 996, top panel) generated against the stimulator donor 297 (bottom panel). The panels indicate the HLA class I and HLA class II surface profile of HLA-altered pan T cells from human donor 297 used in the subsequent co-culture assay.

FIG. 9 depicts that RFX5 knockouts survived better than or equal to B2M knockouts from human donor 297 against all allogeneic effector cells tested. FIG. 9 shows the survival of pan T cells with the indicated genetic modifications after co-culture with unpurified allogeneic effector cells (top left and top middle panel), purified allogeneic effector T cells (bottom left), purified allogeneic effector NK cells (bottom middle), or resting primary NK cells from two human donors (right top and right bottom panels). The viability of all co-culture samples was normalized to the target cells without effectors (dotted line=1). Effectors: T-297-500R Mixture=PBMCs from donor 500 expanded for 2 weeks by priming with irradiated donor 297 PBMCs (87% T cells, 10% NKT cells, <2% NK cells); T-297-996R Mixture=PBMCs from donor 996 expanded for 2 weeks by priming with irradiated donor 297 PBMCs (72% T cells, 3% NKT cells, 22% NK cells), the T-297-996R Mixture was separated into T-297-996R isolated T cells (97% T cells and <2% NK and NKT cells) and isolated NK cells (94% NK cells, 3% NKT cells, 3% T cells); EN021 and NK697 Naive NK cells=Unprimed NK cells isolated from PBMCs of two random human donors.

FIG. 10 depicts that RFX5 knockout limited the allogeneic-induced-activation of T cells (CD3⁺CD8⁺ and CD3⁺CD4⁺ allogeneic effector cells). FIG. 10 shows the activation (41BB⁺) of allogeneic effector CD8⁺ (left panels) and CD4⁺ T cells (right panels) from two human donors (donor 500 and donor 996) in response to 24 hr stimulation by pan T cells with the indicated genetic modifications at various E:T ratios (from left to right: 1:10, 1:5, 1:2, 1:1, 2:1, 5:1, 10:1, 20:1). Negative controls were autologous pan T cells from the effector human donor.

FIG. 11 depicts D149399 T cell expansion after CRISPR knockout, with no detrimental effect of RFX, CIITA, or B2M knockout. Data showed viability, average diameter, and fold expansion of HLA-altered T cells during the generation and expansion process. CRISPR editing and CD3/CD28 activation occurred on Day 1.

FIG. 12 depicts D151100 T cell expansion after CRISPR knockout, with no detrimental effect of RFX or B2M knockout. Data showed viability, average diameter, and fold expansion of HLA-altered T cells during the generation and expansion process. CRISPR editing and CD3/CD28 activation occurred on Day 1.

FIG. 13 depicts D144786 T cell expansion after CRISPR knockout, with no detrimental effect of RFX or B2M knockout. Data showed viability, average diameter, and fold expansion of HLA-altered T cells during the generation and expansion process. CRISPR editing and CD3/CD28 activation occurred on Day 1.

FIG. 14 depicts that PGP1 iPSCs were edited by CRISPR/Cas12a to generate B2M disrupted cells using the B2M-2 crRNA. Expression of B2M is shown relative to control unedited iPSCs. In combination with Table 2, it shows that CRISPR/Cas12a can be used to edit B2M, RFX5, RFXANK, RFXAP, or CIITA in PGP1 iPSCs and B2M or RFX5 in γδT cell-derived iPSCs.

FIG. 15 depicts generation and phenotype of B2M and co-stimulatory knockout T cells from human donor RD01000079 (Donor 079). Results for the gene editing process to generate B2M knockout pan T cells with additional co-stimulatory gene knockouts are shown. Flow cytometry phenotyping was performed 11 days after CRISPR editing and expansion.

FIG. 16 depicts generation and phenotype of B2M and co-stimulatory knockout T cells (from human donor D327084, “Donor 084”). Results for the gene editing process to generate B2M knockout pan T cells with additional co-stimulatory gene knockouts are shown. Flow cytometry phenotyping was performed 11 days after CRISPR editing and expansion.

FIG. 17 depicts that CD58 knockout combined with B2M knockout results in less specific lysis and improved cell viability compared to B2M knockout only when HLA-altered T cells are co-cultured with resting NK cells. The specific lysis (left panel) and normalized viability (right panel) of pan T cells from two human donors (D327084 and RD01000079) with the indicated genetic modifications after co-culture with resting primary NK cells were shown. Effector: NK079, Targets: D327084 and RD01000079.

FIG. 18 depicts that various co-stimulatory molecule knockouts combined with B2M knockouts in T cells reduced specific lysis from NK cells. The reduction in specific lysis of pan T cells from one human donor (D327084) with the indicated genetic modifications after co-culture with resting primary NK cells at E:T=1 was shown. Reduction in specific lysis was normalized relative to B2M knockout only pan T cells. Effector donors: NK021 and NK079.

FIG. 19 depicts generation of RFX5 and CD58 knockout T cells. FIG. 19 shows results for the gene editing process to generate RFX5, CD58, and RFX5/CD58 knockout T cells. Flow cytometry phenotyping performed 14 days after CRISPR editing and expansion was shown. NTC=unedited control.

FIG. 20 depicts that CD58 knockout improved viability compared to unedited T cells in co-culture with alloreactive effector T cells. The survival of pan T cells with the indicated genetic modifications after 24 hr co-culture with allogeneic effector T cells from two human donors (D146500 and D151200) were shown. Autologous indicated target cells were unedited, expanded pan T cells from the same human donor used as the effector.

FIG. 21 depicts that CD58 knockout in addition to RFX5 knockout in T cells induced less activation (CD137+) of alloreactive CD4⁺ T cells than RFX5 knockout alone. FIG. 21 shows the activation of allogeneic effector CD4⁺ T cells from two human donors (D146500 and D151200) after 24 hr co-culture with pan T cells containing the indicated genetic modifications. The bars of each ratio condition from left to right represent: RFX5 knockout, RFX knockout/CD58 knockout, CD58 knockout, and NTC, which is the non targeted (unedited) control. Effector alone is shown at the end of the bar figure.

FIG. 22 depicts that CD58 knockout in addition to RFX5 knockout in T cells induced less activation (CD137+) of alloreactive CD8⁺ T cells than RFX5 knockout alone. FIG. 22 showed the activation of allogeneic effector CD8⁺ T cells from two human donors (D146500 and D151200) after 24 hr co-culture with pan T cells containing the indicated genetic modifications. The bars of each ratio condition from left to right represent: RFX5 knockout, RFX knockout/CD58 knockout, CD58 knockout, and NTC, which is the non targeted (unedited) control. Effector alone is shown at the end of the bar figure.

FIG. 23 depicts CD58 knockout in addition to RFX5 knockout in T cells improved viability compared to RFX5 knockout in co-cultures with NK cells. FIG. 23 showed the survival of pan T cells with the indicated genetic modifications or K562 cells (positive control) after 24 hr co-culture with resting NK cells from two human donors (NK079 and NK567).

FIG. 24 depicts CD58 knockout in addition to RFX5 knockout in T cells induces less NK cell (CD137+) activation compared to RFX5 knockout alone. FIG. 24 showed the activation of NK cells from two donors (NK079 and NK567) after 24 hr co-culture with pan T cells containing the indicated genetic modifications. The bars of each ratio condition from left to right represent: RFX5 knockout, RFX5 knockout/CD58 knockout, CD58 knockout, NTC, and K562. Effector alone is shown at the end of the bar figure.

FIG. 25 depicts that CD58 shRNAs tested in Jurkat and primary T cells showed knockdown of CD58 surface protein. FIG. 25 shows CD58 expression measured by flow cytometry in primary human pan T cells (top) and Jurkats (bottom) transduced with lentiviruses containing CD58 shRNAs. FIG. 25 discloses SEQ ID NOs: 60-67 and 60-67, respectively, in order of appearance.

FIG. 26 depicts B2M editing efficiency with Cas12a and WT MAD7 in iPSCs. Cas12a (top panels) or MAD7 (bottom panels) RNP was formed with gRNA B2M_12A_2. The flow plots shown are gated on live, single cells. Signals Reference: E082949.

FIG. 27 depicts an RFX5 gRNA tiling screen in iPSCs. The editing efficiency of each gRNA tested to knockout the RFX5 gene is shown. Signals Reference: E085286.

FIGS. 28A-28B depict the optimization of RFX gRNA structure. The editing efficiencies of the top two RFX5 gRNAs with optimization to the gRNA structure are shown. Specifically, FIG. 28A shows the editing efficiency of the RFX5 Exon9 gRNA2 sequence, and FIG. 28B shows the editing efficiency of the RFX5 Exon10 gRNA1 sequence. Three repeat sequences were tested as well as 20 bp and 21 bp spacer sequence lengths. Signals Reference: E110898.

FIG. 29 depicts a CD58 gRNA tiling screen in iPSCs. The editing efficiency of each gRNA tested to knockout the CD58 gene is shown. Signals Reference: E127262.

FIG. 30 depicts pulse code optimization of editing efficiency in three γδT-iPSC clones with gRNA RFX5_Exon9_gRNA 2 20 bp. Signals Reference: E152036.

FIG. 31 depicts CAR knock-in into RFX5 with gRNA RFX5_Exon10_gRNA1 20 bp. The editing efficiency of CAR knock-in is shown with gRNA RFX5_Exon10_gRNA1 20 bp. Four separate reactions were performed with either 300 bp or 500 bp homology arms in the DNA donor template and with and without M3814. The flow plots shown are gated on live, single cells, and the CAR positive cells were determined by comparing the edited samples to the no RNP negative control. Signals Reference: E145675.

FIG. 32 depicts CAR knock-in into RFX5 with gRNA RFX5_Exon9_gRNA 2 20 bp. The editing efficiency of CAR knock-in is shown with gRNA RFX5_Exon9_gRNA 2 20 bp with 500 bp homology arms in the DNA donor template and with and without M3814. The flow plots shown are gated on live, single cells, and the CAR positive cells were determined by comparing the edited samples to the no RNP negative control. Signals Reference: E145675.

FIG. 33 depicts the pulse code optimization of CAR knock-in into RFX5. The editing efficiency of CAR knock-in is shown and was achieved with two pulse codes on the Lonza Nucleofector with gRNA RFX5_Exon9_gRNA 2 20 bp with and without M3814. The flow plots shown are gated on live, single cells. Signals Reference: E150713.

FIG. 34 depicts iPSC HLA class I expression in cells edited with MAD7 and gRNA RFX5_Exon9_gRNA 2 20 bp. The edited cells (left panel) had decreased expression of HLA class I compared to the unedited cells (right panel). The flow plots shown are gated on live, single cells. Signals Reference: E154516.

FIG. 35 depicts iPSC CD58 expression in cells edited with MAD7 and gRNA CD58_Exon2_gRNA 9 21 bp. The edited cells (left panel) had decreased expression of CD58 compared to the unedited cells (right panel). The flow plots shown are gated on live, single cells. Signals Reference: E132854.

FIG. 36 depicts the generation of clonal cells with CAR knock-in into RFX5. CAR knock-in into RFX5 with gRNA RFX5_Exon9_gRNA 2 20 bp was achieved. The bulk edited cells were single-cell sorted to produce clonal CAR⁺ cells that maintained high expression of pluripotency markersSSEA-3, SSEA-4, OCT3/4, and SOX2. The surface markers SSEA-1 and CD34 that are not expressed in iPSCs remain low after editing and cloning. The flow plots shown are gated on live, single cells. A representative clone, Clone D5, has nearly 100% CAR expression determined by flow cytometry. Clone D5 has a 12 bp deletion. Signals Reference: E150740 & E164103.

FIG. 37 depicts the generation of clonal cells with CAR knock-in into RFX5. CAR knock-in into RFX5 with gRNA RFX5_Exon9_gRNA 2 20 bp was achieved. The bulk edited cells were single-cell sorted to produce clonal CAR⁺ cells that maintained high expression of pluripotency markersSSEA-3, SSEA-4, OCT3/4, and SOX2. The surface markers SSEA-1 and CD34 that are not expressed in iPSCs remain low after editing and cloning. The flow plots shown are gated on live, single cells. A representative clone, Clone C3, has nearly 100% CAR expression determined by flow cytometry. Clone C3 has a 15 bp deletion. Signals Reference: E150740 & E164103.

FIG. 38 depicts the editing efficiencies of MAD7 gRNAs split into crRNA and tracrRNA. The split gRNAs were formed by adding equimolar mixture of the split tracrRNA with relevant crRNA and incubating for 15 minutes at room temperature prior to RNP formation. Indel frequency of MAD7 with unmodified crRNA, AltR modified crRNA, and split gRNAs 3, 4, and 5 targeting the two RFX5 and CD58 loci are shown. Signals Reference: E164852.

FIGS. 39A-39B depict that CD58 knockout improves the ability of RFX5 knockout cells to evade alloreactive effector T cells. Gene edited or control T cells (targets) were co-cultured with alloreactive effector T cells at the indicated E:Ts in an overnight cytotoxicity assay. Normalized target viability was calculated as: % live targets at E:T/% live targets alone, where a value of 1.0 indicates complete evasion of cytotoxicity. Top panel (FIG. 39A) shows data from one representative experiment with a single donor. Bottom panel (FIG. 39B) shows aggregate data at E:T=10 from multiple experiments with several target and effector donors.

FIGS. 40A-40B depict that CD58 knockout improves the ability of RFX5 knockout cells to evade primary NK cells. Gene edited or control T cells (targets) were co-cultured with primary NK cells at the indicated E:Ts in an overnight cytotoxicity assay. Normalized target viability was calculated as: % live targets at E:T/% live targets alone, where a value of 1.0 indicates complete evasion of cytotoxicity. Top panel (FIG. 40A) shows data from one representative experiment with a single human donor. Bottom panel (FIG. 40B) shows aggregate data at E:T=10 from multiple experiments with several target and effector human donors.

FIG. 41 depicts a diagram of the dual CAR and CD58 miR-shRNA Expression System, a single vector where a single pol II promoter drives expression of a transcript encoding both the CAR and knockdown of endogenous CD58 via CD58 miR-shRNA. The CD58 miR-shRNA will be processed for RNAi by Drosha and Dicer and then loaded into RISC (RNA-induced silencing complex) for silencing of the endogenous CD58 gene. The CAR portion will be translated to protein for CAR molecule expression.

FIG. 42 depicts the FACs gating strategy for evaluating CAR expression and knockdown of endogenous CD58 using 55 different dual CAR and CD58 miR-shRNA constructs.

FIG. 43 depicts the different CD58 miR-shRNA constructs transduction and evaluation of CD58 knockdown. The top panel depicts an initial round screening 55 different miR-shRNA constructs and a control CAR (without a miR-shRNA). CD58% is the MFI of CD58 for each construct/MFI of CD58 for the control CAR. The bottom panel depicts a follow up screen of the top 5 miR-shRNAs transduced into RFX5 knockout primary T cells along with 5 control conditions. Percentages above the bars are the knockdown efficiencies, calculated as (CAR⁺ CD58 MFI of each construct/(CAR⁺ CD58 MFI NTC CAR −CAR⁺ CD58 MFI CD58 knockout_RFX5 knockout)).

FIG. 44 depicts that the top two dual CAR and CD58 miR-shRNA expression systems lead to efficient CAR expression and knockdown of endogenous CD58, as measured by surface flow cytometry staining. CAR⁺ cells were enriched prior to flow cytometry analysis and gated on live cells.

FIG. 45 depicts the flow cytometry gating strategy for analysis of the co-culture experiments shown in FIGS. 46A-46C.

FIGS. 46A-46C depict that CD58 knockdown improves survival of RFX5 knockout cells when challenged with alloreactive effector T cells or NK cells. Top panel (FIG. 46A) shows data from one representative experiment with a single target human donor co-cultured with a single allogeneic effector T cell human donor. Bottom panels (FIGS. 46B-46C) show aggregate data with an Area under the Curve (AUC) calculation from multiple experiments with several target and effector human donors.

### 7. DETAILED DESCRIPTION

Cell-based therapies continue to face numerous challenges that limit their clinical applications. Such challenges include, for example, as summarized in Bashor et al., Nature Reviews Drug Discovery (2022):21; 655-675, “Engineering the next generation of cell-based therapeutics.” The present disclosure addresses for the first time these and other challenges in the field of cell therapy.

The inventors provide herein, inter alia, methods of hypoimmunogenicity, such as bioengineering methodologies and materials, including hypoimmunogenicity (such as engineering hypoimmunogenicity) methodologies and materials useful in, for example, genetically modifying and/or otherwise altering at least one target gene or gene product, processes for producing engineered hypoimmunogenic cells, manufacturing of engineered hypoimmunogenic cellular compositions, and uses thereof. The present disclosure provides, in part, a method of engineering hypoimmunogenicity, comprising genetically modifying at least one target gene (e.g., a regulatory factor X (RFX) gene, a B2M gene, a CD58 gene, a CIITA gene) of a human cell or a cell to reduce expression of the protein coded by the target gene in the human cell or the cell, and forming at least one embryoid body to produce at least one engineered hypoimmunogenic cell. In some embodiments, the human cell is an immunogenic human cell. In some embodiments, the human cell is an induced pluripotent stem (iPS) human cell, for example, an iPS human cell generated by reprogramming an immunogenic γδT cell. In some embodiments, the cell is a rodent, porcine, primate, monkey, ape, or human cell. In some embodiments, the cell is an immunogenic rodent, porcine, primate, monkey, ape, or human cell. In some embodiments, the cell is an immunogenic human cell. In some embodiments, the cell is an induced pluripotent stem (iPS) cell, for example, an iPS cell generated by reprogramming an immunogenic γδT cell. The present disclosure is partly based on the discovery that the presently disclosed engineered hypoimmunogenic cells were able to evade the allogeneic host versus graft immune response.

### 7.1 Definitions

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. The range of quantity, level, value, number, frequency, percentage, dimension, size, amount, weight or length can be ±15%, ±10%, ±9%, ±8%, ±7%, ±6%, ±5%, ±4%, ±3%, ±2%, or ±1% to a reference quantity, level, value, number, frequency, percentage, dimension, size, amount, weight or length. The term “about” in relation to a reference numerical value can include the numerical value itself and a range of values, for example, plus or minus 10% from that numerical value. In some embodiments, the amount “about 10” includes 10 and any amounts from 9 to 11. In some cases, the numerical disclosed throughout can be “about” that numerical value even without specifically mentioning the term “about.”

Unless otherwise indicated, the terms “at least,” “at most,” or “about” preceding a series of elements is to be understood to refer to every element in the series.

As used herein and in the appended claims, the singular forms “a,” “an,” and “the” include plural reference unless the context clearly dictates otherwise.

As used herein, and unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “MHC molecule” refers to a major histocompatibility complex (MHC) found on the cell surface which displays peptide fragments of non-self proteins. MHC class I molecules and MHC II class molecules are two classes of MHC molecules normally found on antigen-presenting cells. MHC class I molecules consist of two polypeptide chains. The alpha chain consists of 3 polypeptides referred to as the alpha-1, alpha-2, and alpha-3 domains. The alpha chain is linked non-covalently via the alpha-3 domain to a betδ-chain which consists of beta-2 microglobulin (B2M). The alpha chain is polymorphic and is encoded, in human, by the HLA gene (i.e., HLA-A, HLA-B, and HLA-C), whereas beta-2 microglobulin is not polymorphic and is encoded by the B2M gene. MHC class II molecules are transmembrane up heterodimers. In humans, there are three MHC class II isotypes: HLA-DR, HLA-DP, and HLA-DQ, encoded by a and R chain genes within the Human Leukocyte Antigen (HLA) locus on chromosome 6.

As used herein, the term “deletion” or “knockout,” refers to a genetic modification wherein a site or region of genomic DNA is removed by any molecular biology method, e.g., methods described herein, e.g., by delivering to a site of genomic DNA an endonuclease and at least one gRNA. The term “deletion” or “knockout” includes deleting all or a portion of the target polynucleotide sequence in a way that interferes with the function of the target polynucleotide sequence. In some embodiments, “deletion” or “knockout” can result in complete or partial loss of expression of the target gene. Any number of nucleotides can be deleted. In some embodiments, a deletion involves the removal of at least one, at least two, at least three, at least four, at least five, at least ten, at least fifteen, at least twenty, at least 25, or more than at least 25 nucleotides. In some embodiments, a deletion involves the removal of 10-50, 25-75, 50-100, 50-200, or more than 100 nucleotides. In some embodiments, a deletion involves the removal of an entire target gene, e.g., an RFX gene. In some embodiments, a deletion involves the removal of part of a target gene, e.g., all or part of a promoter and/or coding sequence of a RFX gene. In some embodiments, a deletion involves the removal of a transcriptional regulator, e.g., a promoter region, of a target gene. In some embodiments, a deletion involves the removal of all or part of a coding region such that the product normally expressed by the coding region is no longer expressed, is expressed as a truncated form, or expressed at a reduced level. In some embodiments, a deletion leads to a decrease in expression of a gene relative to an unmodified cell. In some embodiments, a knockout can be achieved by altering a target polynucleotide sequence by inducing an indel in the target polynucleotide sequence in a functional domain of the target polynucleotide sequence (e.g., a DNA binding domain). The term “disruption” or “disrupted” refers to an alteration that results in a gene product that does not exhibit wildtype function and/or level of activity. In some aspects, a disruption refers to an alteration of a gene whereby the disrupted gene results in production of such a non-wildtype gene product. As used herein, “disruption” refers to RNA interference, which includes disruption of the gene's mRNA transcript via expression of an introduced miR-adapted shRNA.

In some embodiments, the disruption truncates a gene, e.g., a B2M gene. In some embodiments, the disruption deletes a gene, e.g., a B2M gene. In some embodiments, the disruption results in the gene producing an inactive protein. In some embodiments, the disruption results in disruption of the reading frame of B2M by multiple out-of-frame deletions. In some embodiments, the disruption results in disruption of the reading frame of B2M by a single out-of-frame deletion. In some embodiments, the disruption results in insertion of about or at least about one, two, three, four, five, six, seven, eight, nine, ten, or more than ten nucleotide(s) or nucleotide base pair(s) (e.g., an insertion that changes the reading frame of a gene (e.g., B2M)). In some embodiments, the disruption results in disruption of the reading frame of B2M. In particular embodiments, the gene is a B2M gene and the disruption results in the B2M gene producing an inactive B2M protein. In some embodiments, the disruption results in the gene expressing a reduced amount of gene product, e.g., a reduced amount of B2M polypeptide. In particular embodiments, the gene is a B2M gene and the disruption results in the B2M gene expressing a reduced amount of B2M protein. In some embodiments, the disruption results in the gene expressing no detectable amount of gene product, e.g., no detectable amount of B2M protein. In some embodiments, the gene is a B2M gene and the disruption results in the B2M gene expressing no detectable amount of B2M protein. A disrupted gene, e.g., a disrupted B2M gene, may refer to a gene comprising an insertion, deletion, or substitution relative to a corresponding wildtype gene such that the disrupted gene expresses a reduced, e.g., no detectable amount of functional protein relative to expression of the wildtype gene. A gene may be disrupted, for example, via a method of inserting, deleting, or substituting at least one nucleotide/nucleic acid in an endogenous gene such that expression of a functional protein from the endogenous gene is reduced or inhibited. In some embodiments, the substitution is performed by a base editor, in which the base editor converts one nucleotide to another by modifying the chemical structure of the nucleotide. In some embodiments, the terms “disruption,” “disrupted,” “knockout,” or “deletion” are used interchangeably in the disclosure. In some embodiments, the at least one gRNA is complementary to and/or hybridizes to a sequence on a target polynucleotide sequence, wherein the target polynucleotide sequence comprises an B2M gene. In some embodiments, the target polynucleotide sequence comprises the sequence set forth in SEQ ID NO: 253. In some embodiments, the gRNA comprises the repeat sequence set forth in SEQ ID NO: 129 (UAAUUUCUACUCUUGUAGAU), optionally in combination with a spacer sequence set forth in SEQ ID NO: 251 (AGUGGGGGUGAAUUCAGUGUA). In some embodiments, the gRNA comprises the sequence set forth in SEQ ID NO: 252.

In some embodiments, the gRNA targeting B2M is a discontinuous or “split” RNA.

In some embodiments, the disruption truncates a gene, e.g., a RFX gene. In some embodiments, the disruption deletes a gene, e.g., a RFX gene. In some embodiments, the disruption results in the gene producing an inactive protein. In some embodiments, the disruption results in disruption of the reading frame of RFX by multiple out-of-frame deletions. In some embodiments, the disruption results in disruption of the reading frame of RFX by a single out-of-frame deletion. In some embodiments, the disruption results in insertion of about or at least about one, two, three, four, five, six, seven, eight, nine, ten, or more than ten nucleotide(s) or nucleotide base pair(s) (e.g., an insertion that changes the reading frame of a gene (e.g., RFX)). In some embodiments, the disruption results in disruption of the reading frame of RFX. In particular embodiments, the gene is a RFX gene and the disruption results in the RFX gene producing an inactive RFX protein. In some embodiments, the disruption results in the gene expressing a reduced amount of gene product, e.g., a reduced amount of RFX polypeptide. In particular embodiments, the gene is a RFX gene and the disruption results in the RFX gene expressing a reduced amount of RFX protein. In some embodiments, the disruption results in the gene expressing no detectable amount of gene product, e.g., no detectable amount of RFX protein. In some embodiments, the gene is a RFX gene and the disruption results in the RFX gene expressing no detectable amount of RFX protein. A disrupted gene, e.g., a disrupted RFX gene, may refer to a gene comprising an insertion, deletion, or substitution relative to a corresponding wildtype gene such that the disrupted gene expresses a reduced, e.g., no detectable amount of functional protein relative to expression of the wildtype gene. A gene may be disrupted, for example, via a method of inserting, deleting, or substituting at least one nucleotide/nucleic acid in an endogenous gene such that expression of a functional protein from the endogenous gene is reduced or inhibited. In some embodiments, the substitution is performed by a base editor, in which the base editor converts one nucleotide to another by modifying the chemical structure of the nucleotide. In some embodiments, the terms “disruption,” “disrupted,” “knockout,” or “deletion” are used interchangeably in the disclosure. In some embodiments, the at least one gRNA is complementary to and/or hybridizes to a sequence on a target polynucleotide sequence, wherein the target polynucleotide sequence comprises an RFX gene. In some embodiments, the gRNA comprises the sequence set forth in SEQ ID NO: 184 (RFX5_Exon9_gRNA 2; AGGAUCCGCUCUGCCCAGUCA), SEQ ID NO: 193 (RFX5_Exon10_gRNA 1; GAUGACCGUUCCCGAGGUGCA), SEQ ID NO: 202 (RFX5_Exon10_gRNA 4; GAGAACCCAGAGGGUGGAGCC), SEQ ID NO: 205 (RFX5_Exon10_gRNA 5; GUACCUCUGCAGAAGAGGACG), SEQ ID NO: 223 (RFX5_Exon11_gRNA 8; AGGGCACCUGAAGAAAGCCUG), SEQ ID NO: 239 (RFX5_Exon9_gRNA 2; AGGAUCCGCUCUGCCCAGUC) or SEQ ID NO: 246 (RFX5_Exon10_gRNA 1; GAUGACCGUUCCCGAGGUGC). In some embodiments, the gRNA comprises the sequence set forth in SEQ ID NO: 239 or 246. In some embodiments, the target polynucleotide sequence comprises the sequence of SEQ ID NO: 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 241, 241, or 248. In some embodiments, the gRNA comprises the repeat sequence set forth in SEQ ID NO: 129, 235, or 237. In some embodiments, the gRNA further comprises a spacer sequence set forth in SEQ ID NO:130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 239, or 246. In some embodiments, the gRNA comprises the sequence set forth in SEQ ID NO: 131, 134, 137, 140, 143, 146, 149, 152, 155, 158, 161, 164, 167, 170, 173, 176, 179, 182, 185, 188, 191, 194, 197, 200, 203, 206, 209, 212, 215, 218, 221, 224, 227, 230, 233, 236, 238, 240, 242, 243, 244, 245, 247, 249, or 250. In some embodiments, the target polynucleotide sequence comprises SEQ ID NO: 141, 186, 195, 204, 207, 225, 241, or 248. In some embodiments, the gRNA comprises the sequence set forth in SEQ ID NOs: 129, 235, or 237. In some embodiments, the gRNA further comprises a spacer sequence set forth in SEQ ID NO: 139, 184, 193, 202, 205, 223, 239, or 246. In some embodiments, the gRNA comprises the sequence set forth in SEQ ID NO: 140, 185, 194, 203, 206, 224, 236, 238, 240, 242, 243, 244, 245, 247, 249, or 250.

In some embodiments, the gRNA targeting RFX5 is a discontinuous or “split” RNA. In some embodiments, the discontinuous or “split” gRNA comprises the sequence set forth in SEQ ID NO: 377, 378, 379, 380, 381, 382, 383, 384, or 385.

In some embodiments, the disruption truncates a gene, e.g., a CD58 gene. In some embodiments, the disruption deletes a gene, e.g., a CD58 gene. In some embodiments, the disruption results in the gene producing an inactive protein. In some embodiments, the disruption results in disruption of the reading frame of CD58 by multiple out-of-frame deletions. In some embodiments, the disruption results in disruption of the reading frame of CD58 by a single out-of-frame deletion. In some embodiments, the disruption results in insertion of about or at least about one, two, three, four, five, six, seven, eight, nine, ten, or more than ten nucleotide(s) or nucleotide base pair(s) (e.g., an insertion that changes the reading frame of a gene (e.g., CD58)). In some embodiments, the disruption results in disruption of the reading frame of CD58. In particular embodiments, the gene is a CD58 gene and the disruption results in the CD58 gene producing an inactive CD58 protein. In some embodiments, the disruption results in the gene expressing a reduced amount of gene product, e.g., a reduced amount of CD58 polypeptide. In particular embodiments, the gene is a CD58 gene and the disruption results in the CD58 gene expressing a reduced amount of CD58 protein. In some embodiments, the disruption results in the gene expressing no detectable amount of gene product, e.g., no detectable amount of CD58 protein. In some embodiments, the gene is a CD58 gene and the disruption results in the CD58 gene expressing no detectable amount of CD58 protein. A disrupted gene, e.g., a disrupted CD58 gene, may refer to a gene comprising an insertion, deletion, or substitution relative to a corresponding wildtype gene such that the disrupted gene expresses a reduced, e.g., no detectable amount of functional protein relative to expression of the wildtype gene. A gene may be disrupted, for example, via a method of inserting, deleting, or substituting at least one nucleotide/nucleic acid in an endogenous gene such that expression of a functional protein from the endogenous gene is reduced or inhibited. In some embodiments, the substitution is performed by a base editor, in which the base editor converts one nucleotide to another by modifying the chemical structure of the nucleotide. In some embodiments, the terms “disruption,” “disrupted,” “knockout,” or “deletion” are used interchangeably in the disclosure. In some embodiments, the at least one gRNA is complementary to and/or hybridizes to a sequence on a target polynucleotide sequence, wherein the target polynucleotide sequence comprises a CD58 gene. In some embodiments, the target polynucleotide sequence comprises SEQ ID NO: 256, 259, 262, 265, 268, 271, 274, 277, 280, 283, 286, 289, 292, 295, 298, 301, 304, 307, 310, 313, 316, 319, 322, 325, 328, 331, 334, 337, 340, 343, 346, 349, 352, 355, 358, 361, 364, 367, 370, 373, or 376. In some embodiments, the gRNA comprises the repeat sequence set forth in SEQ ID NO: 129. In some embodiments, the gRNA further comprises a spacer sequence comprising the sequence of SEQ ID NO: 254, 257, 260, 263, 266, 269, 272, 275, 278, 281, 284, 287, 290, 293, 296, 299, 302, 305, 308, 311, 314, 317, 320, 323, 326, 329, 332, 335, 338, 341, 344, 347, 350, 353, 356, 359, 362, 365, 368, 371, or 374. In some embodiments, the gRNA comprises the sequence of SEQ ID NO: 255, 258, 261, 264, 267, 270, 273, 276, 279, 282, 285, 288, 291, 294, 297, 300, 303, 306, 309, 312, 315, 318, 321, 324, 327, 330, 333, 336, 339, 342, 345, 348, 351, 354, 357, 360, 363, 366, 369, 372, or 375. In some embodiments, the target polynucleotide sequence comprises SEQ ID NO: 256, 271, 274, 280, 304, or 328. In some embodiments, the gRNA comprises the sequence of SEQ ID NO: 129. In some embodiments, the gRNA further comprises a spacer sequence comprising the sequence of SEQ ID NO: 254, 269, 272, 278, 302, or 326. In some embodiments, the gRNA comprises the sequence of SEQ ID NO: 255, 270, 273, 279, or 327.

In some embodiments, the gRNA targeting CD58 is a discontinuous or “split” RNA. In some embodiments, the discontinuous or “split” gRNA comprises the sequence set forth in SEQ ID NO: 377, 378, 379, 386, 387, or 388.

In some embodiments, the gRNA both disrupts a gene (e.g., via indel formation resulting in non-functional expression of the gene) and introduces another polynucleotide, e.g., a gene for a chimeric antigen receptor (CAR) and/or a miR-adapted shRNA. In some embodiments, the gRNA targets RFX5. In some embodiments, the gRNA is used to knock-in a transgene containing a promoter and CAR into a target gene (e.g., one or more of a RFX gene, a CD58 gene, a CIITA gene, and/or a B2M gene) resulting in CAR expression on surface of the hypoimmunogenic cell (such as an engineered hypoimmunogenic cell) or the iPS human cell or the iPS cell that can be detected by flow cytometry. In some embodiments, the gRNA is used to knock-in a miR-adapted shRNA that targets CD58. In some embodiments, the miRNA comprises the sequence set forth in SEQ ID NO: 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or 128.

In some embodiments, shRNA is used to disrupt the CD58 gene. In some embodiments, the shRNA comprises the sequence set forth in SEQ ID NO: 60, 61, 62, 63, 64, 65, 66, or 67. In some embodiments, the shRNA comprises the sequence set forth in SEQ ID NO: 60, 63, or 64.

As used herein, the term “endonuclease” generally refers to an enzyme that cleaves phosphodiester bonds within a polynucleotide. In some embodiments, an endonuclease specifically cleaves phosphodiester bonds within a DNA polynucleotide. In some embodiments, an endonuclease is a zinc finger nuclease (ZFN), transcription activator like effector nuclease (TALEN), homing endonuclease (HE), meganuclease, MegaTAL, or a CRISPR (clustered regularly interspaced short palindromic repeat)-associated endonuclease. CRISPR clusters contain spacers, sequences complementary to antecedent mobile elements, and target invading nucleic acids. CRISPR clusters are transcribed and processed into CRISPR RNA (crRNA). In some embodiments, an endonuclease is an RNA-guided endonuclease. In certain aspects, the RNA-guided endonuclease is a CRISPR nuclease, e.g., a Type II CRISPR Cas9 endonuclease or a Type V CRISPR Cpf1 (or Cas12a) endonuclease. CRISPR-Cas systems may be characterized as Class 1 or Class 2 systems. Class 1 systems are characterized by multi-subunit effector; that is, comprising multiple Cas proteins. Class 1 systems may be further characterized as Types I, III and IV. Class 2 systems are characterized by a single effector protein having multiple domains. Class 2 systems may be further characterized as Types II, V and VI. For example, Class 2 type II systems include Cas9 while Class 2 type V systems include Cpf1 (Cas12a). Further examples of Cas proteins include, but are not limited to, Cas9 proteins, Cas9-like proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof. In some embodiments, an endonuclease is a Cas1, Cas1B, Cas2, Cas3, Cas4, Cas5, Cash, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4, Cpf1 (also known as Cas12a), MAD7, MAD2 endonuclease, or a homolog thereof, a recombination of the naturally occurring molecule thereof, a codon-optimized version thereof, or a modified version thereof, or combinations thereof. Examples of Cas proteins include, but are not limited to, MAD7, MAD2, Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c. In some embodiments, an endonuclease may introduce one or more single-stranded breaks (SSBs) and/or one or more double-stranded breaks (DSBs).

As used herein, the terms “Cas12” or “Cas12 protein” refer to any Cas12 protein including, but not limited to, Cas12 protein such as Cas12a, Cas12b, Cas12c, Cas12d, Cas12e. In some embodiments, a Cas12 protein has an amino acid sequence which is at least 85% (or at least 90%, or at least 95%, or at least 96%, or at least 97%, or at least 98%, or at least 99%) identical to the amino acid sequence of a functional Cas12 protein. In some embodiments, the Cas12 protein may be a Cas12 polypeptide substantially identical to the protein found in nature, or a Cas12 polypeptide having at least about 85% sequence identity (or at least about 90% sequence identity, or at least about 95% sequence identity, or at least about 96% sequence identity, or at least about 97% sequence identity, or at least about 98% sequence identity, or at least about 99% sequence identity) to the Cas12 protein found in nature and having substantially the same biological activity. Examples of Cas12a proteins include, but are not limited to, FnCas12a, AsCas12a, LbCas12a, Lb5Cas12a, HkCas12a, OsCas12a, TsCas12a, BbCas12a, BoCas12a or Lb4Cas12a. Examples of Cas12b proteins include, but are not limited to, AacCas12b, Aac2Cas12b, AkCas12b, AmCas12b, AhCas12b, and AcCas12b.

In some embodiments, the term “Cpf endonuclease” means an RNA-guided DNA endonuclease associated with CRISPR that cleaves a target DNA sequence when coupled with a guide RNA. The Cpf endonuclease is guided by the guide RNA(s) to recognize and cleave a specific target site in double stranded DNA in the genome of a cell. In some embodiments, the CRISPR-Cpf system employs an Acidaminococcus sp. Cpf1 endonuclease, a Lachnospiraceae sp. Cpf1 endonuclease, or a Francisella novicide Cpf1 endonuclease or variant thereof. The Cpf1-crRNA complex cleaves target DNA by identification of a protospacer adjacent motif (PAM) 5′-TTTN for the Acidaminococcus sp. Cpf1 endonuclease and Lachnospiraceae sp. Cpf1 endonuclease, and a PAM sequence 5′-TTN for the Francisella novicide Cpf1. After identification of the PAM, Cpf1 introduces sticky-end DNA double-stranded break of 4-5 nucleotides overhang distal to the 3′ end of the targeted PAM which is then repaired by either non-homologous end joining (NHEJ) or homology-directed repair (HDR). It is understood that the term “Cpf1 endonuclease” encompasses variants thereof.

As known to an ordinarily skilled person in the art the term “Mad endonuclease” means an RNA-guided DNA endonuclease associated with CRISPR that cleaves a target DNA sequence when coupled with a guide RNA. The Mad endonuclease is guided by the guide RNA(s) to recognize and cleave a specific target site in double stranded DNA in the genome of a cell. CRISPR-Mad systems are closely related to the Type V (Cpf1-like) of Class-2 family of CAS enzymes. In some embodiments, the CRISPR-Mad system employs an Eubacterium rectale MAD7 endonuclease or variant thereof. In some embodiments, MAD7 is a Class 2 type V-A CRISPR family identified in Eubacterium rectale. The MAD7-crRNA complex cleaves target DNA by identification of a protospacer adjacent motif (PAM) 5′-YTTN. After identification of the PAM, MAD7 introduces sticky-end DNA double-stranded break of 4-5 nucleotides overhang to the 3′ end of the targeted PAM which is then repaired by either non-homologous end joining (NHEJ) or homology-directed repair (HDR). It is understood that the term “Mad endonuclease” encompasses variants thereof. In some embodiments, the B2M target motif identified or used for CRISPR-Cpf1 (Cas12a) system is the same B2M target motif when using MAD7. In some embodiments, the same guide nucleic acid or guide RNA can be used with a Cpf1 (or Cas12a) and a MAD7 nuclease.

As used herein, the term “guide RNA” or “gRNA” generally refers to short ribonucleic acid that can interact with, e.g., bind to, an endonuclease and bind, or hybridize to a target genomic site or region. In some embodiments, a gRNA is a single-molecule guide RNA (sgRNA). In some embodiments, a gRNA may comprise a spacer extension region. In some embodiments, a gRNA may comprise a tracrRNA extension region. In some embodiments, a gRNA is single-stranded. In some embodiments, a gRNA comprises naturally occurring nucleotides. In some embodiments, a gRNA is a chemically modified gRNA. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, a gRNA may be pre-complexed with a DNA endonuclease. In some embodiments, a gRNA sequence comprises AltR1 and/or AltR2. In some embodiments, AltR1 and AltR2 are proprietary (IDT) modifications used to increase the stability of short RNAs (e.g., gRNA). Modifications for nucleic acids such as RNA and gRNA, for example, can be found in U.S. Pat. No. 9,840,702, incorporated by reference herein. A gRNA can be constructed as a single RNA oligonucleotide that is the combination of a repeat sequence followed by a spacer sequence, wherein specificity to the genomic target location is conferred by complementary binding of the spacer to genomic DNA. A split gRNA can be constructed as two RNA oligonucleotides, composed of a tracrRNA and a crRNA, in which the tracrRNA contains a portion of the repeat sequence and the crRNA contains a portion of the repeat sequence followed by the spacer sequence, for example.

As used herein, the term “genetic modification” generally refers to genetically edited or manipulated genomic DNA of a gene, mRNA transcribed from the gene, or transcription of the gene in a cell, which results in the reduction of expression level of a gene product, for example, a protein encoded by the gene.

The terms “decreased,” “reduced,” and “lower” are all used herein interchangeably to mean a decrease by a statistically significant amount (e.g., two standard deviations (2SD) below normal). In some embodiments, “decreased,” “reduced,” or “lower,” means a decrease by at least about 5% as compared to a reference level, for example a decrease by at least about: 5%, 10%, 15%, 20%, 25%, 30%, 35%, 40%, 45%, 50%, 55%, 60%, 65%, 70%, 75%, 80%, 85%, 90%, 95%, or 100% as compared to a reference level. In some embodiments, “decreased,” “reduced,” or “lower,” is any decrease between 10-100% as compared to a reference level. In some embodiments, “decreased,” “reduced,” or “lower,” means a decrease by at least about: 1-fold, 2-fold, 3-fold, 4-fold, 5-fold, 6-fold, 7-fold, 8-fold, 9-fold, or 10-fold as compared to a reference level. In some embodiments, decreased or reduced expression results in undetectable levels of the target gene or target polynucleotide sequence in a cell or population of cells as determined by a method used by those skilled in the art or a method disclosed in the disclosure (e.g., FACS). In some embodiments, reduced expression of RFX is reduced relative to a reference. In some embodiments, the reference is iPSCs or a population of iPSCs without genetic modification of the gene (e.g., RFX gene). In some embodiments, the reference is immunogenic human cells or a population of immunogenic human cells without genetic modification of the gene.

In some embodiments, the terms “increased,” “enhanced,” and “elevated” are all used herein interchangeably to mean an increase by at least about 5% as compared to a reference level, for example an increase by at least about: 5%, 10%, 15%, 20%, 25%, 30%, 35%, 40%, 45%, 50%, 55%, 60%, 65%, 70%, 75%, 80%, 85%, 90%, 95%, or 100% as compared to a reference level. In some embodiments, “increased,” “enhanced,” or “elevated,” is any increase between 10-100% as compared to a reference level. In some embodiments, “increased,” “enhanced,” or “elevated,” means an increase by at least about: 1-fold, 2-fold, 3-fold, 4-fold, 5-fold, 6-fold, 7-fold, 8-fold, 9-fold, or 10-fold as compared to a reference level.

As used herein, the term “polynucleotide,” which may be used interchangeably with the term “nucleic acid” generally refers to a biomolecule that comprises two or more nucleotides. Typically, a polynucleotide of the disclosure is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. In some embodiments, a polynucleotide is a hybrid DNA/RNA molecule. In some embodiments, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated. In some embodiments, a polynucleotide comprises at least two, at least five, at least ten, at least twenty, at least 30, at least 40, at least 50, at least 100, at least 200, at least 250, at least 500, or any number of nucleotides. In some embodiments, a polynucleotide is a site or region of genomic DNA. In some embodiments, a polynucleotide is an endogenous gene that is comprised within the genome of a cell. In some embodiments, a polynucleotide is an exogenous polynucleotide that is not integrated into genomic DNA. In some embodiments, a polynucleotide is an exogenous polynucleotide that is integrated into genomic DNA. In some embodiments, a polynucleotide is a plasmid or an adeno-associated viral vector. In some embodiments, a polynucleotide is a circular or linear molecule.

As used herein, “cell culture medium” (also referred to herein as a “culture medium” or “culture” or “medium”) is a medium for culturing cells containing nutrients that maintain cell viability and support proliferation. The cell culture medium may contain any of the following in any appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. Some non-limiting examples are provided herein.

As used herein, “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

As used herein, the term “differentiate,” “differentiation,” or the like refers to the process by which an unspecialized (or uncommitted) or less specialized cell acquires the features of a specialized cell such as, for example, a blood cell or a muscle cell. A differentiated or differentiation-induced cell is one that has taken on a more specialized (or committed) position within the lineage of a cell. A cell is committed when it has proceeded in the differentiation pathway to a point where, under normal circumstances, it will continue to differentiate into a specific cell type or subset of cell types, and cannot, under normal circumstances, differentiate into a different cell type or revert to a less differentiated cell type.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “exogenous” is intended to mean that the referenced molecule or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously.

As used herein, the term “induced pluripotent stem cells” or, “iPSCs,” refers to stem cells produced from differentiated adult cells that have been induced or changed (i.e., reprogrammed) into cells capable of differentiating into tissues of all three germ or dermal layers: mesoderm, endoderm, and ectoderm.

As used herein, the term “isolated” or the like when used in reference to a cell is intended to mean a cell that is substantially free of at least one component as the referenced cell is found in nature. The term includes a cell that is removed from some or all components as it is found in its natural environment. The term also includes a cell that is removed from at least one, some or all components as the cell is found in non-naturally occurring environments. Therefore, an isolated cell is partly or completely separated from other substances as it is found in nature or as it is grown, stored or subsisted in non-naturally occurring environments. Specific examples of isolated cells include partially pure cells, substantially pure cells, and cells cultured in a medium that is non-naturally occurring.

As used herein, the term “purify” or the like refers to increased purity. For example, the purity can be increased to at least 50%, 60%, 70%, 80%, 90%, 95%, 99%, or 100% (e.g., as compared to a reference).

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “population” when used with reference to T lymphocytes refers to a group of cells including two or more T lymphocytes. The isolated population of T lymphocytes can have only one type of T lymphocyte, or two or more types of T lymphocyte. The isolated population of T lymphocytes can be a homogeneous population of one type of T lymphocyte or a heterogeneous population of two or more types of T lymphocyte. The isolated population of T lymphocytes can also be a heterogeneous population having T lymphocytes and at least a cell other than a T lymphocyte, e.g., a B cell, a macrophage, a neutrophil, an erythrocyte, a hepatocyte, an endothelial cell, an epithelial cell, a muscle cell, a brain cell, etc. The heterogeneous population can have from 0.01% to about 100% T lymphocyte. Accordingly, an isolated population of T lymphocytes can have at least 50%, 60%, 70%, 80%, 90%, 95%, 98%, or 99% T lymphocytes. The isolated population of T lymphocytes can include only one type of T lymphocytes, or a mixture of more than one type of T lymphocytes. The isolated population of T lymphocytes can include one or more, or all of, the different types of T lymphocytes, including but not limited to those disclosed herein. An isolated population of T lymphocytes can include all known types of T lymphocytes. In an isolated population of T lymphocytes that includes more than one type of T lymphocytes, the ratio of each type of T lymphocyte can range from 0.01% to 99.99%. The isolated population also can be a clonal population of T lymphocytes, in which all the T lymphocytes of the population are clones of a single T lymphocyte.

A “recombinant” polynucleotide is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

As used herein, “reprogramming,” refers to a process that alters or reverses the differentiation state of a somatic cell. The cell can be either partially or terminally differentiated prior to reprogramming. Reprogramming encompasses complete reversion of the differentiation state of a somatic cell (e.g., a T cell) to a pluripotent state. Reprogramming also encompasses partial reversion of the differentiation state of a somatic cell to a state that renders the cell more susceptible to complete reprogramming to a pluripotent state when subjected to additional manipulations such as those described herein. Such contacting may result in expression of particular genes by the cells, which expression contributes to reprogramming. In some embodiments of the disclosure, reprogramming of a somatic cell causes the somatic cell to be a pluripotent and ES-like state. The resulting cells are referred to herein as reprogrammed pluripotent somatic cells or induced pluripotent stem cells (iPSCs). In some embodiments, reprogramming also encompasses partial reversion of the differentiation state of a somatic cell to a multipotent state.

Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent. In some embodiments, the methods described herein contribute to establishing the pluripotent state by reprogramming. In some embodiments, the methods described herein may be practiced on cells that fully differentiated and/or particular types of cells (e.g., γδT cells), rather than on cells that are already multipotent or pluripotent.

As used herein, “reprogramming factor” refers to a gene, RNA, or protein that promotes or contributes to cell reprogramming, e.g., in vitro. Examples of reprogramming factors of interest for reprogramming somatic cells to pluripotency in vitro are Oct3/4, Klf4, c-Myc, Nanog, Sox2, and Lin28, and any gene/protein that can substitute for one or more of these in a method of reprogramming somatic cells, e.g., in vitro.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T lymphocyte can be any T lymphocyte, such as a cultured T lymphocyte, e.g., a primary T lymphocyte, or a T lymphocyte from a cultured T cell line, e.g., Jurkat, SupT1, etc., or a T lymphocyte obtained from a mammal. The T lymphocyte can be CD3⁺ cells. The T lymphocyte can be any type of T lymphocyte and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδT cells), and the like. A T lymphocyte can be T regulatory cell, which includes nTregs (natural Tregs), iTregs (inducible Tregs), CD8⁺ Treg, Tr1 regulatory cells, and Th3 cells. Additional types of helper T cells include cells such as Th3 (Treg), Th17, Th9, or T follicular helper (Tfh)cells. Additional types of memory T cells include cells such as central memory T cells (T_{CM} cells), effector memory T cells (TEM cells and TEMRA cells). The T lymphocyte can also refer to a genetically engineered T lymphocyte, such as a T lymphocyte modified to express a T cell receptor (TCR) or a chimeric antigen receptor (CAR). The T lymphocyte can also be differentiated from a stem cell, definitive hemogenic endothelium, a CD34+ cell, an HSC (hematopoietic stem and progenitor cell), a hematopoietic multipotent progenitor cell, or a T cell progenitor cell.

As used herein, the term “γδT cells” refers to T cells having T cell receptor comprising a γ-chain and a δ-chain on their surfaces.

As used herein, the term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments, the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions”. To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated when being co-cultured with other cells by irradiation or treatment with an anti-mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage, enhance proliferation capacity and promote maturation to a specialized cell type, such as an effector cell.

As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium. In some embodiments, a feeder-free environment is free of both feeder and stromal cells and is also not pre-conditioned by the cultivation of feeder cells.

The term “pluripotency associated gene” refers to a gene whose expression under normal conditions (e.g., in the absence of genetic engineering or other manipulation designed to alter gene expression) occurs in and is typically restricted to pluripotent stem cells, and is crucial for their functional identity as such. It will be appreciated that the polypeptide encoded by a gene functionally associated with pluripotency may be present as a maternal factor in the oocyte. The gene may be expressed by at least some cells of the embryo, e.g., throughout at least a portion of the preimplantation period and/or in germ cell precursors of the adult.

The term “pluripotency factor” is used refer to the expression product of pluripotency associated gene, e.g., a polypeptide encoded by the gene. In some embodiments, the pluripotency factor is one that is normally substantially not expressed in somatic cell types that constitute the body of an adult animal (with the exception of germ cells or precursors thereof). For example, the pluripotency factor may be one whose average level in ES cells is at least 50-fold or 100-fold greater than its average level in those terminally differentiated cell types present in the body of an adult mammal. In some embodiments, the pluripotency factor is one that is essential to maintain the viability or pluripotent state of ES cells in vivo and/or ES cells derived using conventional methods. Thus, if the gene encoding the factor is knocked out or inhibited (i.e., its expression is eliminated or substantially reduced), the ES cells are not formed, die or, in some embodiments, differentiate. In some embodiments, inhibiting expression of a gene whose function is associated with pluripotency in an ES cell (resulting in, e.g., a reduction in the average steady state level of RNA transcript and/or protein encoded by the gene by at least 50%, 60%, 70%, 80%, 90%, 95%, or more) results in a cell that is viable but no longer pluripotent. In some embodiments the gene is characterized in that its expression in an ES cell decreases (resulting in, e.g., a reduction in the average steady state level of RNA transcript and/or protein encoded by the gene by at least 50%, 60%, 70%, 80%, 90%, 95%, or more) when the cell differentiates into a terminally differentiated cell.

A “pluripotency inducing gene” as used herein, refers to a gene whose expression, contributes to reprogramming somatic cells to a pluripotent state. “Pluripotency inducing factor” refers to an expression product of a pluripotency inducing gene. A pluripotency inducing factor may, but need not be, a pluripotency factor. Expression of an exogenously introduced pluripotency inducing factor may be transient, i.e., it may be needed during at least a portion of the reprogramming process in order to induce pluripotency and/or establish a stable pluripotent state but afterwards not required to maintain pluripotency. For example, the factor may induce expression of endogenous genes whose function is associated with pluripotency. These genes may then maintain the reprogrammed cells in a pluripotent state.

“Polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

### 7.2 Abbreviations

A list of abbreviations used in the present disclosure is provided in Table 1 below.

TABLE 1
Abbreviations

| Abbreviations | Definitions |
|---|---|
| bFGF | Basic fibroblastic growth factor |
| bp | Base pairs |
| Complete RPMI medium | RPMI + 10% FBS + 1x Pen/Strep |
| CRISPR | Clustered regularly interspaced short |
| | palindromic repeats |
| crRNA | Crispr RNA |
| DAPI | 4′,6-diamidino-2-phenylindole |
| ddH₂O | Double distilled water |
| DMEM | Dulbecco's Modified Eagle Medium |
| DPBS | Dulbecco's phosphate buffered saline |
| ECM | Extracellular matrix |
| FACS | Fluorescence-activated cell sorting |
| FACS buffer | DPBS + 2% FBS |
| 6-FAM | 6-Carboxyfluorescein |
| FBS | Foetal bovine serum |
| FMO | Fluorescence minus one |
| FP/F | Forward Primer |
| gRNA | Guide RNA |
| shRNA | Short hairpin RNA |
| IU | International Units |
| IDTE | Integrated DNA Technologies |
| KLF4 | Kruppel-like factor 4 |
| KO | Knock out or knocked out |
| mAb | Monoclonal antibody |
| MEF | Mouse embryonic fibroblasts |
| Oct-3 | Octamer-binding transcription factor-3 |
| PCR | Polymerase Chain Reaction |
| Pen/Strep | Penicillin and streptomycin |
| PBMCs | Peripheral blood mononuclear cells |
| rhIL-2 | Recombinant human Interleukin-2 |
| rhIL-15 | Recombinant human Interleukin-15 |
| rpm | Revolutions per minute |
| RPMI | Roswell Park Memorial Institute medium |
| RV/R | Reverse primer |
| SeV | Sendai virus |
| SSEA-4 | Stage-specific embryonic antigen-4 |
| MOI | Multiplicity of Infection |
| SOX2 | Sex determining region Y-box 2 |
| TRG | T cell receptor gamma locus |
| TRD | T cell receptor delta locus |
| Tg | Transgenic |
| x g | Gravity (or g-force) |
| V | Voltage |
| Zol | Zoledronic acid monohydrate |

### 7.3 Cells for Engineering Hypoimmunogenicity

Cells for use in the methods of the present disclosure can come from all cells and tissues, and particularly mammalian cells and tissues. Suitable cells may have human, ape, monkey, porcine, or rodent origin and may be primary cells or cultured cells. In some embodiments, the cells that are modified using the methods of the present disclosure are human cells.

It should be noted that all cell types are contemplated herein, and preferred cell types include immune cells, such as T cells, natural killer (NK) cells, and B cells, and induced pluripotent stem cells (iPSCs). Other suitable cells include bone marrow stem cells and adult reserve stem cells. In some embodiments, the cells that are modified using the methods of the present disclosure are T cells. In some embodiments, the cells that are modified using the methods of the present disclosure are NK cells. In some embodiments, the cells that are modified using the methods of the present disclosure are iPSCs. In some embodiments, the cells that are modified using the methods of the present disclosure are hematopoietic stem cells (HSCs).

In some embodiments, T cells that are modified using methods of the present disclosure are alpha-beta T cells. In some embodiments, T cells that are modified using hypoimmunogenicity engineering methods of the present disclosure are gamma-delta T cells. In some embodiments, T cells comprise CD8⁺ T cells, and/or CD4⁺ T cells.

### Isolation/Enrichment of Donor Cells

In some embodiments, cells used in the methods of the present disclosure are obtained from a donor. The cells may be allogeneic or non-autologous (“non-self”) with respect to the recipient to whom the cells are administered. In some embodiments, the cells are obtained from a mammalian subject. In other embodiments, the cells are obtained from a primate subject. In some embodiments, the cells are obtained from a human subject.

In some embodiments, the cells used in the methods of the present disclosure are lymphocytes (e.g., T cells, NK cells). Lymphocytes can be obtained from sources such as, but not limited to, peripheral blood mononuclear cells (PBMCs), bone marrow, lymph nodes tissue, cord blood, thymus issue, tissue from a site of infection, ascites, pleural effusion, spleen tissue, and tumors. Lymphocytes may also be generated by differentiation of stem cells. In some embodiments, lymphocytes can be obtained from blood collected from a subject using techniques generally known to the skilled person, such as sedimentation, e.g., FICOLL™ separation.

Cells from the circulating blood of a subject can be obtained by apheresis. An apheresis device typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

T cells can be isolated from PBMCs by lysing the red blood cells and depleting the monocytes. As an example, T cells can be sorted by centrifugation through a PERCOLL™ gradient. In some embodiments, after isolation of PBMC, both cytotoxic and helper T lymphocytes can be sorted into naïve, memory, and effector T cell subpopulations either before or after activation, expansion, and/or genetic modification.

In some embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof can be enriched using either positive or negative selection techniques.

In some embodiments, the immune cells (e.g., T cells, NK cells) can also be differentiated from stem cells, such as cord blood stem cells, progenitor cells, bone marrow stem cells, hematopoietic stem cells (HSCs) and induced pluripotent stem cells (iPSCs).

### 7.4 Methods of Hypoimmunogenicity

The inventors provide herein, inter alia, methods of hypoimmunogenicity, such as bioengineering methodologies and materials, including hypoimmunogenicity (such as engineering hypoimmunogenicity) methodologies and materials useful in, for example, genetically modifying and/or otherwise altering at least one target gene or gene product, processes for producing hypoimmunogenic cells (such as engineered hypoimmunogenic cells), manufacturing of hypoimmunogenic cellular compositions (such as engineered hypoimmunogenic cellular compositions), hypoimmunogenic cell systems (such as engineered hypoimmunogenic cell systems) and uses thereof. In one aspect, provided herein is a method of hypoimmunogenicity (such as engineering hypoimmunogenicity).

In some embodiments, the immunogenic cell is a rodent, porcine, monkey, primate, ape, or human immunogenic cell. In some embodiments, the immunogenic cell is an immunogenic human cell.

In some embodiments, the method comprises genetically modifying (e.g., genetically modifying as disclosed in Section 7.5) at least one target gene (e.g., a regulatory factor X (RFX) gene, a B2M gene, a CD58 gene, a CIITA gene, a TNFRSF14 gene, a TNFRSF1A gene, a TNFRSF1B gene, an ICAM1 gene) of at least one human cell or cell. In some embodiments, genetically modifying the at least one target gene reduces expression of the protein encoded by the at least one target gene in the human cell or cell. In some embodiments, genetically modifying the at least one target gene results in a cell or human cell having hypoimmunogenicity.

In some embodiments, the method further comprises subjecting the genetically modified human cell or genetically modified cell to an immune system, and determining immunogenicity of the genetically modified human cell or genetically modified cell, wherein the immunogenicity is altered as compared to a human cell or a cell, where the at least one gene is not genetically modified.

In some embodiments, the method further comprises subjecting the genetically modified cell or the genetically modified human cell to an immune system, and determining immunogenicity of the genetically modified cell or genetically modified human cell, wherein the immunogenicity is altered as compared to an unmodified cell or an unmodified human cell, where the only difference between the genetically modified cell or the such genetically modified human cell and the unmodified cell or the unmodified human cell is that the at least one gene is not genetically modified in the cell or the human cell.

In some embodiments, the method further comprises administering the hypoimmunogenic cell, such as the engineered hypoimmunogenic cell, to a subject.

In some embodiments, the method further comprises forming at least one embryoid body or multicellular body from the genetically modified human cell or genetically modified cell to produce at least one hypoimmunogenic cell (such as an engineered hypoimmunogenic cell), subjecting the hypoimmunogenic cell (such as the engineered hypoimmunogenic cell) to an immune system, and determining immunogenicity of the hypoimmunogenic cell (such as the engineered hypoimmunogenic cell), wherein the immunogenicity is altered as compared to an unmodified human cell or an unmodified cell where the at least one target gene is not genetically modified.

In some embodiments, the method further comprises forming at least one embryoid body or multicellular body from the genetically modified cell or the genetically modified human cell to produce at least one hypoimmunogenic cell (such as an engineered hypoimmunogenic cell), subjecting the genetically modified cell or the genetically modified human cell to an immune system, and determining immunogenicity of the genetically modified cell or the genetically modified human cell, wherein the immunogenicity is altered as compared to an unmodified cell or an unmodified human cell, where the only difference between the genetically modified cell, such as a genetically modified human cell, and the unmodified cell or the unmodified human cell, is that the at least one gene is not genetically modified in the unmodified cell or the unmodified human cell.

In some embodiments, the embryoid body is made into a single cell suspension prior to exposing to an immune system for immunogenicity testing. The embryoid body can be made by any method known to one of ordinary skill in the art, such as the methods disclosed in Pettinato et al., Engineering Strategies for the Formation of Embryoid Bodies from Human Pluripotent Stem Cells, Stem Cells and Development, Volume 24, Number 14, 2015. Nonlimiting exemplary methods include suspension culture (e.g., bacterial-grade dish culture or methylcellulose culture), hanging drop culture, conical tube culture, round bottomed 96-well plate culture (including low adherence multiwell plates), spinner bioreactor culture, slow turning lateral vessel, and micromold gel culture.

In some embodiments, the methods further comprise introducing a chimeric antigen receptor (CAR) into the hypoimmunogenic cell (such as the engineered hypoimmunogenic cell), or the iPS human cell, optionally into an endogenous target gene such as RFX, CD58, CIITA, and/or B2M.

In some embodiments, the methods further comprise introducing a CAR into the hypoimmunogenic cells (such as the engineered hypoimmunogenic cells) or the iPS human cells described herein such that the CAR is expressed on the surface of the cells (such as the engineered hypoimmunogenic cells) or the iPS human cells and is detectable by flow cytometry. In some embodiments, the methods further comprise using a gRNA to knock-in a transgene containing a promoter and CAR into a target gene (e.g., one or more of a RFX gene, a CD58 gene, a CIITA gene, and/or a B2M gene) resulting in CAR expression on surface of the hypoimmunogenic cell (such as the engineered hypoimmunogenic cell) or the iPS human cell that can be detected by flow cytometry.

In some embodiments, the methods further comprise knocking out one or more target genes in the hypoimmunogenic cell (such as the engineered hypoimmunogenic cell) or the iPS human cell or the iPS cell, e.g., via a gRNA, optionally while knocking in a transgene containing a promoter and CAR into a target gene (e.g., one or more of a RFX gene, a CD58 gene, a CIITA gene, and/or a B2M gene). In some embodiments, the methods further comprise introduction of a dual CAR and target gene miR-shRNA expression system as described herein that enables expression of a CAR and knockdown of an endogenous target gene (e.g., one or more of a RFX gene, a CD58 gene, a CIITA gene, and/or a B2M gene) from a single vector such that the CAR is detectable on the surface of the hypoimmunogenic cell (such as the engineered hypoimmunogenic cell) by flow cytometry. In some embodiments, the gRNA targets RFX5 and is used to knock-in a miR-adapted shRNA that targets CD58. In some embodiments, the miRNA comprises the sequence set forth in SEQ ID NO: 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or 128.

In some embodiments, the methods further comprise knocking out one or more target genes in the hypoimmunogenic cell (such as the engineered hypoimmunogenic cell) or the iPS human cell or the iPS cell, e.g., via a shRNA. In some embodiments, shRNA is used to disrupt the CD58 gene. In some embodiments, the shRNA comprises the sequence set forth in SEQ ID NOs: 60, 61, 62, 63, 64, 65, 66, or 67. In some embodiments, the shRNA comprises the sequence set forth in SEQ ID NOs: 60, 63, or 64.

In some embodiments, the human cell is an immunogenic human cell. In some embodiments, the human cell is an induced pluripotent stem (iPS) human cell reprogrammed from an immunogenic human cell.

In some embodiments, the hypoimmunogenic cell (such as the engineered hypoimmunogenic cell) is a T cell. In some embodiments, the hypoimmunogenic cell (such as the engineered hypoimmunogenic cell) is a T effector cell. In some embodiments, the hypoimmunogenic cell (such as the engineered hypoimmunogenic cell) is not a T regulatory cell. In some embodiments, the hypoimmunogenic cell (such as the engineered hypoimmunogenic cell) does not have a C45RA⁺CD27⁻CD28⁻CCR7⁻CD62L⁻ phenotype. In some embodiments, the hypoimmunogenic cell (such as the engineered hypoimmunogenic cell) is not a natural killer cell. In some embodiments, the hypoimmunogenic cell (such as the engineered hypoimmunogenic cell) is a hypoimmunogenic human cell (such as the engineered hypoimmunogenic human cell).

In some embodiments, the hypoimmunogenic cell (such as the engineered hypoimmunogenic cell) or the iPS human cell does not comprise a genetically modified, e.g., disrupted or knocked out: a) CISH (Cytokine Inducible SH2 Containing Protein) gene; b) adenosine A2A (ADORA2A) gene; c) TGF beta receptor gene; d) HLA class I gene, e.g., HLA A, B, C, E, F, G; e) HLA class II gene; f) NLRC5 (NOD-Like Receptor Family CARD Domain Containing 5) gene; g) CD38 gene; h) thioredoxin interacting protein (TXNIP) gene; i) ITGB3 (Integrin Subunit Beta 3) gene; j) IL17A gene; k) DGKA (diacylglycerol kinase alpha) gene; 1) DGKZ (diacylglycerol kinase zeta) gene; m) PD1 gene; n) TRGC1 (T-cell receptor gamma constant 1) gene; o) TRGC1 (T-cell receptor gamma constant 2) gene; and/or p) TRDC (T-cell receptor delta constant) gene.

In some embodiments, the hypoimmunogenic cell (such as the engineered hypoimmunogenic cell) or the iPS human cell is not TCR null, for example, is not TCR alpha, beta, gamma and/or delta null. For example, in certain embodiments, the TCR locus, e.g., TCR alpha, beta, gamma or delta locus, is not disrupted or knocked out, for example does not comprise an insertion, e.g., a CAR insertion.

In some embodiments, the hypoimmunogenic cell (such as the engineered hypoimmunogenic cell) or the iPS human cell does not comprise: a) an exogenous NICD (Notch Intracellular Domain) coding sequence, e.g., an NICD1 coding sequence; c) an exogenous CD47 coding sequence or increased CD47 expression relative to the wild type (non-engineered) iPS human cell; d) an exogenous sequence that encodes a cell surface protein that binds on the surface of a phagocytic or cytolytic immune cell, wherein said binding results in activation of the hypoimmunogenic cell (such as the engineered hypoimmunogenic cell), e.g., T-cell; e) an exogenous CR1 coding sequence; f) an exogenous CD24 coding sequence; g) an exogenous DUX4 (Double Homeobox 4) coding sequence; h) an exogenous nucleotide sequence operably linked to a promoter derived from a human FOXP3 gene; i) an exogenous CD3 complex cell surface coding sequence or increased expression of a CD3 complex cell surface gene relative to the wild type (non-engineered) iPS human cell; j) an exogenous NKG2C (Natural-Killer Receptor Group 2, member C) coding sequence or increased expression of NKG2C relative to the wild type (non-engineered) iPS human cell; k) an exogenous NKG2D (Natural-Killer Receptor Group 2, member D) coding sequence or increased expression of NKG2D relative to the wild type (non-engineered) iPS human cell; 1) an exogenous PD-L1 coding sequence or increased expression of PD-L1 relative to the wild type (non-engineered) iPS human cell; m) an exogenous CTLA-4 coding sequence or increased expression of CTLA-4 relative to the wild type (non-engineered) iPS human cell; n) an exogenous CD16 coding sequence or increased expression of CD16 relative to the wild type (non-engineered) iPS human cell; o) an exogenous HLA-A coding sequence; p) an exogenous HLA-B coding sequence; q) an exogenous HLA-C coding sequence; r) an exogenous HLA-D coding sequence; s) an exogenous HLA-E coding sequence; t) an exogenous HLA-F coding sequence; u) an exogenous HLA-G coding sequence; v) an exogenous C1-inhibitor coding sequence; x) an exogenous IL35 coding sequence; and/or y) an IL15/IL15 Receptor alpha (IL15Ra) fusion protein, e.g., an IL15/IL15Ra fusion protein, wherein the IL15Ra portion lacks an intracellular domain.

In some embodiments, the hypoimmunogenic cell (such as the engineered hypoimmunogenic cell) or the iPS human cell comprises a CAR knock-in into an endogenous target gene, e.g., one or more of an RFX gene, a CD58 gene, a CIITA gene, and/or a B2M gene. In some embodiments, the hypoimmunogenic cell (such as the engineered hypoimmunogenic cell) or the iPS human cell comprises a transgene containing a promoter and CAR that has been knocked into one or more of an RFX gene, a CD58 gene, a CIITA gene, and/or a B2M gene resulting in CAR expression on the cell surface such that the CAR can be detected by flow cytometry. In some embodiments, the transgene can be knocked in by using a gRNA as described herein.

In some embodiments, the hypoimmunogenic cell (such as the engineered hypoimmunogenic cell) or the iPS human cell or the iPS cell comprises a knockout of an endogenous target gene, i.e., a knockout of one or more of an RFX gene, a CD58 gene, a CIITA gene, and/or a B2M gene, and a knock-in of a CAR. In some embodiments, the CAR knock-in and target gene knockout are accomplished by introduction of a dual CAR and target gene miR-shRNA expression system as described herein that enables expression of a CAR and knockdown of an endogenous target gene (e.g., one or more of an RFX gene, a CD58 gene, a CIITA gene, and/or a B2M gene) from a single vector. In some embodiments, the gRNA targets RFX5 and is used to knock-in a miR-adapted shRNA that targets CD58. In some embodiments, the miRNA comprises the sequence set forth in SEQ ID NO: 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or 128.

In some embodiments, the hypoimmunogenic cell (such as the engineered hypoimmunogenic cell) or the iPS human cell or the iPS cell comprises a knockout of one or more target genes in the hypoimmunogenic cell (such as the engineered hypoimmunogenic cell) or the iPS human cell or the iPS cell, e.g., via a shRNA. In some embodiments, shRNA is used to disrupt the CD58 gene. In some embodiments, the shRNA comprises the sequence set forth in SEQ ID NOs: 60, 61, 62, 63, 64, 65, 66, or 67. In some embodiments, the shRNA comprises the sequence set forth in SEQ ID NOs: 60, 63, or 64.

### 7.4.1 Target Genes

In some embodiments, the target gene is a regulatory factor X (RFX) gene. In some embodiments, genetically modifying the RFX gene eliminates or reduces the RFX protein expression.

Regulatory factor X (also known in as RFX) refers to members of the regulatory factor X (RFX) family of transcription factors. Human RFX proteins are encoded by RFX genes. Members of RFX gene family includes, but not limited to, RFX5, RFXANK and RFXAP. Human regulatory factor X5 or RFX5 is encoded by RFX5 gene (e.g., NCBI Entrez Gene: 5993). Human regulatory factor X associated ankyrin containing protein or RFXANK is encoded by RFXANK gene (e.g., NCBI Entrez Gene: 8625). Human regulatory factor X associated protein or RFXAP is encoded by RFXAP gene (e.g., NCBI Entrez Gene: 5994). In some embodiments, the methods disclosed herein comprise genetically modifying an RFX gene selected from the group consisting of RFX5, RFXANK and RFXAP.

In some embodiments, the present disclosure provides a method comprising genetically modifying a regulatory factor X (RFX) gene of at least one human cell or at least one cell. In some embodiments, genetically modifying the RFX gene reduces expression of the RFX protein in the human cell or the cell. In some embodiments, genetically modifying the RFX gene results in a cell having hypoimmunogenicity. In some embodiments, the method further comprises subjecting the genetically modified human cell or genetically modified cell to an immune system, and determining immunogenicity of the genetically modified human cell or genetically modified cell, wherein the immunogenicity is altered as compared to a human cell or cell where the at least one gene is not genetically modified. In some embodiments, the only difference between the genetically modified human cell or the genetically modified cell and the human cell or cell where the at least one gene is not genetically modified is that one or more of the RFX gene and/or the B2M gene and/or the CD58 gene and/or the CIITA gene has not been genetically modified in the unmodified human cell or unmodified cell.

In some embodiments, the method further comprises forming at least one embryoid body or multicellular body from the genetically modified human cell or genetically modified cell to produce at least one hypoimmunogenic cell (such as an engineered hypoimmunogenic cell), subjecting the hypoimmunogenic cell (such as the engineered hypoimmunogenic cell) to an immune system, and determining immunogenicity of the hypoimmunogenic cell (such as the engineered hypoimmunogenic cell), wherein the immunogenicity is altered as compared to a human cell or a cell where the RFX gene is not genetically modified. In some embodiments, the only difference between the hypoimmunogenic cell (such as the engineered hypoimmunogenic cell) and the human cell or cell where the at least one gene is not genetically modified is that one or more of the RFX gene and/or the B2M gene and/or the CD58 gene and/or the CIITA gene has not been genetically modified in the unmodified human cell or unmodified cell.

In some embodiments, the method further comprises genetically modifying at least one of a B2M gene, a CD58 gene, a CIITA gene (e.g., genetically modifying the RFX gene and the B2M gene, genetically modifying the RFX gene and the CD58 gene, genetically modifying the RFX gene and the CIITA gene). In some embodiments, the method further comprises genetically modifying at least one of a TNFRSF14 (also known as HVEM) gene, a TNFRSF1A (also known as TNFR1) gene, a TNFRSF1B (also known as TNFR2) gene, and an ICAM1 gene.

In some embodiments, the target gene is a B2M gene. In some embodiments, genetically modifying the B2M gene eliminates or reduces the B2M protein expression.

The terms “beta-2 microglobulin,” “B2M,” or “β2m” refer to the beta chain component of MHC class I molecules. Human beta-2 microglobulin is encoded by the B2M gene (e.g., NCBI Gene ID 567). Expression of beta-2 microglobulin is necessary for assembly and function of MHC class I molecules on the cell surface.

In some embodiments, the present disclosure provides a method comprising genetically modifying a B2M gene of at least one human cell or at least one cell. In some embodiments, genetically modifying the B2M gene reduces expression of the B2M protein in the human cell or the cell. In some embodiments, genetically modifying the B2M gene results in a cell having hypoimmunogenicity. In some embodiments, the method further comprises subjecting the genetically modified human cell or a genetically modified cell to an immune system, and determining immunogenicity of the genetically modified human cell or the genetically modified cell, wherein the immunogenicity is altered as compared to a human cell or a cell where the at least one gene is not genetically modified. In some embodiments, the only difference between the genetically modified human cell or the genetically modified cell and the human cell or cell where the at least one gene is not genetically modified is that one or more of the RFX gene and/or the B2M gene and/or the CD58 gene and/or the CIITA gene has not been genetically modified in the unmodified human cell or unmodified cell.

In some embodiments, the method further comprises forming at least one embryoid body or multicellular body from the genetically modified human cell or the genetically modified cell to produce at least one hypoimmunogenic cell (such as an engineered hypoimmunogenic cell), subjecting the hypoimmunogenic cell (such as the engineered hypoimmunogenic cell) to an immune system, and determining immunogenicity of the hypoimmunogenic cell, wherein the immunogenicity is altered as compared to a human cell or a cell where the B2M gene is not genetically modified. In some embodiments, the only difference between the hypoimmunogenic cell (such as the engineered hypoimmunogenic cell) and the human cell or cell where the at least one gene is not genetically modified is that one or more of the RFX gene and/or the B2M gene and/or the CD58 gene and/or the CIITA gene has not been genetically modified in the unmodified human cell or unmodified cell.

In some embodiments, the method further comprises genetically modifying at least one of a RFX gene, a CD58 gene, and a CIITA gene (e.g., genetically modifying the RFX gene and the B2M gene, genetically modifying the B2M gene and the CD58 gene, genetically modifying the B2M gene and the CIITA gene). In some embodiments, the method further comprises genetically modifying at least one of a TNFRSF14 (also known as HVEM) gene, a TNFRSF1A (also known as TNFR1) gene, a TNFRSF1B (also known as TNFR2) gene, and an ICAM1 gene.

In some embodiments, the target gene is a CD58 gene. In some embodiments, genetically modifying the CD58 gene eliminates or reduces the CD58 protein expression.

As used herein, the terms “CD58” or “LFA-3” refer to a ligand of the T lymphocyte CD2 protein, and functions in adhesion and activation of T lymphocytes. Human CD58 is encoded by CD58 gene (e.g., NCBI Entrez Gene: 965). It is known that Cd2 (the CD58 receptor) is important for monocyte and dendritic cell function (see for example Crawford et al., J Immunol, 1999 Dec. 1; 163(11):5920-8. and Crawford et al., Blood. 2003 Sep. 1; 102(5):1745-52.)

In some embodiments, the present disclosure provides a method comprising genetically modifying a CD58 gene of at least one human cell or at least one cell. In some embodiments, genetically modifying the CD58 gene reduces expression of the CD58 protein in the human cell or the cell. In some embodiments, genetically modifying the CD58 gene results in a cell having hypoimmunogenicity. In some embodiments, the method further comprises subjecting the genetically modified human cell or the genetically modified cell to an immune system, and determining immunogenicity of the genetically modified human cell or the cell, wherein the immunogenicity is altered as compared to a human cell or a cell where the at least one gene is not genetically modified. In some embodiments, the only difference between the genetically modified human cell or the genetically modified cell and the human cell or the cell where the at least one gene is not genetically modified is that one or more of the RFX gene and/or the B2M gene and/or the CD58 gene and/or the CIITA gene has not been genetically modified in the unmodified human cell or unmodified cell.

In some embodiments, the method further comprises forming at least one embryoid body or multicellular body from the genetically modified human cell or the genetically modified cell to produce at least one hypoimmunogenic cell (such as an engineered hypoimmunogenic cell), subjecting the hypoimmunogenic cell (such as the engineered hypoimmunogenic cell) to an immune system, and determining immunogenicity of the hypoimmunogenic cell (such as the engineered hypoimmunogenic cell), wherein the immunogenicity is altered as compared to a human cell where the CD58 gene is not genetically modified. In some embodiments, the only difference between the hypoimmunogenic cell (such as the engineered hypoimmunogenic cell) and the human cell or cell where the at least one gene is not genetically modified is that one or more of the RFX gene and/or the B2M gene and/or the CD58 gene and/or the CIITA gene has not been genetically modified in the unmodified human cell or unmodified cell.

In some embodiments, the method further comprises genetically modifying at least one of a RFX gene, a B2M gene, and a CIITA gene (e.g., genetically modifying the CD58 gene and the B2M gene, genetically modifying the CD58 gene and the RFX gene, genetically modifying the CD58 gene and the CIITA gene). In some embodiments, the method further comprises genetically modifying at least one of a TNFRSF14 (also known as HVEM) gene, a TNFRSF1A (also known as TNFR1) gene, a TNFRSF1B (also known as TNFR2) gene, and an ICAM1 gene.

In some embodiments, the method disclosed herein further comprises genetically modifying a CIITA gene, in addition to at least one of the target gene (e.g., a RFX gene, a B2M gene, and/or a CD58 gene). In some embodiments, genetically modifying the CIITA gene eliminates or reduces the CIITA protein expression. In some embodiments, the method further comprises genetically modifying at least one of a TNFRSF14 (also known as HVEM) gene, a TNFRSF1A (also known as TNFR1) gene, a TNFRSF1B (also known as TNFR2) gene, and an ICAM1 gene.

As used herein, the terms “class II major histocompatibility complex transactivator” or “CIITA” refer to a CIITA protein that is essential for transcriptional activity of the HLA class II promoter. Human CIITA is encoded by CIITA gene (e.g., NCBI Entrez Gene: 4261). Mutations in the CIITA gene have been associated with bare lymphocyte syndrome type II (also known as hereditary MHC class II deficiency or HLA class II-deficient combined immunodeficiency).

### 7.4.2 Immunogenic Cells and Immunogenic Human Cells

In some embodiments, the immunogenic cell is a rodent, porcine, primate, monkey, ape, or human immunogenic cell. In some embodiments, the immunogenic cell is an immunogenic human cell.

In some embodiments, the immunogenic cell is allogeneic or non-MHC matched to cells, receptors, or polypeptides of the immune system to which the engineered hypoimmunogenic cell is administered or subjected to.

In some embodiments, the immunogenic human cell is allogeneic or non-HLA matched to cells, receptors, or polypeptides of the immune system to which the hypoimmunogenic cell (such as an engineered hypoimmunogenic cell), is administered or subjected to.

In some embodiments, the immunogenic cell triggers and/or provides for an immune response. In one aspect, the immunogenic cell provides for an innate immune response, specific or adaptive immune response, or combinations thereof. In another aspect of the invention, the immunogenic cell is allogeneic or non-HLA matched to cells, receptors, or polypeptides of the immune system which it triggers or is provided to. In some embodiments, the immune system is an in vitro immune system. In some embodiments, the immune system is an in vivo immune system. In some embodiments, the immune system is an in vivo immune system of a human subject.

In some embodiments, the immunogenic cell or the immunogenic human cell is a non-immune effector cell. In some embodiments, the immunogenic cell or the immunogenic human cell is an immune effector cell.

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune effector cells are T cells. In some embodiments, the T cells are CD4⁺/CD8⁻, CD4⁻/CD8⁺, CD4⁺/CD8⁺, CD4⁻/CD8⁻, or combinations thereof. In some embodiments, the T cells produce IL-2, TFN, and/or TNF upon binding to the target cells. In some embodiments, the CD8⁺ T cells lyse antigen-specific target cells upon binding to the target cells.

In some embodiments, the immune effector cells are NK cells. In other embodiments, the immune effector cells can be established cell lines, for example, NK-92 cells.

In some embodiments, the immune effector cells are differentiated from a stem cell, such as a hematopoietic stem cell, a pluripotent stem cell, an iPS, or an embryonic stem cell.

### 7.4.3 iPS Cells and iPs Human Cells

In some embodiments, the cell is an induced pluripotent stem (iPS) cell. In some embodiments, the iPS cell is reprogrammed from an immunogenic cell (e.g., an immunogenic cell disclosed herein).

In some embodiments, the human cell is an induced pluripotent stem (iPS) human cell. In some embodiments, the iPS human cell is reprogrammed from an immunogenic human cell (e.g., an immunogenic human cell disclosed herein).

Any suitable methods known in the art can be used for reprogramming immunogenic cells into iPS cells or immunogenic human cells into iPS human cells. In some embodiments, the iPS cells or iPS human cells are produced by the methods disclosed in WO2021/257679 (PCT/US2021/037594) or in US2021/0395697, each of which is incorporated herein by reference in its entirety.

In some embodiments, the iPS cell or iPS human cell is reprogrammed from an immunogenic human cell comprising a heterodimeric T-cell receptor comprising a γ chain and a 6 chain. In some embodiments, the iPS cell or iPS human cell is reprogrammed from an γδT cell. In some embodiments, the iPS cell or iPS human cell has rearrangement genes of TRG and TRD gene loci. In some embodiments, the iPS cell or iPS human cell does not produce PCR products from TCRG and TCRD gene loci.

In some embodiments, the iPS cell or iPS human cell is not derived from an up T cell. In some embodiments, the iPS cell or iPS human cell does not have rearrangement genes of TRA and TRB gene loci. In some embodiments, the iPS cell or iPS human cell does not produce PCR products from TCRA and TCRB gene loci.

In some embodiments, the iPS cell or iPS human cell is negative for a Sendai virus (SeV) vector.

In some embodiments, the iPS cell or iPS human cell is genomically stable with no loss of a chromosome. In some embodiments, the genomic stability of the iPS cell or iPS human cell is determined by Karyotyping analysis.

In some embodiments, the iPS cell or iPS human cell can grow and maintain in feeder free medium after adoption.

In some embodiments, the iPS cell or iPS human cell expresses one or more reprogramming factors, and comprises a nucleotide sequence encoding rearrangement of TRG and TRD genes. In some embodiments, the reprogramming factors are selected from a group consisting of Oct3/4, Sox2, Klf4, c-Myc, and Lin28. In some embodiments, the reprogramming factors comprise Oct3/4, Sox2, Klf4, and c-Myc. In some embodiments, the reprogramming factors are Oct3/4, Sox2, KLF4, c-Myc, and Lin28. In some embodiments, the reprogramming factors are Oct3/4, Sox2, Klf4, and c-Myc.

In some embodiments, the iPS cell or iPS human cell is a pluripotent cell that expresses one or more reprogramming factors, wherein (i) the pluripotent cell comprises a nucleotide sequence encoding rearrangement of TRG and TRD genes or has rearrangement genes of TRG and TRD gene loci, (ii) the reprogramming factors are selected from a group consisting of Oct3/4, Sox2, Klf4, c-Myc, and Lin28, (iii) the iPS cell or iPS human cell is negative for a Sendai virus (SeV) vector; (iv) the iPS cell or iPS human cell is reprogrammed from an γδT cell, but not from an up T cell; (v) the iPS cell or iPS human cell does not produce PCR products from TCRA and TCRB gene loci; (vi) the iPS cell or iPS human cell is genomically stable with no loss of a chromosome, e.g., as determined by Karyotyping analysis; and/or (vii) the iPS cell or iPS human cell can grow and maintain in feeder free medium after adoption.

Methods for identifying reprogrammed mammalian somatic cells with a less differentiated state or a pluripotent state are known in the art. For example, in some embodiments, reprogrammed somatic cells are identified by selecting for cells that express the appropriate selectable marker. In some embodiments, reprogrammed somatic cells are further assessed for pluripotency characteristics. The presence of pluripotency characteristics indicates that the somatic cells have been reprogrammed to a pluripotent state.

Differentiation status of cells is a continuous spectrum, with terminally differentiated state at one end of this spectrum and de-differentiated state (pluripotent state) at the other end. Reprogramming, as used herein, refers to a process that alters or reverses the differentiation status of a somatic cell, which can be either partially or terminally differentiated. Reprogramming includes complete reversion, as well as partial reversion, of the differentiation status of a somatic cell. In other words, the term “reprogramming,” as used herein, encompasses any movement of the differentiation status of a cell along the spectrum toward a less-differentiated state. For example, reprogramming includes reversing a multipotent cell back to a pluripotent cell, reversing a terminally differentiated cell back to either a multipotent cell or a pluripotent cell. In some embodiments, reprogramming of a somatic cell turns the somatic cell all the way back to a pluripotent state. In some embodiments, reprogramming of a somatic cell turns the somatic cell back to a multipotent state. The term “less-differentiated state,” as used herein, is thus a relative term and includes a completely de-differentiated state and a partially differentiated state.

The term “pluripotency characteristics” refers to many characteristics associated with pluripotency, including, for example, the ability to differentiate into all types of cells and an expression pattern distinct for a pluripotent cell, including expression of pluripotency genes, expression of other ES cell markers, and on a global level, a distinct expression profile known as “stem cell molecular signature” or “stemness.”

Thus, to assess reprogrammed somatic cells for pluripotency characteristics, one may analyze such cells for different growth characteristics and ES cell-like morphology. In some embodiments, cells may be injected subcutaneously into immunocompromised SCID mice to induce teratomas (a standard assay for ES cells). ES-like cells can be differentiated into embryoid bodies (another ES specific feature). Moreover, ES-like cells can be differentiated in vitro by adding certain growth factors known to drive differentiation into specific cell types. Self-renewing capacity, marked by induction of telomerase activity, is another pluripotency characteristics that can be monitored.

In some embodiments, functional assays of the reprogrammed somatic cells may be conducted by introducing them into blastocysts to determine whether the cells are capable of giving rise to all cell types. If the reprogrammed cells are capable of forming a few cell types of the body, they are multipotent; if the reprogrammed cells are capable of forming all cell types of the body including germ cells, they are pluripotent.

In other embodiments, the expression of an individual pluripotency gene in the reprogrammed somatic cells may be examined to assess their pluripotency characteristics.

Additionally, one may assess the expression of other ES cell markers. Stage-specific embryonic 1 5 antigens-1, -3, and -4 (SSEA-1, SSEA-3, SSEA-4) are glycoproteins specifically expressed in early embryonic development and are markers for ES cells (Solter and Knowles, 1978, Proc. Natl. Acad. Sci. USA 75:5565-5569; Kannagi et al., 1983, EMBO J 2:2355-2361).

Elevated expression of the enzyme Alkaline Phosphatase (AP) is another marker associated with undifferentiated embryonic stem cells (Wobus et al., 1984, Exp. Cell 152:212-219; Pease et al., 1990, Dev. Biol. 141:322-352). Other stem/progenitor cells markers include the intermediate neurofilament nestin (Lendahl et al., 1990, Cell 60:585-595; Dah-Istrand et al., 1992, J. Cell Sci. 103:589-597), the membrane glycoprotein prominin/AC133 (Weigmann et al., 1997, Proc. Natl. Acad. USA 94:12425-12430; Corbeil et al., 1998, Blood 91:2625-22626), the transcription factor Tcf-4 (Korinek et al, 1998, Nat. Genet. 19: 379-383; Lee et al., 1999, J. Biol. Chem. 274.1566-1572), and the transcription factor Cdx1 (Duprey et al., 1988, Genes Dev. 2:1647-1654; Subramania'n et al., 1998, Differentiation 64:11-18).

In some embodiments, expression profiling of the reprogrammed somatic cells may be used to assess their pluripotency characteristics. Pluripotent cells, such as embryonic stem cells, and multipotent cells, such as adult stem cells, are known to have a distinct pattern of global gene expression profile. This distinct pattern is termed “stem cell molecular signature”, or “stemness”. See, for example, Ramalho-Santos et al., Science 298: 597-600 (2002); Ivanova et al., Science 298: 601-604.

Somatic cells may be reprogrammed to gain either a complete set of the pluripotency characteristics and are thus pluripotent. Alternatively, somatic cells may be reprogrammed to gain only a subset of the pluripotency characteristics. In another alternative, somatic cells may be reprogrammed to be multipotent.

### 7.4.4 Hypoimmunogenicity

In some embodiments, immunogenicity of the hypoimmunogenic cell, (such as the engineered hypoimmunogenic cell) is determined by subjecting the cells to an immune system. In some embodiments, the immunogenicity is altered as compared to a human cell (e.g., an immunogenic cell or an iPS human cell) or a cell where the at least one target gene is not genetically modified. In some embodiments, the only difference between the genetically modified human cell or the genetically modified cell and the unmodified human cell or the unmodified cell is that the at least one target gene is not genetically modified in the unmodified human cell or the unmodified cell.

In some embodiments, the hypoimmunogenic cell (such as the engineered hypoimmunogenic cell) is administered to an allogeneic or non-MHC matched subject. In some embodiments, the hypoimmunogenic cell (such as the engineered hypoimmunogenic cell) is administered to an allogeneic or non-HLA matched subject.

In some embodiments, altering the immunogenicity comprises balancing, reducing, or neutralizing the immunogenicity (such as reducing or neutralizing the immunogenicity) or the immune response as compared to an unmodified cell or a population of unmodified cells (e.g., compared to immunogenic human cells or iPS human cells where the at least one target gene is not genetically modified). In some embodiments, the only difference between the genetically modified cell or genetically modified population of modified cells and the genetically unmodified cell or population of genetically unmodified cells is that the at least one target gene is not genetically modified in the unmodified cell or the population of unmodified cells (e.g., compared to immunogenic cells or iPS cells where the at least one target gene is not genetically modified).

In some embodiments, the reduced immunogenicity of the hypoimmunogenic cell (such as the engineered hypoimmunogenic cell) comprises one or more of the following: i) a reduced or ablated myeloid cell response to the hypoimmunogenic cell (such as an engineered hypoimmunogenic cell) upon the cell's presence in an allogeneic or non-MHC matched subject, as compared to a cell corresponding to the cell that was modified but without said genetic modification(s); ii) a reduced or ablated T cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iii) a reduced or ablated natural killer (NK) cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iv) a reduced or ablated neutralizing antibody response to the hypoimmunogenic cell (such as the engineered hypoimmunogenic cell) upon the cell's presence in an allogeneic or non-MHC matched subject, as compared to a cell corresponding to the cell that was modified but without said genetic modification(s); v) a reduced or ablated MHC class II mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); vi) a reduced or ablated neutralizing MHC class I mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); and vii) a reduced or ablated allogeneic host versus graft rejection of to the hypoimmunogenic cell (such as the engineered hypoimmunogenic cell) upon the cell's presence in an allogeneic subject, as compared to a cell corresponding to the cell that was modified but without said genetic modification(s).

In some embodiments, a population of hypoimmunogenic cells (such as engineered hypoimmunogenic cells) of the disclosure have reduced immunogenicity or reduced immune response by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower) as compared to a population of unmodified cells (e.g., compared to cells where the at least one target gene is not genetically modified). In some embodiments, the only difference between the population of hypoimmunogenic cells (such as engineered hypoimmunogenic cells) and the population of unmodified cells is that at least one target gene is not genetically modified in the population of unmodified cells (e.g., compared to cells where the at least one target gene is not genetically modified).

In some embodiments, altering the immunogenicity comprises reducing or neutralizing a myeloid cell response to the hypoimmunogenic cells (such as engineered hypoimmunogenic cells) (e.g., cells having at least one target gene genetically modified). In some embodiments, a population of hypoimmunogenic cells (such as engineered hypoimmunogenic cells) of the disclosure (e.g., cells having at least one target gene genetically modified) have reduced myeloid cell response by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower) as compared to a population of unmodified cells (e.g., compared to cells where the at least one target gene is not genetically modified). In some embodiments, the only difference between the population of hypoimmunogenic cells (such as engineered hypoimmunogenic cells) and the population of unmodified cells is that at least one target gene is not genetically modified in the population of unmodified cells (e.g., cells where the at least one target gene is not genetically modified).

In some embodiments, altering the immunogenicity comprises reducing or neutralizing a T cell response to the hypoimmunogenic cells (such as engineered hypoimmunogenic cells) (e.g., cells having at least one target gene genetically modified). In some embodiments, a population of hypoimmunogenic cells (such as engineered hypoimmunogenic cells) of the disclosure (e.g., cells having at least one target gene genetically modified) have reduced T cell response by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that at least one target gene is not genetically modified in the population of unmodified cells (e.g., cells where the at least one target gene is not genetically modified).

In some embodiments, altering the immunogenicity comprises reducing or neutralizing a natural killer cell response to the hypoimmunogenic cells (such as engineered hypoimmunogenic cells) (e.g., cells having at least one target gene genetically modified). In some embodiments, a population of hypoimmunogenic cells (such as engineered hypoimmunogenic cells) of the disclosure (e.g., cells having at least one target gene genetically modified) have reduced natural killer cell response by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that at least one target gene is not genetically modified in the population of unmodified cells (e.g., cells where the at least one target gene is not genetically modified).

In some embodiments, altering the immunogenicity comprises reducing or neutralizing an antibody response to the hypoimmunogenic cells (such as engineered hypoimmunogenic cells) (e.g., cells having at least one target gene genetically modified). In some embodiments, a population of hypoimmunogenic cells (such as engineered hypoimmunogenic cells) of the disclosure (e.g., cells having at least one target gene genetically modified) have reduced antibody response by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that at least one target gene is not genetically modified in the population of unmodified cells (e.g., cells where the at least one target gene is not genetically modified).

In some embodiments, altering the immunogenicity comprises reducing or neutralizing an allogeneic host versus graft rejection. In some embodiments, a population of hypoimmunogenic cells (such as engineered hypoimmunogenic cells) of the disclosure (e.g., cells having at least one target gene genetically modified) have reduced allogeneic host versus graft rejection by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that at least one target gene is not genetically modified in the population of unmodified cells (e.g., cells where the at least one target gene is not genetically modified).

In some embodiments, the method comprises genetically modifying the RFX gene. In some embodiments, altering the immunogenicity comprises reducing or ablating MHC class II mediated response to the hypoimmunogenic cell (such as engineered hypoimmunogenic cells) (e.g., cells having genetically modified RFX gene). In some embodiments, a population of hypoimmunogenic cells (such as engineered hypoimmunogenic cells) of the disclosure (e.g., cells having genetically modified RFX gene) have reduced MHC class II mediated response by about or at least about 50%, 55%, 60%, 65%, 70%, 75%, 80%, 85%, 90%, 95%, 100% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that the RFX gene is not genetically modified in the population of unmodified cells (e.g., cells where the at least one target gene is not genetically modified).

In some embodiments, altering the immunogenicity comprises reducing or neutralizing MHC class I mediated response to the hypoimmunogenic cells (such as engineered hypoimmunogenic cells) (e.g., cells having genetically modified RFX gene). In some embodiments, a population of hypoimmunogenic cells (such as engineered hypoimmunogenic cells) of the disclosure have reduced MHC class I mediated response by about or at least about 5%, 10%, 15%, 20%, 25%, 30%, 35%, 40%, 45%, 50%, 55%, 60%, 65%, 70%, 75%, 80%, 85%, 90%, 95% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that the RFX gene is not genetically modified in the population of unmodified cells (e.g., cells where the at least one target gene is not genetically modified).

In some embodiments, expression of HLA class II molecules (e.g., HLA-DP, HLA-DM, HLA-DOA, HLA-DOB, HLA-DQ, and HLA-DR) is reduced (e.g., partially or completely) or ablated in the presently disclosed hypoimmunogenic cell (such as engineered hypoimmunogenic cells) (e.g., cells having genetically modified RFX gene). In some embodiments, expression of the HLA class II molecules is not detected in a population of genetically modified cells of the disclosure (e.g., not detected by a conventional method (e.g., FACS)). In some embodiments, the expression of the HLA class II molecules in a population of genetically modified cells (e.g., cells having genetically modified RFX gene) is reduced by about or at least about 50%, 55%, 60%, 65%, 70%, 75%, 80%, 85%, 90%, 95%, 100% (lower) as compared to the expression of HLA class II molecules in a population of unmodified cells (e.g., cells where the at least one target gene is not genetically modified). In some embodiments, the only difference between the population of hypoimmunogenic cells (such as engineered hypoimmunogenic cells) and the population of unmodified cells is that the RFX gene is not genetically modified in the population of unmodified cells (e.g., cells where the at least one target gene is not genetically modified).

In some embodiments, expression of HLA-A, HLA-B, and/or HLA-C is reduced (e.g., partially) in the presently disclosed hypoimmunogenic cell (such as engineered hypoimmunogenic cells) (e.g., cells having genetically modified RFX gene). In some embodiments, the expression of HLA-A in a population of genetically modified cells (e.g., cells having genetically modified RFX gene) is reduced by about or at least about 5%, 10%, 15%, 20%, 25%, 30%, 35%, 40%, 45%, 50%, 55%, 60%, 65%, 70%, 75%, 80%, 85%, 90%, 95% (lower) as compared to the expression of HLA-A in a population of unmodified cells (e.g., cells where the at least one target gene is not genetically modified). In some embodiments, the only difference between the population of hypoimmunogenic cells (such as engineered hypoimmunogenic cells) and the population of unmodified cells is that the RFX gene is not genetically modified in the population of unmodified cells (e.g., cells where the at least one target gene is not genetically modified). In some embodiments, the expression of HLA-B in a population of genetically modified cells (e.g., cells having genetically modified RFX gene) is reduced by about or at least about 5%, 10%, 15%, 20%, 25%, 30%, 35%, 40%, 45%, 50%, 55%, 60%, 65%, 70%, 75%, 80%, 85%, 90%, 95% (lower) as compared to the expression of HLA-B in a population of unmodified cells (e.g., cells where the at least one target gene is not genetically modified). In some embodiments, the only difference between the population of hypoimmunogenic cells (such as engineered hypoimmunogenic cells) and the population of unmodified cells is that the RFX gene is not genetically modified in the population of unmodified cells (e.g., cells where the at least one target gene is not genetically modified). In some embodiments, the expression of HLA-C in a population of genetically modified cells (e.g., cells having genetically modified RFX gene) is reduced by about or at least about 5%, 10%, 15%, 20%, 25%, 30%, 35%, 40%, 45%, 50%, 55%, 60%, 65%, 70%, 75%, 80%, 85%, 90%, 95% (lower) as compared to the expression of HLA-C in a population of unmodified cells (e.g., cells where the at least one target gene is not genetically modified). In some embodiments, the only difference between the population of hypoimmunogenic cells (such as engineered hypoimmunogenic cells) and the population of unmodified cells is that the RFX gene is not genetically modified in the population of unmodified cells (e.g., cells where the at least one target gene is not genetically modified).

In some embodiments, expression of HLA-E is reduced (e.g., partially) in the presently disclosed hypoimmunogenic cell (such as engineered hypoimmunogenic cells) (e.g., cells having genetically modified RFX gene). In some embodiments, expression of HLA-E remains detectable (e.g., by FACS). In some embodiments, the expression of HLA-E in a population of genetically modified cells (e.g., cells having genetically modified RFX gene) is reduced by about or at least about 5%, 10%, 15%, 20%, 25%, 30%, 35%, 40%, 45%, 50%, 55%, 60%, 65%, 70%, 75%, 80%, 85%, 90%, 95% (lower) as compared to the expression of HLA-E in a population of unmodified cells (e.g., cells where the at least one target gene is not genetically modified). In some embodiments, the only difference between the population of hypoimmunogenic cells (such as engineered hypoimmunogenic cells) and the population of unmodified cells is that the RFX gene is not genetically modified in the population of unmodified cells (e.g., cells where the at least one target gene is not genetically modified).

In some embodiments, the method comprises genetically modifying the B2M gene. In some embodiments, altering the immunogenicity comprises reducing or ablating MHC class I mediated response to the hypoimmunogenic cell (such as engineered hypoimmunogenic cells) (e.g., cells having genetically modified B2M gene). In some embodiments, a population of hypoimmunogenic cells (such as engineered hypoimmunogenic cells) of the present disclosure (e.g., cells having genetically modified B2M gene) have reduced MHC class I mediated response by about or at least about 50%, 55%, 60%, 65%, 70%, 75%, 80%, 85%, 90%, 95%, 100% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that the B2M gene is not genetically modified in the population of unmodified cells (e.g., cells where the at least one target gene is not genetically modified).

In some embodiments, expression of HLA class I molecules (e.g., HLA-A, HLA-B, HLA-C, or HLA-E) is reduced (e.g., partially or completely), ablated, or non-detectable (e.g., by FACS) in the presently disclosed genetically modified hypoimmunogenic cells (such as engineered hypoimmunogenic cells) (e.g., cells having genetically modified B2M gene). In some embodiments, the expression of HLA-A in a population of genetically modified cells (e.g., cells having genetically modified B2M gene) is reduced by about or at least about 50%, 55%, 60%, 65%, 70%, 75%, 80%, 85%, 90%, 95%, 100% (lower) as compared to the expression of HLA-A in a population of unmodified cells (e.g., cells where the at least one target gene is not genetically modified). In some embodiments, the only difference between the population of hypoimmunogenic cells (such as engineered hypoimmunogenic cells) and the population of unmodified cells is that the B2M gene is not genetically modified in the population of unmodified cells (e.g., cells where the at least one target gene is not genetically modified). In some embodiments, the expression of HLA-B in a population of genetically modified cells (e.g., cells having genetically modified B2M gene) is reduced by about or at least about 50%, 55%, 60%, 65%, 70%, 75%, 80%, 85%, 90%, 95%, 100% (lower) as compared to the expression of HLA-B in a population of unmodified cells (e.g., cells where the at least one target gene is not genetically modified). In some embodiments, the only difference between the population of hypoimmunogenic cells (such as engineered hypoimmunogenic cells) and the population of unmodified cells is that the B2M gene is not genetically modified in the population of unmodified cells (e.g., cells where the at least one target gene is not genetically modified). In some embodiments, the expression of HLA-C in a population of genetically modified cells (e.g., cells having genetically modified B2M gene) is reduced by about or at least about 50%, 55%, 60%, 65%, 70%, 75%, 80%, 85%, 90%, 95%, 100% (lower) as compared to the expression of HLA-C in a population of unmodified cells. In some embodiments, the expression of HLA-E in a population of genetically modified cells (e.g., cells having genetically modified B2M gene) is reduced by about or at least about 50%, 55%, 60%, 65%, 70%, 75%, 80%, 85%, 90%, 95%, 100% (lower) as compared to the expression of HLA-E in a population of unmodified cells (e.g., cells where the at least one target gene is not genetically modified). In some embodiments, the only difference between the population of hypoimmunogenic cells (such as engineered hypoimmunogenic cells) and the population of unmodified cells is that the B2M gene is not genetically modified in the population of unmodified cells (e.g., cells where the at least one target gene is not genetically modified).

In some embodiments, the method comprises genetically modifying the CD58 gene. In some embodiments, genetically modifying the CD58 gene alters the immunogenicity in the cells. In some embodiments, genetically modifying the CD58 gene reduces or ablates a costimulatory immune cell response. In some embodiments, genetically modifying the CD58 gene impairs the formation of an immune synapse. In some embodiments, genetically modifying the CD58 gene leads to impaired recognition by patient (host) T cells, NK cells, and myeloid cells. In some embodiments, a population of hypoimmunogenic cells (such as engineered hypoimmunogenic cells) of the present disclosure (e.g., cells having genetically modified CD58 gene) have reduced costimulatory immune cell response by about or at least about 5%, 10%, 15%, 20%, 25%, 30%, 35%, 40%, 45%, 50%, 55%, 60%, 65%, 70%, 75%, 80%, 85%, 90%, 95%, 100% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that the CD58 gene is not genetically modified in the population of unmodified cells (e.g., cells where the at least one target gene is not genetically modified). In some embodiments, a population of hypoimmunogenic cells (such as engineered hypoimmunogenic cells) of the present disclosure (e.g., cells having genetically modified CD58 gene) have reduced formation of immune synapse by about or at least about 5%, 10%, 15%, 20%, 25%, 30%, 35%, 40%, 45%, 50%, 55%, 60%, 65%, 70%, 75%, 80%, 85%, 90%, 95%, 100% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that the CD58 gene is not genetically modified in the population of unmodified cells (e.g., cells where the at least one target gene is not genetically modified).

In some embodiments, the method comprises further genetically modifying the CIITA gene, in combination with genetically modifying at least one of RFX gene, B2M gene, and CD58 gene. In some embodiments, genetically modifying the CIITA gene further alters the immunogenicity in the cells. In some embodiments, altering the immunogenicity comprises reducing or ablating MHC class II mediated response to the hypoimmunogenic cells (such as engineered hypoimmunogenic cells) (e.g., cells having genetically modified CIITA gene). In some embodiments, a population of hypoimmunogenic cells (such as engineered hypoimmunogenic cells) of the present disclosure have reduced MHC class II mediated response by about or at least about 50%, 55%, 60%, 65%, 70%, 75%, 80%, 85%, 90%, 95%, 100% (lower) as compared to a population of unmodified cells (e.g., cells where the at least one target gene is not genetically modified). In some embodiments, the only difference between the population of hypoimmunogenic cells (such as engineered hypoimmunogenic cells) and the population of unmodified cells is that the CIITA gene is not genetically modified in the population of unmodified cells (e.g., cells where the at least one target gene is not genetically modified).

In some embodiments, expression of HLA class II molecules (e.g., HLA-DP, HLA-DM, HLA-DOA, HLA-DOB, HLA-DQ, and HLA-DR) is further reduced (e.g., partially completely) or ablated in the presently disclosed hypoimmunogenic cells (such as engineered hypoimmunogenic cells) (e.g., cells having genetically modified CIITA gene). In some embodiments, expression of the HLA class II molecules is not detected in a population of genetically modified cells of the disclosure (e.g., not detected by a conventional method (e.g., FACS)). In some embodiments, the expression of the HLA class II molecules in a population of genetically modified cells (e.g., cells having genetically modified CIITA gene) is reduced by about or at least about 50%, 55%, 60%, 65%, 70%, 75%, 80%, 85%, 90%, 95%, 100% (lower) as compared to the expression of HLA class II molecules in a population of unmodified cells (e.g., cells where the at least one target gene is not genetically modified). In some embodiments, the only difference between the genetically modified cells and the population of unmodified cells is that the CIITA gene is not genetically modified in the population of unmodified cells (e.g., cells where the at least one target gene is not genetically modified).

In some embodiments, the reduced immunogenicity of the hypoimmunogenic cell (such as the engineered hypoimmunogenic cell) comprises one or more of the following: i) a reduced or ablated myeloid cell response to the hypoimmunogenic cell (such as an engineered hypoimmunogenic cell) upon the cell's presence in an allogeneic or non-MHC matched subject, as compared to a cell corresponding to the cell that was modified but without said genetic modification(s); ii) a reduced or ablated T cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iii) a reduced or ablated natural killer (NK) cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iv) a reduced or ablated neutralizing antibody response to the hypoimmunogenic cell (such as the engineered hypoimmunogenic cell) upon the cell's presence in an allogeneic or non-MHC matched subject, as compared to a cell corresponding to the cell that was modified but without said genetic modification(s); v) a reduced or ablated MHC class II mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); vi) a reduced or ablated neutralizing MHC class I mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); and vii) a reduced or ablated allogeneic host versus graft rejection of to the hypoimmunogenic cell (such as the engineered hypoimmunogenic cell) upon the cell's presence in an allogeneic subject, as compared to a cell corresponding to the cell that was modified but without said genetic modification(s).

In some embodiments, in the hypoimmunogenic cell (such as the engineered hypoimmunogenic cell): i) expression of HLA class II molecules is reduced or ablated; ii) expression of HLA-A, HLA-B, and/or HLA-C is reduced; and iii) expression of HLA-E is reduced but remains detectable.

In some embodiments, expressions of HLA class I and II molecules are detected by FACS.

In some embodiments, cells are assessed for immunogenicity using any suitable method known to a skilled artisan. In some embodiments, a cell is analyzed for the presence of antibodies on the cell surface, e.g., by staining with an anti-IgM antibody. In some embodiments, immunogenicity is assessed by a PBMC cell lysis assay. In some embodiments, a population of cell is incubated with peripheral blood mononuclear cells (PBMCs) and then assessed for lysis of the cells by the PBMCs. In some embodiments, immunogenicity is assessed by a natural killer (NK) cell lysis assay. In some embodiments, a population of cells is incubated with NK cells and then assessed for lysis of the cells by the NK cells. In some embodiments, immunogenicity is assessed by a CD8⁺ T cell lysis assay. In some embodiments, a population of cells is incubated with CD8⁺ T cells and then assessed for lysis of the cells by the CD8⁺ T cells. In some embodiments, a genetically modified cell of the disclosure or a population thereof has increased viability or increased survival rate as compared to an unmodified cell or a population of unmodified cells (e.g., compared to immunogenic human cells or immunogenic cells or iPS human cells or iPS cells where the RFX gene is not genetically modified). In some embodiments, the only difference between the genetically modified cell and the unmodified cell or the population of unmodified cells is that the RFX gene (and optionally the B2M gene and/or the CIITA gene and/or the CD58 gene) is not genetically modified in the unmodified cell or the population of unmodified cells. In some embodiments, a population of genetically modified cells of the disclosure have increased viability or increased survival rate of about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (higher) as compared to a population of unmodified cells (e.g., cells where the at least one target gene is not genetically modified). In some embodiments, the only difference between the genetically modified cell and the unmodified cell or the population of unmodified cells is that one or more of the RFX gene and/or the B2M gene and/or the CIITA gene and/or the CD58 gene is not genetically modified in the population of unmodified cells. In some embodiments, cells are assessed for increased viability or increased survival rate using any suitable method known to a skilled artisan. In some embodiments, cell viability or survival rate is determined using flow cytometry, high content imaging, tetrazolium reduction (MTT) assay, resazurin reduction assay, protease viability marker assay, and/or ATP detection assay.

### 7.4.5 Chimeric Antigen Receptors (CARs) Knock-In Systems

In some embodiments, a chimeric antigen receptor (CAR) can be introduced into the hypoimmunogenic cell (such as the engineered hypoimmunogenic cell) or the iPS human cell, optionally into an endogenous target gene such as RFX, CD58, CIITA, and/or B2M.

In some embodiments, the methods further comprise introducing a CAR into the hypoimmunogenic cells (such as engineered hypoimmunogenic cells) described herein such that the CAR is expressed on the surface of the hypoimmunogenic cells (such as engineered hypoimmunogenic cells) and is detectable by flow cytometry. In some embodiments, the methods further comprise using a gRNA to knock-in a transgene containing a promoter, a CAR and/or a miR-adapted shRNA into an endogenous target gene (e.g., one or more of an RFX gene, a CD58 gene, a CIITA gene, and/or a B2M gene) resulting in CAR expression on surface of the hypoimmunogenic cell (such as the engineered hypoimmunogenic cell) that can be detected by flow cytometry.

In some embodiments, the methods further comprise knocking out one or more target genes, e.g., via a gRNA, miRNA, shRNA, miR-adapted shRNA, or other RNA interference (RNAi)-based method, in combination with the knock-in of a CAR. In some embodiments, the knockout comprises an indel formation resulting in non-functional expression of the gene.

In some embodiments, the methods further comprise introduction of a dual CAR and target gene miR-shRNA expression system as described herein that enables expression of a CAR and knockdown of an endogenous target gene (e.g., one or more of an RFX gene, a CD58 gene, a CIITA gene, and/or a B2M gene) from a single vector such that the CAR is detectable on the surface of the hypoimmunogenic cell (such as the engineered hypoimmunogenic cell) or the iPS cell or the iPS human cell by flow cytometry. In some embodiments, the gRNA is used to knock-in a miR-adapted shRNA that targets CD58. In some embodiments, the gRNA targets RFX5. In some embodiments, the miRNA comprises the sequence set forth in SEQ ID NO: 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or 128.

In some embodiments, the methods further comprise knocking out one or more target genes in the hypoimmunogenic cell (such as the engineered hypoimmunogenic cell) or the iPS human cell or the iPS cell, e.g., via a shRNA. In some embodiments, shRNA is used to disrupt the CD58 gene. In some embodiments, the shRNA comprises the sequence set forth in SEQ ID NOs: 60, 61, 62, 63, 64, 65, 66, or 67. In some embodiments, the shRNA comprises the sequence set forth in SEQ ID NOs: 60, 63, or 64.

In some embodiments, the hypoimmunogenic cell (such as the engineered hypoimmunogenic cell) or the iPS human cell comprises a CAR knock-in into an endogenous target gene, e.g., one or more of an RFX gene, a CD58 gene, a CIITA gene, and/or a B2M gene. In some embodiments, the hypoimmunogenic cell (such as the engineered hypoimmunogenic cell) or the iPS human cell comprises a transgene containing a promoter and CAR that has been knocked into one or more of an RFX gene, a CD58 gene, a CIITA gene, and/or a B2M gene resulting in CAR expression on the cell surface such that the CAR can be detected by flow cytometry. In some embodiments, the transgene can be knocked in by using a gRNA as described herein.

In some embodiments, the hypoimmunogenic cell (such as the engineered hypoimmunogenic cell) or the iPS human cell or the iPS cell comprises a knockout of an endogenous target gene, i.e., a knockout of one or more of an RFX gene, a CD58 gene, a CIITA gene, and/or a B2M gene, and a knock-in of a CAR. In some embodiments, the CAR knock-in and target gene knockout are accomplished by introduction of a dual CAR and target gene miR-shRNA expression system as described herein that enables expression of a CAR and knockdown of an endogenous target gene (e.g., one or more of an RFX gene, a CD58 gene, a CIITA gene, and/or a B2M gene) from a single vector. In some embodiments, the gRNA is used to knock-in a miRNA that targets CD58. In some embodiments, the miRNA comprises the sequence set forth in SEQ ID NO: 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or 128.

In some embodiments, the hypoimmunogenic cell (such as the engineered hypoimmunogenic cell) or the iPS human cell or the iPS cell comprises a knockout of one or more target genes in the hypoimmunogenic cell (such as the engineered hypoimmunogenic cell) or the iPS human cell or the iPS cell, e.g., via a shRNA. In some embodiments, shRNA is used to disrupt the CD58 gene. In some embodiments, the shRNA comprises the sequence set forth in SEQ ID NOs: 60, 61, 62, 63, 64, 65, 66, or 67. In some embodiments, the shRNA comprises the sequence set forth in SEQ ID NOs: 60, 63, or 64.

Challenges in chimeric antigen receptor engineering and some potential options for addressing such challenges are known to the ordinarily skilled artisan and the present engineering approaches include advances in cell engineering including chimeric antigen receptor cellular approaches. See, for example, Sotilo E. et al. Cancer Discov. 2015 5(12): 1282-1295; Gardner R. et al. Blood 2016 127(20): 2406-2410; and Majzner R G et al. Cancer Discov. 2020 May 10(5): 702-723

### 7.5 Genetic Modification

In some embodiments, genetically modifying a target gene (e.g., an REX gene, a B2M gene, a CIITA gene, a CD58 gene) eliminates or reduces expression of the protein encoded by the gene.

In some embodiments, genetically modifying the target gene eliminates expression of the protein encoded by the gene. In some embodiments, genetically modifying the target gene reduces (e.g., partially or completely) expression of the protein encoded by the gene. In some embodiments, expression of the protein encoded by the gene is not detected in a population of genetically modified cells of the disclosure. In some embodiments, the expression of the protein encoded by the gene in a population of genetically modified cells is reduced by about or at least about 50%, 55%, 60%, 65%, 70%, 75%, 80%, 85%, 90%, 95%, 100% (lower) as compared to the expression of the protein encoded by the gene in a population of unmodified cells.

Any suitable methods known in the art can be used for genetically modifying the gene in a cell as disclosed herein (e.g., a cell as disclosed in Section 7.3 or 7.4) a human cell disclosed herein (e.g., an immunogenic human cell, or an iPS human cell disclosed in Section 7.4).

In some embodiments, genetically modifying a target gene comprises modifying the genomic DNA sequence of the gene; repressing transcription or translation of the mRNA of the gene through RNA interference (RNAi) system; or reducing or ablating transcription of the gene through recruiting or directing transcriptional repressors to the gene.

### 7.5.1 Modifying Genomic DNA Sequence

In some embodiments, genetically modifying a target gene comprises modifying the genomic DNA sequence of a target gene. In some embodiments, modifying the genomic DNA sequence of the gene includes methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ). NHEJ directly joins the DNA ends resulting from a double-strand break, sometimes with the loss or addition of nucleotide sequence, which may disrupt gene expression. HDR utilizes a homologous sequence, or donor sequence, as a template for inserting a defined DNA sequence at the break point. The homologous sequence can be in the endogenous genome, such as a sister chromatid. Alternatively, the donor sequence can be an exogenous polynucleotide, such as a plasmid, a single-strand oligonucleotide, a double-stranded oligonucleotide, a duplex oligonucleotide or a virus, that has regions (e.g., left and right homology arms) of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome (Cho and Greenberg, Nature, 2015, 518, 174-76; Kent et al., Nature Structural and Molecular Biology, 2015, 22(3):230-7; Mateos-Gomez et al., Nature, 2015, 518, 254-57; Ceccaldi et al., Nature, 2015, 528, 258-62).

Each of these genome editing mechanisms can be used to create desired genetic modifications. A step in the genome editing process can be to create one or two DNA breaks, the latter as double-strand breaks or as two single-stranded breaks, in the target locus at near the site of intended mutation or alteration. This can be achieved via the use of an endonuclease, as described herein.

In some embodiments, a target gene of the disclosure (e.g., an RFX gene, a B2M gene, a CIITA gene, a CD58 gene) is disrupted or at least partially deleted via a CRISPR-Cas system. In some embodiments, the CRISPR/Cas system that is used to alter target polynucleotide sequences in cells include RNA binding proteins, endo- and exo-nucleases, helicases, and/or polymerases. In some embodiments, the CRISPR-endonuclease system comprises an endonuclease and at least one guide nucleic acid that directs DNA cleavage of the endonuclease by hybridizing to a recognition site (or target motif of a target polynucleotide) in the genomic DNA. In some embodiments, the CRISPR-endonuclease system comprises an endonuclease and at least one ribonucleic acid (e.g., guide RNA (gRNA)) that directs DNA cleavage of the endonuclease by hybridizing to a recognition site (or target motif of a target polynucleotide) in the genomic DNA. In some embodiments, the CRISPR system is a Type I, II, III, IV, V, and/or VI system(s). In some embodiments, the CRISPR system is a Type II CRISPR/Cas9 system. In some embodiments, the CRISPR system is a Type V CRISPR/Cpf1 (or Cas12a) system. In some embodiments, the CRISPR system is a CRISPR-MAD7 system. In some embodiments, the CRISPR system includes an endonuclease, e.g., Cas9, Cpf1, or MAD7, and one or two noncoding RNAs-crisprRNA (crRNA) and trans-activating RNA (tracrRNA) to target the cleavage of DNA.

CRISPR systems, including various guide designs such as those described in the following publications, are known to an ordinarily skilled artisan. Exemplary CRISPR systems are described in WO 2017/106569; WO 2015/139139; Zetsche B et al. Cpf1 is a single RNA-guided endonuclease of a Class 2 CRISPR system. Cell. 2015 Oct. 22; 163(3):759-71; Jedrzejczyk D J et al. CRISPR-Cas12a nucleases function with structurally engineered crRNAs: SynThetic trAcrRNA. Sci Rep. 2022 Jul. 16; 12(1):12193; EP3642334A1; U.S. Pat. Nos. 9,790,490; 11,180,751; US20210348156; EP3502253; EP3283625; U.S. Ser. No. 10/337,028; WO 2019/046540; and WO 2017/127807.

In some embodiments, methods of genome editing of the disclosure uses at least one and/or any ribonucleic acid (e.g., guide RNA or gRNA) that is capable of directing an endonuclease (Cas protein) to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, the CRISPR RNA (crRNA) is or comprises about 17-20 nucleotide sequence complementary to the target DNA (target motif of a target polynucleotide). In some embodiments, tracr RNA serves as a binding scaffold for the endonuclease (e.g., Cas9, Cpf1, MAD7, or any other endonuclease of the disclosure). In some embodiments, a single ribonucleic acid comprises a guide RNA (gRNA) that directs the endonuclease or Cas protein to and hybridizes to a target motif of the target polynucleotide sequence in a cell. In some embodiments, at least one of the ribonucleic acids comprises a guide RNA that directs the endonuclease or Cas protein to and hybridizes to a target motif of the target polynucleotide sequence in a cell. In some embodiments, both of the one to two ribonucleic acids comprise a guide RNA that directs the endonuclease or Cas protein to and hybridizes to a target motif of the target polynucleotide sequence in a cell. In some embodiments, the at least one ribonucleic acid(s) of the present disclosure can be selected to hybridize to a variety of different target motifs, for example, different target motifs within a target polynucleotide. In some embodiments, the at least one ribonucleic acid(s) of the present disclosure can be selected to hybridize to a variety of different target motifs depending on the particular CRISPR/Cas system employed, and the sequence of the target polynucleotide, as will be appreciated by those skilled in the art. In some embodiments, the at least one ribonucleic acid(s) (e.g., one to two ribonucleic acids) can also be selected to minimize hybridization with nucleic acid sequences other than the target polynucleotide sequence. In some embodiments, the at least one ribonucleic acid(s) (e.g., one to two ribonucleic acids) hybridizes to a target motif that contains at least two mismatches when compared with all other genomic nucleotide sequences in the cell. In some embodiments, the at least one ribonucleic acid(s) (e.g., one to two ribonucleic acids) hybridizes to a target motif that contains at least one mismatch when compared with all other genomic nucleotide sequences in the cell. In some embodiments, the at least one ribonucleic acid(s) (e.g., one to two ribonucleic acids) are designed to hybridize to a target motif immediately adjacent to a deoxyribonucleic acid motif recognized by the endonuclease or Cas protein. In some embodiments, the at least one ribonucleic acid(s) (e.g., one to two ribonucleic acids) is designed to hybridize to a target motif immediately adjacent to a deoxyribonucleic acid motif recognized by the endonuclease or Cas protein which flank a mutant allele located between the target motifs.

In some embodiments, methods of genome editing of the disclosure can be used with a tracr RNA. In some embodiments, methods of genome editing of the disclosure can be used without a tracr RNA. In some embodiments, methods of genome editing of the disclosure can be used with discontinuous or split RNAs, such as for example and not limitation, discontinuous or split gRNAs.

In some embodiments, the at least one ribonucleic acid (e.g., guide RNA) is complementary to and/or hybridize to a sequence on the same strand of a target polynucleotide sequence (e.g., an RFX gene, a B2M gene, a CIITA gene, a CD58 gene). In some embodiments, the at least one ribonucleic acid (e.g., guide RNA) is complementary to and/or hybridize to a sequence on the opposite strand of a target polynucleotide sequence. In some embodiments the at least one ribonucleic acid (e.g., guide RNA) is not complementary to and/or do not hybridize to a sequence on the opposite strand of a target polynucleotide sequence. In some embodiments, the at least one ribonucleic acid (e.g., guide RNA) is complementary to and/or hybridize to overlapping target motifs of a target polynucleotide sequence. In some embodiments the at least one ribonucleic acid (e.g., guide RNA) is complementary to and/or hybridize to offset target motifs of a target polynucleotide sequence.

In some embodiments, the at least one ribonucleic acid is complementary to and/or hybridizes to a sequence on the same strand of a target polynucleotide sequence, wherein the target polynucleotide sequence comprises a B2M gene. In some embodiments, the at least one ribonucleic acid is a gRNA. In some embodiments, the target polynucleotide sequence comprises the sequence set forth in SEQ ID NO: 253. In some embodiments, the gRNA comprises the sequence set forth in SEQ ID NO: 129 (UAAUUUCUACUCUUGUAGAU), optionally in combination with a spacer sequence set forth in SEQ ID NO: 251 (AGUGGGGGUGAAUUCAGUGUA). In some embodiments, the gRNA comprises the sequence set forth in SEQ ID NO: 252.

In some embodiments, the at least one ribonucleic acid is complementary to and/or hybridizes to a sequence on the same strand of a target polynucleotide sequence, wherein the target polynucleotide sequence comprises an RFX gene. In some embodiments, the at least one ribonucleic acid is a gRNA. In some embodiments, the gRNA comprises the sequence set forth in SEQ ID NO: 184 (RFX5_Exon9_gRNA 2; AGGAUCCGCUCUGCCCAGUCA), SEQ ID NO: 193 (RFX5_Exon10_gRNA 1; GAUGACCGUUCCCGAGGUGCA), SEQ ID NO: 202 (RFX5_Exon10_gRNA 4; GAGAACCCAGAGGGUGGAGCC), SEQ ID NO: 205 (RFX5_Exon10_gRNA 5; GUACCUCUGCAGAAGAGGACG), SEQ ID NO: 223 (RFX5_Exon11_gRNA 8; AGGGCACCUGAAGAAAGCCUG), SEQ ID NO: 239 (RFX5_Exon9_gRNA 2; AGGAUCCGCUCUGCCCAGUC) or SEQ ID NO: 246 (RFX5_Exon10_gRNA 1; GAUGACCGUUCCCGAGGUGC). In some embodiments, the gRNA comprises the sequence set forth in SEQ ID NO: 239 or 246. In some embodiments, the gRNA targets a genomic region comprising SEQ ID NO: 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 241, 241, or 248. In some embodiments, the gRNA comprises the repeat sequence set forth in SEQ ID NO: 129, 235, or 237. In some embodiments, the gRNA further comprises a spacer sequence set forth in SEQ ID NO: 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 239, or 246. In some embodiments, the gRNA comprises the sequence set forth in SEQ ID NO: 131, 134, 137, 140, 143, 146, 149, 152, 155, 158, 161, 164, 167, 170, 173, 176, 179, 182, 185, 188, 191, 194, 197, 200, 203, 206, 209, 212, 215, 218, 221, 224, 227, 230, 233, 236, 238, 240, 242, 243, 244, 245, 247, 249, or 250. In some embodiments, the target polynucleotide sequence comprises SEQ ID NO: 141, 186, 195, 204, 207, 225, 241, or 248. In some embodiments, the gRNA comprises the repeat sequence set forth in SEQ ID NOs: 129, 235, or 237. In some embodiments, the gRNA further comprises a spacer sequence set forth in SEQ ID NO: 139, 184, 193, 202, 205, 223, 239, or 246. In some embodiments, the gRNA comprises the sequence set forth in SEQ ID NO: 140, 185, 194, 203, 206, 224, 236, 238, 240, 242, 243, 244, 245, 247, 249, or 250.

In some embodiments, the gRNA targeting RFX5 is a discontinuous or “split” RNA. In some embodiments, the discontinuous or “split” gRNA comprises the sequence set forth in SEQ ID NO: 377, 378, 379, 380, 381, 382, 383, 384, or 385.

In some embodiments, the at least one ribonucleic acid is complementary to and/or hybridizes to a sequence on the same strand of a target polynucleotide sequence, wherein the target polynucleotide sequence comprises a CD58 gene. In some embodiments, the at least one ribonucleic acid is a gRNA. In some embodiments, the target polynucleotide sequence comprises SEQ ID NO: 256, 259, 262, 265, 268, 271, 274, 277, 280, 283, 286, 289, 292, 295, 298, 301, 304, 307, 310, 313, 316, 319, 322, 325, 328, 331, 334, 337, 340, 343, 346, 349, 352, 355, 358, 361, 364, 367, 370, 373, or 376. In some embodiments, the gRNA comprises the sequence set forth in SEQ ID NO: 129. In some embodiments, the gRNA further comprises a spacer sequence comprising the sequence of SEQ ID NO: 254, 257, 260, 263, 266, 269, 272, 275, 278, 281, 284, 287, 290, 293, 296, 299, 302, 305, 308, 311, 314, 317, 320, 323, 326, 329, 332, 335, 338, 341, 344, 347, 350, 353, 356, 359, 362, 365, 368, 371, or 374. In some embodiments, the gRNA comprises the sequence of SEQ ID NO: 255, 258, 261, 264, 267, 270, 273, 276, 279, 282, 285, 288, 291, 294, 297, 300, 303, 306, 309, 312, 315, 318, 321, 324, 327, 330, 333, 336, 339, 342, 345, 348, 351, 354, 357, 360, 363, 366, 369, 372, or 375. In some embodiments, the target polynucleotide sequence comprises SEQ ID NO: 256, 271, 274, 280, 304, or 328. In some embodiments, the gRNA comprises the sequence of SEQ ID NO: 129. In some embodiments, the gRNA further comprises a spacer sequence comprising the sequence of SEQ ID NO: 254, 269, 272, 278, 302, or 326. In some embodiments, the gRNA comprises the sequence of SEQ ID NO: 255, 270, 273, 279, or 327.

In some embodiments, the gRNA targeting CD58 is a discontinuous or “split” RNA. In some embodiments, the discontinuous or “split” gRNA comprises the sequence set forth in SEQ ID NO: 377, 378, 379, 386, 387, or 388.

In some embodiments, the CRISPR endonuclease is a Cas9, and/or a Cpf1, e.g., L. bacterium ND2006 Cpf1 and/or Acidaminococcus sp. BV3L6 Cpf1, and/or a MAD7, and in various embodiments CRISPR/MAD7 is used. In some embodiments, since MAD7 is a Cas12a-like endonuclease, the target motif and/or the guide nucleic acid (e.g., gRNA) used or identified for Cpf1 or Cas-12a is the same as the target motif and/or the guide nucleic acid (e.g., gRNA) used for MAD7. In some embodiments, the target motif identified or used for CRISPR-Cpf1 system is the same target motif used for CRISPR-MAD7 system. In some embodiments, the guide nucleic acid (e.g., gRNA) identified or used for CRISPR-Cpf1 system is the same guide nucleic acid (e.g., gRNA) used for CRISPR-MAD7 system. In some embodiments, the target motif and the guide nucleic acid (e.g., gRNA) identified or used for CRISPR-Cpf1 system is the same target motif and the same guide nucleic acid (e.g., gRNA) used for CRISPR-MAD7 system. In some embodiments, the CRISPR endonuclease is MAD7. In some embodiments, the nuclease used in the methods of the disclosure is Inscripta's MAD7™ Nuclease. In some embodiments, the nuclease used in the methods of the disclosure is an Inscripta's nuclease. In some embodiments, methods incorporating the Inscripta MAD7™ Nuclease are methods of using MAD7™ as disclosed in WO2021/1186269, WO2021/119563, WO2022/146497, and WO2022/150269, which are incorporated herein by reference in their entirety. In some embodiments, the CRISPR endonuclease is a Cas9 (CRISPR associated protein 9). In some embodiments, the Cas9 endonuclease is from Streptococcus pyogenes. In some embodiments, other Cas9 homologs is used, e.g., S. aureus Cas9, N. meningitidis Cas9, S. thermophilus CRISPR 1 Cas9, S. thermophilus CRISPR 3 Cas9, or T. denticola Cas9. In some embodiments, the endonuclease is Cas1, Cas1B, Cas2, Cas3, Cas4, Cas5, Cash, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and/or Cpf1 endonuclease. In some embodiments, wild-type variants may be used. In some embodiments, modified versions (e.g., a homolog thereof, a recombination of the naturally occurring molecule thereof, codon-optimized thereof, or modified versions thereof) of an endonuclease can be used. In some embodiments, the endonuclease is any one or more endonuclease of the disclosure. In some embodiments, the endonuclease is any one or more endonucleases known to a skilled person. In some embodiments, exogenous Cas protein can be introduced into the cell in polypeptide form. In some embodiments, a Cas protein can be conjugated to or fused to a cell-penetrating polypeptide or cell-penetrating peptide. As used herein, “cell-penetrating polypeptide” and “cell-penetrating peptide” refer to a polypeptide or peptide, respectively, which facilitates the uptake of molecule into a cell. In some embodiments, the cell-penetrating polypeptides can contain a detectable label.

In some embodiments, the endonuclease or a Cas protein can be conjugated to or fused to a charged protein (e.g., that carries a positive, negative, or overall neutral electric charge). Such linkage may be covalent. In some embodiments, the endonuclease or Cas protein can be fused to a superpositively charged GFP to significantly increase the ability of the Cas protein to penetrate a cell (Cronican et al. ACS Chem Biol. 2010; 5(8):747-52). In some embodiments, the endonuclease or Cas protein can be fused to a protein transduction domain (PTD) to facilitate its entry into a cell. Exemplary PTDs include Tat, oligoarginine, and penetratin. In some embodiments, the endonuclease or Cas protein comprises a Cas polypeptide fused to a cell-penetrating peptide.

In some embodiments, the endonuclease is linked to at least one nuclear localization signal (NLS). The at least one NLS can be located at or within 50 amino acids of the amino-terminus of the endonuclease and/or at least one NLS can be located at or within 50 amino acids of the carboxy-terminus of the endonuclease.

In some embodiments, the CRISPR-endonuclease system comprises an RNA-guided endonuclease. In some embodiments, an RNA-guided endonuclease comprises an amino acid sequence having at least about 10%, at least about 15%, at least about 20%, at least about 30%, at least about 40%, at least about 50%, at least about 60%, at least about 70%, at least about 75%, at least about 80%, at least about 85%, at least about 90%, at least about 95%, at least about 99%, or 100% amino acid sequence identity to a wild-type endonuclease, e.g., Cpf1, MAD7, Cas9, and/or any other endonuclease of the disclosure. In some embodiments, the endonuclease comprises about or at least about 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids. In some embodiments, the endonuclease comprises at most about: 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids. In some embodiments, the endonuclease comprises at least about: 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids in a HNH nuclease domain of the endonuclease. In some embodiments, the endonuclease comprises at most about: 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids in a HNH nuclease domain of the endonuclease. In some embodiments, the endonuclease comprises at least about: 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids in a RuvC nuclease domain of the endonuclease. In some embodiments, the endonuclease comprises at most about: 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids in a RuvC nuclease domain of the endonuclease. The present disclosure provides a guide RNAs (gRNAs) that can direct the activities of an associated endonuclease to a specific target site within a polynucleotide. In some embodiments, a guide RNA comprises a spacer sequence that hybridizes to a target nucleic acid sequence of interest, and a CRISPR repeat sequence. In some embodiments, for example in CRISPR Type II systems, the gRNA also comprises a second RNA called the tracrRNA sequence. In some embodiments, in the CRISPR Type II guide RNA (gRNA), the CRISPR repeat sequence and tracrRNA sequence hybridize to each other to form a duplex. In some embodiments, in CRISPR Type V systems, the gRNA comprises a crRNA that forms a duplex. In some embodiments, a gRNA can bind an endonuclease, such that the gRNA and endonuclease form a complex. The gRNA can provide target specificity to the complex by virtue of its association with the endonuclease.

In some embodiments, a tracrRNA sequence comprises nucleotides that hybridize to a CRISPR repeat sequence in a cell. A tracrRNA sequence and a CRISPR repeat sequence may form a duplex, i.e., a base-paired double-stranded structure. Together, the tracrRNA sequence and the CRISPR repeat can bind to an RNA-guided endonuclease. In some embodiments, at least a part of the tracrRNA sequence can hybridize to the CRISPR repeat sequence. In some embodiments, the tracrRNA sequence can be at least about 30%, about 40%, about 50%, about 60%, about 65%, about 70%, about 75%, about 80%, about 85%, about 90%, about 95%, or 100% complementary to the CRISPR repeat sequence. In some embodiments, a tracrRNA sequence can have a length from about 7 nucleotides to about 100 nucleotides. For example, the tracrRNA sequence can be from about 7 nucleotides (NTs) to about 50 NTs, from about 7 NTs to about 40 NTs, from about 7 NTs to about 30 NTs, from about 7 NTs to about 25 NTs, from about 7 NTs to about 20 NTs, from about 7 NTs to about 15 NTs, from about 8 NTs to about 40 NTs, from about 8 NTs to about 30 NTs, from about 8 NTs to about 25 NTs, from about 8 NTs to about 20 NTs, from about 8 NTs to about 15 NTs, from about 15 NTs to about 100 NTs, from about 15 NTs to about 80 NTs, from about 15 NTs to about 50 NTs, from about 15 NTs to about 40 NTs, from about 15 NTs to about 30 NTs or from about 15 NTs to about 25 NTs long. In some embodiments, the tracrRNA sequence can be approximately 9 nucleotides in length. In some embodiments, the tracrRNA sequence can be approximately 12 nucleotides.

In some embodiments, the tracrRNA sequence can be at least about 60% identical to a reference tracrRNA (e.g., wild type, tracrRNA from S. pyogenes) sequence over a stretch of at least 6, 7, or 8 contiguous nucleotides. For example, a tracrRNA sequence can be at least about 65% identical, about 70% identical, about 75% identical, about 80% identical, about 85% identical, about 90% identical, about 95% identical, about 98% identical, about 99% identical or 100% identical to a reference tracrRNA sequence over a stretch of at least 6, 7, or 8 contiguous nucleotides.

In some embodiments, the Cas protein or the endonuclease can be introduced into a cell containing the target polynucleotide sequence in the form of a nucleic acid encoding the Cas protein or the endonuclease (e.g., Cas9, Cpf1, MAD7, or any endonuclease or Cas protein of the disclosure). In some embodiments, the method includes a technique to introduce a nucleic acid into γδiPSC cells. The process of introducing the nucleic acids into cells can be achieved by any suitable technique. Suitable techniques include, but are not limited to, transfection (e.g., neon transfection, calcium phosphate or lipid-mediated transfection), electroporation, and transduction or infection using a viral vector. In some embodiments, nucleic acids are introduced into cells using a non-viral system (e.g., neon transfection). In some embodiments, nucleic acids are introduced into cells using a viral system (e.g., adenoassociated virus). In some embodiments, the method includes electroporation of a cell (e.g., as disclosed in Section 7.3 or 7.4) or a human cell (e.g., an immunogenic human cell, an iPS human cell disclosed in Section 7.4) to introduce genetic material including, for example, DNA, RNA, and/or mRNA. In some embodiments a technique to introduce a protein or nucleic acid can include introducing a protein or nucleic acid via electroporation; microinjection; viral delivery; exosomes; liposomes; biolistics; jet injection; hydrodynamic injection; ultrasound; magnetic field-mediated gene transfer; electric pulse-mediated gene transfer; use of nanoparticles including, for example, lipid-based nanoparticles; incubation with a endosomolytic agent; use of cell-penetrating peptides; or any other suitable technique. In some embodiments, the method includes electroporation of a human cell including, for example, using a Neon transfection system (Thermo Fisher Scientific Inc.).

In some embodiments, the nucleic acid comprises DNA. In some embodiments, the nucleic acid comprises a modified DNA. In some embodiments, the nucleic acid comprises mRNA. In some embodiments, the nucleic acid comprises a modified mRNA.

In some embodiments, the Cas protein or endonuclease is complexed with at least one ribonucleic acid (e.g., one to two ribonucleic acid(s)). In some embodiments, the Cas protein or endonuclease is complexed with two ribonucleic acids. In some embodiments, the Cas protein or endonuclease is complexed with one ribonucleic acid. In some embodiments, the Cas protein or endonuclease is encoded by a modified nucleic acid.

In some embodiments, endonuclease and gRNA can each be administered separately to a cell. In some embodiments, the endonuclease can be pre-complexed with one or more guide RNAs, or one or more crRNA together with a tracrRNA. The pre-complexed material can then be administered to a cell. Such pre-complexed material is known as a ribonucleoprotein particle (RNP). The endonuclease in the RNP can be, for example, a Cpf1 endonuclease, a MAD7 endonuclease, a Cas9 endonuclease, or any endonuclease of the disclosure. In some embodiments, the endonuclease can be flanked at the N-terminus, the C-terminus, or both the N-terminus and C-terminus by one or more nuclear localization signals (NLSs). In some embodiments, the weight ratio of genome-targeting nucleic acid to endonuclease in the RNP can be 1:1, 2:1, 1:2, or any suitable ratio.

In some embodiments, the gRNA can be a double-molecule guide RNA. In some embodiments, the gRNA can be a single-molecule guide RNA (sgRNA). In some embodiments, a gRNA can be constructed as a single RNA oligonucleotide that is the combination of a repeat sequence followed by a spacer sequence, wherein specificity to the genomic target location is conferred by complementary binding of the spacer to genomic DNA. A split gRNA can be constructed as two RNA oligonucleotides, composed of a tracrRNA and a crRNA, in which the tracrRNA contains a portion of the repeat sequence and the crRNA contains a portion of the repeat sequence followed by the spacer sequence.

In some embodiments, a gRNA comprises a sequence that hybridizes to a sequence in a target polynucleotide. In some embodiments, the nucleotide sequence of the gRNA can vary depending on the sequence of the target nucleic acid of interest. In some embodiments, a gRNA comprises a variable length sequence with 17-30 nucleotides, in which at least a portion of the sequence hybridizes to a sequence in a target polynucleotide. In some embodiments, a gRNA sequence can be designed to hybridize to a target polynucleotide that is located 5′ of a PAM of the endonuclease used in the system.

In some embodiments, a gRNA comprises another moiety (e.g., a stability control sequence, an endoribonuclease binding sequence, or a ribozyme). The moiety can decrease or increase the stability of a nucleic acid targeting nucleic acid. In some embodiments, the moiety can be a transcriptional terminator segment (i.e., a transcription termination sequence). In some embodiments, the moiety can function in a eukaryotic cell. The moiety can function in a prokaryotic cell. In some embodiments, the moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

In some embodiments, the portion of the gRNA that hybridizes to a sequence or a target motif in a target polynucleotide is referred to as a spacer. In some embodiments, the portion of the gRNA that hybridizes to a sequence or a target motif in a target polynucleotide (spacer) comprises about 15, 16, 17, 18, 19, 20, 21, 22, 23, 24, 25, or more than about 25 nucleotides. In some embodiments, the portion of the gRNA that hybridizes to a sequence or a target motif in a target polynucleotide comprises less than about 25 nucleotides. In some embodiments, the portion of the gRNA that hybridizes to a sequence or a target motif in a target polynucleotide, or the gRNA comprises more than about 20 nucleotides. In some embodiments, the portion of the gRNA that hybridizes to a sequence or a target motif in a target polynucleotide, or the gRNA comprises about or at least about: 5, 10, 15, 16, 17, 18, 19, 20, 21, 22, 23, 24, 25, 30, 35, 40, 45, 50 or more nucleotides. In some embodiments, the portion of the gRNA that hybridizes to a sequence or a target motif in a target polynucleotide, or the gRNA comprises at most about: 5, 10, 15, 16, 17, 18, 19, 20, 21, 22, 23, 24, 25, 30, 35, 40, 45, 50 or more nucleotides. In some embodiments, the sequence or target motif in a target polynucleotide sequence comprises about, at least about, or at most about 20 bases immediately 5′ of the first nucleotide of the PAM.

In some embodiments, the portion of the gRNA that hybridizes to a sequence or a target motif in a target polynucleotide has a length of at least about 6 nucleotides (NTs). In some embodiments, the portion of the gRNA that hybridizes to a sequence or a target motif in a target polynucleotide, or the gRNA is about or at least about 6 NTs, about or at least about 10 NTs, about or at least about 15 NTs, about or at least about 18 NTs, about or at least about 19 NTs, about or at least about 20 NTs, about or at least about 21 NTs, about or at least about 22 NTs, about or at least about 23 NTs, about or at least about 24 NTs, about or at least about 25 NTs, about or at least about 30 NTs, about or at least about 35 NTs, about or at least about 40 NTs, about or at least about 45 NTs, about or at least about 50 NTs, or more than about 50 NTs. In some embodiments, the portion of the gRNA that hybridizes to a sequence or a target motif in a target polynucleotide, or the gRNA is from about 6 NTs to about 40 NTs, from about 6 NTs to about 35 NTs, from about 6 NTs to about 30 NTs, from about 6 NTs to about 29 NTs, from about 6 NTs to about 28 NTs, from about 6 NTs to about 27 NTs, from about 6 NTs to about 26 NTs, from about 6 NTs to about 25 NTs, from about 6 NTs to about 24 NTs, from about 6 NTs to about 23 NTs, from about 6 NTs to about 22 NTs, from about 6 NTs to about 21 NTs, from about 6 NTs to about 20 NTs, from about 10 NTs to about 50 NTs, from about 10 NTs to about 40 NTs, from about 10 NTs to about 35 NTs, from about 10 NTs to about 30 NTs, from about 10 NTs to about 30 NTs, from about 10 NTs to about 29 NTs, from about 10 NTs to about 28 NTs, from about 10 NTs to about 27 NTs, from about 10 NTs to about 26 NTs, from about 10 NTs to about 25 NTs, from about 10 NTs to about 24 NTs, from about 10 NTs to about 23 NTs, from about 10 NTs to about 22 NTs, from about 10 NTs to about 21 NTs, from about 10 NTs to about 20 NTs, from about 19 NTs to about 23 NTs, from about 19 NTs to about 24 NTs, from about 19 NTs to about 25 NTs, from about 19 NTs to about 30 NTs, from about 19 NTs to about 35 NTs, from about 19 NTs to about 40 NTs, from about 19 NTs to about 45 NTs, from about 19 NTs to about 50 NTs, from about 19 NTs to about 60 NTs, from about 20 NTs to about 25 NTs, from about 20 NTs to about 30 NTs, from about 20 NTs to about 35 NTs, from about 20 NTs to about 40 NTs, from about 20 NTs to about 45 NTs, from about 20 NTs to about 50 NTs, or from about 20 NTs to about 60 NTs.

In some embodiments, the percent complementarity between the gRNA or a portion of the gRNA (e.g., spacer or crRNA) and the target polynucleotide is about or at least about 30%, about or at least about 40%, about or at least about 50%, about or at least about 60%, about or at least about 65%, about or at least about 70%, about or at least about 75%, about or at least about 80%, about or at least about 85%, about or at least about 90%, about or at least about 95%, about or at least about 97%, about or at least about 98%, about or at least about 99%, or 100%. In some embodiments, the percent complementarity between the gRNA or a portion of the gRNA and the target polynucleotide is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some embodiments, the length of the portion of the gRNA and the target nucleic acid can differ by 1 to 6 nucleotides, which may be thought of as a bulge or bulges.

In some embodiments, a gRNA is modified or chemically modified. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, chemical modifications enhance stability, reduce the likelihood or degree of innate immune response, and/or enhance other attributes, as described in the art.

In some embodiments, a modified gRNA comprises a modified backbone, for example, phosphorothioates, phosphotriesters, morpholinos, methyl phosphonates, short chain alkyl or cycloalkyl intersugar linkages or short chain heteroatomic or heterocyclic intersugar linkages.

In some embodiments, a modified gRNA comprises one or more substituted sugar moieties, e.g., one of the following at the 2′ position: OH, SH, SCH₃, F, OCN, OCH₃, OCH₃ O(CH₂)n CH₃, O(CH₂)n NH₂, or O(CH₂)n CH₃, where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; 2′-O-(2-methoxyethyl); 2′-methoxy (2′-O—CH₃); 2′-propoxy (2′-OCH₂ CH₂CH₃); and 2′-fluoro (2′-F). Similar modifications may also be made at other positions on the gRNA, for example, the 3′ position of the sugar on the 3′ terminal nucleotide and/or the 5′ position of 5′ terminal nucleotide. In some examples, both a sugar and an internucleoside linkage, i.e., the backbone, of the nucleotide units can be replaced with different groups.

In some embodiments, a gRNA includes, additionally or alternatively, nucleobase (or “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, 5-methylcytosine (also referred to as 5-methyl-2′ deoxycytosine or 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine.

In some embodiments, modified nucleobases can include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

In some embodiments, genetically modifying the genomic DNA sequence of a target gene can be performed using a zinc finger nuclease (ZFN). Zinc finger nucleases (ZFNs) are modular proteins comprised of an engineered zinc finger DNA binding domain linked to the catalytic domain of the type II endonuclease FokI. Because FokI functions as a dimer, a pair of ZFNs is engineered to bind to cognate target “half-site” sequences on opposite DNA strands and with precise spacing between them to enable the catalytically active FokI dimer to form. Upon dimerization of the FokI domain, a DNA double-strand break is generated between the ZFN half-sites as the initiating step in genome editing.

In some embodiments, the DNA binding domain of each ZFN is comprised of 3-6 zinc fingers of the abundant Cys2-His2 architecture, with each finger primarily recognizing a triplet of nucleotides on one strand of the target DNA sequence, although cross-strand interaction with a fourth nucleotide can also occur. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. ZFNs can be readily re-targeted to almost any genomic address simply by modifying individual fingers. In some embodiments, proteins of 4-6 fingers are used, recognizing 12-18 bp respectively. Hence, a pair of ZFNs will typically recognize a combined target sequence of 24-36 bp, not including the typical 5-7 bp spacer between half-sites. The binding sites can be separated further with larger spacers, including 15-17 bp.

A variety of ZFN-based systems have been described in the art, modifications thereof are regularly reported, and numerous references describe rules and parameters that are used to guide the design of ZFNs; see, e.g., Segal et al., Proc Natl Acad Sci, 1999 96(6):2758-63; Dreier B et al., J Mol Biol., 2000, 303(4):489-502; Liu Q et al., J Biol Chem., 2002, 277(6):3850-6; Dreier et al., J Biol Chem., 2005, 280(42):35588-97; and Dreier et al., J Biol Chem. 2001, 276(31):29466-78.

In some embodiments, genetically modifying the genomic DNA sequence of a target gene can be performed using a Transcription Activator-Like Effector Nuclease (TALEN). TALEN represent another format of modular nucleases whereby, as with ZFNs, an engineered DNA binding domain is linked to the FokI nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs are comprised of tandem arrays of 33-35 amino acid repeats, with each repeat recognizing a single base pair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. A variety of TALEN-based systems have been described in the art, and modifications thereof are regularly reported; see, e.g., Boch, Science, 2009 326(5959):1509-12; Mak et al., Science, 2012, 335(6069):716-9; and Moscou et al., Science, 2009, 326(5959):1501. The use of TALENs based on the “Golden Gate” platform, or cloning scheme, has been described by multiple groups; see, e.g., Cermak et al., Nucleic Acids Res., 2011, 39(12):e82; Li et al., Nucleic Acids Res., 2011, 39(14):6315-25; Weber et al., PLoS One., 2011, 6(2):e16765; Wang et al., J Genet Genomics, 2014, 41(6):339-47; and Cermak T et al., Methods Mol Biol., 2015 1239:133-59.

In some embodiments, genetically modifying the genomic DNA sequence of a target gene can be performed using a Homing Endonuclease (HE). Homing endonucleases (HEs) are sequence-specific endonucleases that have long recognition sequences (14-44 base pairs) and cleave DNA with high specificity-often at sites unique in the genome. There are at least six known families of HEs as classified by their structure, including GIY-YIG, His-Cis box, H—N—H, PD-(D/E)xK, and Vsr-like that are derived from a broad range of hosts, including eukarya, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. A variety of HE-based systems have been described in the art, and modifications thereof are regularly reported; see, e.g., the reviews by Steentoft et al., Glycobiology, 2014, 24(8):663-80; Belfort and Bonocora, Methods Mol Biol., 2014, 1123:1-26; and Hafez and Hausner, Genome, 2012, 55(8):553-69.

In some embodiments, genetically modifying the genomic DNA sequence of a target gene can be performed using a MegaTAL or Tev-mTALEN platforms. The MegaTAL platform and Tev-mTALEN platform use a fusion of TALE DNA binding domains and catalytically active HEs, taking advantage of both the tunable DNA binding and specificity of the TALE, as well as the cleavage sequence specificity of the HE; see, e.g., Boissel et al., Nucleic Acids Res., 2014, 42: 2591-2601; Kleinstiver et al., G3, 2014, 4:1155-65; and Boissel and Scharenberg, Methods Mol. Biol., 2015, 1239: 171-96.

In some embodiments, the MegaTev architecture is the fusion of a meganuclease (Mega) with the nuclease domain derived from the GIY-YIG homing endonuclease I-Teel (Tev). The two active sites are positioned ~30 bp apart on a DNA substrate and generate two DSBs with non-compatible cohesive ends; see, e.g., Wolfs et al., Nucleic Acids Res., 2014, 42, 8816-29. It is anticipated that other combinations of existing nuclease-based approaches will evolve and be useful in achieving the targeted genome modifications described herein.

### 7.5.1 RNAi Technology and Transcriptional Repression

In some embodiments, genetically modifying a target gene comprises reducing mRNA of the target gene through RNA interference (RNAi) system. RNA interference (RNAi) is the biological process of mRNA degradation induced by complementary sequences double-stranded (ds) small interfering RNAs (siRNA) and suppression of target gene expression. Any suitable RNAi system known in the art can be used for reducing mRNA of a target gene. See, for example, Xu et al., Comprehensive Biotechnology. 2019: 560-575 for a review of RNAi technology.

In some embodiments, the RNAi system comprises synthetic siRNAs, short hairpin RNAs (shRNAs), dicer-produced siRNAs, endoribonuclease-prepared short interfering RNAs (esiRNAs), microRNAs and mimics, pro-siRNAs, miR-adapted shRNAs, or a combination thereof.

In some embodiments, genetically modifying a target gene comprises reducing or ablating transcription of the target gene (e.g., transcriptional repression). In some embodiments, genetically modifying a target gene comprises recruiting or directing a transcriptional repressor to the target gene. Transcriptional repressors are chromatin-modifying proteins that can repress transcription of a gene. The repressor protein works by binding to the promoter region of the gene(s), which prevents the production of mRNA.

Any suitable transcriptional repressors known in the art can be used with the presently disclosed subject matter. Non-limiting examples of transcriptional repressors include Kruppel-associated box (KRAB) repressor domains, and methyl-CpG binding protein 2 (MeCP2). Transcriptional repression can also occur through steric hinderance of the RNA polymerase complex initiation or elongation phases.

In some embodiments, the gRNA is used to knock-in a miR-adapted shRNA that targets CD58. In some embodiments, the miRNA comprises the sequence set forth in SEQ ID NO: 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or 128.

In some embodiments, the methods comprise knocking out one or more target genes in the hypoimmunogenic cell (such as the engineered hypoimmunogenic cell) or the iPS human cell or the iPS cell, e.g., via a shRNA. In some embodiments, shRNA is used to disrupt the CD58 gene. In some embodiments, the shRNA comprises the sequence set forth in SEQ ID NOs: 60, 61, 62, 63, 64, 65, 66, or 67. In some embodiments, the shRNA comprises the sequence set forth in SEQ ID NOs: 60, 63, or 64.

### 7.6 Cell Populations

The present disclosure further provides a non-naturally occurring hypoimmunogenic cell (such as an engineered hypoimmunogenic cell), which is produced by a presently disclosed method (e.g., a method of engineering hypoimmunogenicity disclosed in Section 7.4. and Section 7.5).

The present disclosure further provides a non-naturally occurring hypoimmunogenic human cell (such as an engineered hypoimmunogenic human cell), which is produced by a presently disclosed method (e.g., a method of engineering hypoimmunogenicity disclosed in Section 7.4. and Section 7.5).

The present disclosure further provides a non-naturally occurring hypoimmunogenic cell (such as an engineered hypoimmunogenic cell), comprising at least one target gene (e.g., a RFX gene, a B2M gene, a CD58 gene, a CIITA gene) that is genetically modified, wherein the genetically modified target gene reduces expression of the protein encoded by the at least one target gene. In some embodiments, the hypoimmunogenic cell (such as the engineered hypoimmunogenic cell) is produced from an embryoid body. In some embodiments, the hypoimmunogenic cell (such as the engineered hypoimmunogenic cell) comprises at least two, at least three, at least four target genes that are genetically modified (e.g., genetically modified RFX gene and B2M gene, genetically modified RFX gene and CD58 gene, genetically modified B2M gene and CIITA gene, genetically modified B2M gene and CD58 gene, genetically modified CD58 gene and CIITA gene, genetically modified RFX gene, B2M gene, and CD58 gene, genetically modified CIITA gene, B2M gene, and CD58 gene).

The present disclosure further provides a non-naturally occurring hypoimmunogenic human cell (such as an engineered hypoimmunogenic human cell), comprising at least one target gene (e.g., a RFX gene, a B2M gene, a CD58 gene, a CIITA gene) that is genetically modified, wherein the genetically modified target gene reduces expression of the protein encoded by the at least one target gene. In some embodiments, the hypoimmunogenic human cell (such as the engineered hypoimmunogenic human cell) is produced from an embryoid body. In some embodiments, the hypoimmunogenic human cell (such as the engineered hypoimmunogenic human cell) comprises at least two, at least three, at least four target genes that are genetically modified (e.g., genetically modified RFX gene and B2M gene, genetically modified RFX gene and CD58 gene, genetically modified B2M gene and CIITA gene, genetically modified B2M gene and CD58 gene, genetically modified CD58 gene and CIITA gene, genetically modified RFX gene, B2M gene, and CD58 gene, genetically modified CIITA gene, B2M gene, and CD58 gene).

The present disclosure further provides a γδT cell-derived induced pluripotent stem (iPS) human cell, comprising at least one target gene (e.g., a RFX gene, a B2M gene, a CD58 gene, a CIITA gene) that is genetically modified, wherein the genetically modified target gene reduces expression of the protein encoded by the at least one target gene. In some embodiments, the iPS human cell comprises at least two, at least three, at least four target genes that are genetically modified (e.g., genetically modified RFX gene and B2M gene, genetically modified RFX gene and CD58 gene, genetically modified B2M gene and CIITA gene, genetically modified B2M gene and CD58 gene, genetically modified CD58 gene and CIITA gene, genetically modified RFX gene, B2M gene, and CD58 gene, genetically modified CIITA gene, B2M gene, and CD58 gene).

In some embodiments, the present disclosure provides a non-naturally occurring hypoimmunogenic human cell (such as an engineered hypoimmunogenic human cell) derived from the γδT cell-derived iPS human cell.

In some embodiments, the non-naturally occurring hypoimmunogenic cell (such as an engineered hypoimmunogenic cell) or the non-naturally occurring hypoimmunogenic human cell (such as the engineered hypoimmunogenic human cell) or the γδT cell-derived induced pluripotent stem (iPS) human cell disclosed herein further comprises at least one of a genetically modified TNFRSF14 (also known as HVEM) gene, a genetically modified TNFRSF1A (also known as TNFR1) gene, a genetically modified TNFRSF1B (also known as TNFR2) gene, and a genetically modified ICAM1 gene.

In some embodiments, a population of hypoimmunogenic cells (such as engineered hypoimmunogenic cells) of the disclosure (e.g., cells having at least one genetically modified target gene) have reduced immunogenicity or reduced immune response, for example, by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower), as compared to a population of unmodified cells. In some embodiments, the only difference between the population of hypoimmunogenic cells (such as engineered hypoimmunogenic cells) and the population of unmodified cells is that the at least one target gene is not genetically modified in the population of unmodified cells.

In some embodiments, a population of hypoimmunogenic cells (such as engineered hypoimmunogenic cells) of the disclosure (e.g., cells having at least one genetically modified target gene) have reduced myeloid cell response, for example, by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower) as compared to a population of unmodified cells. In some embodiments, the only difference between the population of hypoimmunogenic cells (such as engineered hypoimmunogenic cells) and the population of unmodified cells is that the at least one target gene is not genetically modified in the population of unmodified cells.

In some embodiments, a population of hypoimmunogenic cells (such as engineered hypoimmunogenic cells) of the disclosure (e.g., cells having at least one genetically modified target gene) have reduced T cell response, for example, by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower) as compared to a population of unmodified cells. In some embodiments, the only difference between the population of hypoimmunogenic cells (such as engineered hypoimmunogenic cells) and the population of unmodified cells is that the at least one target gene is not genetically modified in the population of unmodified cells.

In some embodiments, a population of hypoimmunogenic cells (such as engineered hypoimmunogenic cells) of the disclosure (e.g., cells having at least one genetically modified target gene) have reduced natural killer cell response, for example, by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower), as compared to a population of unmodified cells. In some embodiments, the only difference between the population of hypoimmunogenic cells (such as engineered hypoimmunogenic cells) and the population of unmodified cells is that the at least one target gene is not genetically modified in the population of unmodified cells.

In some embodiments, a population of hypoimmunogenic cells (such as engineered hypoimmunogenic cells) of the disclosure (e.g., cells having at least one genetically modified target gene) have reduced antibody response, for example, by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower), as compared to a population of unmodified cells. In some embodiments, the only difference between the population of hypoimmunogenic cells (such as engineered hypoimmunogenic cells) and the population of unmodified cells is that the at least one target gene is not genetically modified in the population of unmodified cells.

In some embodiments, a population of hypoimmunogenic cells (such as engineered hypoimmunogenic cells) of the disclosure (e.g., cells having at least one genetically modified target gene) have reduced allogeneic host versus graft rejection, for example, by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower), as compared to a population of unmodified cells. In some embodiments, the only difference between the population of hypoimmunogenic cells (such as engineered hypoimmunogenic cells) and the population of unmodified cells is that the at least one target gene is not genetically modified in the population of unmodified cells.

In some embodiments, a population of hypoimmunogenic cells (such as engineered hypoimmunogenic cells) of the disclosure (e.g., cells having a genetically modified RFX gene) have reduced MHC class II mediated response, for example, by about or at least about 50%, 55%, 60%, 65%, 70%, 75%, 80%, 85%, 90%, 95%, 100% (lower), as compared to a population of unmodified cells. In some embodiments, the only difference between the population of hypoimmunogenic cells (such as engineered hypoimmunogenic cells) and the population of unmodified cells is that the RFX gene is not genetically modified in the population of unmodified cells.

In some embodiments, a population of hypoimmunogenic cells (such as engineered hypoimmunogenic cells) of the disclosure (e.g., cells having a genetically modified RFX gene) have reduced MHC class I mediated response, for example, by about or at least about 5%, 10%, 15%, 20%, 25%, 30%, 35%, 40%, 45%, 50%, 55%, 60%, 65%, 70%, 75%, 80%, 85%, 90%, 95% (lower), as compared to a population of unmodified cells. In some embodiments, the only difference between the population of hypoimmunogenic cells (such as engineered hypoimmunogenic cells) and the population of unmodified cells is that the RFX gene is not genetically modified in the population of unmodified cells.

In some embodiments, the expression of the HLA class II molecules in a population of hypoimmunogenic cells (such as engineered hypoimmunogenic cells) of the disclosure (e.g., cells having a genetically modified RFX gene) is reduced, for example, by about or at least about 50%, 55%, 60%, 65%, 70%, 75%, 80%, 85%, 90%, 95%, 100% (lower), as compared to the expression of HLA class II molecules in a population of unmodified cells. In some embodiments, the only difference between the population of hypoimmunogenic cells (such as engineered hypoimmunogenic cells) and the population of unmodified cells is that the RFX gene is not genetically modified in the population of unmodified cells.

In some embodiments, the expression of HLA class I molecules in a population of hypoimmunogenic cells (such as engineered hypoimmunogenic cells) of the disclosure (e.g., cells having a genetically modified RFX gene) is reduced by about or at least about 5%, 10%, 15%, 20%, 25%, 30%, 35%, 40%, 45%, 50%, 55%, 60%, 65%, 70%, 75%, 80%, 85%, 90%, 95% (lower) as compared to the expression of HLA class I molecules in a population of unmodified cells. In some embodiments, the only difference between the population of hypoimmunogenic cells (such as engineered hypoimmunogenic cells) and the population of unmodified cells is that the RFX gene is not genetically modified in the population of unmodified cells.

In some embodiments, a population of hypoimmunogenic cells (such as engineered hypoimmunogenic cells) of the present disclosure (e.g., cells having a genetically modified B2M gene) have reduced MHC class I mediated response by about or at least about 50%, 55%, 60%, 65%, 70%, 75%, 80%, 85%, 90%, 95%, 100% (lower) as compared to a population of unmodified cells. In some embodiments, the only difference between the population of hypoimmunogenic cells (such as engineered hypoimmunogenic cells) and the population of unmodified cells is that the B2M gene is not genetically modified in the population of unmodified cells.

In some embodiments, the expression of HLA class I molecules in a population of hypoimmunogenic cells (such as engineered hypoimmunogenic cells) of the disclosure (e.g., cells having a genetically modified B2M gene) is reduced by about or at least about 50%, 55%, 60%, 65%, 70%, 75%, 80%, 85%, 90%, 95%, 100% (lower) as compared to the expression of HLA class I molecules in a population of unmodified cells. In some embodiments, the only difference between the population of hypoimmunogenic cells (such as engineered hypoimmunogenic cells) and the population of unmodified cells is that the B2M gene is not genetically modified in the population of unmodified cells.

In some embodiments, a population of hypoimmunogenic cells (such as engineered hypoimmunogenic cells) of the present disclosure (e.g., cells having a genetically modified CIITA gene) have reduced MHC class II mediated response by about or at least about 50%, 55%, 60%, 65%, 70%, 75%, 80%, 85%, 90%, 95%, 100% (lower) as compared to a population of unmodified cells. In some embodiments, the only difference between the population of hypoimmunogenic cells (such as engineered hypoimmunogenic cells) and the population of unmodified cells is that the CIITA gene is not genetically modified in the population of unmodified cells.

In some embodiments, the expression of HLA class II molecules in a population of hypoimmunogenic cells (such as engineered hypoimmunogenic cells) of the disclosure (e.g., cells having a genetically modified CIITA gene) is reduced by about or at least about 50%, 55%, 60%, 65%, 70%, 75%, 80%, 85%, 90%, 95%, 100% (lower) as compared to the expression of HLA class II molecules in a population of unmodified cells. In some embodiments, the only difference between the population of hypoimmunogenic cells (such as engineered hypoimmunogenic cells) and the population of unmodified cells is that the CIITA gene is not genetically modified in the population of unmodified cells.

In some embodiments, a population of hypoimmunogenic cells (such as engineered hypoimmunogenic cells) of the present disclosure (e.g., cells having a genetically modified CD58 gene) have a reduced or ablated costimulatory immune cell response. In some embodiments, a population of hypoimmunogenic cells (such as engineered hypoimmunogenic cells) of the present disclosure (e.g., cells having a genetically modified CD58 gene) have impaired formation of an immune synapse. In some embodiments, a population of hypoimmunogenic cells (such as engineered hypoimmunogenic cells) of the present disclosure (e.g., cells having a genetically modified CD58 gene) have impaired recognition by patient (host) T-cells, NK cells, and myeloid cells.

In some embodiments, the population of hypoimmunogenic cells (such as engineered hypoimmunogenic cells) are blood cells. In some embodiments, the blood cells are suitably peripheral blood mononuclear cells (PBMCs), and may include all types of blood cells existing on an entire differentiation process from hematopoietic stem cells to final differentiation into peripheral blood. In some embodiments, the blood cells include, for example, hematopoietic stem cells, lymphoid stem cells, lymphoid dendritic cell progenitor cells, lymphoid dendritic cells, T lymphocyte progenitor cells, T cells, B lymphocyte progenitor cells, B cells, plasma cells, NK progenitor cells, NK cells, monocytes, and macrophages.

In some embodiments, the population of hypoimmunogenic cells (such as engineered hypoimmunogenic cells) can be peripheral blood mononuclear cells (PBMC), peripheral blood leukocytes (PBL), tumor infiltrating lymphocytes (TIL), or a combination thereof. In some embodiments, the population of hypoimmunogenic cells (such as engineered hypoimmunogenic cells) are peripheral blood mononuclear (PBMC) cells.

In some embodiments, the population of hypoimmunogenic cells (such as engineered hypoimmunogenic cells) are T cells. In some embodiments, the population of hypoimmunogenic cells (such as engineered hypoimmunogenic cells) can be selected from the group consisting of CD4⁺/CD8⁺ double positive T cells, cytotoxic T cells, Th3 (Treg) cells, Th9 cells, Thap helper cells, Tfh cells, stem memory TSCM cells, central memory TCM cells, effector memory TEM cells, effector memory TEMRA cells, gamma delta T cells and any combination thereof.

In some embodiments, the population of hypoimmunogenic cells (such as engineered hypoimmunogenic cells) is derived from a cell type that is easily accessible and requires minimal invasion, such as a fibroblast, a skin cell, a cord blood cell, a peripheral blood cell, and a renal epithelial cell.

In some embodiments, the population of hypoimmunogenic cells (such as engineered hypoimmunogenic cells) are terminally differentiated cells. In some embodiments, the population of hypoimmunogenic cells (such as engineered hypoimmunogenic cells) are terminally differentiated T cells. In some embodiments, the population of hypoimmunogenic cells (such as engineered hypoimmunogenic cells) are terminally differentiated PBMC cells. In some embodiments, the population of hypoimmunogenic cells (such as engineered hypoimmunogenic cells) are terminally differentiated yS T cells.

The population of hypoimmunogenic cells (such as engineered hypoimmunogenic cells) of the present disclosure may be derived from a mammal, preferably a human, but include and are not limited to non-human primates, murines (i.e., mice and rats), canines, felines, equines, bovines, ovines, porcines, caprines, etc.

In some embodiments, the population of hypoimmunogenic cells (such as engineered hypoimmunogenic cells) are mammal cells.

In some embodiments, the population of hypoimmunogenic cells (such as engineered hypoimmunogenic cells) are human cells.

In some embodiments, the population of hypoimmunogenic cells (such as engineered hypoimmunogenic cells) are human PBMC cells.

In some embodiments, the population of hypoimmunogenic cells (such as engineered hypoimmunogenic cells) do not comprise a BCMA-CAR. In some embodiments, the population of hypoimmunogenic cells do not comprise an MHC class I chain-related (MIC)-CAR, e.g., a MICA and/or MICB CAR. In some embodiments, the population of hypoimmunogenic cells (such as engineered hypoimmunogenic cells) do not comprise a CAR that comprises a signaling domain from the cytoplasmic domain of a signal transducing protein specific to T and/or NK cell activation or functioning.

In some embodiments, the hypoimmunogenic cell (such as the engineered hypoimmunogenic cell) is a T cell. In some embodiments, the hypoimmunogenic cell (such as an engineered hypoimmunogenic cell) is a T effector cell. In some embodiments, the hypoimmunogenic cell (such as the engineered hypoimmunogenic cell) is not a T regulatory cell. In some embodiments, the hypoimmunogenic cell (such as an engineered hypoimmunogenic cell) does not have a C45RA⁺CD27⁻CD28⁻CCR7⁻CD62L⁻ phenotype. In some embodiments, the hypoimmunogenic cell (such as the engineered hypoimmunogenic cell) is not a natural killer cell.

In some embodiments, the hypoimmunogenic cell (such as the engineered hypoimmunogenic cell) or the iPS human cell does not comprise a genetically modified, e.g., disrupted or knocked out: a) CISH (Cytokine Inducible SH2 Containing Protein) gene; b) adenosine A2A (ADORA2A) gene; c) TGF beta receptor gene; d) HLA class I gene, e.g., HLA A, B, C, E, F, G; e) HLA class II gene; f) NLRC5 (NOD-Like Receptor Family CARD Domain Containing 5) gene; g) CD38 gene; h) thioredoxin interacting protein (TXNIP) gene; i) ITGB3 (Integrin Subunit Beta 3) gene; j) IL17A gene; k) DGKA (diacylglycerol kinase alpha) gene; 1) DGKZ (diacylglycerol kinase zeta) gene; m) PD1 gene; n) TRGC1 (T-cell receptor gamma constant 1) gene; o) TRGC1 (T-cell receptor gamma constant 2) gene; and/or p) TRDC (T-cell receptor delta constant) gene.

In some embodiments, the hypoimmunogenic cell (such as the engineered hypoimmunogenic cell) or the iPS human cell is not TCR null, for example, is not TCR alpha, beta, gamma and/or delta null. For example, in certain embodiments, the TCR locus, e.g., TCR alpha, beta, gamma or delta locus, is not disrupted or knocked out, for example does not comprise an insertion, e.g., a CAR insertion.

In some embodiments, the hypoimmunogenic cell (such as the engineered hypoimmunogenic cell) or the iPS human cell does not comprise: a) an exogenous NICD (Notch Intracellular Domain) coding sequence, e.g., an NICD1 coding sequence; c) an exogenous CD47 coding sequence or increased CD47 expression relative to the wild type (non-engineered) iPS human cell; d) an exogenous sequence that encodes a cell surface protein that binds on the surface of a phagocytic or cytolytic immune cell, wherein said binding results in activation of the hypoimmunogenic cell (such as the engineered hypoimmunogenic cell), e.g., T-cell; e) an exogenous CR1 coding sequence; f) an exogenous CD24 coding sequence; g) an exogenous DUX4 (Double Homeobox 4) coding sequence; h) an exogenous nucleotide sequence operably linked to a promoter derived from a human FOXP3 gene; i) an exogenous CD3 complex cell surface coding sequence or increased expression of a CD3 complex cell surface gene relative to the wild type (non-engineered) iPS human cell; j) an exogenous NKG2C (Natural-Killer Receptor Group 2, member C) coding sequence or increased expression of NKG2C relative to the wild type (non-engineered) iPS human cell; k) an exogenous NKG2D (Natural-Killer Receptor Group 2, member D) coding sequence or increased expression of NKG2D relative to the wild type (non-engineered) iPS human cell; l) an exogenous PD-L1 coding sequence or increased expression of PD-L1 relative to the wild type (non-engineered) iPS human cell; m) an exogenous CTLA-4 coding sequence or increased expression of CTLA-4 relative to the wild type (non-engineered) iPS human cell; n) an exogenous CD16 coding sequence or increased expression of CD16 relative to the wild type (non-engineered) iPS human cell; o) an exogenous HLA-A coding sequence; p) an exogenous HLA-B coding sequence; q) an exogenous HLA-C coding sequence; r) an exogenous HLA-D coding sequence; s) an exogenous HLA-E coding sequence; t) an exogenous HLA-F coding sequence; u) an exogenous HLA-G coding sequence; v) an exogenous C1-inhibitor coding sequence; x) an exogenous IL35 coding sequence; and/or y) an IL15/IL15 Receptor alpha (IL15Ra) fusion protein, e.g., an IL15/IL15Ra fusion protein, wherein the IL15Ra portion lacks an intracellular domain.

### 7.7 Compositions

The present disclosure further provides a composition comprising the presently disclosed non-naturally occurring hypoimmunogenic cell (such as the engineered hypoimmunogenic cell) or the presently disclosed non-naturally occurring hypoimmunogenic human cell (such as the engineered hypoimmunogenic human cell). The present disclosure further provides a composition comprising the presently disclosed iPS human cells or cells differentiated therefrom. In some embodiments, the composition is a pharmaceutical composition, which further comprises a pharmaceutically acceptable carrier. Pharmaceutical compositions provided herein can be formulated to be compatible with the intended method or route of administration.

Suitable pharmaceutically acceptable carriers include, but are not limited to, antioxidants (e.g., ascorbic acid), preservatives (e.g., benzyl alcohol, methyl parabens, p-hydroxybenzoate), emulsifying agents, suspending agents, dispersing agents, solvents, buffers, lubricants, fillers, and/or diluents. For example, a suitable vehicle may be physiological saline solution. Typical buffers that can be used include, but are not limited to pharmaceutically acceptable weak acids, weak bases, or mixtures thereof. Buffer components can also include water soluble reagents such as phosphoric acid, tartaric acids, succinic acid, citric acid, acetic acid, and salts thereof.

A vehicle may contain other pharmaceutically acceptable excipients for modifying or maintaining the pH, osmolarity, viscosity, or stability of the pharmaceutical composition. In a specific embodiment, the vehicle is an aqueous buffer. In a specific embodiment, a vehicle comprises, for example, sodium chloride.

Pharmaceutical compositions provided herein may contain still other pharmaceutically acceptable formulation agents for modifying or maintaining the rate of administration of the produced hypoimmunogenic cells (such as engineered hypoimmunogenic cells) or hypoimmunogenic human cells (such as engineered hypoimmunogenic human cells) described herein. Such formulation agents include, for example, those substances known to those skilled in the art in preparing sustained-release or controlled release formulations. Regarding pharmaceutically acceptable formulation agents, see, for example, Remington's Pharmaceutical Sciences, 18th Ed. (1990, Mack Publishing Co., Easton, Pa. 18042) pages 1435-1712, and The Merck Index, 12th Ed. (1996, Merck Publishing Group, Whitehouse, NJ).

In some embodiments, a pharmaceutical composition is provided in a single-use container (e.g., a single-use vial, ampoule, syringe, or autoinjector). In a specific embodiment, a pharmaceutical composition is provided in a multi-use container (e.g., a multi-use vial or cartridge). Any drug delivery apparatus may be used to deliver hypoimmunogenic cells (such as engineered hypoimmunogenic cells) or hypoimmunogenic human cells (such as engineered hypoimmunogenic human cells) or pharmaceutical composition described herein, including intravenous infusion.

A pharmaceutical composition can be formulated to be compatible with its intended route of administration as described herein.

Pharmaceutical compositions can also include carriers to protect the composition against degradation or elimination from the body. Various antibacterial and antifungal agents, for example, parabens, chlorobutanol, ascorbic acid, thimerosal, can be included in the pharmaceutical composition.

### 8. EMBODIMENTS

The present disclosure provides the following non-limiting embodiments.

In one set of embodiments (embodiment set A), provided are:

A1. A method of hypoimmunogenicity (such as engineering hypoimmunogenicity), comprising:
a) genetically modifying a regulatory factor X (RFX) gene of at least one immunogenic human cell, wherein genetically modifying the RFX gene reduces expression of the RFX protein in the immunogenic human cell;
b) forming at least one embryoid body or multicellular body from the cell of a) to produce at least one hypoimmunogenic cell (such as an engineered hypoimmunogenic cell) or;
c) subjecting the hypoimmunogenic cell (such as the engineered hypoimmunogenic cell) to an immune system; and
d) determining immunogenicity of the hypoimmunogenic cell (such as the engineered hypoimmunogenic cell), wherein the immunogenicity is altered as compared to an immunogenic human cell where the RFX gene is not genetically modified,
optionally wherein step a) further comprises genetically modifying one or more of a class II major histocompatibility complex transactivator (CIITA) gene, a beta-2-microglobulin (B2M) gene, and a CD58 gene of the immunogenic human cell.
   A2. A method of hypoimmunogenicity (such as engineering hypoimmunogenicity), comprising:
a) reprogramming an immunogenic human cell to produce an induced pluripotent stem (iPS) human cell, wherein the immunogenic human cell comprises a heterodimeric T-cell receptor comprising a γ chain and a δ chain;
b) genetically modifying a regulatory factor X (RFX) gene of the iPS human cell, wherein genetically modifying the RFX gene reduces expression of the RFX protein by the iPS human cell;
c) forming at least one embryoid body from the cell of step b) to produce at least one hypoimmunogenic cell (such as an engineered hypoimmunogenic cell);
d) subjecting the hypoimmunogenic cell (such as the engineered hypoimmunogenic cell) to an immune system; and
e) determining immunogenicity of the hypoimmunogenic cell (such as the engineered hypoimmunogenic cell), wherein the immunogenicity is altered as compared to an iPS human cell where the RFX gene is not genetically modified,
optionally wherein step b) further comprises genetically modifying one or more of a class II major histocompatibility complex transactivator (CIITA) gene, a beta-2-microglobulin (B2M) gene, and a CD58 gene of the iPS human cell.
   A3. A method of hypoimmunogenicity (such as engineering hypoimmunogenicity), comprising:
a) genetically modifying a regulatory factor X (RFX) gene of an immunogenic human cell to produce a hypoimmunogenic cell (such as the engineered hypoimmunogenic cell), wherein genetically modifying the RFX gene reduces expression of the RFX protein by the immunogenic human cell;
b) subjecting the hypoimmunogenic cell (such as the engineered hypoimmunogenic cell) to an immune system; and
c) determining immunogenicity of the hypoimmunogenic cell (such as the engineered hypoimmunogenic cell), wherein the immunogenicity is altered as compared to an immunogenic human cell where the RFX gene is not genetically modified,
optionally wherein step a) further comprises genetically modifying one or more of a class II major histocompatibility complex transactivator (CIITA) gene, a beta-2-microglobulin (B2M) gene, and a CD58 gene of the immunogenic human cell.
   A4. A method of producing an hypoimmunogenic cell (such as an engineered hypoimmunogenic cell) from an immunogenic cell, comprising: (i) genetically modifying a regulatory factor X (RFX) gene in the immunogenic cell, wherein genetically modifying the RFX gene reduces expression of the RFX protein in said cell, and (ii) optionally further genetically modifying one or more genes selected from a class II major histocompatibility complex transactivator (CIITA) gene, a beta-2-microglobulin (B2M) gene, and a CD58 gene in said immunogenic cell, wherein genetically modifying said one or more genes reduces expression of the corresponding one or more proteins in said immunogenic cell, wherein said method results in production of the hypoimmunogenic cell (such as the engineered hypoimmunogenic cell), which has one or more of the following properties: a) having a reduced immunogenicity upon the hypoimmunogenic cell's (such as the engineered hypoimmunogenic cell's) presence in an allogeneic or non-MHC matched subject as compared to a corresponding immunogenic cell, but without the genetic modification(s) of (i) and (ii); b) causing a reduced immune response to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii); and c) causing a reduced alloreactive T cell cytotoxicity to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii).
   A5. A method of producing a hypoimmunogenic cell (such as an engineered hypoimmunogenic cell) from an immunogenic cell, comprising:
a) reprogramming the immunogenic cell to produce an induced pluripotent stem (iPS) cell;
b) (i) genetically modifying a regulatory factor X (RFX) gene in the iPS cell produced in step (a), wherein genetically modifying the RFX gene reduces expression of the RFX protein in said iPS cell, and (ii) optionally further genetically modifying one or more genes selected from a class II major histocompatibility complex transactivator (CIITA) gene, a beta-2-microglobulin (B2M) gene, and a CD58 gene in said iPS cell, wherein genetically modifying said one or more genes reduces expression of the corresponding one or more proteins in said iPS cell; and
c) optionally, differentiating the cell produced in step (b); wherein said method results in production of the hypoimmunogenic cell (such as the engineered hypoimmunogenic cell) which has one or more of the following properties: 1) having a reduced immunogenicity upon the hypoimmunogenic cell's, such as the engineered hypoimmunogenic cell's, presence in an allogeneic or non-MHC matched subject, as compared to a corresponding iPS cell, or a cell corresponding to the cell produced in step (c), but without the genetic modification(s) of step (b); 2) causing a reduced immune response to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b); and 3) causing a reduced alloreactive T cell cytotoxicity to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b).
   A6. The method of any one of embodiments A1-A5, wherein the hypoimmunogenic cell (such as the engineered hypoimmunogenic cell) comprises a T-cell receptor (TCR) comprising a γ chain and a 6 chain.
   A7. The method of any one of claims A1-A6, wherein the immunogenic human cell or immunogenic cell is an immune cell, optionally selected from T cells, natural killer (NK) cells, B cells, and hematopoietic stem cells (HSCs).
   A8. The method of any one of embodiments A1-A7, wherein the reduced immunogenicity of the hypoimmunogenic cell (such as the engineered hypoimmunogenic cell) comprises one or more of the following: i) a reduced or ablated myeloid cell response to the hypoimmunogenic cell (such as an engineered hypoimmunogenic cell) upon the cell's presence in an allogeneic or non-MHC matched subject, as compared to a cell corresponding to the cell that was modified but without said genetic modification(s); ii) a reduced or ablated T cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iii) a reduced or ablated natural killer (NK) cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iv) a reduced or ablated neutralizing antibody response to the hypoimmunogenic cell (such as the engineered hypoimmunogenic cell) upon the cell's presence in an allogeneic or non-MHC matched subject, as compared to a cell corresponding to the cell that was modified but without said genetic modification(s); v) a reduced or ablated MHC class II mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); vi) a reduced or ablated neutralizing MHC class I mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); and vii) a reduced or ablated allogeneic host versus graft rejection of to the hypoimmunogenic cell (such as the engineered hypoimmunogenic cell) upon the cell's presence in an allogeneic subject, as compared to a cell corresponding to the cell that was modified but without said genetic modification(s).
   A9. The method of any one of embodiments A4-A8, wherein the immunogenic cell is a human cell.
   A10. The method of embodiment A9, wherein in the hypoimmunogenic cell (such as the engineered hypoimmunogenic cell): i) expression of HLA class II molecules is reduced or ablated; ii) expression of HLA-A, HLA-B, and/or HLA-C is reduced; and iii) expression of HLA-E is reduced but remains detectable.
   A11. The method of any one of embodiments A4-A10, wherein the method comprises forming at least one embryoid body or multicellular body from the genetically modified cell to produce the hypoimmunogenic cell (such as the engineered hypoimmunogenic cell).
   A12. The method of any one of embodiments A4-A11, further comprising determining immunogenicity of the hypoimmunogenic cell (such as the engineered hypoimmunogenic cell).
   A13. The method of any one of embodiments A1-A12, further comprising administering the hypoimmunogenic cell (such as the engineered hypoimmunogenic cell) to an allogeneic or non-MHC matched subject.
   A14. The method of any one of embodiments A1-A13, wherein the immunogenicity of the hypoimmunogenic cell (such as the engineered hypoimmunogenic cell) is altered as compared to an immunogenic cell or an immunogenic human cell or a human iPS cell or an iPS cell, where the only difference between the hypoimmunogenic cell (such as the engineered hypoimmunogenic cell) and the immunogenic cell or the immunogenic human cell or the human iPS cell or the iPS cell is that the RFX gene and optionally one or more of the CIITA gene, the B2M gene, and the CD58 gene is not genetically modified in the immunogenic cell or the immunogenic human cell or the human iPS cell.
   A15. The method of any one of embodiments A1 to A14, wherein the immunogenic human cell or the immunogenic cell is allogeneic or non-HLA matched or non-MHC matched to cells, receptors, or polypeptides of the immune system of a recipient subject.
   A16. The method of any one of embodiments A1 to A3 and A6-A15, wherein altering the immunogenicity comprises balancing, reducing, or neutralizing the immunogenicity, such as reducing or neutralizing the immunogenicity.
   A17. The method of any one of embodiments A1 to A3 and A6-A16, wherein altering the immunogenicity comprises reducing or neutralizing a myeloid cell response to the hypoimmunogenic cells (such as the engineered hypoimmunogenic cells).
   A18. The method of any one of embodiments A1 to A3 and A6-A17, wherein altering the immunogenicity comprises reducing or neutralizing a T cell response to the hypoimmunogenic cells (such as the engineered hypoimmunogenic cells).
   A19. The method of any one of embodiments A1 to A3 and A6-A18, wherein altering the immunogenicity comprises reducing or neutralizing a natural killer cell response to the hypoimmunogenic cells (such as the engineered hypoimmunogenic cell).
   A20. The method of any one of embodiments A1 to A3 and A6-A19, wherein altering the immunogenicity comprises reducing or neutralizing an antibody response to the hypoimmunogenic cells (such as the engineered hypoimmunogenic cells).
   A21. The method of any one of embodiments A1 to A3 and A6-20, wherein altering the immunogenicity comprises reducing or neutralizing an allogeneic host versus graft rejection.
   A22. The method of any one of embodiments A1 to A3 and A6-A21, wherein altering the immunogenicity comprises one or more of the following in the hypoimmunogenic cell (such as the engineered hypoimmunogenic cell):
a) expression of HLA class II molecules are reduced or ablated;
b) expression of HLA-A, HLA-B, and/or HLA-C are reduced; and
c) expression of HLA-E is reduced but remains detectable.
   A23. The method of any one of embodiments A1 to A3 and A6-A22, wherein altering the immunogenicity comprises reducing or ablating MHC class II mediated response to the hypoimmunogenic cell (such as the engineered hypoimmunogenic cell).
   A24. The method of any one of embodiments A1 to A23, wherein altering the immunogenicity comprises reducing or neutralizing MHC class I mediated response to the hypoimmunogenic cell (such as the engineered hypoimmunogenic cell).
   A25. The method of any one of embodiments A1 to A24, wherein the RFX gene is RFX5, RFXANK or RFXAP,
   A26. The method of any one of embodiments A1 to A25, wherein two or more of RFX5, RFXANK or RFXAP are genetically modified.
   A27. The method of any one of embodiments A1 to A26, wherein each of RFX5, RFXANK, and RFXAP are genetically modified.
   A28. The method of any one of embodiments A1 to A27, further comprising genetically modifying a CD58 gene, wherein genetically modifying the CD58 gene eliminates or reduces the CD58 protein expression.
   A29. The method of embodiment A28, wherein genetically modifying the CD58 gene reduces or ablates costimulatory immune cell response, and/or impairs the formation of an immune synapse.
   A30. The method of any one of embodiments A1 to A29, further comprising genetically modifying a B2M gene, wherein genetically modifying the B2M gene results in reducing or ablating expression of HLA class I molecules on the hypoimmunogenic cell (such as the engineered hypoimmunogenic cell), optionally the HLA class I molecules are selected from the group consisting of HLA-A, HLA-B, HLA-C, HLA-E, and combinations thereof.
   A31. The method of any one of embodiments A1 to A30, further comprising genetically modifying a CIITA gene, wherein genetically modifying the CIITA gene results in reducing or ablating expression of HLA class II molecules on the hypoimmunogenic cell (such as the engineered hypoimmunogenic cell).
   A32. The method of any one of embodiments A1 to A31, wherein genetically modifying the RFX gene comprises:
(i) modifying the DNA sequence of the RFX gene, optionally through a CRISPR-Cas system;
(ii) repressing transcription or translation of the RFX mRNA through RNAi system, optionally the RNAi system comprises shRNA, siRNA, miR-adapted shRNA, or a combination thereof; or
(iii) reducing or ablating transcription of the RFX gene, optionally through recruiting or directing transcriptional repressors to the RFX gene,
   A33. The method of any one of embodiments A1 to A32, wherein genetically modifying the CIITA gene and/or the B2M gene and/or the CD58 gene comprises: (i) modifying the DNA sequence of the CIITA gene and/or the B2M gene and/or the CD58 gene, optionally through a CRISPR-Cas system; (ii) repressing transcription or translation of the CIITA gene and/or the B2M gene and/or the CD58 gene through a RNAi system, optionally wherein the RNAi system comprises shRNA, siRNA, miR-adapted shRNA, or a combination thereof; or (iii) reducing or ablating transcription of the CIITA gene and/or the B2M gene and/or the CD58 gene, optionally through recruiting or directing transcriptional repressors to the CIITA gene and/or the B2M gene and/or the CD58 gene.
   A34. The method of any one of embodiments A1 to A33, wherein the method further comprises genetically modifying at least one of a TNFRSF14 gene, a TNFRSF1A gene, a TNFRSF1B gene, an ICAM1 gene, and a herpesvirus entry mediator (HVEM) gene.
   A35. A non-naturally occurring hypoimmunogenic human cell (such as an engineered hypoimmunogenic human cell) 1 produced by the method of any one of embodiments A1 to A34.
   A36. A non-naturally occurring hypoimmunogenic human cell (such as an engineered hypoimmunogenic human cell), comprising a genetically modified regulatory factor X (RFX) gene, wherein the genetically modified RFX gene reduces expression of the RFX protein, and the hypoimmunogenic human cell (such as the engineered hypoimmunogenic human cell) is produced from an embryoid body; optionally the hypoimmunogenic human cell (such as the engineered hypoimmunogenic human cell) further comprises one or more of a genetically modified class II major histocompatibility complex transactivator (CIITA) gene, a genetically modified beta-2-microglobulin (B2M) gene, and a genetically modified CD58 gene.
   A37. A composition comprising the hypoimmunogenic human cell (such as an engineered hypoimmunogenic human cell) of embodiment A35 or A36.
   A38. A γδT cell-derived induced pluripotent stem (iPS) human cell, comprising a genetically modified regulatory factor X (RFX) gene, wherein the genetically modified RFX gene reduces expression of the RFX protein; optionally the iPS human cell further comprises one or more of a genetically modified class II major histocompatibility complex transactivator (CIITA) gene, a genetically modified beta-2-microglobulin (B2M) gene, and a genetically modified CD58 gene.
   A39. A composition comprising the iPS human cell of embodiment A38.
   A40. A method of hypoimmunogenicity (such as engineering hypoimmunogenicity), comprising:
a) a step for performing a function of genetically modifying a regulatory factor X (RFX) gene of at least one immunogenic human cell, wherein genetically modifying the RFX gene reduces expression of the RFX protein in the immunogenic human cell;
b) a step for performing a function of forming at least one embryoid body or multicellular body from the cell of a) to produce at least one hypoimmunogenic cell (such as an engineered hypoimmunogenic cell);
c) a step for performing a function of subjecting the hypoimmunogenic cell (such as the engineered hypoimmunogenic cell) to an immune system; and
d) a step for performing a function of determining immunogenicity of the hypoimmunogenic cell (such as the engineered hypoimmunogenic cell), wherein the immunogenicity is altered as compared to an immunogenic human cell where the RFX gene is not genetically modified,
optionally wherein step a) further comprises a step for performing a function of genetically modifying a class II major histocompatibility complex transactivator (CIITA) gene, a beta-2-microglobulin (B2M) gene, and/or a CD58 gene of the immunogenic human cell.
   A41. A method of hypoimmunogenicity (such as engineering hypoimmunogenicity), comprising:
a) a step for performing a function of reprogramming an immunogenic human cell to produce an induced pluripotent stem (iPS) human cell, wherein the immunogenic human cell comprises a heterodimeric T-cell receptor comprising a γ chain and a δ chain;
b) a step for performing a function of genetically modifying a regulatory factor X (RFX) gene of the iPS human cell, wherein genetically modifying the RFX gene reduces expression of the RFX protein by the iPS human cell;
c) a step for performing a function of forming at least one embryoid body from the cell of step b) to produce at least one hypoimmunogenic cell (such as an engineered hypoimmunogenic cell);
d) a step for performing a function of subjecting the hypoimmunogenic cell (such as the engineered hypoimmunogenic cell) to an immune system; and
e) a step for performing a function of determining immunogenicity of the hypoimmunogenic cell (such as the engineered hypoimmunogenic cell), wherein the immunogenicity is altered as compared to an iPS human cell where the RFX gene is not genetically modified,
optionally wherein step b) further comprises a step for performing a function of genetically modifying a class II major histocompatibility complex transactivator (CIITA) gene, a beta-2-microglobulin (B2M) gene, and/or a CD58 gene of the iPS human cell.
   A42. A method of hypoimmunogenicity (such as engineering hypoimmunogenicity), comprising:
a) a step for performing a function of genetically modifying a regulatory factor X (RFX) gene of an immunogenic human cell to produce a hypoimmunogenic cell (such as an engineered hypoimmunogenic cell), wherein genetically modifying the RFX gene reduces expression of the RFX protein by the immunogenic human cell;
b) a step for performing a function of subjecting the hypoimmunogenic cell (such as the engineered hypoimmunogenic cell) to an immune system; and
c) a step for performing a function of determining immunogenicity of the hypoimmunogenic cell (such as the engineered hypoimmunogenic cell), wherein the immunogenicity is altered as compared to an immunogenic human cell where the RFX gene is not genetically modified,
optionally wherein step a) further comprises a step for performing a function of genetically modifying a class II major histocompatibility complex transactivator (CIITA) gene, a beta-2-microglobulin (B2M) gene, and/or a CD58 gene of the immunogenic human cell.
   A43. A non-naturally occurring hypoimmunogenic human cell (such as an engineered hypoimmunogenic human cell), comprising a means for reducing expression of an RFX protein through a genetically modified RFX gene, and/or a means for altering immunogenicity of an immune system to the hypoimmunogenic human cell (such as the engineered hypoimmunogenic human cell) as compared to an immunogenic cell where the RFX gene is not genetically modified; optionally wherein the hypoimmunogenic human cell (such as the engineered hypoimmunogenic human cell) further comprises a means for reducing expression of a CIITA protein, a B2M protein, and/or a CD58 protein through a genetically modified CIITA gene, a genetically modified B2M gene, and/or a genetically modified CD58 gene.
   A44. A γδT cell-derived induced pluripotent stem (iPS) human cell, comprising a means for reducing expression of an RFX protein through a genetically modified RFX gene, and/or a means for altering immunogenicity of an immune system to the iPS human cell as compared to an iPS human cell where the RFX gene is not genetically modified; optionally wherein the iPS human cell further comprises a means for reducing expression of a CIITA protein, a B2M protein, and/or a CD58 protein through a genetically modified CIITA gene, a genetically modified B2M gene, and/or a genetically modified CD58 gene.

In one set of embodiments (embodiment set B), provided are:

B1. A method of hypoimmunogenicity (such as engineering hypoimmunogenicity), comprising:
a) reprogramming an immunogenic human cell to produce an induced pluripotent (iPS) human cell, wherein the immunogenic human cell comprises a heterodimeric T-cell receptor comprising a γ chain and a δ chain;
b) genetically modifying a beta-2-microglobulin (B2M) gene of the iPS human cell, wherein genetically modifying the B2M gene reduces expression of the B2M protein by the iPS human cell;
c) forming at least one embryoid body or multicellular body from the cell of step b) to produce at least one hypoimmunogenic cell (such as an engineered hypoimmunogenic cell);
d) subjecting the hypoimmunogenic cell (such as the engineered hypoimmunogenic cell) to an immune system; and
e) determining immunogenicity of the hypoimmunogenic cell (such as the engineered hypoimmunogenic cell), wherein the immunogenicity is altered as compared to an iPS human cell where the B2M gene is not genetically modified,
optionally wherein step b) further comprises genetically modifying one or more of a class II major histocompatibility complex transactivator (CIITA) gene, a regulatory factor X (RFX) gene, and a CD58 gene of the iPS human cell.
   B2. A method of hypoimmunogenicity (such as engineering hypoimmunogenicity), comprising:
a) genetically modifying a beta-2-microglobulin (B2M) gene of at least one immunogenic human cell, wherein genetically modifying the B2M gene reduces expression of the B2M by the immunogenic human cell;
b) forming at least one embryoid body or multicellular body from the cell of a) to produce at least one hypoimmunogenic cell (such as an engineered hypoimmunogenic cell);
c) subjecting the hypoimmunogenic cell (such as the engineered hypoimmunogenic cell) to an immune system; and
d) determining immunogenicity of the hypoimmunogenic cell (such as the engineered hypoimmunogenic cell), wherein the immunogenicity is altered as compared to an immunogenic human cell where the B2M gene is not genetically modified,
optionally wherein step a) further comprises genetically modifying one or more of a class II major histocompatibility complex transactivator (CIITA) gene, a regulatory factor X (RFX) gene, and a CD58 gene of the immunogenic human cell.
   B3. A method of hypoimmunogenicity (such as engineering hypoimmunogenicity), comprising:
a) genetically modifying a beta-2-microglobulin (B2M) gene of an immunogenic human cell to produce a hypoimmunogenic cell (such as an engineered hypoimmunogenic cell), wherein genetically modifying the B2M gene reduces expression of the B2M protein by the immunogenic human cell;
b) subjecting the hypoimmunogenic cell (such as the engineered hypoimmunogenic cell) to an immune system; and
c) determining immunogenicity of the hypoimmunogenic cell (such as the engineered hypoimmunogenic cell), wherein the immunogenicity is altered as compared to an immunogenic human cell where the B2M gene is not genetically modified,
optionally wherein step a) further comprises genetically modifying one or more of a class II major histocompatibility complex transactivator (CIITA) gene, a regulatory factor X (RFX) gene, and a CD58 of the immunogenic human cell.
   B4. A method of producing a hypoimmunogenic cell (such as an engineered hypoimmunogenic cell) from an immunogenic cell, comprising: (i) genetically modifying a beta-2-microglobulin (B2M) gene in the immunogenic cell, wherein genetically modifying the B2M gene reduces expression of the B2M protein in said cell, and (ii) optionally further genetically modifying one or more genes selected from a class II major histocompatibility complex transactivator (CIITA) gene, a regulatory factor X (RFX) gene, and a CD58 gene in said immunogenic cell, wherein genetically modifying said one or more genes reduces expression of the corresponding one or more proteins in said immunogenic cell, wherein said method results in production of the hypoimmunogenic cell (such as the engineered hypoimmunogenic cell), which has one or more of the following properties: a) having a reduced immunogenicity upon the hypoimmunogenic cell's (such as the engineered hypoimmunogenic cell's) presence in an allogeneic or non-MHC matched subject as compared to a corresponding immunogenic cell, but without the genetic modification(s) of (i) and (ii); b) causing a reduced immune response to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii); and c) causing a reduced alloreactive T cell cytotoxicity to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii).
   B5. A method of producing a hypoimmunogenic cell (such as an engineered hypoimmunogenic cell) from an immunogenic cell, comprising: a) reprogramming the immunogenic cell to produce an induced pluripotent stem (iPS) cell; b) (i) genetically modifying a beta-2-microglobulin (B2M) gene in the iPS cell produced in step (a), wherein genetically modifying the B2M gene reduces expression of the B2M protein in said iPS cell, and (ii) optionally further genetically modifying one or more genes selected from a class II major histocompatibility complex transactivator (CIITA) gene, a regulatory factor X (RFX) gene, and a CD58 gene in said iPS cell, wherein genetically modifying said one or more genes reduces expression of the corresponding one or more proteins in said iPS cell; and c) optionally, differentiating the cell produced in step (b); wherein said method results in production of the hypoimmunogenic cell (such as the engineered hypoimmunogenic cell), which has one or more of the following properties: 1) having a reduced immunogenicity upon the hypoimmunogenic cell's, such as the engineered hypoimmunogenic cell's, presence in an allogeneic or non-MHC matched subject, as compared to a corresponding iPS cell, or a cell corresponding to the cell produced in step (c), but without the genetic modification(s) of step (b); 2) causing a reduced immune response to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b); and 3) causing a reduced alloreactive T cell cytotoxicity to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b).
   B6. The method of any one of embodiments B1-B5, wherein the hypoimmunogenic cell (such as an engineered hypoimmunogenic cell) comprises a T-cell receptor (TCR) comprising a γ chain and a 6 chain.
   B7. The method of any one of embodiments B1-B6, wherein the immunogenic cell or the human immunogenic cell is an immune cell, optionally selected from T cells, natural killer (NK) cells, B cells, and hematopoietic stem cells (HSCs).
   B8. The method of any one of embodiments B1-B7, wherein the reduced immunogenicity of the hypoimmunogenic cell (such as the engineered hypoimmunogenic cell) comprises one or more of the following: i) a reduced or ablated myeloid cell response to the hypoimmunogenic cell (such as an engineered hypoimmunogenic cell) upon the cell's presence in an allogeneic or non-MHC matched subject, as compared to a cell corresponding to the cell that was modified but without said genetic modification(s); ii) a reduced or ablated T cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iii) a reduced or ablated natural killer (NK) cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iv) a reduced or ablated neutralizing antibody response to the hypoimmunogenic cell (such as the engineered hypoimmunogenic cell) upon the cell's presence in an allogeneic or non-MHC matched subject, as compared to a cell corresponding to the cell that was modified but without said genetic modification(s); v) a reduced or ablated MHC class II mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); vi) a reduced or ablated neutralizing MHC class I mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); and vii) a reduced or ablated allogeneic host versus graft rejection of to the hypoimmunogenic cell (such as the engineered hypoimmunogenic cell) upon the cell's presence in an allogeneic subject, as compared to a cell corresponding to the cell that was modified but without said genetic modification(s).
   B9. The method of any one of embodiments B4-B8, wherein the immunogenic cell is a human cell.
   B10. The method of embodiment B9, wherein in the hypoimmunogenic cell (such as the engineered hypoimmunogenic cell): i) expression of HLA class II molecules is reduced or ablated; ii) expression of HLA-A, HLA-B, and/or HLA-C is reduced; and iii) expression of HLA-E is reduced but remains detectable.
   B11. The method of any one of embodiments B4-B10, wherein the method comprises forming at least one embryoid body or multicellular body from the genetically modified cell to produce the hypoimmunogenic cell (such as the engineered hypoimmunogenic cell).
   B12. The method of any one of embodiments B4-B11, wherein the method further comprises determining immunogenicity of the hypoimmunogenic cell (such as the engineered hypoimmunogenic cell).
   B13. The method of any one of embodiments B1-B12, wherein the method further comprises administering the hypoimmunogenic cell (such as the engineered hypoimmunogenic cell) to an allogeneic or non-MHC matched subject.
   B14. The method of any one of embodiments B1-B13, wherein the immunogenicity of the hypoimmunogenic cell (such as the engineered hypoimmunogenic cell) is altered as compared to an immunogenic cell (such as the immunogenic human cell) or an iPS human cell or an iPS cell where the only difference between the hypoimmunogenic cell (such as the engineered hypoimmunogenic cell) and the immunogenic cell (such as an immunogenic human cell) or the iPS human cell or the iPS cell is that the B2M gene and optionally one or more of the RFX gene, the CIITA gene, and the CD58 gene is not genetically modified in the immunogenic cell (such as the immunogenic human cell) or the iPS human cell or the iPS cell.
   B15. The method of any one of embodiments B1 to B14, wherein the immunogenic human cell or the immunogenic cell is allogeneic or non-HLA matched or non-MHC matched to cells, receptors, or polypeptides of the immune system of a recipient subject.
   B16. The method of any one of embodiments Bi to B3 and B6 to B15, wherein altering the immunogenicity comprises balancing, reducing, or neutralizing the immunogenicity, such as reducing or neutralizing the immunogenicity.
   B17. The method of any one of embodiments Bi to B3 and B6 to B16, wherein altering the immunogenicity comprises reducing or neutralizing a myeloid cell response to the hypoimmunogenic cells (such as the engineered hypoimmunogenic cells).
   B18. The method of any one of embodiments B1 to B3 and B6 to B17, wherein altering the immunogenicity comprises reducing or neutralizing a T cell response to the hypoimmunogenic cells (such as the engineered hypoimmunogenic cells).
   B19. The method of any one of embodiments Bi to B3 and B6 to B18, wherein altering the immunogenicity comprises reducing or neutralizing a natural killer cell response to the hypoimmunogenic cells (such as the engineered hypoimmunogenic cells).
   B20. The method of any one of embodiments Bi to B3 and B6 to B19, wherein altering the immunogenicity comprises reducing or neutralizing an antibody response to the hypoimmunogenic cells (such as the engineered hypoimmunogenic cell).
   B21. The method of any one of embodiments B1 to B3 and B6 to B20, wherein altering the immunogenicity comprises reducing or neutralizing an allogeneic host versus graft rejection.
   B22. The method of any one of embodiments B1 to B3 and B6 to B21, wherein altering the immunogenicity comprises reducing or ablating expression of HLA class I molecules on the hypoimmunogenic cell (such as the engineered hypoimmunogenic cell).
   B23. The method of any one of embodiments B1 to B3 and B6 to B22, further comprising genetically modifying a RFX gene, wherein the RFX gene is RFX5, RFXANK or RFXAP
   B24. The method of embodiment B23, wherein two or more of RFX5, RFXANK or RFXAP are genetically modified.
   B25. The method of embodiment B23 or B24, wherein each of RFX5, RFXANK, and RFXAP are genetically modified.
   B26. The method of any one of embodiments B23-B25, wherein genetically modifying the RFX gene results in one or more of the following in the hypoimmunogenic cell (such as the engineered hypoimmunogenic cell):
a) expression of HLA class II molecules are reduced or ablated; or
b) expression of HLA-A, HLA-B, and/or HLA-C are reduced.
   B27. The method of any one of embodiments B23-B26, wherein genetically modifying the RFX gene results in reducing or ablating MHC class II mediated response to the hypoimmunogenic cell (such as the engineered hypoimmunogenic cell).
   B28. The method of any one of embodiments B23 to B27, wherein genetically modifying the RFX gene results in reducing or neutralizing MHC class I mediated response to the hypoimmunogenic cell (such as the engineered hypoimmunogenic cell).
   B29. The method of any one of embodiments B1 to B28, further comprising genetically modifying a CIITA gene, wherein genetically modifying the CIITA gene results in reducing or ablating expression of HLA class II molecules on the hypoimmunogenic cell (such as the engineered hypoimmunogenic cell).
   B30. The method of any one of embodiments B1 to B29, further comprising genetically modifying a CD58 gene, wherein genetically modifying the CD58 gene eliminates or reduces the CD58 expression.
   B31. The method of embodiment B30, wherein genetically modifying the CD58 gene reduces or ablates a co-stimulatory immune cell response, and/or impairs the formation of an immune synapse.
   B32. The method of any one of embodiments B1 to B31, wherein genetically modifying the B2M gene comprises:
(i) modifying the DNA sequence of the B2M gene, optionally through a CRISPR-Cas system;
(ii) repressing transcription of the B2M mRNA through RNAi system, optionally the RNAi system comprises shRNA, siRNA, or miR-adapted shRNA; or
(iii) reducing or ablating transcription of the B2M gene, optionally through recruiting or directing transcriptional repressors to the B2M gene.
   B33. The method of any one of embodiments B1 to B32, wherein genetically modifying the CIITA gene and/or the RFX gene and/or the CD58 gene comprises: (i) modifying the DNA sequence of the CIITA gene and/or the RFX gene and/or the CD58 gene, optionally through a CRISPR-Cas system; (ii) repressing transcription or translation of the CIITA gene and/or the RFX gene and/or the CD58 gene through a RNAi system, optionally wherein the RNAi system comprises shRNA, siRNA, miR-adapted shRNA, or a combination thereof; or (iii) reducing or ablating transcription of the CIITA gene and/or the RFX gene and/or the CD58 gene, optionally through recruiting or directing transcriptional repressors to the CIITA gene and/or the RFX gene and/or the CD58 gene.
   B34. The method of any one of embodiments B1 to B33, wherein the method further comprises genetically modifying at least one of a TNFRSF14 gene, a TNFRSF1A gene, a TNFRSF1B gene, an ICAM1 gene, and a herpesvirus entry mediator (HVEM) gene.
   B35. A non-naturally occurring hypoimmunogenic human cell (such as an engineered hypoimmunogenic human cell) produced by the method of any one of embodiments B1 to B34.
   B36. A non-naturally occurring hypoimmunogenic human cell (such as an engineered hypoimmunogenic human cell), comprising a genetically modified B2M gene, wherein the genetically modified B2M gene reduces expression of the B2M protein, and the hypoimmunogenic human cell (such as the engineered hypoimmunogenic human cell) is produced from an embryoid body; optionally the hypoimmunogenic human cell (such as or the engineered hypoimmunogenic human cell) further comprises one or more of a genetically modified CIITA gene, a genetically modified RFX gene, and a genetically modified CD58 gene.
   B37. A composition comprising the hypoimmunogenic human cell (such as the engineered hypoimmunogenic human cell) of embodiment B35 or B36.
   B38. A γδT cell-derived induced pluripotent stem (iPS) human cell, comprising a genetically modified B2M gene, wherein the genetically modified B2M gene reduces expression of the B2M protein; optionally the iPS human cell further comprises one or more of a genetically modified CIITA gene, a genetically modified RFX gene, and a genetically modified CD58 gene.
   B39. A composition comprising the iPS human cell of embodiment B38.
   B40. A method of hypoimmunogenicity (such as engineering hypoimmunogenicity), comprising:
a) a step for performing a function of genetically modifying a B2M gene of at least one immunogenic human cell, wherein genetically modifying the B2M gene reduces expression of the B2M protein in the immunogenic human cell;
b) a step for performing a function of forming at least one embryoid body or multicellular body from the cell of a) to produce at least one hypoimmunogenic cell (such as an engineered hypoimmunogenic cell);
c) a step for performing a function of subjecting the hypoimmunogenic cell (such as the engineered hypoimmunogenic cell) to an immune system; and
d) a step for performing a function of determining immunogenicity of the hypoimmunogenic cell (such as the engineered hypoimmunogenic cell), wherein the immunogenicity is altered as compared to an immunogenic human cell where the B2M gene is not genetically modified,
optionally wherein step a) further comprises a step for performing a function of genetically modifying a RFX gene, a CIITA gene, and/or a CD58 gene of the immunogenic human cell.
   B41. A method of hypoimmunogenicity (such as engineering hypoimmunogenicity), comprising:
a) a step for performing a function of reprogramming an immunogenic human cell to produce an induced pluripotent stem (iPS) human cell, wherein the immunogenic human cell comprises a heterodimeric T-cell receptor comprising a γ chain and a δ chain;
b) a step for performing a function of genetically modifying a B2M gene of the iPS human cell, wherein genetically modifying the B2M gene reduces expression of the B2M protein by the iPS human cell;
c) a step for performing a function of forming at least one embryoid body from the cell of step b) to produce at least one hypoimmunogenic cell (such as an engineered hypoimmunogenic cell);
d) a step for performing a function of subjecting the hypoimmunogenic cell (such as the engineered hypoimmunogenic cell) to an immune system; and
e) a step for performing a function of determining immunogenicity of the hypoimmunogenic cell (such as the engineered hypoimmunogenic cell), wherein the immunogenicity is altered as compared to an iPS human cell where the B2M gene is not genetically modified,
optionally wherein step b) further comprises a step for performing a function of genetically modifying a RFX gene, a CIITA gene, and/or a CD58 gene of the iPS human cell.
   B42. A method of hypoimmunogenicity (such as engineering hypoimmunogenicity), comprising:
a) a step for performing a function of genetically modifying a B2M gene of an immunogenic human cell to produce a hypoimmunogenic cell (such as an engineered hypoimmunogenic cell), wherein genetically modifying the B2M gene reduces expression of the B2M protein by the immunogenic human cell;
b) a step for performing a function of subjecting the hypoimmunogenic cell (such as the engineered hypoimmunogenic cell) to an immune system; and
c) a step for performing a function of determining immunogenicity of the hypoimmunogenic cell (such as the engineered hypoimmunogenic cell), wherein the immunogenicity is altered as compared to an immunogenic human cell where the B2M gene is not genetically modified,
optionally wherein step a) further comprises a step for performing a function of genetically modifying a RFX gene, a CIITA gene, and/or a CD58 gene of the immunogenic human cell.
   B43. A non-naturally occurring hypoimmunogenic human cell (such as an engineered hypoimmunogenic human cell), comprising a means for reducing expression of a B2M protein through a genetically modified B2M gene, and/or a means for altering immunogenicity of an immune system to the hypoimmunogenic human cell (such as the engineered hypoimmunogenic human cell) as compared to an immunogenic human cell where the B2M gene is not genetically modified; optionally wherein the hypoimmunogenic human cell (such as the engineered hypoimmunogenic human cell) further comprises a means for reducing expression of a RFX protein, a CD58 protein, and/or a CIITA protein through a genetically modified RFX gene, a genetically modified CD58 gene, and/or a genetically modified CIITA gene.
   B44. A γδT cell-derived induced pluripotent stem (iPS) human cell, comprising a means for reducing expression of a B2M protein through a genetically modified B2M gene, and/or a means for altering immunogenicity of an immune system to the iPS human cell as compared to an iPS human cell where the B2M gene is not genetically modified; optionally wherein the iPS human cell further comprises a means for reducing expression of a RFX protein, a CD58 protein, and/or a CIITA protein through a genetically modified RFX gene, a genetically modified CD58 gene, and/or a genetically modified CIITA gene.

In one set of embodiments (embodiment set C), provided are:

C1. A method of hypoimmunogenicity (such as engineering hypoimmunogenicity), comprising:
a) genetically modifying a CD58 gene of at least one immunogenic human cell, wherein genetically modifying the CD58 gene reduces expression of the CD58 protein by the immunogenic human cell;
b) forming at least one embryoid body or multicellular body from the cell of a) to produce at least one hypoimmunogenic cell (such as an engineered hypoimmunogenic cell);
c) subjecting the hypoimmunogenic cell (such as the engineered hypoimmunogenic cell) to an immune system; and
d) determining immunogenicity of the hypoimmunogenic cell (such as the engineered hypoimmunogenic cell), wherein the immunogenicity is altered as compared to an immunogenic human cell where the CD58 gene is not genetically modified,
optionally wherein step a) further comprises genetically modifying one or more of a class II major histocompatibility complex transactivator (CIITA) gene, a regulatory factor X (RFX) gene, and a beta-2-microglobulin (B2M) gene of the immunogenic human cell.

C2. A method of hypoimmunogenicity (such as engineering hypoimmunogenicity), comprising:
a) reprogramming an immunogenic human cell to produce an induced pluripotent (iPS) human cell, wherein the immunogenic human cell comprises a heterodimeric T-cell receptor comprising a γ chain and a δ chain;
b) genetically modifying a CD58 gene of the iPS human cell, wherein genetically modifying the CD58 gene reduces expression of the CD58 protein by the iPS human cell;
c) forming at least one embryoid body from the cell of step b) to produce at least one hypoimmunogenic cell (such as an engineered hypoimmunogenic cell);
d) subjecting the hypoimmunogenic cell (such as the engineered hypoimmunogenic cell) to an immune system; and
e) determining immunogenicity of the hypoimmunogenic cell (such as the engineered hypoimmunogenic cell), wherein the immunogenicity is altered as compared to an iPS human cell where the CD58 gene is not genetically modified,
optionally wherein step b) further comprises genetically modifying one or more of a class II major histocompatibility complex transactivator (CIITA) gene, a regulatory factor X (RFX) gene, and a beta-2-microglobulin (B2M) gene of the immunogenic human cell of the iPS human cell.

C3. A method of hypoimmunogenicity (such as engineering hypoimmunogenicity), comprising:
a) genetically modifying a CD58 gene of an immunogenic human cell to produce a hypoimmunogenic cell (such as an engineered hypoimmunogenic cell), wherein genetically modifying the CD58 gene reduces expression of the CD58 protein by the immunogenic human cell;
b) subjecting the hypoimmunogenic cell (such as the engineered hypoimmunogenic cell) to an immune system; and
c) determining immunogenicity of the hypoimmunogenic cell (such as the engineered hypoimmunogenic cell), wherein the immunogenicity is altered as compared to an immunogenic human cell where the CD58 gene is not genetically modified,
optionally wherein step a) further comprises genetically modifying one or more of a class II major histocompatibility complex transactivator (CIITA) gene, a regulatory factor X (RFX) gene, and a beta-2-microglobulin (B2M) gene of the immunogenic human cell.
   C4. A method of producing a hypoimmunogenic cell (such as an engineered hypoimmunogenic cell) from an immunogenic cell, comprising: (i) genetically modifying a CD58 gene in the immunogenic cell, wherein genetically modifying the CD58 gene reduces expression of the CD58 protein in said cell, and (ii) optionally further genetically modifying one or more genes selected from a class II major histocompatibility complex transactivator (CIITA) gene, a regulatory factor X (RFX) gene, and a beta-2-microglobulin (B2M) gene in said immunogenic cell, wherein genetically modifying said one or more genes reduces expression of the corresponding one or more proteins in said immunogenic cell, wherein said method results in production of the hypoimmunogenic cell (such as the engineered hypoimmunogenic cell), which has one or more of the following properties: a) having a reduced immunogenicity upon the hypoimmunogenic cell's (such as the engineered hypoimmunogenic cell's) presence in an allogeneic or non-MHC matched subject as compared to a corresponding immunogenic cell, but without the genetic modification(s) of (i) and (ii); b) causing a reduced immune response to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii); and c) causing a reduced alloreactive T cell cytotoxicity to said hypoimmunogenic cell (such as the engineered hypoimmunogenic cell) upon its presence in an allogeneic or non-MHC matched subject as compared to a corresponding immunogenic cell, but without the genetic modification(s) of (i) and (ii).
   C5. A method of producing a hypoimmunogenic cell (such as an engineered hypoimmunogenic cell) from an immunogenic cell, comprising: a) reprogramming the immunogenic cell to produce an induced pluripotent stem (iPS) cell; b) (i) genetically modifying a CD58 gene in the iPS cell in step (a), wherein genetically modifying the CD58 gene reduces expression of the CD58 protein in said iPS cell, and (ii) optionally further genetically modifying one or more genes selected from a class II major histocompatibility complex transactivator (CIITA) gene, a regulatory factor X (RFX) gene, and a beta-2-microglobulin (B2M) gene in said iPS cell, wherein genetically modifying said one or more genes reduces expression of the corresponding one or more proteins in said iPS cell; and c) optionally, differentiating the cell produced in step (b); wherein said method results in production of the hypoimmunogenic cell (such as the engineered hypoimmunogenic cell), which has one or more of the following properties: 1) having a reduced immunogenicity upon the hypoimmunogenic cell's, such as the engineered hypoimmunogenic cell's, presence in an allogeneic or non-MHC matched subject, as compared to a corresponding iPS cell, or a cell corresponding to the cell produced in step (c), but without the genetic modification(s) of step (b); 2) causing a reduced immune response to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b); and 3) causing a reduced alloreactive T cell cytotoxicity to said hypoimmunogenic cell, such as the engineered hypoimmunogenic cell, upon its presence in an allogeneic or non-MHC matched subject, as compared to a corresponding iPS cell or a cell corresponding to the cell produced in step (c), but without the genetic modification(s) of step (b).
   C6. The method of any one of embodiments C1-C5, wherein the hypoimmunogenic cell (such as the engineered hypoimmunogenic cell) comprises a T-cell receptor (TCR) comprising a γ chain and a 6 chain.
   C7. The method of any one of embodiments C1-C6, the immunogenic cell or the human immunogenic cell is an immune cell, optionally selected from T cells, natural killer (NK) cells, B cells, and hematopoietic stem cells (HSCs).
   C8. The method of any one of embodiments C1-C7, wherein the reduced immunogenicity of the hypoimmunogenic cell (such as the engineered hypoimmunogenic cell) comprises one or more of the following: i) a reduced or ablated myeloid cell response to the hypoimmunogenic cell (such as an engineered hypoimmunogenic cell) upon the cell's presence in an allogeneic or non-MHC matched subject, as compared to a cell corresponding to the cell that was modified but without said genetic modification(s); ii) a reduced or ablated T cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iii) a reduced or ablated natural killer (NK) cell response to the hypoimmunogenic cell (such as the engineered hypoimmunogenic cell) upon the cell's presence in an allogeneic or non-MHC matched subject, as compared to a cell corresponding to the cell that was modified but without said genetic modification(s); iv) a reduced or ablated neutralizing antibody response to the hypoimmunogenic cell (such as the engineered hypoimmunogenic cell) upon the cell's presence in an allogeneic or non-MHC matched subject, as compared to a cell corresponding to the cell that was modified but without said genetic modification(s); v) a reduced or ablated MHC class II mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); vi) a reduced or ablated neutralizing MHC class I mediated response to the hypoimmunogenic cell (such as the engineered hypoimmunogenic cell) upon the cell's presence in an allogeneic or non-MHC matched subject, as compared to a cell corresponding to the cell that was modified but without said genetic modification(s); and vii) a reduced or ablated allogeneic host versus graft rejection of to the hypoimmunogenic cell (such as the engineered hypoimmunogenic cell) upon the cell's presence in an allogeneic subject, as compared to a cell corresponding to the cell that was modified but without said genetic modification(s).
   C9. The method of any one of embodiments C4-C8, wherein the immunogenic cell is a human cell.
   C10. The method of embodiment C9, wherein in the hypoimmunogenic cell (such as the engineered hypoimmunogenic cell): i) expression of HLA class II molecules is reduced or ablated; ii) expression of HLA-A, HLA-B, and/or HLA-C is reduced; and iii) expression of HLA-E is reduced but remains detectable.
   C11. The method of any one of embodiments C4-C10, wherein the method comprises forming at least one embryoid body or multicellular body from the genetically modified cell to produce the hypoimmunogenic cell (such as the engineered hypoimmunogenic cell).
   C12. The method of any one of embodiments C4-C11, wherein the method further comprises determining immunogenicity of the hypoimmunogenic cell (such as the engineered hypoimmunogenic cell).
   C13. The method of any one of embodiments C1-C12, wherein the method further comprises administering the hypoimmunogenic cell (such as the engineered hypoimmunogenic cell) to an allogeneic or non-MHC matched subject.
   C14. The method of any one of embodiments C1-C13, wherein the immunogenicity of the hypoimmunogenic cell (such as the engineered hypoimmunogenic cell) is altered as compared to an immunogenic cell (such as the immunogenic human cell) or an iPS human cell or an iPS cell where the only difference between the hypoimmunogenic cell (such as the engineered hypoimmunogenic cell) and the immunogenic cell (such as the immunogenic human cell) or the iPS human cell or the iPS cell is that the CD58 gene and optionally one or more of the RFX gene, the CIITA gene, and the B2M gene is not genetically modified in the immunogenic cell (such as the immunogenic human cell) or the iPS human cell or the iPS cell.
   C15. The method of any one of embodiments C1 to C3 and C6 to C14, wherein the immunogenic human cell or the immunogenic cell is allogeneic or non-HLA matched or non-MHC matched to cells, receptors, or polypeptides of the immune system of a recipient subject.
   C16. The method of any one of embodiments C1 to C3 and C6 to C15, wherein altering the immunogenicity comprises balancing, reducing, or neutralizing the immunogenicity, such as reducing or neutralizing the immunogenicity.
   C17. The method of any one of embodiments C1 to C3 and C6 to C16, wherein altering the immunogenicity comprises reducing or neutralizing a myeloid cell response to the hypoimmunogenic cells (such as the engineered hypoimmunogenic cells).
   C18. The method of any one of embodiments C1 to C3 and C6 to C17, wherein altering the immunogenicity comprises reducing or neutralizing a T cell response to the hypoimmunogenic cells (such as the engineered hypoimmunogenic cell).
   C19. The method of any one of embodiments C1 to C3 and C6 to C18, wherein altering the immunogenicity comprises reducing or neutralizing a natural killer cell response to the hypoimmunogenic cells (such as the engineered hypoimmunogenic cells).
   C20. The method of any one of embodiments C1 to C3 and C6 to C19, wherein altering the immunogenicity comprises reducing or neutralizing an allogeneic host versus graft rejection.
   C21. The method of any one of embodiments C1 to C3 and C6 to C20, wherein altering the immunogenicity comprises reducing or ablating a co-stimulatory immune cell response, and/or impairing the formation of an immune synapse.
   C22. The method of any one of embodiments C1 to C3 and C6 to C21, further comprising genetically modifying a RFX gene, wherein the RFX gene is RFX5, RFXANK, or RFXAP.
   C23. The method of embodiment C22, wherein two or more of RFX5, RFXANK or RFXAP are genetically modified.
   C24. The method of embodiment C22 or C23, wherein each of RFX5, RFXANK, and RFXAP are genetically modified.
   C25. The method of any one of embodiments C22-C24, wherein genetically modifying the RFX gene results in one or more of the following in the hypoimmunogenic cell (such as the engineered hypoimmunogenic cell):
a) expression of HLA class II molecules are reduced or ablated;
b) expression of HLA-A, HLA-B, and/or HLA-C are reduced; and
c) expression of HLA-E is reduced but remains detectable.
   C26. The method of any one of embodiments C22-C25, wherein genetically modifying the RFX gene results in reducing or ablating MHC class II mediated response to the hypoimmunogenic cell (such as the engineered hypoimmunogenic cell).
   C27. The method of any one of embodiments C22 to C26, wherein genetically modifying the RFX gene results in reducing or neutralizing MHC class I mediated response to the hypoimmunogenic cell (such as the engineered hypoimmunogenic cell).
   C28. The method of any one of embodiments C1 to C27, further comprising genetically modifying a B2M gene, wherein genetically modifying the B2M gene results in reducing or ablating expression of HLA class I molecules.
   C29. The method of any one of embodiments C1 to C28, further comprising genetically modifying a CIITA gene, wherein genetically modifying the CIITA gene results in reducing or ablating expression of HLA class II molecules on the hypoimmunogenic cell (such as the engineered hypoimmunogenic cell).
   C30. The method of any one of embodiments C1 to C29, wherein genetically modifying the CD58 gene comprises:
(i) modifying the DNA sequence of the CD58 gene, optionally through a CRISPR-Cas system;
(ii) repressing transcription or translation of the CD58 mRNA through RNAi system, optionally the RNAi system comprises shRNA, siRNA, or miR-adapted shRNA; or
(iii) reducing or ablating transcription of the CD58 gene, optionally through recruiting or directing transcriptional repressors to the CD58 gene.
   C31. The method of any one of embodiments C1-C30, wherein genetically modifying the CIITA gene and/or the B2M gene and/or the RFX gene comprises: (i) modifying the DNA sequence of the CIITA gene and/or the B2M gene and/or the RFX gene, optionally through a CRISPR-Cas system; (ii) repressing transcription or translation of the CIITA gene and/or the B2M gene and/or the RFX gene through a RNAi system, optionally wherein the RNAi system comprises shRNA, siRNA, miR-adapted shRNA, or a combination thereof; or (iii) reducing or ablating transcription of the CIITA gene and/or the B2M gene and/or the RFX gene, optionally through recruiting or directing transcriptional repressors to the CIITA gene and/or the B2M gene and/or the RFX gene.
   C32. The method of any one of embodiments C1 to C31, wherein the method further comprises genetically modifying at least one of a TNFRSF14 gene, a TNFRSF1A gene, a TNFRSF1Bgene, an ICAM1 gene, and a herpesvirus entry mediator (HVEM) gene.
   C33. A non-naturally occurring hypoimmunogenic human cell (such as an engineered hypoimmunogenic human cell) produced by the method of any one of embodiments C1 to C32.
   C34. A non-naturally occurring hypoimmunogenic human cell (such as an engineered hypoimmunogenic human cell), comprising a genetically modified CD58 gene, wherein the genetically modified CD58 gene reduces expression of the CD58 protein, and the hypoimmunogenic human cell (such as the engineered hypoimmunogenic human cell) is produced from an embryoid body; optionally the hypoimmunogenic human cell (such as the engineered hypoimmunogenic human cell) further comprises one or more of a genetically modified CIITA gene, a genetically modified RFX gene, and a genetically modified B2M gene.
   C35. A composition comprising the hypoimmunogenic human cell (such as the engineered hypoimmunogenic human cell) of embodiment C33 or C34.
   C36. A γδT cell-derived induced pluripotent stem (iPS) human cell, comprising a genetically modified CD58 gene, wherein the genetically modified CD58 gene reduces expression of the CD58 protein; optionally the iPS human cell further comprises one or more of a genetically modified CIITA gene, a genetically modified RFX gene, and a genetically modified B2M gene.
   C37. A composition comprising the iPS human cell of embodiment C36.
   C38. A method of hypoimmunogenicity (such as engineering hypoimmunogenicity), comprising:
a) a step for performing a function of genetically modifying a CD58 gene of at least one immunogenic human cell, wherein genetically modifying the CD58 gene reduces expression of the CD58 protein in the immunogenic human cell;
b) a step for performing a function of forming at least one embryoid body or multicellular body from the cell of a) to produce at least one hypoimmunogenic cell (such as the engineered hypoimmunogenic cell);
c) a step for performing a function of subjecting the hypoimmunogenic cell (such as the engineered hypoimmunogenic cell) to an immune system; and
d) a step for performing a function of determining immunogenicity of the hypoimmunogenic cell (such as the engineered hypoimmunogenic cell), wherein the immunogenicity is altered as compared to an immunogenic human cell where the CD58 gene is not genetically modified,
optionally wherein step a) further comprises a step for performing a function of genetically modifying a RFX gene, a CIITA gene, and/or a B2M gene of the immunogenic human cell.
   C39. A method of hypoimmunogenicity (such as engineering hypoimmunogenicity), comprising:
a) a step for performing a function of reprogramming an immunogenic human cell to produce an induced pluripotent stem (iPS) human cell, wherein the immunogenic human cell comprises a heterodimeric T-cell receptor comprising a γ chain and a δ chain;
b) a step for performing a function of genetically modifying a CD58 gene of the iPS human cell, wherein genetically modifying the CD58 gene reduces expression of the CD58 protein by the iPS human cell;
c) a step for performing a function of forming at least one embryoid body from the cell of step b) to produce at least one hypoimmunogenic cell (such as the engineered hypoimmunogenic cell);
d) a step for performing a function of subjecting the hypoimmunogenic cell (such as the engineered hypoimmunogenic cell) to an immune system; and
e) a step for performing a function of determining immunogenicity of the hypoimmunogenic cell (such as the engineered hypoimmunogenic cell), wherein the immunogenicity is altered as compared to an iPS human cell where the B2M gene is not genetically modified,
optionally wherein step b) further comprises a step for performing a function of genetically modifying a RFX gene, a CIITA gene, and/or a B2M gene of the iPS human cell.
   C40. A method of hypoimmunogenicity (such as engineering hypoimmunogenicity), comprising:
a) a step for performing a function of genetically modifying a CD58 gene of an immunogenic human cell to produce a hypoimmunogenic cell (such as an engineered hypoimmunogenic cell), wherein genetically modifying the CD58 gene reduces expression of the CD58 protein by the immunogenic human cell;
b) a step for performing a function of subjecting the hypoimmunogenic cell (such as the engineered hypoimmunogenic cell) to an immune system; and
c) a step for performing a function of determining immunogenicity of the hypoimmunogenic cell (such as the engineered hypoimmunogenic cell), wherein the immunogenicity is altered as compared to an immunogenic human cell where the CD58 gene is not genetically modified,
optionally wherein step a) further comprises a step for performing a function of genetically modifying a RFX gene, a CIITA gene, and/or a B2M gene of the immunogenic human cell.
   C41. A non-naturally occurring hypoimmunogenic human cell (such an engineered hypoimmunogenic human cell), comprising a means for reducing expression of a CD58 protein through a genetically modified CD58 gene, and/or a means for altering immunogenicity of an immune system to the hypoimmunogenic human cell (such as the engineered hypoimmunogenic human cell) as compared to an immunogenic human cell where the CD58 gene is not genetically modified; optionally wherein the hypoimmunogenic human cell (such as the engineered hypoimmunogenic human cell) further comprises a means for reducing expression of a CIITA protein, a B2M protein, and/or an RFX protein through a genetically modified CIITA gene, a genetically modified B2M gene, and/or a genetically modified RFX gene.
   C42. A γδT cell-derived induced pluripotent stem (iPS) human cell, comprising a means for reducing expression of a CD58 protein through a genetically modified CD58 gene, and/or a means for altering immunogenicity of an immune system to the iPS human cell as compared to an iPS human cell where the CD58 gene is not genetically modified; optionally wherein the iPS human cell further comprises a means for reducing expression of a CIITA protein, a B2M protein, and/or an RFX protein through a genetically modified CIITA gene, a genetically modified B2M gene, and/or a genetically modified RFX gene.

### 9. EXAMPLES

The following is a description of various methods and materials used in the studies. They are put forth so as to provide those of ordinary skill in the art with a complete disclosure and description of how to make and use the present invention, and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### 9.1 Example 1: RFX, B2M, and CIITA Gene Editing for Evading the Allogeneic Host Versus Graft Immune Response

Preparation of Cas9:gRNA RNP complexes. Alt-R crRNA listed in Table 1 specific for indicated genes were synthesized (IDT) and dissolved in nuclease free duplex buffer (IDT #11-05-01-12) at 100 μM. To generate guide RNA (gRNA), an equal volume of 100 μM tracrRNA (IDT #1072534) was added to each crRNA and the solution was heated to 95° C. for 5 mins and allowed to slowly cool to room temperature before storing at −20° C. RNP complexes were prepared fresh on the day of nucleofection by mixing a ratio of 60 pmol (2 μl of 5 μg/μl) Cas9 (Thermo #A36499) with 150 pmol (3 μl of 50 μM) thawed gRNA separately for each gRNA. After incubation for 10 mins at room temperature, master mixes for each target gene were prepared by pooling equal volumes of RNPs (two separate RNPs pooled for B2M and three separate RNPs pooled for RFX5, RFXANK, RFXAP, or CIITA). Immediately before nucleofection, a volume of 6 μl of RNP mix was prepared by either combining 3 μl of RNP master mix from two separate master mixes (for double gene knockout) or by aliquoting 6 μl from one RNP master mix (for single gene knockout). This final volume of 6 μl was added to 20 μl of cells in nucleofection buffer.

TABLE 1
List of CRISPR/Cas9 crRNAs

| Sequence ID | Target Gene | Target Sequence | SEQ ID NO. |
|---|---|---|---|
| Hs.Cas9.B2M.1.AA | B2M | CGTGAGTAAACCTGAATCTT | 1 |
| Hs.Cas9.B2M.1.AB | B2M | AAGTCAACTTCAATGTCGGA | 2 |
| Hs.Cas9.CIITA.1.AA | CIITA | AGTCGCTCACTGGTCCCACT | 3 |
| Hs.Cas9.CIITA.1.AB | CIITA | CCGTGGACAGTGAATCCACT | 4 |
| Hs.Cas9.CIITA.1.AC | CIITA | CTCTCACCGATCACTTCATC | 5 |
| Hs.Cas9.RFX5.1.AA | RFX5 | GTGGTTGCGGATCCACCTAT | 6 |
| Hs.Cas9.RFX5.1.AB | RFX5 | ACCTCGAGATGAACTGGTGG | 7 |
| Hs.Cas9.RFX5.1.AC | RFX5 | GAACCCTTTAGGTCAAGTCC | 8 |
| Hs.Cas9.RFXANK.1.AA | RFXANK | CTCTCACCAACCGGCAGCGA | 9 |
| Hs.Cas9.RFXANK.1.AB | RFXANK | AACGGTCTCAATCTCTCCAA | 10 |
| Hs.Cas9.RFXANK.1.AC | RFXANK | GTCTGGATGAGGTCTTCTGC | 11 |
| Hs.Cas9.RFXAP.1.AA | RFXAP | GTTAGACACTTCGGACCCTC | 12 |
| Hs.Cas9.RFXAP.1.AB | RFXAP | CGTTAGGTACCTGTGCGAAG | 13 |
| Hs.Cas9.RFXAP.1.AC | RFXAP | GACGAGGAGACTCACTCGGG | 14 |

| | |
|---|---|
| Negative control crRNAs #1 | IDT #1072544 |
| Negative control crRNAs #2 | IDT #1072545 |
| Negative control crRNAs #3 | IDT #1072546 |

Generation of HLA class I and class II altered human T cells using CRISPR/Cas9. Human T cells were isolated from peripheral blood mononuclear cells (PBMCs) by negative selection (StemCell #17951) and rested overnight in TexMACS (Miltenyi #170-076-307), 30 IU/ml hIL-21S (Miltenyi #130-097-748), and 100 IU/ml Penicillin+100 μg/ml Streptomycin (Gibco #15140-122), herein referred to as “media,” at 1×10⁶ cells/ml. The following day, T cells were collected, washed once with PBS, and nucleofected with 6 μl of Cas9:gRNA RNP complexes specific for the indicated genes in 20 μl of P3 Buffer (Lonza #V4SP-3096) with 4 μM electroporation enhancer (IDT #1075916) in 96 well cuvettes (Lonza #V4SP-3096) using the EH-115 program on the Lonza 4D system. Immediately after nucleofection, T cells were recovered in 200 μl warm media for 2 hours at 37 C. T cells were then activated with a 1:17.5 dilution of TransAct (Miltenyi #130-019-011) in media at approximately 1×10⁶ cells/ml. T cells were expanded in culture by addition of fresh media every 2-3 days for an additional 14 days, at which point surface expression of HLA class I and class II was measured by flow cytometry before cryopreserving cells in Cryostor CS10 (Sigma #C2874-100ML).

Preparation of Cas12a:crRNA RNP complexes for editing of human T cells. Alt-R crRNA listed in Table 2 was synthesized (IDT) and dissolved in nuclease free duplex buffer (IDT #11-05-01-12) at 200 uM. RNP complexes were prepared fresh on the day of nucleofection by mixing a ratio of 126 pmol (2 μl of 10 μg/μl) Cas12a (IDT #1081068) with 630 pmol (3.15 μl of 200 μM) thawed crRNA separately for each crRNA. After incubation for 10 mins at room temperature, RNP for each target gene were ready for nucleofection. Immediately before nucleofection, a volume of 5.15 μl of RNP mix and 0.85 μl nuclease free duplex buffer was prepared by either combining 3 μl of RNP master mix from two separate master mixes (for double gene knockout) or by aliquoting 5.15 μl from one RNP master mix (for single gene knockout). This final volume of 6 μl was added to 20 μl of cells in nucleofection buffer.

List of CRISPR/Cas12a crRNAs.

| Target Gene | crRNA target sequence | SEQ ID NO | crRNA Label |
|---|---|---|---|
| B2M | AGTGGGGGTGAATTCAGTGTA | 15 | B2M-2 |
| B2M | CCGATATTCCTCAGGTACTCCAAA | 16 | B2M-3 |
| B2M | ATCCATCCGACATTGAAGTTGACT | 17 | B2M-4 |
| B2M | CTCACGTCATCCAGCAGAGAATGG | 18 | B2M-5 |
| B2M | AATTCTCTCTCCATTCTTCAGTAA | 19 | B2M-6 |
| B2M | ACTTTCCATTCTCTGCTGGATGAC | 20 | B2M-7 |
| B2M | AGCAAGGACTGGTCTTTCTATCTC | 21 | B2M-8 |
| B2M | CATTCTCTGCTGGATGACGTGAGT | 22 | B2M-9 |
| B2M | TGGCCTGGAGGCTATCCAGCGTGA | 23 | B2M-12 |
| B2M | CTGAATTGCTATGTGTCTGGGTTT | 24 | B2M-24 |
| B2M | TATCTCTTGTACTACACTGAATTC | 25 | B2M-10 |
| B2M | TCACAGCCCAAGATAGTTAAGTGG | 26 | B2M-11 |
| B2M | TCCACTGTCTTTTTCATAGATCGA | 27 | B2M-17 |
| CIITA | CCTTGGGGCTCTGACAGGTA | 28 | CIITA_10877363 |
| CIITA | TCTGCAGCCTTCCCAGAGGA | 29 | CIITA_10901506 |
| CIITA | CCAGGTCTTCCACATCCTTC | 30 | CIITA_10909145 |
| CIITA | GGGAAAGCCTGGGGGCCTGAG | 31 | CIITA-10915602 |
| CIITA | CAAGGACTTCAGCTGGGGGA | 32 | CIITA_10916428 |
| RFX5 | CTTTGGCAAAGGGAGAGGTA | 33 | RFX5_151342281 |
| RFX5 | GCTGGTGGAGCCTGCCCACT | 34 | RFX5_151342693 |
| RFX5 | AGGGGACCAAGGGAATTTTAT | 35 | RFX5_151342349 |
| RFX5 | AGGGCACCTGAAGAAAGCCTG | 36 | RFX5_151342957 |
| RFX5 | CCTGGACCTGGACCTGGGCC | 37 | RFX5_151342843 |
| RFX5 | GCATCACTTGCTGTATCCTCTATA | 38 | RFX5-1 |
| RFX5 | TGACAATGACAAGCTGTATCTCTA | 39 | RFX5-5 |
| RFX5 | AGGGCACCTGAAGAAAGCCTGGGG | 40 | RFX5-14 |
| RFX5 | GCTGGTGGAGCCTGCCCACTGGCC | 41 | RFX5-6 |
| RFXANK | CCTGCACCCCTGAGCCTGTGAATC | 42 | RFXANK-2 |
| RFXANK | CCTGCCCCATCTCAGTGCAACAGG | 43 | RFXANK-1 |
| RFXANK | CCCATGGAGCTTACCCAGCCTGCA | 44 | RFXANK-5 |
| RFXAP | GAGCAAAGACAACAGCAGTTTCCA | 45 | RFXAP-1 |
| RFXAP | GAACAAGTGTTAAATCAAAAAAGA | 46 | RFXAP-2 |
| RFXAP | ACAATGGAGAGTATGTTATCTGCA | 47 | RFXAP-4 |
| RFXAP | CTGGGATAAATGATAAAAATGCAA | 48 | RFXAP-7 |
| RFXAP | TACTTGTCCTTGTACATCTTGTTG | 49 | RFXAP-10 |
| RFXAP | CCGCGCTGCCAGTCGAGGCAGTCC | 50 | RFXAP-11 |
| RFXAP | ACGTTTCCCGCGCTGCCAGTCGAG | 51 | RFXAP-16 |
| RFXAP | TTGCACATCCACGGTTTGCGCTGC | 52 | RFXAP-18 |

Exemplary generation of HLA class I and class II altered human T cells using CRISPR/Cas12a (Cpf1). Human T cells were isolated from PBMCs by negative selection (StemCell #17951) and rested overnight in TexMACS (Miltenyi #170-076-307), and 30 IU/ml hIL-21S (Miltenyi #130-097-748), herein referred to as “media,” at 1×10⁶ cells/ml. The following day, T cells were collected, washed once with PBS, and nucleofected with 6 μl of Cas12a:crRNA RNP complexes specific for the indicated genes in 20 μl of P3 Buffer (Lonza #V4SP-3096) with 4 uM electroporation enhancer (IDT #1075916) in 96 well cuvettes (Lonza #V4SP-3096) using the EH-115 program on the Lonza 4D system. Immediately after nucleofection, T cells were recovered in 200 μl warm media for 2 hrs at 37 C. T cells were then activated with a 1:17.5 dilution of TransAct (Miltenyi #130-019-011) in media at approximately 1×10⁶ cells/ml. T cells were expanded in culture by addition of fresh media every 2-3 days for an additional 14 days, at which point surface expression of HLA class I and class II was measured by flow cytometry with the antibodies in Table 6 before cryopreserving cells in Cryostor CS10 (Sigma #C2874-100ML). The percent reduction in HLA class I was calculated as the % HLA class I negative cells, and the percent reduction in HLA class II was calculated by the formula: (1-(Sample % HLA class II positive cells/Control % HLA class II positive cells))*100.

Preparation of Cas12a:crRNA RNP complexes for editing human iPSCs. Alt-R crRNA was synthesized (IDT) and dissolved in nuclease free duplex buffer (IDT #11-05-01-12) at 200 μM. RNP complexes were prepared fresh on the day of nucleofection by mixing a ratio of 63 pmol (1 μl of 10 μg/μl) Alt-R® A.s. Cas12a (Cpf1) V3 (IDT #1081068) with 315 pmol (1.58 μl of 200 μM) crRNA in P3 Buffer (Lonza #V4SP-3096) with 3 μM electroporation enhancer (IDT #1076301) for a total volume of 10 μL.

Generation of HLA class I and class II altered human iPSCs. Human iPS cells were either obtained commercially (PGP1) or generated by reprogramming isolated γδT cells to iPSCs (Clone D). For gene editing experiments, iPSCs were pretreated with 10 μM Y-27632 ROCK inhibitor (STEMCELL Technologies #72302) in StemFlex Medium (Gibco #A3349401). The iPSCs were collected (0.5×10⁶ cells per reaction) and resuspended in 10 μL of P3 buffer with 3 μM electroporation enhancer. The cells were combined with 10 μL of Cas12a:crRNA RNP complex and nucleofected in 96 well cuvettes (Lonza #V4SP-3096) using the CB-150 program on the Lonza 4D system. The iPSCs were then transferred to one well of 24 well plate coated with 0.5 μg/cm² of iMatrix-511 (Takara #T304) containing StemFlex Medium with 10 μM Y-27632 ROCK inhibitor. The iPSCs were expanded to 6 well plates two days post nucleofection and the media was changed daily for 7 days, at which pluripotency markers and surface expression of HLA class I were measured by flow cytometry with the antibodies in Table 6. HLA class I editing efficiency was calculated as the % B2M negative cells, and HLA class II editing efficiency was measured by the ICE tool (Synthego) to analyze sanger sequencing results (Azenta Life Sciences).

TABLE 3
List of antibodies used in this study.

| Specificity | Clone | Company |
|---|---|---|
| Pan-HLA class I | W6/32 | BioLegend |
| CD3 | OKT3 | BioLegend |
| CD8 | RPA-T8 | BioLegend |
| Pan-HLA class II | Tu39 | BioLegend |
| CD4 | OKT4 | BioLegend |
| HLA-E | 3D12 | BioLegend |
| B2M | 2M2 | BioLegend |
| CD56 | 5.1.H11 | BioLegend |
| SSEA-4 | MC813-70 | BD Biosciences |
| SSEA-3 | MC-631 | BD Biosciences |
| CD107a | H4A3 | BioLegend |
| 4-1BB | 4B4-1 | BD Biosciences |
| LD-NIR | n/a | Thermo Fisher |

Generation of allogeneic effector T cells. PBMCs from a non-HLA matched human donor were stimulated with irradiated (40 Gy) PBMCs from the human donor used to make HLA class I and II negative T cells at a 1:1 ratio in media [TexMACS (Miltenyi #170-076-307) and 100 IU/ml Penicillin+100 μg/ml Streptomycin (Gibco #15140-122)], without IL-2 at 2×10⁶ cells/ml. After 2 days, an equal volume of media containing 60 IU/ml hIL-21S (Miltenyi #130-097-748) was added to achieve a final concentration of 30 IU/ml IL-21S. After 5 additional days of culture, cells were washed, resuspended into media with 30 IU/ml IL-21S and restimulated with another round of irradiated (40 Gy) PBMCs from the human donor used to make HLA class I and II negative T cells at a 1:1 ratio. Following another 2 days of culture, an equal volume of media was added and IL-21S was supplied to 200 IU/ml. Two days later, fresh media and 200 IU/ml IL-21S was added to dilute the cells to 0.5×10⁶ cells/ml, and cells were cultured for another 3 days before being cryopreserved in CS10. In some experiments, alloreactive effector cells were separated into purified T cells (mixture of CD4⁺ and CD8⁺) and purified NK cells (CD56+CD3−) with an EasySep CD56+ isolation kit (STEMCELL Technologies #17855).

Isolation of primary NK cells. Human NK cells were isolated from leukapheresis (StemExpress and HemaCare) using the NK cell Isolation kit (Miltenyi #130-092-657) and program on a CliniMACS Prodigy (Miltenyi Biotec). NK cells were cryopreserved at 10⁶ cells/mL in Cryostor CS10 (Sigma #C2874-100ML).

Allogeneic response assay. Cryopreserved HLA-altered T cells (“targets”) were thawed and rested overnight in RPMI+L-glutamine (Gibco #11875-093), 10% FBS (Gibco #16140-071), 100 IU/ml Penicillin+100 μg/ml Streptomycin (Gibco #15140-122), 1 mM Sodium Pyruvate (Gibco #11360-070), 10 mM HEPES (Gibco #15630-080), and 55 μM 2-mercaptoethanol (Gibco #21985-023), herein referred to as “assay media,” supplemented with 30 IU/ml hIL-21S (Miltenyi #130-097-748), at 1×10⁶ cells/ml. The next day, target T cells were washed to remove IL-2 and seeded in 96 well U-bottom plates in assay media at 10,000 cells/well for cytotoxicity and 100,000 cells/well for CD107a assays. Allogeneic effector T cells were also thawed and rested one day prior to experiment setup in assay media supplemented with 30 IU/ml hIL-21S. Primary NK cells were also thawed and rested one day prior to experiment setup in assay media supplemented with 0.2 ng/mL IL-2 (Gibco #PHC0026). The next day, allogeneic effector T cells or NK cells were labelled with 1 μM Cell Trace Violet (Thermo #C34557) in PBS for 20 mins at 37° C., washed twice with assay media, and seeded with targets for cytotoxicity assays (various E:Ts) and for CD107a assays (0.5 E:T). CD107a-PE (BioLegend #328608, clone H4A3) was added at 1:200 to CD107a assay wells. Cells were analyzed by flow cytometry 4 hours (CD107a) or 18-24 hours (cytotoxicity) after plating. In some assays, activation of effector cells was measured by flow cytometry staining for 4-1BB (BD Bioscience, clone 4B4-1) at the conclusion of the cytotoxicity assay. Normalized target viability was calculated as: % live targets at E:T/% live targets alone.

The present disclosure discovered that a knockout of a single gene, either RFX5, RFXANK, or RFXAP, can evade most of the allogeneic immune response by completely evading CD4⁺ T cell responses against HLA class II, partially evading CD8⁺ T cell responses against HLA class I, and limiting the activation of NK “missing-self” rejection.

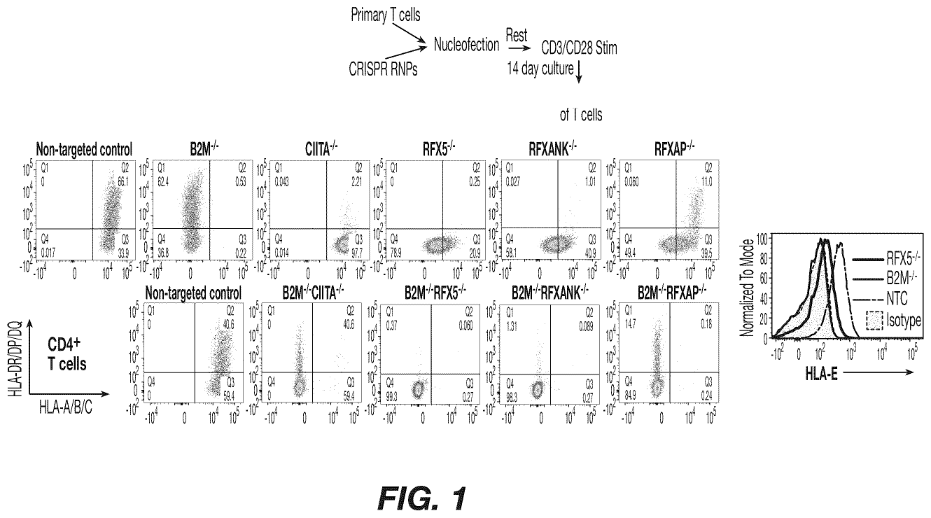

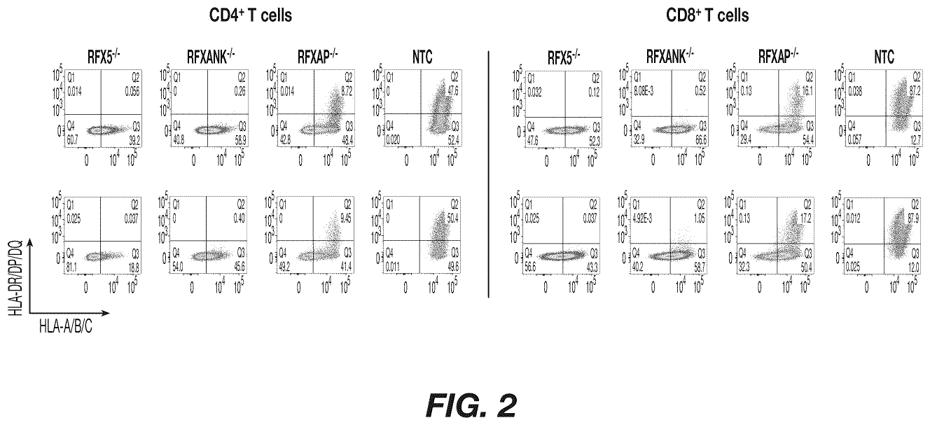

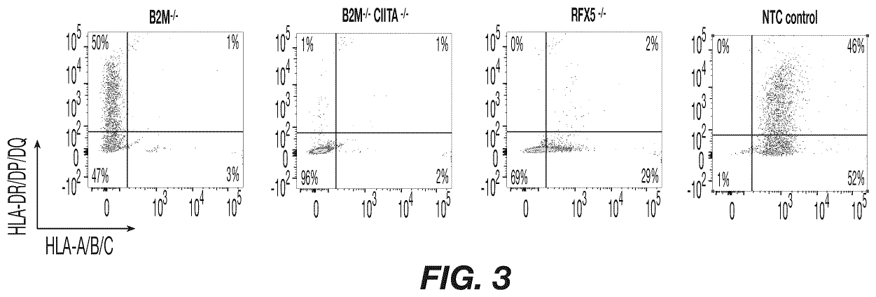

RFX knockout led to strong down-regulation of HLA class II and moderate down-regulation of HLA class I. As shown in FIG. 1, combined knockout of B2M and RFX5, B2M and RFXANK, B2M and RFXAP, or B2M and CIITA resulted in HLA class I and II deficient cells, while knockout of RFX5, RFXANK, or RFXAP individually resulted in cells that lacked HLA class II surface expression and had reduced, but not absent, HLA class I expression, including HLA-E. See Table 4 below identifying the HLA class of the target genes. Similar results were obtained with CD8⁺ T cells. RFX knockout T cells from additional human donors also had down-regulation of HLA class I and II molecules (FIG. 2). RFX5 knockout T cells generated with Cpf1 also had down-regulation of HLA class I and II molecules (FIG. 3).

TABLE 4
HLA classification of target genes.

| | HLA Class Mechanistically Related to |
|---|---|
| Target Gene | Target Gene |
| B2M | Class I |
| CIITA | Class II |
| RFX5 | Class I & II |
| RFXANK | Class I & II |
| RFXAP | Class I & II |

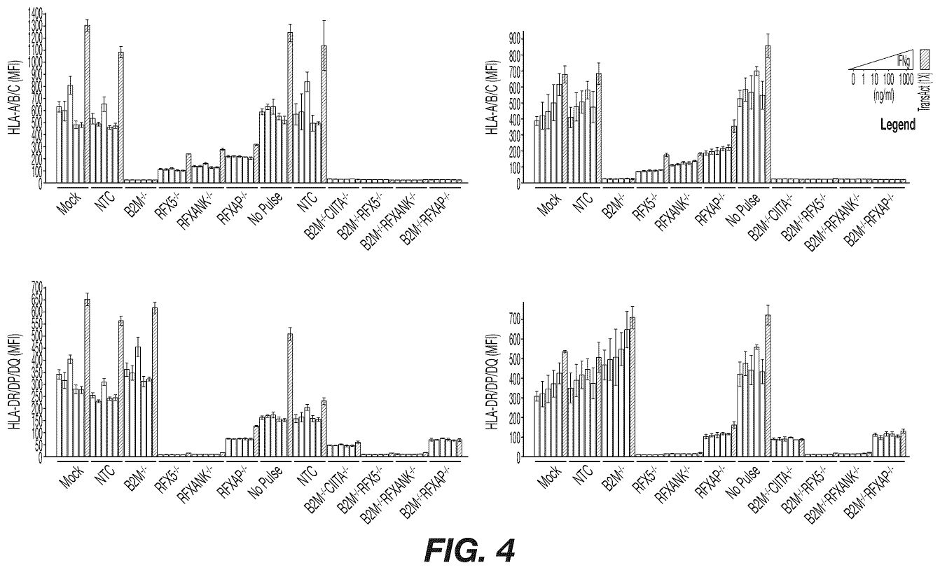

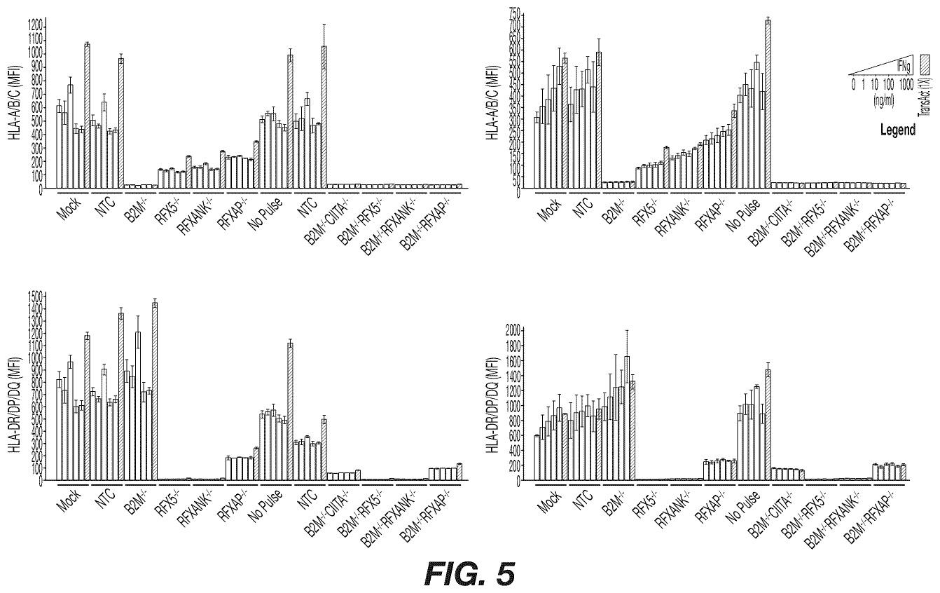

Down-regulation of HLA class I and II after RFX knockout was largely stable after a short (24 hr) stimulation. 14 days after the generation of HLA class I and II altered T cells from two human donors (D151100 and D144786), the cells were cryopreserved, thawed, and then stimulated with IFN-gamma or CD3/CD28 stimulation (TransAct) as indicated. 24 hrs later the cells were analyzed for surface expression of pan HLA class I and class II on CD4⁺ T cells (FIG. 4) and CD8⁺ T cells (FIG. 5). The results suggested that knockouts of B2M and CIITA created completely stable loss of HLA class I and class II, respectively. Knockouts of RFX genes created largely stable reduction in HLA class I and II genes, with a small increase in HLA class I genes after stimulation of the cells. The expression of HLA class I in RFX knockouts after stimulation was still only ~25% of the corresponding level of expression in unmodified, stimulated T cells.

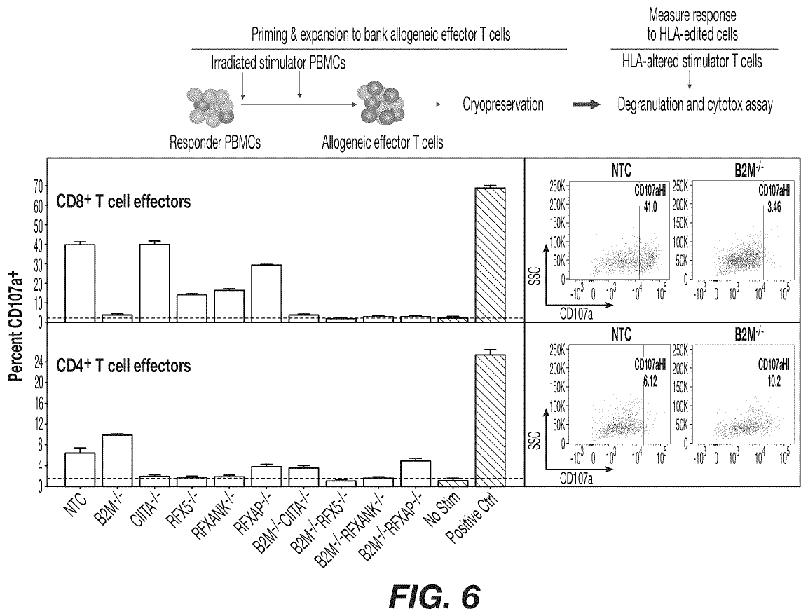

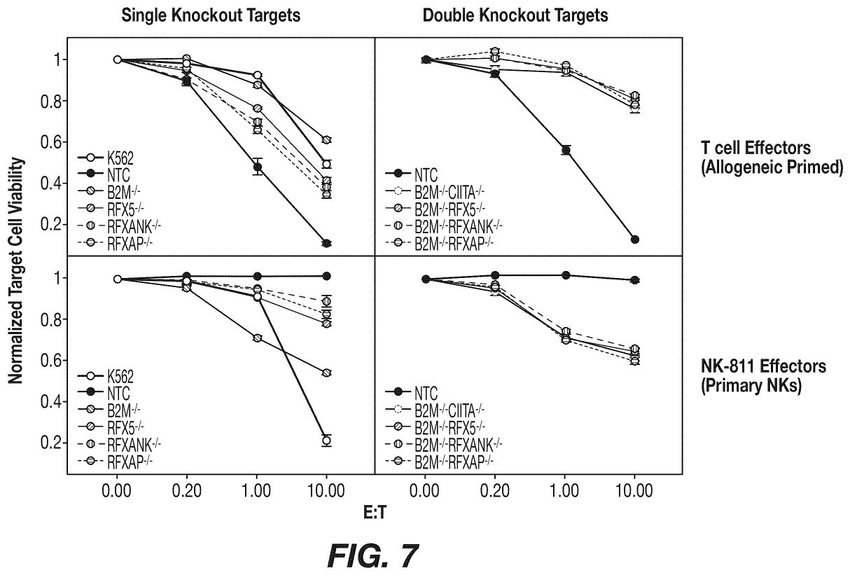

Down-regulation of HLA class I and II after RFX knockout subverted most of the CD4 T cell and about half of the CD8 T cell allogeneic response, while minimizing NK missing-self response. FIG. 6 shows that HLA-altered T cells avoided allogeneic effector T cell cytotoxic responses. As compared to unedited (NTC) T cells, HLA-altered T cells exhibited diminished ability to induce degranulation of allogeneic effector T cells (FIG. 6). FIG. 7 shows that RFX knockout T cells have an ability to balance evasion of both allogeneic T cells and NK cells. As compared to unedited (NTC) T cells, HLA-altered T cells showed enhanced ability to survive challenge with allogeneic effector T cells (FIG. 7). In particular, RFX knockout T cells were able to survive about twice as well as unedited (NTC) cells when co-cultured with allogeneic effector T cells (FIG. 7). As compared to full HLA class I deficient cells (B2M knockout), which are highly susceptible to NK missing-self lysis, RFX knockout T cells showed enhanced ability to survive challenge with primary NK cells (FIG. 7).

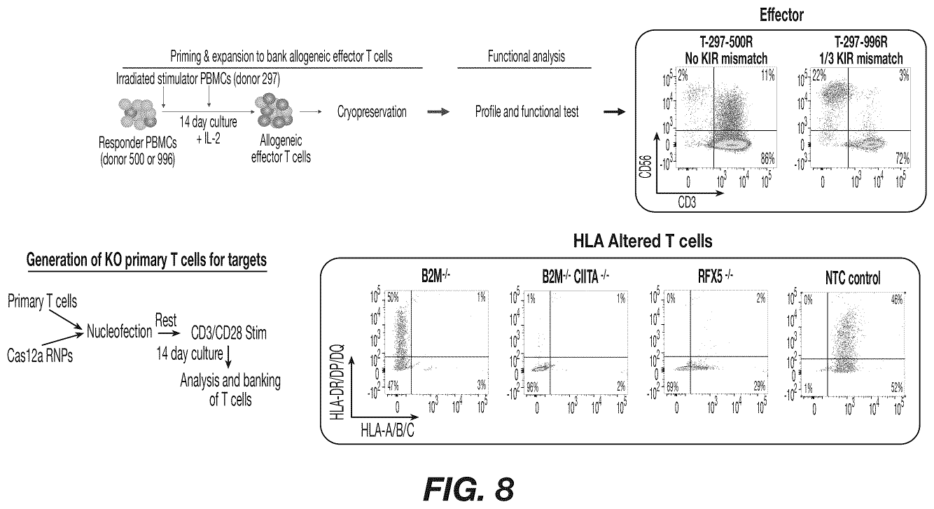

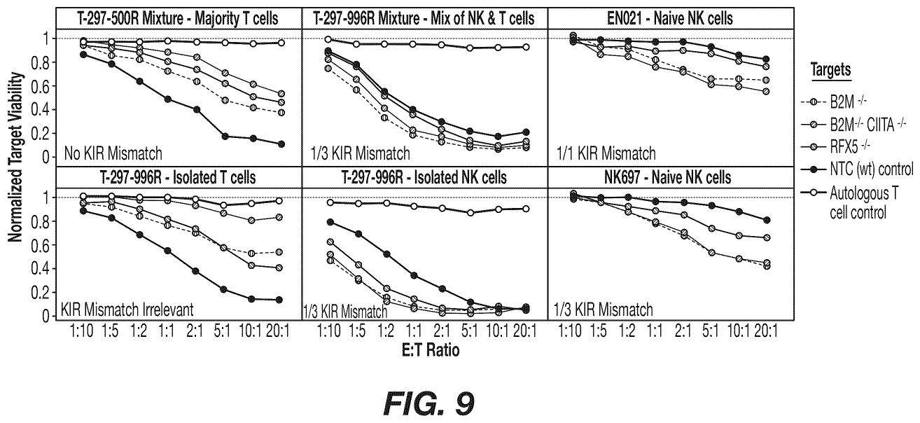

Human donor 297 (also referred to as ‘Donor 147297’) RFX5 knockout T cells survived better than or equal to B2M knockout T cells against all allogeneic effector cells tested. FIG. 8 shows the process to generate allogeneic effector T cells against human donor 297. Data indicated high purity of T cells in human donor 500 allogeneic effector T cells (T-297-500R; 87% T cells, 2% NK cells, and 11% NKT cells), but significant presence of NK cells in human donor 996 allogeneic effector T cells (T-297-996R; 72% T cells, 22% NK cells, 3% NKT cells) (FIG. 8). When the T-297-500R mixture was co-cultured with human donor 297 target cells, HLA-altered T cells showed enhanced ability to survive as compared to unedited (NTC) T cells (FIG. 9, top left panel). However, when the T-297-996R mixture was co-cultured with human donor 297 target cells, HLA-altered T cells showed a slightly worse ability to survive as compared to unedited (NTC) T cells (FIG. 9, top middle panel). Separate purification of the T cells (to 97% purity) and NK cells (to 94% purity) from the T-297-996R mixture demonstrated that, as compared to unedited (NTC) T cells, HLA-altered T cells survived better when co-cultured with purified T cells (FIG. 9, bottom left panel) and worse when co-cultured with purified NK cells (FIG. 9, bottom middle panel). These data indicate that NK cells in the T-297-996R mixture were likely expanded due to a KIR mismatch and further activated by the lack of HLA class I molecules in human donor 297 HLA-altered T cells. Regardless, human donor 297 RFX5 knockout T cells survived better than or equal to B2M knockout T cells in all co-cultures tested, including when incubated with resting (naive) NK cells from two additional human donors (FIG. 9, right panels).

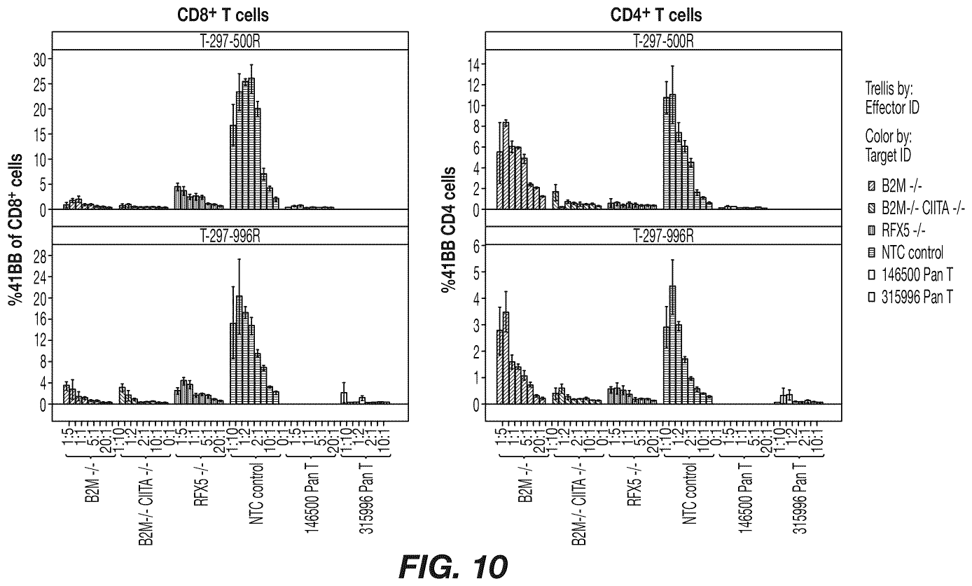

RFX5 knockout limited activation of allogeneic effector CD8⁺CD4⁺ allogeneic T cells in co-cultures with human donor 297 T cells (FIG. 10). As compared to unedited (NTC) T cells, HLA-altered T cells showed diminished ability to induce activation of allogeneic effector T cells. RFX5 knockout T cells showed an ability to limit most of the effector CD8⁺ T cell activation and all of the effector CD4⁺ T cell activation, down to a level that was similar to autologous pan T cells from the effector human donor which served as negative controls.

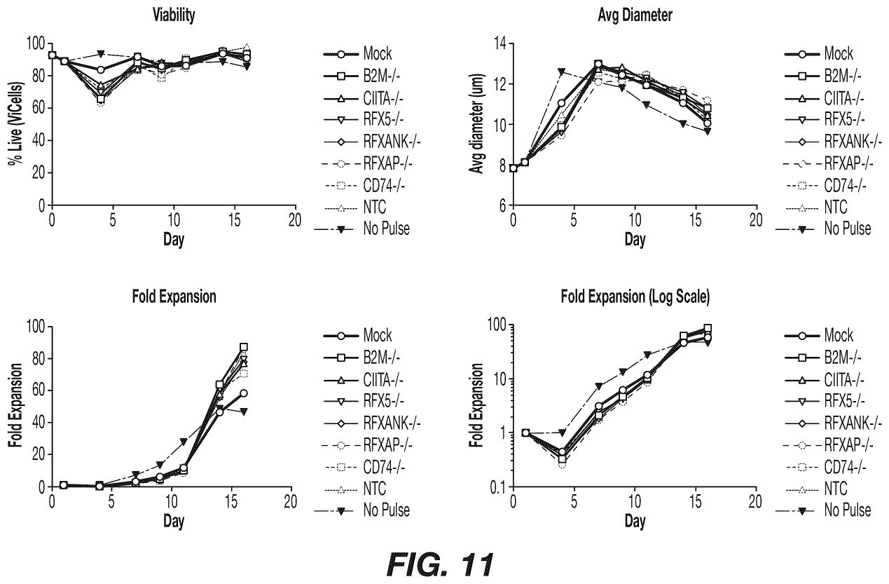

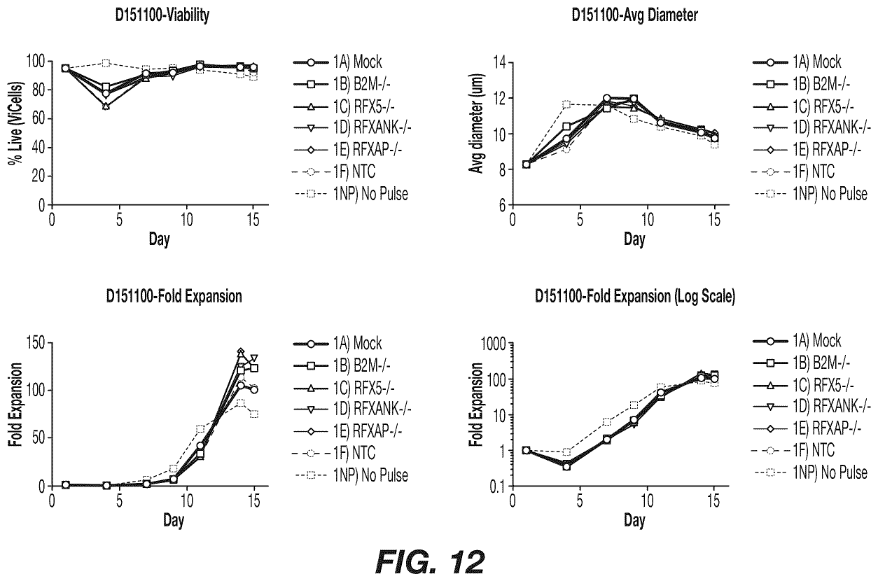

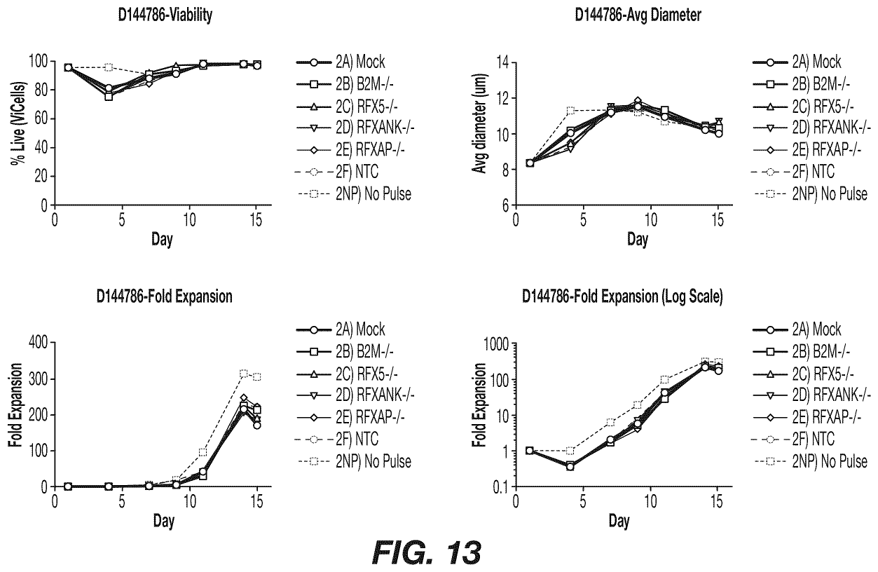

RFX knockout did not impair proliferation or viability of primary T cells. There was no detrimental effect of RFX5 knockout or other gene disruptions tested on T cell expansion from three separate human donors (FIGS. 11-13). A significant temporary effect of the electroporation was observed (no pulse as compared to NTC), but no effects from individual gene disruptions were observed (FIGS. 11-13).

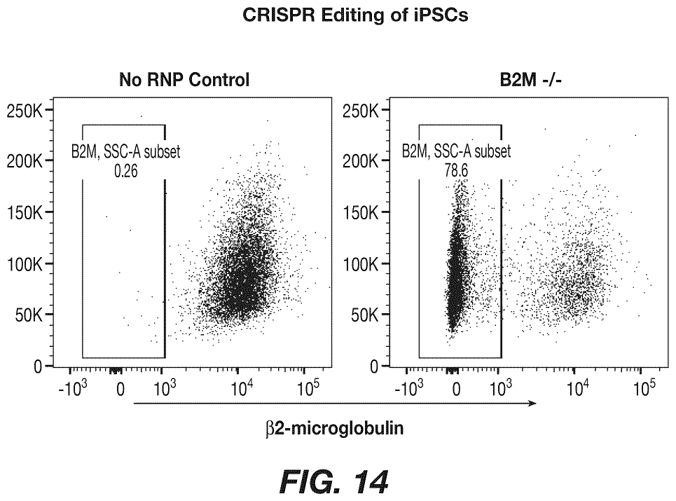

Editing of B2M, RFX5, RFXANK, RFXAP, or CIITA is possible in PGP1 iPSCs with Cpf1. FIG. 14 shows that B2M knockout PGP1 iPSCs can be created with Cpf1 and loss of B2M protein can be detected by flow cytometry. Table 5 shows that editing of B2M, RFX5, RFXANK, RFXAP, and CIITA is possible in PGP1 iPSCs with Cpf1 and that editing of B2M and RFX5 is possible in γδ-derived iPSCs with Cpf1. Table 6 shows the efficiency of targeting two genes simultaneously in primary human T cells using Cpf1.

TABLE 5
Single CRISPR/Cas 12a crRNA editing efficiency in primary human T cells and iPSCs

| | | | | | | | | | Percent |
|---|---|---|---|---|---|---|---|---|---|
| | | | | Percent | Percent | Percent | Percent | Percent | editing |
| | | | | reduction of | reduction of | reduction of | reduction of | editing | efficiency in |
| | | | | HLA class I | HLA class II | HLA class I | HLA class II | efficiency in | γδ-derived |
| Target Gene | crRNA target sequence | SEQ ID NO | crRNA label | of CD8⁺ T cells | on CD8⁺ T cells | on CD4⁺ T cells | on CD4⁺ T cells | PGP1 iPSCs | iPSCs |
| B2M | AGTGGGGGTGAATTCAGTGTA | 15 | B2M-2 | 99.4 | — | 99.1 | — | 78.6 | 53 |
| B2M | CCGATATTCCTCAGGTACTCCAAA | 16 | B2M-3 | 93.2 | — | 89.6 | — | 6.6 | — |
| B2M | ATCCATCCGACATTGAAGTTGACT | 17 | B2M-4 | 98.8 | — | 99.0 | — | 0.0 | — |
| B2M | CTCACGTCATCCAGCAGAGAATGG | 18 | B2M-5 | 51.0 | — | 47.1 | — | 1.9 | — |
| B2M | AATTCTCTCTCCATTCTTCAGTAA | 19 | B2M-6 | 86.0 | — | 79.9 | — | 0.0 | — |
| B2M | ACTTTCCATTCTCTGCTGGATGAC | 20 | B2M-7 | 61.4 | — | 57.7 | — | 0.0 | — |
| B2M | AGCAAGGACTGGTCTTTCTATCTC | 21 | B2M-8 | 93.5 | — | 87.8 | — | 0.0 | — |
| B2M | CATTCTCTGCTGGATGACGTGAGT | 22 | B2M-9 | 0.7 | — | 0.1 | — | 0.0 | — |
| B2M | TGGCCTGGAGGCTATCCAGCGTGA | 23 | B2M-12 | 65.6 | — | 63.3 | — | 0.0 | — |
| B2M | CTGAATTGCTATGTGTCTGGGTTT | 24 | B2M-24 | 98.3 | — | 98.3 | — | 0.0 | — |
| B2M | TATCTCTTGTACTACACTGAATTC | 25 | B2M-10 | 95.8 | — | 94.7 | — | 0.0 | — |
| B2M | TCACAGCCCAAGATAGTTAAGTGG | 26 | B2M-11 | 85.0 | — | 72.2 | — | 0.0 | — |
| B2M | TCCACTGTCTTTTTCATAGATCGA | 27 | B2M-17 | 31.1 | — | 10.6 | — | 0.0 | — |
| CIITA | CCTTGGGGCTCTGACAGGTA | 28 | CIITA_10877363 | — | 15.0 | — | 18.0 | 45.0 | — |
| CIITA | TCTGCAGCCTTCCCAGAGGA | 29 | CIITA_10901506 | — | 75.0 | — | 72.0 | 49.0 | — |
| CIITA | CCAGGTCTTCCACATCCTTC | 30 | CIITA_10909145 | — | 98.3 | — | 97.9 | 29.0 | — |
| CIITA | GGGAAAGCCTGGGGGCCTGAG | 31 | CIITA-10915602 | — | 97.1 | — | 95.0 | 55.0 | — |
| CIITA | CAAGGACTTCAGCTGGGGGA | 32 | CIITA_10916428 | — | 97.8 | — | 94.0 | 26.0 | — |
| RFX5 | CTTTGGCAAAGGGAGAGGTA | 33 | RFX5_151342281 | 1.7 | 0.0 | 1.7 | 8.5 | 0.0 | — |
| RFX5 | GCTGGTGGAGCCTGCCCACT | 34 | RFX5_151342693 | 6.8 | 19.6 | 10.1 | 29.0 | 11.0 | — |
| RFX5 | AGGGGACCAAGGGAATTTTAT | 35 | RFX5_151342349 | 14.1 | 3.7 | 14.3 | 8.0 | 49.0 | 90 |
| RFX5 | AGGGCACCTGAAGAAAGCCTG | 36 | RFX5_151342957 | 44.1 | 46.6 | 45.4 | 54.0 | 21.0 | 82 |
| RFX5 | CCTGGACCTGGACCTGGGCC | 37 | RFX5_151342843 | 35.6 | 57.0 | 40.8 | 32.0 | 7.0 | — |
| RFX5 | GCATCACTTGCTGTATCCTCTATA | 38 | RFX5-1 | 0.3 | 23.0 | 0.6 | 0.0 | 17.0 | — |
| RFX5 | TGACAATGACAAGCTGTATCTCTA | 39 | RFX5-5 | 80.1 | 96.7 | 86.9 | 95.0 | 0.0 | — |
| RFX5 | AGGGCACCTGAAGAAAGCCTGGGG | 40 | RFX5-14 | 43.1 | 59.4 | 44.7 | 43.0 | 18.0 | — |
| RFX5 | GCTGGTGGAGCCTGCCCACTGGCC | 41 | RFX5-6 | 2.7 | 26.0 | 3.6 | 62.0 | 0.0 | — |
| RFXANK | CCTGCACCCCTGAGCCTGTGAATC | 42 | RFXANK-2 | 7.6 | 3.1 | 6.2 | 0.0 | 34.0 | — |
| RFXANK | CCTGCCCCATCTCAGTGCAACAGG | 43 | RFXANK-1 | 71.6 | 98.0 | 77.5 | 95.6 | 32.0 | — |
| RFXANK | CCCATGGAGCTTACCCAGCCTGCA | 44 | RFXANK-5 | 46.6 | 74.1 | 47.7 | 70.0 | 70.0 | — |
| RFXAP | GAGCAAAGACAACAGCAGTTTCCA | 45 | RFXAP-1 | 16.4 | 58.0 | 28.4 | 50.0 | 6.0 | — |
| RFXAP | GAACAAGTGTTAAATCAAAAAAGA | 46 | RFXAP-2 | 20.1 | 45.0 | 31.2 | 58.0 | 0.0 | — |
| RFXAP | ACAATGGAGAGTATGTTATCTGCA | 47 | RFXAP-4 | 40.2 | 56.0 | 42.8 | 62.0 | 5.0 | — |
| RFXAP | CTGGGATAAATGATAAAAATGCAA | 48 | RFXAP-7 | 1.9 | 12.0 | 2.2 | 24.5 | 0.0 | — |
| RFXAP | TACTTGTCCTTGTACATCTTGTTG | 49 | RFXAP-10 | 29.5 | 82.6 | 29.6 | 76.7 | 0.0 | — |
| RFXAP | CCGCGCTGCCAGTCGAGGCAGTCC | 50 | RFXAP-11 | 7.7 | 12.1 | 10.7 | 26.0 | 0.0 | — |
| RFXAP | ACGTTTCCCGCGCTGCCAGTCGAG | 51 | RFXAP-16 | 18.5 | 30.0 | 18.9 | 34.0 | 0.0 | — |
| RFXAP | TTGCACATCCACGGTTTGCGCTGC | 52 | RFXAP-18 | 5.7 | 15.0 | 6.2 | 18.0 | 0.0 | |

( ) not applicable or not tested

TABLE 6
Double CRISPR/Cas12a gene editing efficiency in primary human T cells

| | | | | | Percent | Percent | Percent | Percent |
|---|---|---|---|---|---|---|---|---|
| | | | | | reduction | reduction | reduction | reduction |
| | | | | | of HLA | of HLA | of HLA | of HLA |
| | | | | | class I | class II | class I | class II |
| Target | crRNA target | crRNA | crRNA target | crRNA | on CD8⁺ T | on CD8⁺ T | on CD4⁺ T | on CD4⁺ T |
| Genes | sequence 1 | label 1 | sequence 2 | label 2 | cells | cells | cells | cells |
| B2M, | ATCCATCCGACATTG | B2M-4 | CCAGGTCTTCCACATC | CIITA_ | 92.3 | 93.3 | 79.0 | 91.0 |
| CIITA | AAGTTGACT | | CTTC | 10909145 | | | | |
| | (SEQ ID NO: 17) | | (SEQ ID NO: 30) | | | | | |
| B2M, | CTGAATTGCTATGTG | B2M- | CCAGGTCTTCCACATC | CIITA_ | 86.4 | 96.0 | 64.0 | 94.3 |
| CIITA | TCTGGGTTT | 24 | CTTC | 10909145 | | | | |
| | (SEQ ID NO: 24) | | (SEQ ID NO: 30) | | | | | |
| B2M, | AGTGGGGGTGAATTC | B2M-2 | CCAGGTCTTCCACATC | CIITA_ | 98.3 | 95.6 | 97.7 | 99.2 |
| CIITA | AGTGTA | | CTTC | 10909145 | | | | |
| | (SEQ ID NO: 15) | | (SEQ ID NO: 30) | | | | | |
| B2M, | ATCCATCCGACATTG | B2M-4 | GGGAAAGCCTGGGGGC | CIITA- | 98.3 | 59.0 | 93.8 | 62.0 |
| CIITA | AAGTTGACT | | CTGAG | 10915602 | | | | |
| | (SEQ ID NO: 17) | | (SEQ ID NO: 31) | | | | | |
| B2M, | CTGAATTGCTATGTG | B2M- | GGGAAAGCCTGGGGGC | CIITA- | 95.6 | 63.0 | 85.5 | 58.0 |
| CIITA | TCTGGGTTT | 24 | CTGAG | 10915602 | | | | |
| | (SEQ ID NO: 24) | | (SEQ ID NO: 31) | | | | | |
| B2M, | AGTGGGGGTGAATTC | B2M-2 | GGGAAAGCCTGGGGGC | CIITA- | 98.8 | 85.0 | 98.8 | 87.0 |
| CIITA | AGTGTA | | CTGAG | 10915602 | | | | |
| | (SEQ ID NO: 15) | | (SEQ ID NO: 31) | | | | | |
| B2M, | ATCCATCCGACATTG | B2M-4 | CAAGGACTTCAGCTGG | CIITA_ | 97.9 | 28.9 | 95.9 | 42.3 |
| CIITA | AAGTTGACT | | GGGA | 10916428 | | | | |
| | (SEQ ID NO: 17) | | (SEQ ID NO: 32) | | | | | |
| B2M, | CTGAATTGCTATGTG | B2M- | CAAGGACTTCAGCTGG | CIITA_ | 94.7 | 39.0 | 89.7 | 48.3 |
| CIITA | TCTGGGTTT | 24 | GGGA | 10916428 | | | | |
| | (SEQ ID NO: 24) | | (SEQ ID NO: 32) | | | | | |
| B2M, | AGTGGGGGTGAATTC | B2M-2 | CAAGGACTTCAGCTGG | CIITA_ | 99.2 | 46.0 | 96.9 | 51.0 |
| CIITA | AGTGTA | | GGGA | 10916428 | | | | |
| | (SEQ ID NO: 15) | | (SEQ ID NO: 108) | | | | | |

### 9.2 Example 2: CD58 and Other Gene Editing for Evading the Allogeneic Host Versus Graft Immune Response

Preparation of CRISPR RNP complexes. Alt-R crRNA listed in Tables 7 and 8 specific for indicated genes were synthesized (IDT) and dissolved in nuclease free duplex buffer (IDT #11-05-01-12) at 100 μM (Cas9) or 200 μM (Cas12a). To generate guide RNA (gRNA) for Cas9, an equal volume of 100 μM tracrRNA (IDT #1072534) was added to each crRNA and the solution was heated to 95° C. for 5 mins and allowed to slowly cool to room temperature before storing at −20° C. For Cas9, RNP complexes were prepared fresh on the day of nucleofection by mixing a ratio of 60 pmol (2 μl of 5 μg/μl) Cas9 (Thermo #A36499) with 150 pmol (3 μl of 50 μM) thawed gRNA separately for each gRNA. For Cas12a, RNP complexes were prepared fresh on the day of nucleofection by mixing a ratio of 126 pmol (2 μl of 10 μg/μl) Cas12a (IDT #1081068) with 630 pmol (3.15 μl of 200 uM) thawed crRNA separately for each crRNA. After incubation for 10 mins at room temperature, RNP mixtures for each sample were prepared by pooling the necessary individual RNPs at amounts which were empirically determined to achieve the highest editing efficiency, up to a volume of 6 μl total RNP mixture, which was added to 20 μl of cells in nucleofection buffer.

TABLE 7
List of CRISPR/Cas9 crRNAs

| | Target | | SEQ |
|---|---|---|---|
| Sequence ID | Gene | Target Sequence | ID NO |
| CD58.1.AB | CD58 | GACCACGCTGAGGACCCCCAG | 53 |
| CD58.1.AD | CD58 | GTCAATGCACAAGTTAGTGT | 54 |
| TNFRSF14.1.AD | HVEM | AAGGAGGACGAGTACCCAGT | 55 |
| TNFRSF14_B | HVEM | ACACAGGGTGTCCTGACTCT | 56 |
| TNFRSF1A.1.AK | TNFR1 | GTTTAATGTATCGCTACCAA | 57 |
| TNFRSF1B.1.AG | TNFR2 | GGAAACTCAAGCCTGCACTC | 58 |
| ICAM1_A | ICAM1 | TGACGTGTGCAGTAATACTG | 59 |

| | |
|---|---|
| Negative control | IDT #1072544 |
| crRNAs #1 | |
| Negative control | IDT #1072545 |
| crRNAs #2 | |
| Negative control | IDT #1072546 |
| crRNAs #3 | |

TABLE 8
List of CRISPR/Cas12a crRNAs

| crRNA target sequence | crRNA Label | SEQ ID NO |
|---|---|---|
| AGTGGGGGTGAATTCAGTGTA | B2M-2 | 15 |
| TGACAATGACAAGCTGTATCTCTA | RFX5-5 | 39 |

Generation of gene edited human T cells using CRISPR. Human T cells were isolated from peripheral blood mononuclear cells (PBMCs) by negative selection (StemCell #17951) and rested overnight in TexMACS (Miltenyi #170-076-307), 30 IU/ml hIL-21S (Miltenyi #130-097-748), herein referred to as “media,” at 1×10⁶ cells/ml. The following day, T cells were collected, washed once with PBS, and nucleofected with 6 μl of RNP complexes specific for the indicated genes in 20 μl of P3 Buffer (Lonza #V4SP-3096) with 4 μM electroporation enhancer (IDT #1075916) in 96 well cuvettes (Lonza #V4SP-3096) using the EH-115 program on the Lonza 4D system. Immediately after nucleofection, T cells were recovered in 200 μl warm media for 2 hours at 37 C. T cells were then activated with a 1:17.5 dilution of TransAct (Miltenyi #130-019-011) in media at approximately 1×10⁶ cells/ml. T cells were expanded in culture by addition of fresh media every 2-3 days for an additional 14 days, at which point surface expression of relevant molecules was measured by flow cytometry before cryopreserving cells in Cryostor CS10 (Sigma #C2874-100ML).

Generation of allogeneic effector T cells. PBMCs from a non-HLA matched human donor were stimulated with irradiated (40 Gy) PBMCs from the human donor used to make HLA class I and II negative T cells at a 1:1 ratio in media [TexMACS (Miltenyi #170-076-307) and 100 IU/ml Penicillin+100 μg/ml Streptomycin (Gibco #15140-122)], without IL-2 at 2×10⁶ cells/ml. After 2 days, an equal volume of media containing 60 IU/ml hIL-21S (Miltenyi #130-097-748) was added to achieve a final concentration of 30 IU/ml IL-21S. After 5 additional days of culture, cells were washed, resuspended into media with 30 IU/ml IL-21S and restimulated with another round of irradiated (40 Gy) PBMCs from the human donor used to make HLA class I and II negative T cells at a 1:1 ratio. Following another 2 days of culture, an equal volume of media was added and IL-21S was supplied to 200 IU/ml. Two days later, fresh media and 200 IU/ml IL-21S was added to dilute the cells to 0.5×10⁶ cells/ml, and cells were cultured for another 3 days before being cryopreserved in CS10. In some experiments, alloreactive effector cells were separated into purified T cells (mixture of CD4+ and CD8+) and purified NK cells (CD56+CD3−) with an EasySep CD56+ isolation kit (STEMCELL Technologies #17855).

Isolation of primary NK cells. Human NK cells were isolated from leukapheresis (StemExpress and HemaCare) using the NK cell Isolation kit (Miltenyi #130-092-657) and program on a CliniMACS Prodigy (Miltenyi Biotec). NK cells were cryopreserved at 10⁶ cells/mL in Cryostor CS10 (Sigma #C2874-100ML).

Allogeneic response assays. Cryopreserved gene edited T cells (“targets”) were thawed and rested overnight in RPMI+L-glutamine (Gibco #11875-093), 10% FBS (Gibco #16140-071), 100 IU/ml Penicillin+100 μg/ml Streptomycin (Gibco #15140-122), 1 mM Sodium Pyruvate (Gibco #11360-070), 10 mM HEPES (Gibco #15630-080), and 55 μM 2-mercaptoethanol (Gibco #21985-023), herein referred to as “assay media,” supplemented with 30 IU/ml hIL-21S (Miltenyi #130-097-748), at 1×10⁶ cells/ml. The next day, target T cells were washed to remove IL-2 and seeded in 96 well U-bottom plates in assay media at 10,000 cells/well for cytotoxicity and 100,000 cells/well for CD107a assays. Allogeneic effector T cells were also thawed and rested one day prior to experiment setup in assay media supplemented with 30 IU/ml hIL-21S. Primary NK cells were also thawed and rested one day prior to experiment setup in assay media supplemented with 0.2 ng/mL IL-2 (Gibco #PHC0026). The next day, allogeneic effector T cells or NK cells were labelled with 1 μM Cell Trace Violet (Thermo #C34557) in PBS for 20 mins at 37° C., washed twice with assay media, and seeded with targets for cytotoxicity assays (various E:Ts). Cells were analyzed by flow cytometry 18-24 hours after plating. In some assays, activation of effector cells was measured by flow cytometry staining for 4-1BB (BD Bioscience, clone 4B4-1) at the conclusion of the cytotoxicity assay. Normalized target viability was calculated as: % live targets at E:T/% live targets alone. Specific lysis was calculated as: 100*(# live targets alone−# live targets at E:T)/# of live targets alone.

CD58 shRNA transduction. CD58 targeting shRNAs in a pLKO.1 lentiviral vector (Millipore Sigma) were transduced into Jurkats and 24 hour activated (TransAct, Miltenyi #130-019-011) primary human T cells at an MOI=10. CD58 expression was measured 4-7 days after transduction. The sequences of CD58 shRNAs, along with knockdown efficiency, are listed in Table 10. Knockdown efficiency was calculated as the percent reduction in geoMFI according to the formula: 100*(gMFI control−gMFI shRNA)/gMFI control.

TABLE 9
List of antibodies used in this study.

| Specificity | Clone | Company |
|---|---|---|
| Pan-HLA class I | W6/32 | BioLegend |
| CD3 | OKT3 | BioLegend |
| CD8 | RPA-T8 | BioLegend |
| Pan-HLA class II | Tu39 | BioLegend |
| CD4 | OKT4 | BioLegend |
| CD58 | 1C3 | BD Biosciences |
| B2M | 2M2 | BioLegend |
| CD56 | 5.1.H11 | BioLegend |
| 4-1BB | 4B4-1 | BD Biosciences |
| LD-NIR | n/a | Thermo Fisher |

TABLE 10
Exemplary CD58 shRNAs evaluated

| | | | | Knockdown |
|---|---|---|---|---|
| | | | Knockdown | efficiency in |
| | | SEQ | efficiency in | primary T cells |
| shRNA ID | Target Sequence | ID NO | Jurkat cells (%) | (%) |
| CD58 | GCCTCACTATCTACAACTTAA | 60 | 69.3 | 38.6 |
| shRNA 8 | | | | |
| CD58 | GTGTTGTGTATGGGAATGTAA | 61 | 40.3 | 7.9 |
| shRNA 7 | | | | |
| CD58 | GAAGACAACAGCATAACTAAA | 62 | 43.5 | 25.3 |
| shRNA 6 | | | | |
| CD58 | ACGTAACTCAACCAGTATATA | 63 | 49.3 | 21.4 |
| shRNA 5 | | | | |
| CD58 | TACTCTTAGCAATCCATTATT | 64 | 54.5 | 28.5 |
| shRNA 4 | | | | |
| CD58 | GCATTGACTAATGGAAGCATT | 65 | −6.6 | 2.4 |
| shRNA 3 | | | | |
| CD58 | GTGCTGTATATGAATGGTATT | 66 | 4.3 | 9.7 |
| shRNA 2 | | | | |
| CD58 | GCGGTCATTCAAGACACAGAT | 67 | 44.6 | 16.2 |
| shRNA 1 | | | | |

CD2 is an important costimulatory receptor on T cells and NK cells, involved in immune synapse formation. The ligand for CD2 is CD58 (LFA3) expressed on APC's and many other cell types. The present disclosure discovered that reduction in CD58 expression conferred resistance to cells that are low/lacking in HLA-I (e.g., B2M knockout) expression from an NK missing-self response. Reduction in CD58 expression also conferred partial resistance to cells that are expressing normal or low levels of HLA-I and HLA-II (e.g., RFX5 knockout) from alloreactive T cell cytotoxicity.

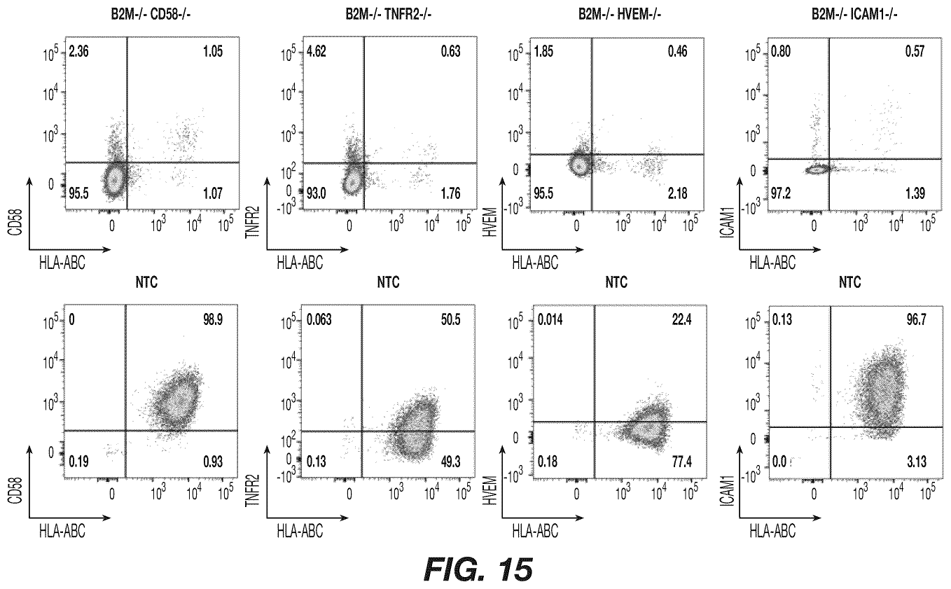

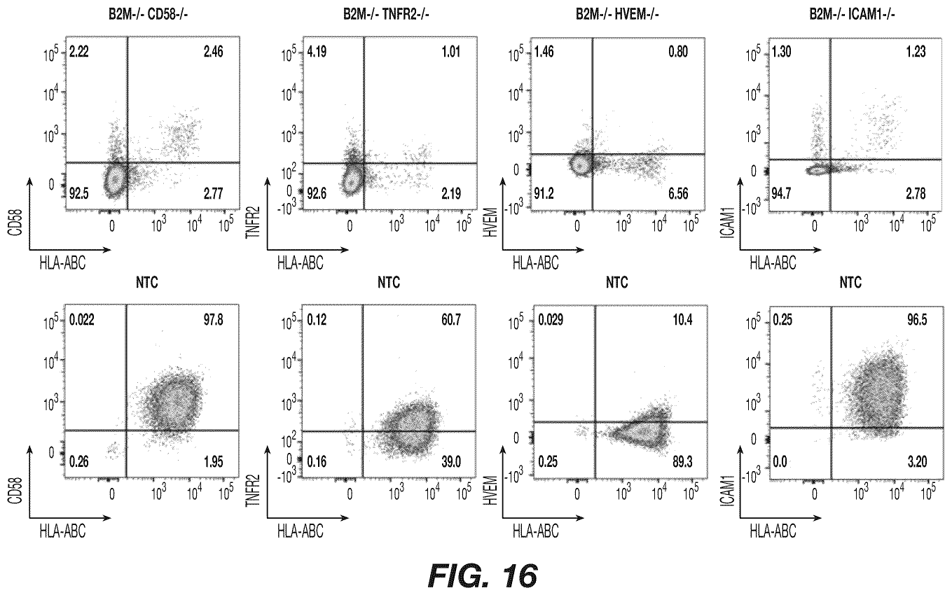

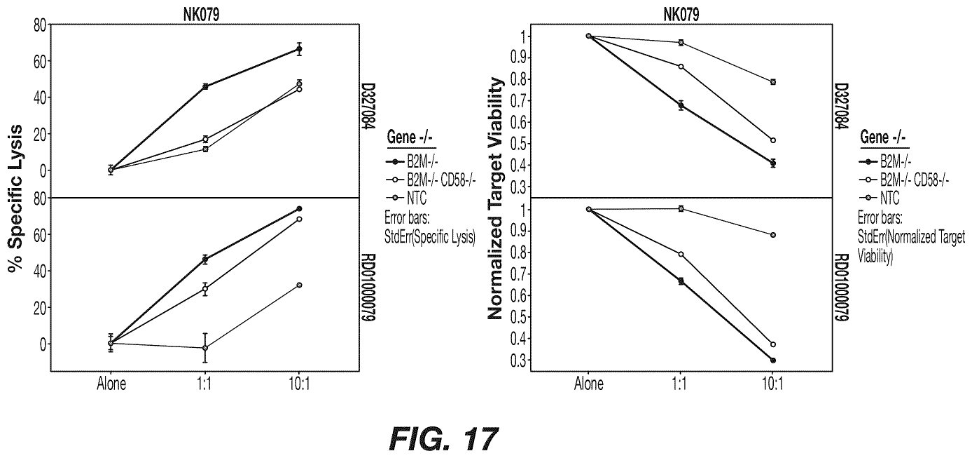

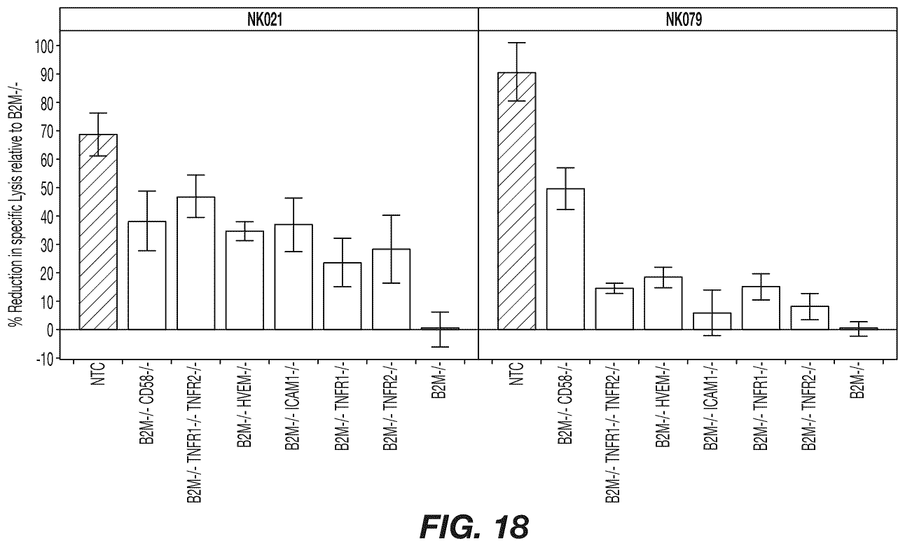

CD58 knockout reduced specific lysis of B2M knockout T cells by primary NK effector cells. FIGS. 15 and 16 show generation and phenotype of B2M and co-stimulatory knockout cells. Populations of cells were generated in which greater than 90% of the cells were double negative for B2M and the additional co-stimulatory gene. CD58 knockout combined with B2M knockout resulted in less specific lysis and improved cell viability compared to B2M knockout only T cells when co-cultured with primary NK cells (FIG. 17). The addition of CD58 knockout to a B2M knockout reverses some of the NK cell cytotoxicity driven by a lack of HLA class I in B2M knockout T cells (FIG. 17). CD58 knockout in a B2M knockout T cell reduced specific lysis from NK cells from multiple human donors (FIG. 18). The data showed that the disruption of several genes, such as CD58, TNFR1, TNFR2, HVEM, and ICAM1, reversed some of the NK cell cytotoxicity driven by a lack of HLA class I in B2M knockout T cells (FIG. 18).

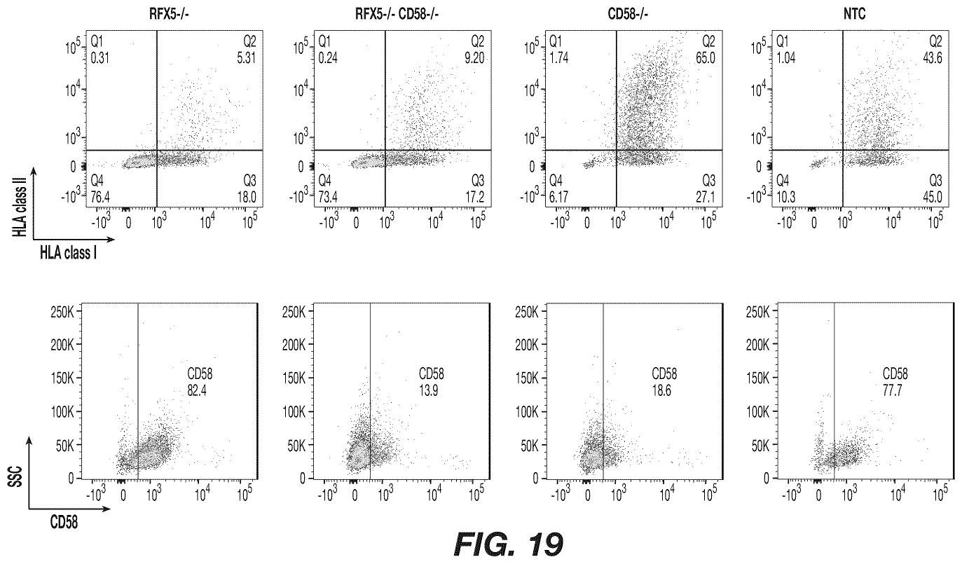

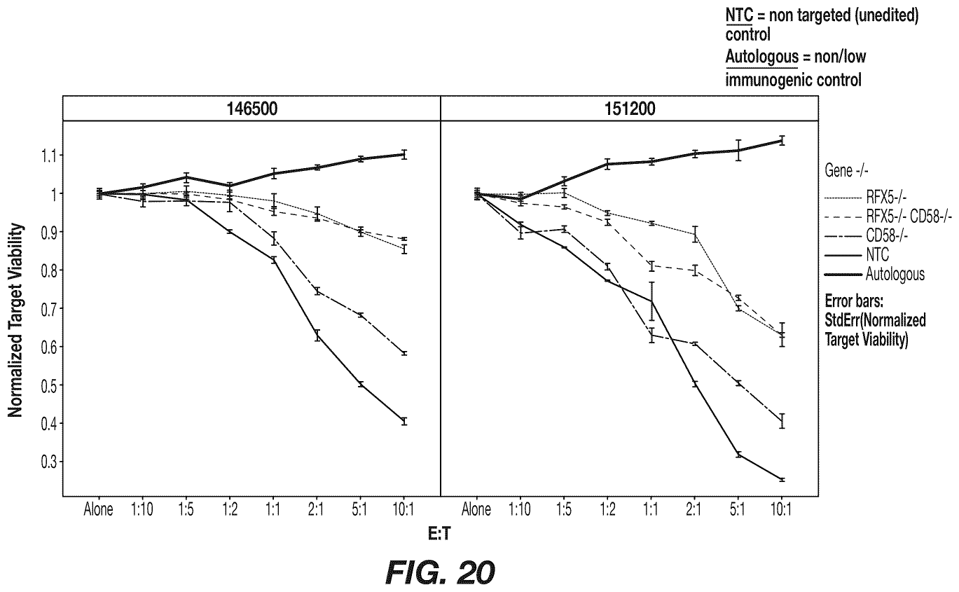

CD58 knockout improved viability compared to unedited T cells in co-culture with alloreactive effector T cells. RFX5 and CD58 knockout pan T cells were generated. The editing efficiency of RFX5 and CD58 was roughly 78-88% and 76-82%, respectively, as calculated by the following formula for RFX5: (1−(Sample % HLA class II positive cells/NTC % HLA class II positive cells))*100, and the following formula for CD58: (1-(Sample % CD58 positive cells/NTC % CD58 positive cells))*100 (FIG. 19). CD58 knockout improved viability compared to unedited (NTC) cells in alloreactive T cell co-culture from two human donors (FIG. 20). As compared to unedited (NTC) T cells, CD58 knockout T cells had an improved ability to survive challenge with allogeneic effector T cells. RFX5 knockout T cells had a strongly enhanced ability to survive compared to unedited cells (FIG. 20).

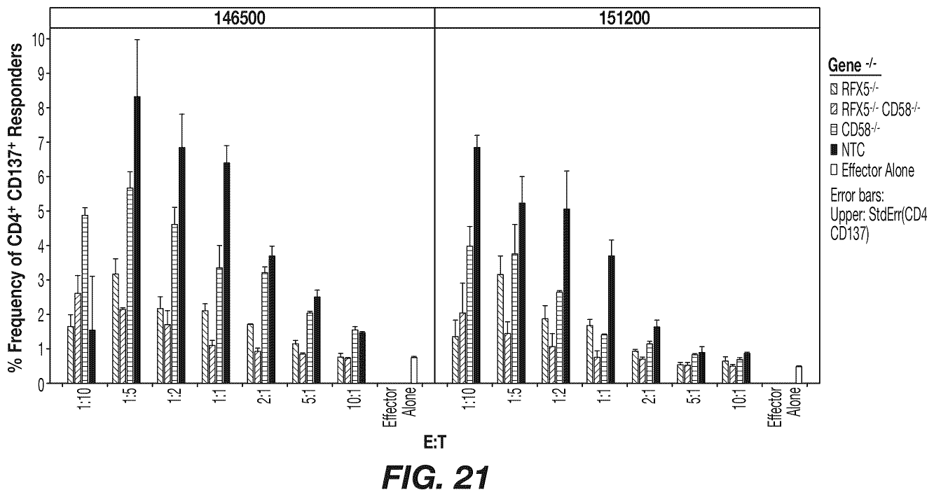

CD58 knockout in addition to RFX5 knockout induced less activation (CD137+) of alloreactive CD4⁺ T cells than RFX5 knockout alone (FIG. 21). As compared to unedited (NTC) T cells, CD58 knockout T cells showed a reduced ability to activate allogeneic CD4⁺ T cells from two human donors. RFX5 knockout T cells showed a strongly reduced ability to activate allogeneic CD4⁺ T cells, and CD58 knockout in addition to RFX5 knockout further reduced the ability to activate allogeneic CD4⁺ T cells at most E:T ratios tested (FIG. 21).

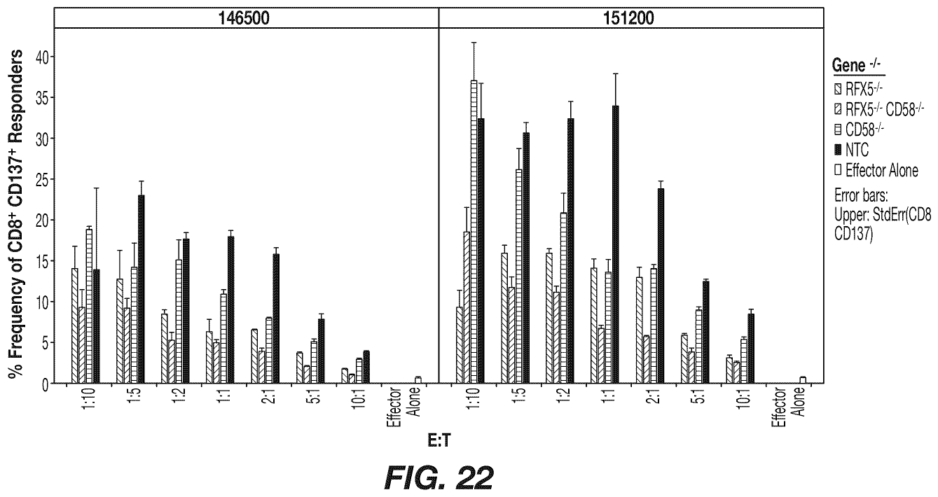

CD58 knockout in addition to RFX5 knockout also induced less activation (CD137+) of alloreactive CD8⁺ T cells than RFX5 knockout alone (FIG. 22). As compared to unedited (NTC) T cells, CD58 knockout T cells showed a reduced ability to activate allogeneic CD8⁺ T cells from two human donors. RFX5 knockout T cells showed a strongly reduced ability to activate allogeneic CD8⁺ T cells, and CD58 knockout in addition to RFX5 knockout further reduced the ability to activate allogeneic CD8⁺ T cells at most E:T ratios tested (FIG. 22).

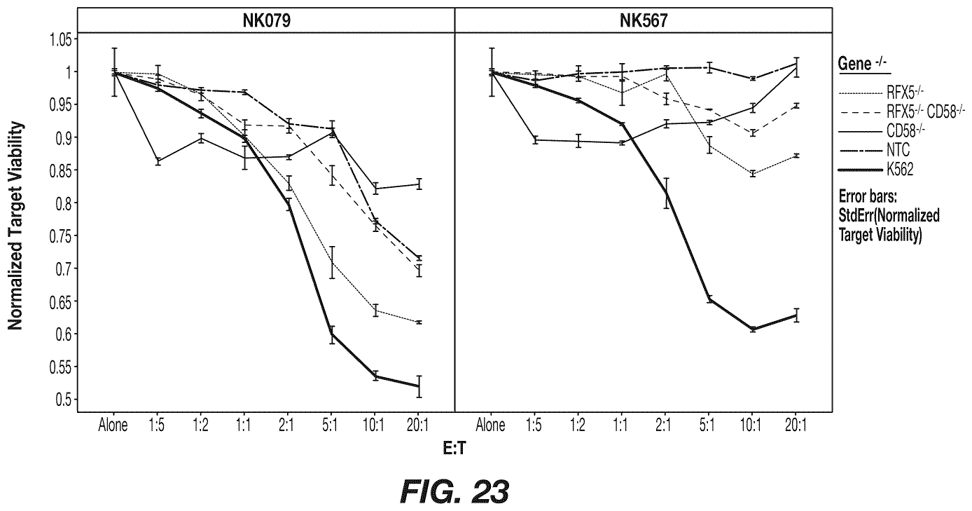

CD58 knockout in addition to RFX5 knockout improved viability compared to RFX5 knockout in co-culture with primary NK from two human donors. The data showed that the addition of CD58 knockout to a RFX5 knockout reversed most of the NK cell cytotoxicity driven by a reduction of HLA class I in RFX5 knockout T cells (FIG. 23).

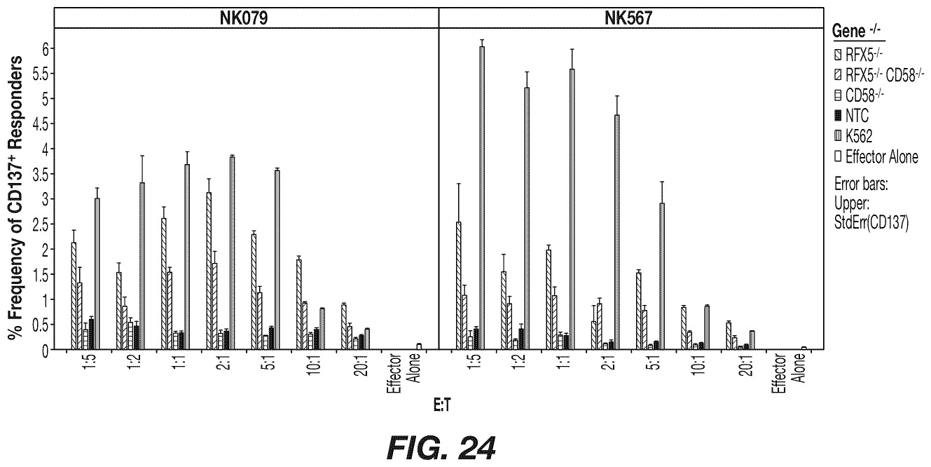

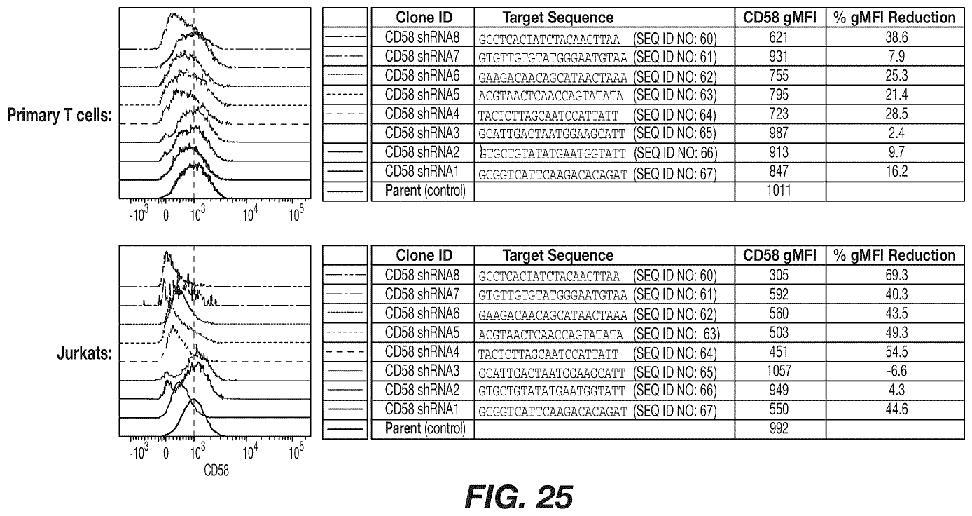

CD58 knockout in addition to RFX5 knockout induced less activation (CD137+) of primary NK cells than RFX5 knockout alone. As compared to unedited (NTC) T cells, RFX5 knockout T cells showed an increased ability to activate NK cells, consistent with a reduction in HLA class I on RFX5 knockout cells. CD58 knockout in combination with RFX5 knockout was shown to partially reduce the activation of NK cells at most E:T ratios tested, consistent with the enhanced ability of RFX5/CD58 double knockout T cells to survive NK cell cytotoxicity relative to RFX5 knockout T cells (FIG. 24). CD58 shRNAs tested in Jurkat and primary T cells showed knockdown of CD58 surface protein with certain shRNA sequences producing a stronger knockdown effect than others (FIG. 25), demonstrating the usefulness and versatility of the present invention for various applications (e.g., to achieve moderate, strong, and lower knockdown effects).

### 9.3 Example 3: Knocking Out B2M with Cas12a and MAD7 in iPSCs

Preparation of RNP complexes. B2M_12A_2 Alt-R crRNA was synthesized (IDT) and dissolved in nuclease free duplex buffer (IDT #11-05-01-12) at 200 PM. RNP complexes were prepared fresh on the day of nucleofection by mixing a ratio of 63 pmol (1 μl of 10 μg/μl) Alt-R® A.s. Cas12a (Cpf1) V3 (IDT #1081068) or WT MAD7 (Aldevron) with 200 pmol (1 μl of 200 μM) crRNA and 100 μg of poly-L-glutamic acid sodium salt (PGA, MW15-50 kD, Sigma p4761). The RNP complex was incubated at room temperature for 30 minutes. P3 Buffer (Lonza #V4SP-3096) with 3 μM electroporation enhancer (IDT #1076301) was added for a total volume of 10 μL.

Generation of HLA class I negative human iPSCs. Human iPS cells, herein referred to as “iPSCs”, were pretreated with 10 μM Y-27632 ROCK inhibitor (STEMCELL Technologies #72302) in Stemfit Basic 04 Complete Type Medium (Ajinomoto Basic04CT). The iPSCs were collected (0.5e6 cells per reaction) and resuspended in 10 μL of P3 buffer with 3 μM electroporation enhancer. The cells were combined with 10 μL of relevant RNP complex and nucleofected in 96 well cuvettes (Lonza #V4SP-3096) using the CA-137 program on the Lonza 4D system. The iPSCs were then transferred to one well of 24 well plate coated with 0.5 μg/cm² of iMatrix-511 (Takara #T304) containing Stemfit Basic 04 Complete Type Medium (Ajinomoto Basic04CT) with 10 μM Y-27632 ROCK inhibitor. The iPSCs were expanded to 6 well plates two days post nucleofection and the media was changed daily for 7 days, at which pluripotency markers and surface expression of HLA class I were measured by flow cytometry.

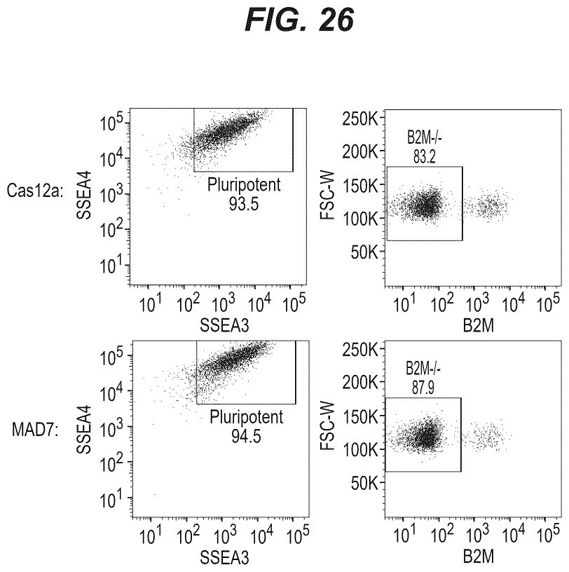

Knocking out B2M with a gRNA in combination with either Cas12a or wildtype MAD7 provided reduced B2M expression while maintaining iPSC pluripotency. FIG. 26 shows the B2M editing efficiency with Cas12a and WT MAD7 in iPSCs. Cas12a or MAD7 RNP was formed with gRNA B2M_12A_2 (Table 11; SEQ ID NO: 252). The flow plots shown are gated on live, single cells. Editing with both RNPs resulted in a reduction in expression of B2M (>80%) while retaining iPSC pluripotency. These results show that B2M knockout can be achieved with high efficiency with gRNA B2M_12A_2 and either Cas12a/Cpf1 or WT MAD7.

TABLE 11
Exemplary B2M gRNAs.

| | | | | | SEQ | | | | |
|---|---|---|---|---|---|---|---|---|---|
| | | | | | ID NO: | | | | |
| | SEQ | | SEQ | | for | | | SEQ ID | |
| | ID NO: | | ID NO: | | Repeat + | Repeat + | | NO: | |
| | for | | for | | Spacer | Spacer | | for | |
| | Repeat | Repeat | Spacer | Spacer | Complete | Complete | Chromosome | Genomic | Genomic |
| | Sequence | Sequence | Sequence | Sequence | Sequence | Sequence | location | Target | Target |
| ID | (5′-3′) | (5′-3′) | (5′-3′) | (5′-3′) | (5′-3′) | (5′-3′) | (hg38) | Region | Region |
| B2M_ | 129 | UAAUUU | 251 | AGUG | 252 | UAAUU | Chr15: | 253 | TTTCAG |
| 12A_2 | | CUACUC | | GGGG | | UCUACU | 44715614- | | TGGGGG |
| | | UUGUAG | | UGAA | | CUUGUA | 44715634 | | TGAATT |
| | | AU | | UUCA | | GAUAG | | | CAGTGT |
| | | | | GUGU | | UGGGG | | | A |
| | | | | A | | GUGAA | | | |
| | | | | | | UUCAGU | | | |
| | | | | | | GUA | | | |

### 9.4 Example 4: Exemplary gRNA Structure Engineering for Knocking Out RFX5

Preparation of RNP complexes. Alt-R crRNAs were synthesized (IDT) and dissolved in nuclease free duplex buffer (IDT #11-05-01-12) at 200 μM. RNP complexes were prepared fresh on the day of nucleofection by mixing a ratio of 63 pmol (1 μl of 10 μg/μl) WT MAD7 (Aldevron) with 200 pmol (1 μl of 200 μM) crRNA and 100 μg of poly-L-glutamic acid sodium salt (PGA, MW15-50 kD, Sigma p4761). The RNP complex was incubated at room temperature for 30 minutes. P3 Buffer (Lonza #V4SP-3096) with 3 μM electroporation enhancer (IDT #1076301) was added for a total volume of 10 μL.

Generation of knockout human iPSCs. Human iPSCs were pretreated with 10 μM Y-27632 ROCK inhibitor (STEMCELL Technologies #72302) in Stemfit Basic 04 Complete Type Medium (Ajinomoto Basic04CT). The iPSCs were collected (0.5e6 cells per reaction) and resuspended in 10 μL of P3 buffer with 3 μM electroporation enhancer. The cells were combined with 10 μL of relevant RNP complex and nucleofected in 96 well cuvettes (Lonza #V4SP-3096) using the CA-137 program on the Lonza 4D system. The iPSCs were then transferred to one well of 24 well plate coated with 0.5 μg/cm² of iMatrix-511 (Takara #T304) containing Stemfit Basic 04 Complete Type Medium (Ajinomoto Basic04CT) with 10 μM Y-27632 ROCK inhibitor. The iPSCs were collected 48 hours post electroporation, the DNA was extracted, and the region around the gRNA target-site was amplified and Sanger sequenced. Editing efficiency was measured by the ICE tool (Synthego) to analyze Sanger sequencing results (GENEWIZ).

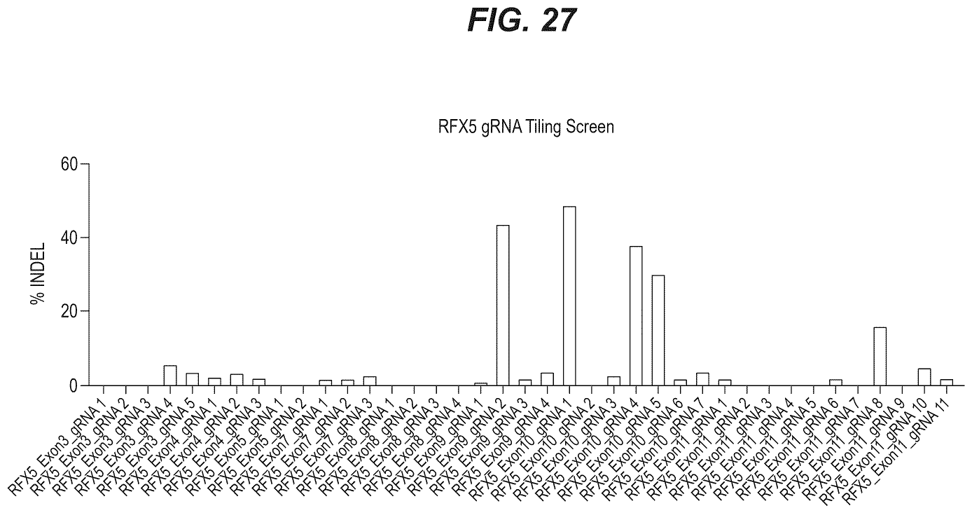

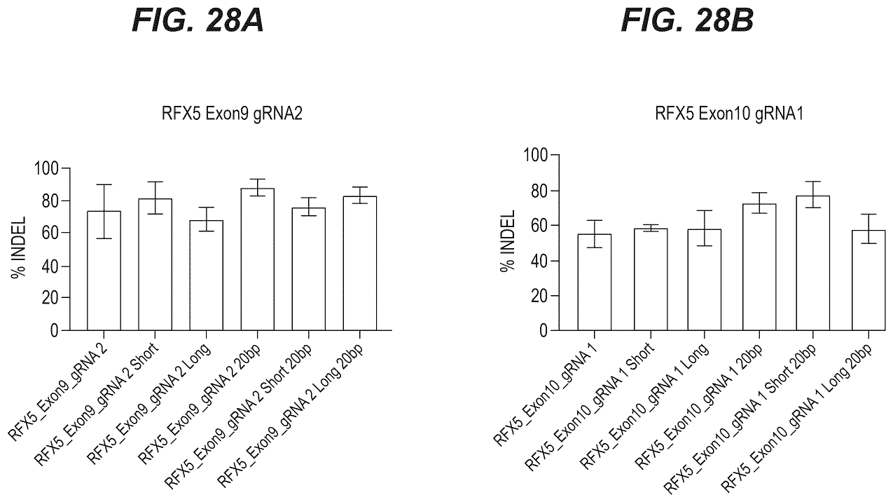

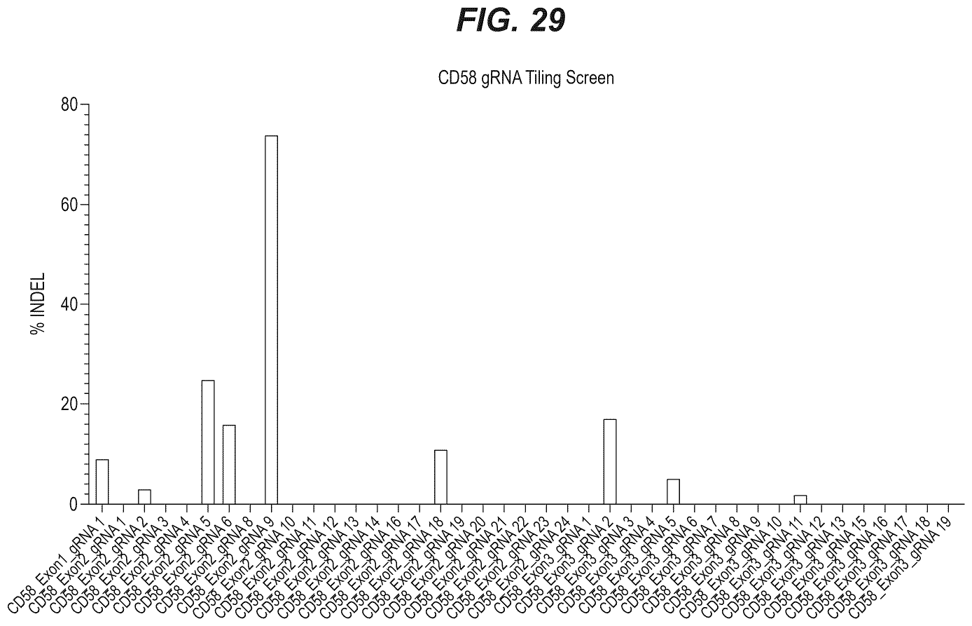

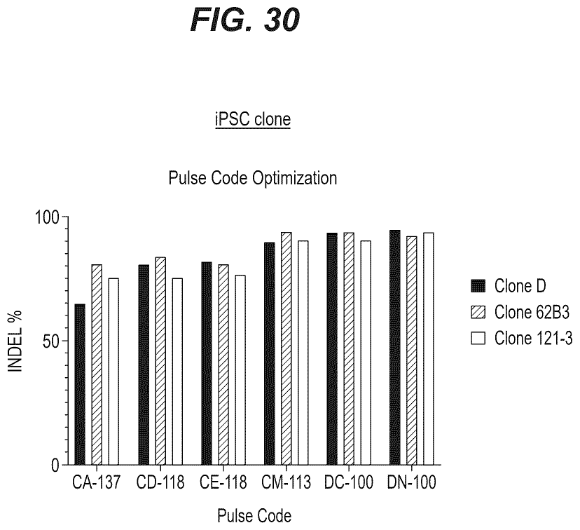

RFX5 knockouts were generated using gRNAs and WT MAD7. FIG. 27 shows a RFX5 gRNA tiling screen in iPSCs. The editing efficiency of each gRNA tested to knockout the RFX5 gene is shown. Some gRNAs tested showed zero or minimal editing. Several gRNAs had moderate editing and the top four had high editing that can be used to efficiently knockout RFX5 with MAD7 in iPSCs (Table 12). FIGS. 28A and 28B show optimization of the gRNA structure to knockout RFX5. The editing efficiencies of the top two RFX5 gRNAs with optimization to the gRNA structure are RFX5 Exon9 gRNA 2 and REX Exon gRNA1. Three repeat sequences were tested as well as 20 bp and 2 bp spacer sequence lengths. High editing efficiencies can be achieved with both gRNAs (RFX5 Exon9 gRNA 2 and REX Exon0 gRNA1) with modifications to the repeat region and varying the length of the target recognition sequence (Table 13).

TABLE 12
Exemplary RFX5 gRNAs.

| | | | | | SEQ | | | | |
|---|---|---|---|---|---|---|---|---|---|
| | | | | | ID NO: | | | | |
| | SEQ | | SEQ | | for | | | SEQ ID | |
| | ID NO: | | ID NO: | | Repeat + | Repeat + | | NO: | |
| | for | | for | | Spacer | Spacer | | for | |
| | Repeat | Repeat | Spacer | Spacer | Complete | Complete | Chromosome | Genomic | Genomic |
| | Sequence | Sequence | Sequence | Sequence | Sequence | Sequence | location | Target | Target |
| ID | (5′-3′) | (5′-3′) | (5′-3′) | (5′-3′) | (5′-3′) | (5′-3′) | (hg38) | Region | Region |
| RFX5_ | 129 | UAAUUU | 130 | CGAAAU | 131 | UAAUUU | Chr1: | 132 | CTTACG |
| Exon3_ | | CUACUC | | GGUACC | | CUACUC | 151346204- | | AAATGG |
| gRNA 1 | | UUGUAG | | UCGGAG | | UUGUAG | 151346224 | | TACCTC |
| | | AU | | CCU | | AUCGAA | | | GGAGCC |
| | | | | | | AUGGUA | | | T |
| | | | | | | CCUCGG | | | |
| | | | | | | AGCCU | | | |
| RFX5_ | 129 | UAAUUU | 133 | UUCAGA | 134 | UAAUUU | Chr1: | 135 | CTTCTT |
| Exon3_ | | CUACUC | | GGCUCC | | CUACUC | 151346209- | | CAGAGG |
| gRNA 2 | | UUGUAG | | GAGGUA | | UUGUAG | 151346229 | | CTCCGA |
| | | AU | | CCA | | AUUUCA | | | GGTACC |
| | | | | | | GAGGCU | | | A |
| | | | | | | CCGAGG | | | |
| | | | | | | UACCA | | | |
| RFX5_ | 129 | UAAUUU | 136 | GAGAUG | 137 | UAAUUU | Chr1: | 138 | CTTGGA |
| Exon3_ | | CUACUC | | UGAUGA | | CUACUC | 151346185- | | GATGTG |
| gRNA 3 | | UUGUAG | | GUACUU | | UUGUAG | 151346205 | | ATGAGT |
| | | AU | | ACG | | AUGAGA | | | ACTTAC |
| | | | | | | UGUGAU | | | G |
| | | | | | | GAGUAC | | | |
| | | | | | | UUACG | | | |
| RFX5_ | 129 | UAAUUU | 139 | GGGCUC | 140 | UAAUUU | Chr1: | 141 | CTTGGG |
| Exon3_ | | CUACUC | | UUAGCA | | CUACUC | 151346289- | | GCTCTT |
| gRNA 4 | | UUGUAG | | UCAGGC | | UUGUAG | 151346309 | | AGCATC |
| | | AU | | UCA | | AUGGGC | | | AGGCTC |
| | | | | | | UCUUAG | | | A |
| | | | | | | CAUCAG | | | |
| | | | | | | GCUCA | | | |
| RFX5_ | 129 | UAAUUU | 142 | AGAGGC | 143 | UAAUUU | Chr1: | 144 | CTTCAG |
| Exon3_ | | CUACUC | | UCCGAG | | CUACUC | 151346206- | | AGGCTC |
| gRNA 5 | | UUGUAG | | GUACCA | | UUGUAG | 151346226 | | CGAGGT |
| | | AU | | UUU | | AUAGAG | | | ACCATT |
| | | | | | | GCUCCG | | | T |
| | | | | | | AGGUAC | | | |
| | | | | | | CAUUU | | | |
| RFX5_ | 129 | UAAUUU | 145 | UUCUGC | 146 | UAAUUU | Chr1: | 147 | TTTGTT |
| Exon4_ | | CUACUC | | ACGGCC | | CUACUC | 151345947- | | CTGCAC |
| gRNA 1 | | UUGUAG | | UUGCUG | | UUGUAG | 151345967 | | GGCCTT |
| | | AU | | UGG | | AUUUCU | | | GCTGTG |
| | | | | | | GCACGG | | | G |
| | | | | | | CCUUGC | | | |
| | | | | | | UGUGG | | | |
| RFX5_ | 129 | UAAUUU | 148 | CCCACA | 149 | UAAUUU | Chr1: | 150 | CTTCCC |
| Exon4_ | | CUACUC | | GCAAGG | | CUACUC | 151345948- | | CACAGC |
| gRNA 2 | | UUGUAG | | CCGUGC | | UUGUAG | 151345968 | | AAGGCC |
| | | AU | | AGA | | AUCCCA | | | GTGCAG |
| | | | | | | CAGCAA | | | A |
| | | | | | | GGCCGU | | | |
| | | | | | | GCAGA | | | |
| RFX5_ | 129 | UAAUUU | 151 | GUUCUG | 152 | UAAUUU | Chr1: | 153 | CTTTGT |
| Exon4_ | | CUACUC | | CACGGC | | CUACUC | 151345946- | | TCTGCA |
| gRNA 3 | | UUGUAG | | CUUGCU | | UUGUAG | 151345966 | | CGGCCT |
| | | AU | | GUG | | AUGUUC | | | TGCTGT |
| | | | | | | UGCACG | | | G |
| | | | | | | GCCUUG | | | |
| | | | | | | CUGUG | | | |
| RFX5_ | 129 | UAAUUU | 154 | GUCUCC | 155 | UAAUUU | Chr1: | 156 | TTTTGT |
| Exon5_ | | CUACUC | | AGUGGU | | CUACUC | 151345111- | | CTCCAG |
| gRNA 1 | | UUGUAG | | GGGUCC | | UUGUAG | 151345131 | | TGGTGG |
| | | AU | | UGA | | AUGUCU | | | GTCCTG |
| | | | | | | CCAGUG | | | A |
| | | | | | | GUGGGU | | | |
| | | | | | | CCUGA | | | |
| RFX5_ | 129 | UAAUUU | 157 | CUGACA | 158 | UAAUUU | Chr1: | 159 | TTTTCT |
| Exon5_ | | CUACUC | | AUGACA | | CUACUC | 151345149- | | GACAAT |
| gRNA 2 | | UUGUAG | | AGCUGU | | UUGUAG | 151345169 | | GACAAG |
| | | AU | | AUC | | AUCUGA | | | CTGTAT |
| | | | | | | CAAUGA | | | C |
| | | | | | | CAAGCU | | | |
| | | | | | | GUAUC | | | |
| RFX5_ | 129 | UAAUUU | 160 | GACUGG | 161 | UAAUUU | Chr1: | 162 | CTTGGA |
| Exon7_ | | CUACUC | | CCCCGG | | CUACUC | 151344420- | | CTGGCC |
| gRNA 1 | | UUGUAG | | CCACCA | | UUGUAG | 151344440 | | CCGGCC |
| | | AU | | AGC | | AUGACU | | | ACCAAG |
| | | | | | | GGCCCC | | | C |
| | | | | | | GGCCAC | | | |
| | | | | | | CAAGC | | | |
| RFX5_ | 129 | UAAUUU | 163 | GUGGCC | 164 | UAAUUU | Chr1: | 165 | CTTGGT |
| Exon7_ | | CUACUC | | GGGGCC | | CUACUC | 151344415- | | GGCCGG |
| gRNA 2 | | UUGUAG | | AGUCCA | | UUGUAG | 151344435 | | GGCCAG |
| | | AU | | AGU | | AUGUGG | | | TCCAAG |
| | | | | | | CCGGGG | | | T |
| | | | | | | CCAGUC | | | |
| | | | | | | CAAGU | | | |
| RFX5_ | 129 | UAAUUU | 166 | CCUGUU | 167 | UAAUUU | Chr1: | 168 | CTTGCC |
| Exon7_ | | CUACUC | | GCCGCC | | CUACUC | 151344496- | | TGTTGC |
| gRNA 3 | | UUGUAG | | CACUCA | | UUGUAG | 151344516 | | CGCCCA |
| | | AU | | GCA | | AUCCUG | | | CTCAGC |
| | | | | | | UUGCCG | | | A |
| | | | | | | CCCACU | | | |
| | | | | | | CAGCA | | | |
| RFX5_ | 129 | UAAUUU | 169 | AGGUCA | 170 | UAAUUU | Chr1: | 171 | CTTTAG |
| Exon8_ | | CUACUC | | AGUCCA | | CUACUC | 151344213- | | GTCAAG |
| gRNA 1 | | UUGUAG | | GGCAGG | | UUGUAG | 151344233 | | TCCAGG |
| | | AU | | GGU | | AUAGGU | | | CAGGGG |
| | | | | | | CAAGUC | | | T |
| | | | | | | CAGGCA | | | |
| | | | | | | GGGGU | | | |
| RFX5_ | 129 | UAAUUU | 172 | ACUUGC | 173 | UAAUUU | Chr1: | 174 | TTTAAC |
| Exon8_ | | CUACUC | | AUCAGA | | CUACUC | 151344269- | | TTGCAT |
| gRNA 2 | | UUGUAG | | UAUUGC | | UUGUAG | 151344289 | | CAGATA |
| | | AU | | UAC | | AUACUU | | | TTGCTA |
| | | | | | | GCAUCA | | | C |
| | | | | | | GAUAUU | | | |
| | | | | | | GCUAC | | | |
| RFX5_ | 129 | UAAUUU | 175 | CUCCUU | 176 | UAAUUU | Chr1: | 177 | CTTCCT |
| Exon8_ | | CUACUC | | AUGCCA | | CUACUC | 151344255- | | CCTTAT |
| gRNA 3 | | UUGUAG | | CUGUAG | | UUGUAG | 151344275 | | GCCACT |
| | | AU | | CAA | | AUCUCC | | | GTAGCA |
| | | | | | | UUAUGC | | | A |
| | | | | | | CACUGU | | | |
| | | | | | | AGCAA | | | |
| RFX5_ | 129 | UAAUUU | 178 | CACUCU | 179 | UAAUUU | Chr1: | 180 | CTTACA |
| Exon8_ | | CUACUC | | CAGAAC | | CUACUC | 151344196- | | CTCTCA |
| gRNA 4 | | UUGUAG | | CCUUUA | | UUGUAG | 151344216 | | GAACCC |
| | | AU | | GGU | | AUCACU | | | TTTAGG |
| | | | | | | CUCAGA | | | T |
| | | | | | | ACCCUU | | | |
| | | | | | | UAGGU | | | |
| RFX5_ | 129 | UAAUUU | 181 | AGUUCC | 182 | UAAUUU | Chr1: | 183 | CTTCAG |
| Exon9_ | | CUACUC | | AUCGUU | | CUACUC | 151343757- | | TTCCAT |
| gRNA 1 | | UUGUAG | | GAGGUC | | UUGUAG | 151343777 | | CGTTGA |
| | | AU | | GCC | | AUAGUU | | | GGTCGC |
| | | | | | | CCAUCG | | | C |
| | | | | | | UUGAGG | | | |
| | | | | | | UCGCC | | | |
| RFX5_ | 129 | UAAUUU | 184 | AGGAUC | 185 | UAAUUU | Chr1: | 186 | TTTCAG |
| Exon9_ | | CUACUC | | CGCUCU | | CUACUC | 151343791- | | GATCCG |
| gRNA 2 | | UUGUAG | | GCCCAG | | UUGUAG | 151343811 | | CTCTGC |
| | | AU | | UCA | | AUAGGA | | | CCAGTC |
| | | | | | | UCCGCU | | | A |
| | | | | | | CUGCCC | | | |
| | | | | | | AGUCA | | | |
| RFX5_ | 129 | UAAUUU | 187 | AGGCCA | 188 | UAAUUU | Chr1: | 189 | CTTAAG |
| Exon9_ | | CUACUC | | UGGGGC | | CUACUC | 151343678- | | GCCATG |
| gRNA 3 | | UUGUAG | | UCGCUG | | UUGUAG | 151343698 | | GGGCTC |
| | | AU | | GUG | | AUAGGC | | | GCTGGT |
| | | | | | | CAUGGG | | | G |
| | | | | | | GCUCGC | | | |
| | | | | | | UGGUG | | | |
| RFX5_ | 129 | UAAUUU | 190 | CUGCUA | 191 | UAAUUU | Chr1: | 192 | CTTCCT |
| Exon9_ | | CUACUC | | CAGCAG | | CUACUC | 151343730- | | GCTACA |
| gRNA 4 | | UUGUAG | | CAUCUC | | UUGUAG | 151343750 | | GCAGCA |
| | | AU | | AUC | | AUCUGC | | | TCTCAT |
| | | | | | | UACAGC | | | C |
| | | | | | | AGCAUC | | | |
| | | | | | | UCAUC | | | |
| RFX5_ | 129 | UAAUUU | 193 | GAUGAC | 194 | UAAUUU | Chr1: | 195 | TTTAGA |
| Exon10_ | | CUACUC | | CGUUCC | | CUACUC | 151343409- | | TGACCG |
| gRNA | | UUGUAG | | CGAGGU | | UUGUAG | 151343429 | | TTCCCG |
| 1 | | AU | | GCA | | AUGAUG | | | AGGTGC |
| | | | | | | ACCGUU | | | A |
| | | | | | | CCCGAG | | | |
| | | | | | | GUGCA | | | |
| RFX5_ | 129 | UAAUUU | 196 | GUUUAG | 197 | UAAUUU | Chr1: | 198 | TTTGGT |
| Exon10_ | | CUACUC | | AUGACC | | CUACUC | 151343404- | | TTAGAT |
| gRNA | | UUGUAG | | GUUCCC | | UUGUAG | 151343424 | | GACCGT |
| 2 | | AU | | GAG | | AUGUUU | | | TCCCGA |
| | | | | | | AGAUGA | | | G |
| | | | | | | CCGUUC | | | |
| | | | | | | CCGAG | | | |
| RFX5_ | 129 | UAAUUU | 199 | GGUUUA | 200 | UAAUUU | Chr1: | 201 | CTTTGG |
| Exon10_ | | CUACUC | | GAUGAC | | CUACUC | 151343403- | | TTTAGA |
| gRNA | | UUGUAG | | CGUUCC | | UUGUAG | 151343423 | | TGACCG |
| 3 | | AU | | CGA | | AUGGUU | | | TTCCCG |
| | | | | | | UAGAUG | | | A |
| | | | | | | ACCGUU | | | |
| | | | | | | CCCGA | | | |
| RFX5_ | 129 | UAAUUU | 202 | GAGAAC | 203 | UAAUUU | Chr1: | 204 | TTTAGA |
| Exon10_ | | CUACUC | | CCAGAG | | CUACUC | 151343369- | | GAACCC |
| gRNA | | UUGUAG | | GGUGGA | | UUGUAG | 151343389 | | AGAGG |
| 4 | | AU | | GCC | | AUGAGA | | | GTGGAG |
| | | | | | | ACCCAG | | | CC |
| | | | | | | AGGGUG | | | |
| | | | | | | GAGCC | | | |
| RFX5_ | 129 | UAAUUU | 205 | GUACCU | 206 | UAAUUU | Chr1: | 207 | TTTGGT |
| Exon10_ | | CUACUC | | CUGCAG | | CUACUC | 151343434- | | ACCTCT |
| gRNA | | UUGUAG | | AAGAGG | | UUGUAG | 151343454 | | GCAGAA |
| 5 | | AU | | ACG | | AUGUAC | | | GAGGAC |
| | | | | | | CUCUGC | | | G |
| | | | | | | AGAAGA | | | |
| | | | | | | GGACG | | | |
| RFX5_ | 129 | UAAUUU | 208 | GGUACC | 209 | UAAUUU | Chr1: | 210 | TTTTGG |
| Exon10_ | | CUACUC | | UCUGCA | | CUACUC | 151343435- | | TACCTC |
| gRNA | | UUGUAG | | GAAGAG | | UUGUAG | 151343455 | | TGCAGA |
| 6 | | AU | | GAC | | AUGGUA | | | AGAGG |
| | | | | | | CCUCUG | | | AC |
| | | | | | | CAGAAG | | | |
| | | | | | | AGGAC | | | |
| RFX5_ | 129 | UAAUUU | 211 | CUUACC | 212 | UAAUUU | Chr1: | 213 | CTTCCT |
| Exon10_ | | CUACUC | | UGGGCC | | CUACUC | 151343337- | | TACCTG |
| gRNA | | UUGUAG | | AGUCUC | | UUGUAG | 151343357 | | GGCCAG |
| 7 | | AU | | UCU | | AUCUUA | | | TCTCTC |
| | | | | | | CCUGGG | | | T |
| | | | | | | CCAGUC | | | |
| | | | | | | UCUCU | | | |
| RFX5_ | 129 | UAAUUU | 214 | AGCUGU | 215 | UAAUUU | Chr1: | 216 | CTTCAG |
| Exon11_ | | CUACUC | | CCUCUU | | CUACUC | 151342735- | | CTGTCC |
| gRNA | | UUGUAG | | GACACC | | UUGUAG | 151342755 | | TCTTGA |
| 1 | | AU | | CUU | | AUAGCU | | | CACCCT |
| | | | | | | GUCCUC | | | T |
| | | | | | | UUGACA | | | |
| | | | | | | CCCUU | | | |
| RFX5_ | 129 | UAAUUU | 217 | AGCUGG | 218 | UAAUUU | Chr1: | 219 | CTTTAG |
| Exon11_ | | CUACUC | | UGGAGC | | CUACUC | 151342693- | | CTGGTG |
| gRNA | | UUGUAG | | CUGCCC | | UUGUAG | 151342713 | | GAGCCT |
| 2 | | AU | | ACU | | AUAGCU | | | GCCCAC |
| | | | | | | GGUGGA | | | T |
| | | | | | | GCCUGC | | | |
| | | | | | | CCACU | | | |
| RFX5_ | 129 | UAAUUU | 220 | CGCUCU | 221 | UAAUUU | Chr1: | 222 | CTTCCG |
| Exon11_ | | CUACUC | | CCACGU | | CUACUC | 151343123- | | CTCTCC |
| gRNA | | UUGUAG | | GCGAGA | | UUGUAG | 151343143 | | ACGTGC |
| 3 | | AU | | GGA | | AUCGCU | | | GAGAG |
| | | | | | | CUCCAC | | | GA |
| | | | | | | GUGCGA | | | |
| | | | | | | GAGGA | | | |
| RFX5_ | 129 | UAAUUU | 223 | AGGGCA | 224 | UAAUUU | Chr1: | 225 | TTTCAG |
| Exon11_ | | CUACUC | | CCUGAA | | CUACUC | 151342958- | | GGCACC |
| gRNA | | UUGUAG | | GAAAGC | | UUGUAG | 151342978 | | TGAAGA |
| 8 | | AU | | CUG | | AUAGGG | | | AAGCCT |
| | | | | | | CACCUG | | | G |
| | | | | | | AAGAAA | | | |
| | | | | | | GCCUG | | | |
| RFX5_ | 129 | UAAUUU | 226 | ACACCC | 227 | UAAUUU | Chr1: | 228 | CTTGAC |
| Exon11_ | | CUACUC | | UUGUCA | | CUACUC | 151342748- | | ACCCTT |
| gRNA | | UUGUAG | | UGUGGU | | UUGUAG | 151342768 | | GTCATG |
| 9 | | AU | | CCU | | AUACAC | | | TGGTCC |
| | | | | | | CCUUGU | | | T |
| | | | | | | CAUGUG | | | |
| | | | | | | GUCCU | | | |
| RFX5_ | 129 | UAAUUU | 229 | GCUGGU | 230 | UAAUUU | Chr1: | 231 | TTTAGC |
| Exon11_ | | CUACUC | | GGAGCC | | CUACUC | 151342694- | | TGGTGG |
| gRNA | | UUGUAG | | UGCCCA | | UUGUAG | 151342714 | | AGCCTG |
| 10 | | AU | | CUG | | AUGCUG | | | CCCACT |
| | | | | | | GUGGAG | | | G |
| | | | | | | CCUGCC | | | |
| | | | | | | CACUG | | | |
| RFX5_ | 129 | UAAUUU | 232 | AGGUGC | 233 | UAAUUU | Chr1: | 234 | CTTCAG |
| Exon11_ | | CUACUC | | CCUGAA | | CUACUC | 151342946- | | GTGCCC |
| gRNA | | UUGUAG | | AGUGGC | | UUGUAG | 151342966 | | TGAAAG |
| 11 | | AU | | UAC | | AUAGGU | | | TGGCTA |
| | | | | | | GCCCUG | | | C |
| | | | | | | AAAGUG | | | |
| | | | | | | GCUAC | | | |
| RFX5_ | 235 | GGAAUU | 184 | AGGAUC | 236 | GGAAUU | Chr1: | 186 | TTTCAG |
| Exon9_ | | UCUACU | | CGCUCU | | UCUACU | 151343791- | | GATCCG |
| gRNA 2 | | CUUGUA | | GCCCAG | | CUUGUA | 151343811 | | CTCTGC |
| Short | | GAU | | UCA | | GAUAGG | | | CCAGTC |
| | | | | | | AUCCGC | | | A |
| | | | | | | UCUGCC | | | |
| | | | | | | CAGUCA | | | |
| RFX5_ | 237 | GUCAAA | 184 | AGGAUC | 238 | GUCAAA | Chr1: | 186 | TTTCAG |
| Exon9_ | | AGACCU | | CGCUCU | | AGACCU | 151343791- | | GATCCG |
| gRNA 2 | | UUGGAA | | GCCCAG | | UUGGAA | 151343811 | | CTCTGC |
| Long | | UUUCUA | | UCA | | UUUCUA | | | CCAGTC |
| | | CUCUUG | | | | CUCUUG | | | A |
| | | UAGAU | | | | UAGAUA | | | |
| | | | | | | GGAUCC | | | |
| | | | | | | GCUCUG | | | |
| | | | | | | CCCAGU | | | |
| | | | | | | CA | | | |
| RFX5_ | 129 | UAAUUU | 239 | AGGAUC | 240 | UAAUUU | Chr1: | 241 | TTTCAG |
| Exon9_ | | CUACUC | | CGCUCU | | CUACUC | 151343791- | | GATCCG |
| gRNA 2 | | UUGUAG | | GCCCAG | | UUGUAG | 151343810 | | CTCTGC |
| 20 bp | | AU | | UC | | AUAGGA | | | CCAGTC |
| | | | | | | UCCGCU | | | |
| | | | | | | CUGCCC | | | |
| | | | | | | AGUC | | | |
| RFX5 | 235 | GGAAUU | 239 | AGGAUC | 242 | GGAAUU | Chr1: | 241 | TTTCAG |
| Exon9_ | | UCUACU | | CGCUCU | | UCUACU | 151343791- | | GATCCG |
| gRNA 2 | | CUUGUA | | GCCCAG | | CUUGUA | 151343810 | | CTCTGC |
| Short | | GAU | | UC | | GAUAGG | | | CCAGTC |
| 20 bp | | | | | | AUCCGC | | | |
| | | | | | | UCUGCC | | | |
| | | | | | | CAGUC | | | |
| RFX5_ | 237 | GUCAAA | 239 | AGGAUC | 243 | GUCAAA | Chr1: | 241 | TTTCAG |
| Exon9_ | | AGACCU | | CGCUCU | | AGACCU | 151343791- | | GATCCG |
| gRNA 2 | | UUGGAA | | GCCCAG | | UUGGAA | 151343810 | | CTCTGC |
| Long | | UUUCUA | | UC | | UUUCUA | | | CCAGTC |
| 20 bp | | CUCUUG | | | | CUCUUG | | | |
| | | UAGAU | | | | UAGAUA | | | |
| | | | | | | GGAUCC | | | |
| | | | | | | GCUCUG | | | |
| | | | | | | CCCAGU | | | |
| | | | | | | C | | | |
| RFX5_ | 235 | GGAAUU | 193 | GAUGAC | 244 | GGAAUU | Chr1: | 195 | TTTAGA |
| Exon10_ | | UCUACU | | CGUUCC | | UCUACU | 151343409- | | TGACCG |
| gRNA 1 | | CUUGUA | | CGAGGU | | CUUGUA | 151343429 | | TTCCCG |
| Short | | GAU | | GCA | | GAUGAU | | | AGGTGC |
| | | | | | | GACCGU | | | A |
| | | | | | | UCCCGA | | | |
| | | | | | | GGUGCA | | | |
| RFX5_ | 237 | GUCAAA | 193 | GAUGAC | 245 | GUCAAA | Chr1: | 195 | TTTAGA |
| Exon10_ | | AGACCU | | CGUUCC | | AGACCU | 151343409- | | TGACCG |
| gRNA 1 | | UUGGAA | | CGAGGU | | UUGGAA | 151343429 | | TTCCCG |
| Long | | UUUCUA | | GCA | | UUUCUA | | | AGGTGC |
| | | CUCUUG | | | | CUCUUG | | | A |
| | | UAGAU | | | | UAGAUG | | | |
| | | | | | | AUGACC | | | |
| | | | | | | GUUCCC | | | |
| | | | | | | GAGGUG | | | |
| | | | | | | CA | | | |
| RFX5_ | 129 | UAAUUU | 246 | GAUGAC | 247 | UAAUUU | Chr1: | 248 | TTTAGA |
| Exon10_ | | CUACUC | | CGUUCC | | CUACUC | 151343409- | | TGACCG |
| gRNA 1 | | UUGUAG | | CGAGGU | | UUGUAG | 151343428 | | TTCCCG |
| 20 bp | | AU | | GC | | AUGAUG | | | AGGTGC |
| | | | | | | ACCGUU | | | |
| | | | | | | CCCGAG | | | |
| | | | | | | GUGC | | | |
| RFX5_ | 235 | GGAAUU | 246 | GAUGAC | 249 | GGAAUU | Chr1: | 248 | TTTAGA |
| Exon10_ | | UCUACU | | CGUUCC | | UCUACU | 151343409- | | TGACCG |
| gRNA 1 | | CUUGUA | | CGAGGU | | CUUGUA | 151343428 | | TTCCCG |
| Short | | GAU | | GC | | GAUGAU | | | AGGTGC |
| 20 bp | | | | | | GACCGU | | | |
| | | | | | | UCCCGA | | | |
| | | | | | | GGUGC | | | |
| RFX5_ | 237 | GUCAAA | 246 | GAUGAC | 250 | GUCAAA | Chr1: | 248 | TTTAGA |
| Exon10_ | | AGACCU | | CGUUCC | | AGACCU | 151343409- | | TGACCG |
| gRNA 1 | | UUGGAA | | CGAGGU | | UUGGAA | 151343428 | | TTCCCG |
| Long | | UUUCUA | | GC | | UUUCUA | | | AGGTGC |
| 20 bp | | CUCUUG | | | | CUCUUG | | | |
| | | UAGAU | | | | UAGAUG | | | |
| | | | | | | AUGACC | | | |
| | | | | | | GUUCCC | | | |
| | | | | | | GAGGUG | | | |
| | | | | | | C | | | |

TABLE 13
RFX5 >5% Editing Efficiency.

| | | | | | SEQ | | | | |
|---|---|---|---|---|---|---|---|---|---|
| | | | | | ID NO: | | | | |
| | SEQ | | SEQ | | for | | | SEQ ID | |
| | ID NO: | | ID NO: | | Repeat + | Repeat + | | NO: | |
| | for | | for | | Spacer | Spacer | | for | |
| | Repeat | Repeat | Spacer | Spacer | Complete | Complete | Chromosome | Genomic | Genomic |
| | Sequence | Sequence | Sequence | Sequence | Sequence | Sequence | location | Target | Target |
| ID | (5′-3′) | (5′-3′) | (5′-3′) | (5′-3′) | (5′-3′) | (5′-3′) | (hg38) | Region | Region |
| RFX5_ | 129 | UAAUU | 139 | GGGCUC | 140 | UAAUUUCU | Chr1: | 141 | CTTGG |
| Exon3_ | | UCUACU | | UUAGCA | | ACUCUUGU | 151346289- | | GGCTC |
| gRNA 4 | | CUUGUA | | UCAGGC | | AGAUGGGC | 151346309 | | TTAGC |
| | | GAU | | UCA | | UCUUAGCA | | | ATCA |
| | | | | | | UCAGGCUC | | | GGCTC |
| | | | | | | A | | | A |
| RFX5_ | 129 | UAAUU | 184 | AGGAUC | 185 | UAAUUUCU | Chr1: | 186 | TTTCA |
| Exon9_ | | UCUACU | | CGCUCU | | ACUCUUGU | 151343791- | | GGAT |
| gRNA 2 | | CUUGUA | | GCCCAG | | AGAUAGGA | 151343811 | | CCGCT |
| | | GAU | | UCA | | UCCGCUCU | | | CTGCC |
| | | | | | | GCCCAGUC | | | CAGTC |
| | | | | | | A | | | A |
| RFX5_ | 129 | UAAUU | 193 | GAUGAC | 194 | UAAUUUCU | Chr1: | 195 | TTTAG |
| Exon10_ | | UCUACU | | CGUUCC | | ACUCUUGU | 151343409- | | ATGA |
| gRNA | | CUUGUA | | CGAGGU | | AGAUGAUG | 151343429 | | CCGTT |
| 1 | | GAU | | GCA | | ACCGUUCC | | | CCCG |
| | | | | | | CGAGGUGC | | | AGGT |
| | | | | | | A | | | GCA |
| RFX5_ | 129 | UAAUU | 202 | GAGAAC | 203 | UAAUUUCU | Chr1: | 204 | TTTAG |
| Exon10_ | | UCUACU | | CCAGAG | | ACUCUUGU | 151343369- | | AGAA |
| gRNA | | CUUGUA | | GGUGG | | AGAUGAGA | 151343389 | | CCCA |
| 4 | | GAU | | AGCC | | ACCCAGAG | | | GAGG |
| | | | | | | GGUGGAGC | | | GTGG |
| | | | | | | C | | | AGCC |
| RFX5_ | 129 | UAAUU | 205 | GUACCU | 206 | UAAUUUCU | Chr1: | 207 | TTTGG |
| Exon10_ | | UCUACU | | CUGCAG | | ACUCUUGU | 151343434- | | TACCT |
| gRNA | | CUUGUA | | AAGAG | | AGAUGUAC | 151343454 | | CTGCA |
| 5 | | GAU | | GACG | | CUCUGCAG | | | GAAG |
| | | | | | | AAGAGGAC | | | AGGA |
| | | | | | | G | | | CG |
| RFX5_ | 129 | UAAUU | 223 | AGGGCA | 224 | UAAUUUCU | Chr1: | 225 | TTTCA |
| Exon11_ | | UCUACU | | CCUGAA | | ACUCUUGU | 151342958- | | GGGC |
| gRNA | | CUUGUA | | GAAAGC | | AGAUAGGG | 151342978 | | ACCTG |
| 8 | | GAU | | CUG | | CACCUGAA | | | AAGA |
| | | | | | | GAAAGCCU | | | AAGC |
| | | | | | | G | | | CTG |
| RFX5_ | 235 | GGAAU | 184 | AGGAUC | 236 | GGAAUUUC | Chr1: | 186 | TTTCA |
| Exon9_ | | UUCUAC | | CGCUCU | | UACUCUUG | 151343791- | | GGAT |
| gRNA 2 | | UCUUGU | | GCCCAG | | UAGAUAGG | 151343811 | | CCGCT |
| Short | | AGAU | | UCA | | AUCCGCUC | | | CTGCC |
| | | | | | | UGCCCAGU | | | CAGTC |
| | | | | | | CA | | | A |
| RFX5_ | 237 | GUCAAA | 184 | AGGAUC | 238 | GUCAAAAG | Chr1: | 186 | TTTCA |
| Exon9_ | | AGACCU | | CGCUCU | | ACCUUUGG | 151343791- | | GGAT |
| gRNA 2 | | UUGGA | | GCCCAG | | AAUUUCUA | 151343811 | | CCGCT |
| Long | | AUUUCU | | UCA | | CUCUUGUA | | | CTGCC |
| | | ACUCUU | | | | GAUAGGAU | | | CAGTC |
| | | GUAGA | | | | CCGCUCUG | | | A |
| | | U | | | | CCCAGUCA | | | |
| RFX5_ | 129 | UAAUU | 239 | AGGAUC | 240 | UAAUUUCU | Chr1: | 241 | TTTCA |
| Exon9_ | | UCUACU | | CGCUCU | | ACUCUUGU | 151343791- | | GGAT |
| gRNA 2 | | CUUGUA | | GCCCAG | | AGAUAGGA | 151343810 | | CCGCT |
| 20 bp | | GAU | | UC | | UCCGCUCU | | | CTGCC |
| | | | | | | GCCCAGUC | | | CAGTC |
| RFX5_ | 235 | GGAAU | 239 | AGGAUC | 242 | GGAAUUUC | Chr1: | 241 | TTTCA |
| Exon9_ | | UUCUAC | | CGCUCU | | UACUCUUG | 151343791- | | GGAT |
| gRNA 2 | | UCUUGU | | GCCCAG | | UAGAUAGG | 151343810 | | CCGCT |
| Short | | AGAU | | UC | | AUCCGCUC | | | CTGCC |
| 20 bp | | | | | | UGCCCAGU | | | CAGTC |
| | | | | | | C | | | |
| RFX5_ | 237 | GUCAAA | 239 | AGGAUC | 243 | GUCAAAAG | Chr1: | 241 | TTTCA |
| Exon9_ | | AGACCU | | CGCUCU | | ACCUUUGG | 151343791- | | GGAT |
| gRNA 2 | | UUGGA | | GCCCAG | | AAUUUCUA | 151343810 | | CCGCT |
| Long | | AUUUCU | | UC | | CUCUUGUA | | | CTGCC |
| 20 bp | | ACUCUU | | | | GAUAGGAU | | | CAGTC |
| | | GUAGA | | | | CCGCUCUG | | | |
| | | U | | | | CCCAGUC | | | |
| RFX5_ | 235 | GGAAU | 193 | GAUGAC | 244 | GGAAUUUC | Chr1: | 195 | TTTAG |
| Exon10_ | | UUCUAC | | CGUUCC | | UACUCUUG | 151343409- | | ATGA |
| gRNA 1 | | UCUUGU | | CGAGGU | | UAGAUGAU | 151343429 | | CCGTT |
| Short | | AGAU | | GCA | | GACCGUUC | | | CCCG |
| | | | | | | CCGAGGUG | | | AGGT |
| | | | | | | CA | | | GCA |
| RFX5_ | 237 | GUCAAA | 193 | GAUGAC | 245 | GUCAAAAG | Chr1: | 195 | TTTAG |
| Exon10_ | | AGACCU | | CGUUCC | | ACCUUUGG | 151343409- | | ATGA |
| gRNA 1 | | UUGGA | | CGAGGU | | AAUUUCUA | 151343429 | | CCGTT |
| Long | | AUUUCU | | GCA | | CUCUUGUA | | | CCCG |
| | | ACUCUU | | | | GAUGAUGA | | | AGGT |
| | | GUAGA | | | | CCGUUCCC | | | GCA |
| | | U | | | | GAGGUGCA | | | |
| RFX5_ | 129 | UAAUU | 246 | GAUGAC | 247 | UAAUUUCU | Chr1: | 248 | TTTAG |
| Exon10_ | | UCUACU | | CGUUCC | | ACUCUUGU | 151343409- | | ATGA |
| gRNA 1 | | CUUGUA | | CGAGGU | | AGAUGAUG | 151343428 | | CCGTT |
| 20 bp | | GAU | | GC | | ACCGUUCC | | | CCCG |
| | | | | | | CGAGGUGC | | | AGGT |
| | | | | | | | | | GC |
| RFX5_ | 235 | GGAAU | 246 | GAUGAC | 249 | GGAAUUUC | Chr1: | 248 | TTTAG |
| Exon10_ | | UUCUAC | | CGUUCC | | UACUCUUG | 151343409- | | ATGA |
| gRNA 1 | | UCUUGU | | CGAGGU | | UAGAUGAU | 151343428 | | CCGTT |
| Short | | AGAU | | GC | | GACCGUUC | | | CCCG |
| 20 bp | | | | | | CCGAGGUG | | | AGGT |
| | | | | | | C | | | GC |
| RFX5_ | 237 | GUCAAA | 246 | GAUGAC | 250 | GUCAAAAG | Chr1: | 248 | TTTAG |
| Exon10_ | | AGACCU | | CGUUCC | | ACCUUUGG | 151343409- | | ATGA |
| gRNA 1 | | UUGGA | | CGAGGU | | AAUUUCUA | 151343428 | | CCGTT |
| Long | | AUUUCU | | GC | | CUCUUGUA | | | CCCG |
| 20 bp | | ACUCUU | | | | GAUGAUGA | | | AGGT |
| | | GUAGA | | | | CCGUUCCC | | | GC |
| | | U | | | | GAGGUGC | | | |

### 9.5 Example 5: Exemplary gRNA Structure Engineering for Knocking Out CD58

Preparation of RNP complexes. Alt-R crRNAs were synthesized (IDT) and dissolved in nuclease free duplex buffer (IDT #11-05-01-12) at 200 μM. RNP complexes were prepared fresh on the day of nucleofection by mixing a ratio of 63 pmol (1 μl of 10 μg/μl) WT MAD7 (Aldevron) with 200 pmol (1 μl of 200 μM) crRNA and 100 μg of poly-L-glutamic acid sodium salt (PGA, MW15-50 kD, Sigma p4761). The RNP complex was incubated at room temperature for 30 minutes. P3 Buffer (Lonza #V4SP-3096) with 3 μM electroporation enhancer (IDT #1076301) was added for a total volume of 10 μL.

Generation of knockout human iPSCs. Human iPSCs were pretreated with 10 μM Y-27632 ROCK inhibitor (STEMCELL Technologies #72302) in Stemfit Basic 04 Complete Type Medium (Ajinomoto Basic04CT). The iPSCs were collected (0.5e6 cells per reaction) and resuspended in 10 μL of P3 buffer with 3 μM electroporation enhancer. The cells were combined with 10 μL of relevant RNP complex and nucleofected in 96 well cuvettes (Lonza #V4SP-3096) using the CA-137 program on the Lonza 4D system. The iPSCs were then transferred to one well of 24 well plate coated with 0.5 μg/cm² of iMatrix-511 (Takara #T304) containing Stemfit Basic 04 Complete Type Medium (Ajinomoto Basic04CT) with 10 μM Y-27632 ROCK inhibitor. The iPSCs were collected 48 hours post electroporation, the DNA was extracted, and the region around the gRNA target-site was amplified and Sanger sequenced. Editing efficiency was measured by the ICE tool (Synthego) to analyze Sanger sequencing results (GENEWIZ).

CD58 knockouts were generated using gRNAs and WT MAD7. FIG. 27 shows a CD58 gRNA tiling screen in iPSCs (Table 14). The editing efficiency of each gRNA tested to knockout the CD58 gene is shown. Some gRNAs tested showed zero or minimal editing. Several gRNAs had moderate editing and the top gRNA had high editing that can be used to efficiently knockout CD58 with MAD7 in iPSCs (Table 15).

TABLE 14
Exemplary CD58 gRNAs.

| | | | | | SEQ | | | | |
|---|---|---|---|---|---|---|---|---|---|
| | | | | | ID NO: | | | | |
| | SEQ | | SEQ | | for | | | SEQ ID | |
| | ID NO: | | ID NO: | | Repeat + | Repeat + | | NO: | |
| | for | | for | | Spacer | Spacer | | for | |
| | Repeat | Repeat | Spacer | Spacer | Complete | Complete | Chromosome | Genomic | Genomic |
| | Sequence | Sequence | Sequence | Sequence | Sequence | Sequence | location | Target | Target |
| ID | (5′-3′) | (5′-3′) | (5′-3′) | (5′-3′) | (5′-3′) | (5′-3′) | (hg38) | Region | Region |
| CD58_ | 129 | UAAUUU | 254 | ACUC | 255 | UAAUU | Chr1: | 256 | CTTCAC |
| Exon | | CUACUC | | ACCA | | UCUACU | 116570897- | | TCACCA |
| 1_gRNA | | UUGUAG | | AAGC | | CUUGUA | 116570917 | | AAGCAG |
| 1 | | AU | | AGUG | | GAUACU | | | TGCAGC |
| | | | | CAGC | | CACCAA | | | A |
| | | | | A | | AGCAGU | | | |
| | | | | | | GCAGCA | | | |
| CD58_ | 129 | UAAUUU | 257 | AUGG | 258 | UAAUU | Chr1: | 259 | CTTCAT |
| Exon | | CUACUC | | UAUC | | UCUACU | 611544334- | | GGTATC |
| 2_gRNA | | UUGUAG | | AGUA | | CUUGUA | 116544354 | | AGTAAT |
| 1 | | AU | | AUAU | | GAUAU | | | ATTTGG |
| | | | | UUGG | | GGUAUC | | | C |
| | | | | C | | AGUAA | | | |
| | | | | | | UAUUU | | | |
| | | | | | | GGC | | | |
| CD58_ | 129 | UAAUUU | 260 | GCGA | 261 | UAAUU | Chr1: | 262 | TTTGGC |
| Exon | | CUACUC | | UUCC | | UCUACU | 116544353- | | GATTCC |
| 2_gRNA | | UUGUAG | | AUUU | | CUUGUA | 116544373 | | ATTTCA |
| 2 | | AU | | CAUA | | GAUGCG | | | TACTCA |
| | | | | CUCA | | AUUCCA | | | T |
| | | | | U | | UUUCAU | | | |
| | | | | | | ACUCAU | | | |
| CD58_ | 129 | UAAUUU | 263 | UAAA | 264 | UAAUU | Chr1: | 265 | CTTTTA |
| Exon | | CUACUC | | GGCA | | UCUACU | 116544522- | | AAGGCA |
| 2_gRNA | | UUGUAG | | CAUU | | CUUGUA | 116544542 | | CATTGC |
| 3 | | AU | | GCUU | | GAUUA | | | TTGGTA |
| | | | | GGUA | | AAGGCA | | | C |
| | | | | C | | CAUUGC | | | |
| | | | | | | UUGGU | | | |
| | | | | | | AC | | | |
| CD58_ | 129 | UAAUUU | 266 | CAUA | 267 | UAAUU | Chr1: | 268 | TTTCCA |
| Exon | | CUACUC | | GGAC | | UCUACU | 116544508- | | TAGGAC |
| 2_gRNA | | UUGUAG | | CUCU | | CUUGUA | 116544528 | | CTCTTT |
| 4 | | AU | | UUUA | | GAUCAU | | | TAAAGG |
| | | | | AAGG | | AGGACC | | | C |
| | | | | C | | UCUUUU | | | |
| | | | | | | AAAGGC | | | |
| CD58_ | 129 | UAAUUU | 269 | UUUA | 270 | UAAUU | Chr1: | 271 | TTTATT |
| Exon_ | | CUACUC | | GACA | | UCUACU | 116544409- | | TAGACA |
| 2_gRNA | | UUGUAG | | CUGU | | CUUGUA | 116544429 | | CTGTGT |
| 5 | | AU | | GUCA | | GAUUU | | | CAGGTA |
| | | | | GGUA | | UAGACA | | | G |
| | | | | G | | CUGUGU | | | |
| | | | | | | CAGGUA | | | |
| | | | | | | G | | | |
| CD58_ | 129 | UAAUUU | 272 | GACA | 273 | UAAUU | Chr1: | 274 | TTTAGA |
| Exon | | CUACUC | | CUGU | | UCUACU | 116544405- | | CACTGT |
| 2_gRNA | | UUGUAG | | GUCA | | CUUGUA | 116544425 | | GTCAGG |
| 6 | | AU | | GGUA | | GAUGAC | | | TAGCCT |
| | | | | GCCU | | ACUGUG | | | C |
| | | | | C | | UCAGGU | | | |
| | | | | | | AGCCUC | | | |
| CD58_ | 129 | UAAUUU | 275 | CAUG | 276 | UAAUU | Chr1: | 277 | TTTCCA |
| Exon | | CUACUC | | UACC | | UCUACU | 116544525- | | TGTACC |
| 2_gRNA | | UUGUAG | | AAGC | | CUUGUA | 116544545 | | AAGCAA |
| 8 | | AU | | AAUG | | GAUCAU | | | TGTGCC |
| | | | | UGCC | | GUACCA | | | T |
| | | | | U | | AGCAAU | | | |
| | | | | | | GUGCCU | | | |
| CD58_ | 129 | UAAUUU | 278 | AAGG | 279 | UAAUU | Chr1: | 280 | TTTAAA |
| Exon | | CUACUC | | CACA | | UCUACU | 116544524- | | GGCACA |
| 2_gRNA | | UUGUAG | | UUGC | | CUUGUA | 116544544 | | TTGCTT |
| 9 | | AU | | UUGG | | GAUAA | | | GGTACA |
| | | | | UACA | | GGCACA | | | T |
| | | | | U | | UUGCUU | | | |
| | | | | | | GGUACA | | | |
| | | | | | | U | | | |
| CD58_ | 129 | UAAUUU | 281 | CCCA | 282 | UAAUU | Chr1: | 283 | TTTTCC |
| Exon | | CUACUC | | ACAA | | UCUACU | 116544566- | | CAACAA |
| 2_gRNA | | UUGUAG | | AUAU | | CUUGUA | 116544586 | | ATATAT |
| 10 | | AU | | AUGG | | GAUCCC | | | GGTGTT |
| | | | | UGUU | | AACAAA | | | G |
| | | | | G | | UAUAU | | | |
| | | | | | | GGUGU | | | |
| | | | | | | UG | | | |
| CD58_ | 129 | UAAUUU | 284 | CUCU | 285 | UAAUU | Chr1: | 286 | TTTTCT |
| Exon | | CUACUC | | CCUA | | UCUACU | 116544593- | | CTCCTA |
| 2_gRNA | | UUGUAG | | GGUU | | CUUGUA | 116544613 | | GGTTTC |
| 11 | | AU | | UCAU | | GAUCUC | | | ATCAGC |
| | | | | CAGC | | UCCUAG | | | T |
| | | | | U | | GUUUCA | | | |
| | | | | | | UCAGCU | | | |
| CD58_ | 129 | UAAUUU | 287 | CCAU | 288 | UAAUU | Chr1: | 289 | TTTTCC |
| Exon | | CUACUC | | AGGA | | UCUACU | 116544507- | | ATAGGA |
| 2_gRNA | | UUGUAG | | CCUC | | CUUGUA | 116544527 | | CCTCTT |
| 12 | | AU | | UUUU | | GAUCCA | | | TTAAAG |
| | | | | AAAG | | UAGGAC | | | G |
| | | | | G | | CUCUUU | | | |
| | | | | | | UAAAG | | | |
| | | | | | | G | | | |
| CD58_ | 129 | UAAUUU | 290 | UGGA | 291 | UAAUU | Chr1: | 292 | CTTATG |
| Exon | | CUACUC | | AUAC | | UCUACU | 116544299- | | GAATAC |
| 2_gRNA | | UUGUAG | | UCAC | | CUUGUA | 116544319 | | TCACCA |
| 13 | | AU | | CAAG | | GAUUG | | | AGCACA |
| | | | | CACA | | GAAUAC | | | T |
| | | | | U | | UCACCA | | | |
| | | | | | | AGCACA | | | |
| | | | | | | U | | | |
| CD58_ | 129 | UAAUUU | 293 | GUAC | 294 | UAAUU | Chr1: | 295 | CTTGGT |
| Exon | | CUACUC | | AUGG | | UCUACU | 116544539- | | ACATGG |
| 2_gRNA | | UUGUAG | | AAAG | | CUUGUA | 116544559 | | AAAGTT |
| 14 | | AU | | UUAC | | GAUGU | | | ACATTC |
| | | | | AUUC | | ACAUGG | | | C |
| | | | | C | | AAAGU | | | |
| | | | | | | UACAUU | | | |
| | | | | | | CC | | | |
| CD58_ | 129 | UAAUUU | 296 | UCCC | 297 | UAAUU | Chr1: | 298 | TTTTTC |
| Exon | | CUACUC | | AACA | | UCUACU | 116544567- | | CCAACA |
| 2_gRNA | | UUGUAG | | AAUA | | CUUGUA | 116544587 | | AATATA |
| 16 | | AU | | UAUG | | GAUUCC | | | TGGTGT |
| | | | | GUGU | | CAACAA | | | T |
| | | | | U | | AUAUA | | | |
| | | | | | | UGGUG | | | |
| | | | | | | UU | | | |
| CD58_ | 129 | UAAUUU | 299 | ACAU | 300 | UAAUU | Chr1: | 301 | CTTAAC |
| Exon | | CUACUC | | CAUC | | UCUACU | 116544369- | | ATCATC |
| 2_gRNA | | UUGUAG | | AGAU | | CUUGUA | 116544389 | | AGATGA |
| 17 | | AU | | GAAG | | GAUACA | | | AGATGA |
| | | | | AUGA | | UCAUCA | | | G |
| | | | | G | | GAUGA | | | |
| | | | | | | AGAUG | | | |
| | | | | | | AG | | | |
| CD58_ | 129 | UAAUUU | 302 | UUGG | 303 | UAAUU | Chr1: | 304 | TTTGTT |
| Exon | | CUACUC | | GAAA | | UCUACU | 116544582- | | GGGAA |
| 2_gRNA | | UUGUAG | | AACA | | CUUGUA | 116544602 | | AAACAG |
| 18 | | AU | | GCUG | | GAUUU | | | CTGATG |
| | | | | AUGA | | GGGAA | | | AA |
| | | | | A | | AAACAG | | | |
| | | | | | | CUGAUG | | | |
| | | | | | | AA | | | |
| CD58_ | 129 | UAAUUU | 305 | UUCU | 306 | UAAUU | Chr1: | 307 | TTTTTT |
| Exon | | CUACUC | | CUCC | | UCUACU | 116544595- | | CTCTCC |
| 2_gRNA | | UUGUAG | | UAGG | | CUUGUA | 116544615 | | TAGGTT |
| 19 | | AU | | UUUC | | GAUUUC | | | TCATCA |
| | | | | AUCA | | UCUCCU | | | G |
| | | | | G | | AGGUU | | | |
| | | | | | | UCAUCA | | | |
| | | | | | | G | | | |
| CD58_ | 129 | UAAUUU | 308 | UCUC | 309 | UAAUU | Chr1: | 310 | TTTTTC |
| Exon | | CUACUC | | UCCU | | UCUACU | 116544594- | | TCTCCT |
| 2_gRNA | | UUGUAG | | AGGU | | CUUGUA | 116544614 | | AGGTTT |
| 20 | | AU | | UUCA | | GAUUCU | | | CATCAG |
| | | | | UCAG | | CUCCUA | | | C |
| | | | | C | | GGUUUC | | | |
| | | | | | | AUCAGC | | | |
| CD58_ | 129 | UAAUUU | 311 | AUCA | 312 | UAAUU | Chr1: | 313 | TTTCAT |
| Exon | | CUACUC | | GCUG | | UCUACU | 116544579- | | CAGCTG |
| 2_gRNA | | UUGUAG | | UUUU | | CUUGUA | 116544599 | | TTTTTC |
| 21 | | AU | | UCCC | | GAUAUC | | | CCAACA |
| | | | | AACA | | AGCUGU | | | A |
| | | | | A | | UUUUCC | | | |
| | | | | | | CAACAA | | | |
| CD58_ | 129 | UAAUUU | 314 | AUAC | 315 | UAAUU | Chr1: | 316 | TTTCAT |
| Exon | | CUACUC | | UCAU | | UCUACU | 116544366- | | ACTCAT |
| 2_gRNA | | UUGUAG | | CUUC | | CUUGUA | 116544386 | | CTTCAT |
| 22 | | AU | | AUCU | | GAUAU | | | CTGATG |
| | | | | GAUG | | ACUCAU | | | A |
| | | | | A | | CUUCAU | | | |
| | | | | | | CUGAUG | | | |
| | | | | | | A | | | |
| CD58_ | 129 | UAAUUU | 317 | UCUC | 318 | UAAUU | Chr1: | 319 | TTTCTC |
| Exon | | CUACUC | | CUAG | | UCUACU | 116544592- | | TCCTAG |
| 2_gRNA | | UUGUAG | | GUUU | | CUUGUA | 116544612 | | GTTTCA |
| 23 | | AU | | CAUC | | GAUUCU | | | TCAGCT |
| | | | | AGCU | | CCUAGG | | | G |
| | | | | G | | UUUCAU | | | |
| | | | | | | CAGCUG | | | |
| CD58_ | 129 | UAAUUU | 320 | AAAA | 321 | UAAUU | Chr1: | 322 | CTTTAA |
| Exon | | CUACUC | | GAGG | | UCUACU | 116544502- | | AAGAG |
| 2_gRNA | | UUGUAG | | UCCU | | CUUGUA | 116544522 | | GTCCTA |
| 24 | | AU | | AUGG | | GAUAA | | | TGGAAA |
| | | | | AAAA | | AAGAG | | | AA |
| | | | | A | | GUCCUA | | | |
| | | | | | | UGGAA | | | |
| | | | | | | AAA | | | |
| CD58_ | 129 | UAAUUU | 323 | CAUU | 324 | UAAUU | Chr1: | 325 | TTTACA |
| Exon | | CUACUC | | GCUC | | UCUACU | 116536096- | | TTGCTC |
| 3_gRNA | | UUGUAG | | CAUA | | CUUGUA | 116536116 | | CATAGG |
| 1 | | AU | | GGAC | | GAUCAU | | | ACAATC |
| | | | | AAUC | | UGCUCC | | | C |
| | | | | C | | AUAGG | | | |
| | | | | | | ACAAUC | | | |
| | | | | | | C | | | |
| CD58_ | 129 | UAAUUU | 326 | CUCA | 327 | UAAUU | Chr1: | 328 | TTTACT |
| Exon | | CUACUC | | CCGC | | UCUACU | 116535960- | | CACCGC |
| 3_gRNA | | UUGUAG | | UGCU | | CUUGUA | 116535980 | | TGCTTG |
| 2 | | AU | | UGGG | | GAUCUC | | | GGATAC |
| | | | | AUAC | | ACCGCU | | | A |
| | | | | A | | GCUUGG | | | |
| | | | | | | GAUACA | | | |
| CD58_ | 129 | UAAUUU | 329 | CAUC | 330 | UAAUU | Chr1: | 331 | CTTCCA |
| Exon | | CUACUC | | UCCC | | UCUACU | 116536199- | | TCTCCC |
| 3_gRNA | | UUGUAG | | ACAC | | CUUGUA | 116536219 | | ACACTA |
| 3 | | AU | | UAAC | | GAUCAU | | | ACTTGT |
| | | | | UUGU | | CUCCCA | | | G |
| | | | | G | | CACUAA | | | |
| | | | | | | CUUGUG | | | |
| CD58_ | 129 | UAAUUU | 332 | GACA | 333 | UAAUU | Chr1: | 334 | TTTTGA |
| Exon | | CUACUC | | ACCU | | UCUACU | 116535967- | | CAACCT |
| 3_gRNA | | UUGUAG | | GUAU | | CUUGUA | 116535987 | | GTATCC |
| 4 | | AU | | CCCA | | GAUGAC | | | CAAGCA |
| | | | | AGCA | | AACCUG | | | G |
| | | | | G | | UAUCCC | | | |
| | | | | | | AAGCAG | | | |
| CD58_ | 129 | UAAUUU | 335 | ACAA | 336 | UAAUU | Chr1: | 337 | TTTGAC |
| Exon | | CUACUC | | CCUG | | UCUACU | 116535966- | | AACCTG |
| 3_gRNA | | UUGUAG | | UAUC | | CUUGUA | 116535986 | | TATCCC |
| 5 | | AU | | CCAA | | GAUACA | | | AAGCAG |
| | | | | GCAG | | ACCUGU | | | C |
| | | | | C | | AUCCCA | | | |
| | | | | | | AGCAGC | | | |
| CD58_ | 129 | UAAUUU | 338 | GUUA | 339 | UAAUU | Chr1: | 340 | TTTAGT |
| Exon | | CUACUC | | UUUA | | UCUACU | 116535952- | | TATTTA |
| 3_gRNA | | UUGUAG | | CUCA | | CUUGUA | 116535972 | | CTCACC |
| 6 | | AU | | CCGC | | GAUGU | | | GCTGCT |
| | | | | UGCU | | UAUUU | | | T |
| | | | | U | | ACUCAC | | | |
| | | | | | | CGCUGC | | | |
| | | | | | | UU | | | |
| CD58_ | 129 | UAAUUU | 341 | CAUU | 342 | UAAUU | Chr1: | 343 | CTTCCA |
| Exon | | CUACUC | | AGUC | | UCUACU | 116536187- | | TTAGTC |
| 3_gRNA | | UUGUAG | | AAUG | | CUUGUA | 116536207 | | AATGCA |
| 7 | | AU | | CACA | | GAUCAU | | | CAAGTT |
| | | | | AGUU | | UAGUCA | | | A |
| | | | | A | | AUGCAC | | | |
| | | | | | | AAGUU | | | |
| | | | | | | A | | | |
| CD58_ | 129 | UAAUUU | 344 | UAAU | 345 | UAAUU | Chr1: | 346 | CTTATA |
| Exon | | CUACUC | | GUAC | | UCUACU | 116536109- | | ATGTAC |
| 3_gRNA | | UUGUAG | | UCAU | | CUUGUA | 116536129 | | TCATGG |
| 8 | | AU | | GGGA | | GAUUA | | | GATTGT |
| | | | | UUGU | | AUGUAC | | | C |
| | | | | C | | UCAUGG | | | |
| | | | | | | GAUUG | | | |
| | | | | | | UC | | | |
| CD58_ | 129 | UAAUUU | 347 | UGCA | 348 | UAAUU | Chr1: | 349 | CTTGTG |
| Exon | | CUACUC | | UUGA | | UCUACU | 116536180- | | CATTGA |
| 3_gRN | | UUGUAG | | CUAA | | CUUGUA | 116536200 | | CTAATG |
| A 9 | | AU | | UGGA | | GAUUGC | | | GAAGCA |
| | | | | AGCA | | AUUGAC | | | T |
| | | | | U | | UAAUG | | | |
| | | | | | | GAAGCA | | | |
| | | | | | | U | | | |
| CD58_ | 129 | UAAUUU | 350 | AAUG | 351 | UAAUU | Chr1: | 352 | CTTCAA |
| Exon | | CUACUC | | CUUC | | UCUACU | 116536179- | | TGCTTC |
| 3_gRNA | | UUGUAG | | CAUU | | CUUGUA | 116536199 | | CATTAG |
| 10 | | AU | | AGUC | | GAUAA | | | TCAATG |
| | | | | AAUG | | UGCUUC | | | C |
| | | | | C | | CAUUAG | | | |
| | | | | | | UCAAUG | | | |
| | | | | | | C | | | |
| CD58_ | 129 | UAAUUU | 353 | AGAU | 354 | UAAUU | Chr1: | 355 | TTTAAG |
| Exon | | CUACUC | | GGAA | | UCUACU | 116536046- | | ATGGAA |
| 3_gRNA | | UUGUAG | | AAUG | | CUUGUA | 116536066 | | AATGAT |
| 11 | | AU | | AUCU | | GAUAG | | | CTTCCA |
| | | | | UCCA | | AUGGA | | | C |
| | | | | C | | AAAUG | | | |
| | | | | | | AUCUUC | | | |
| | | | | | | CAC | | | |
| CD58_ | 129 | UAAUUU | 356 | GCAA | 357 | UAAUU | Chr1: | 358 | CTTAGC |
| Exon | | CUACUC | | UCCA | | UCUACU | 116536004- | | AATCCA |
| 3_gRNA | | UUGUAG | | UUAU | | CUUGUA | 116536024 | | TTATTT |
| 12 | | AU | | UUAA | | GAUGCA | | | AATACA |
| | | | | UACA | | AUCCAU | | | A |
| | | | | A | | UAUUU | | | |
| | | | | | | AAUACA | | | |
| | | | | | | A | | | |
| CD58_ | 129 | UAAUUU | 359 | UUCC | 360 | UAAUU | Chr1: | 361 | TTTTTT |
| Exon | | CUACUC | | AGAG | | UCUACU | 116536214- | | CCAGAG |
| 3_gRNA | | UUGUAG | | UCUC | | CUUGUA | 116536234 | | TCTCTT |
| 13 | | AU | | UUCC | | GAUUUC | | | CCATCT |
| | | | | AUCU | | CAGAGU | | | C |
| | | | | C | | CUCUUC | | | |
| | | | | | | CAUCUC | | | |
| CD58_ | 129 | UAAUUU | 362 | AAAU | 363 | UAAUU | Chr1: | 364 | CTTAAA |
| Exon | | CUACUC | | AUAU | | UCUACU | 116536069- | | ATATAT |
| 3_gRNA | | UUGUAG | | ACUG | | CUUGUA | 116536089 | | ACTGGT |
| 15 | | AU | | GUUG | | GAUAA | | | TGAGTT |
| | | | | AGUU | | AUAUA | | | A |
| | | | | A | | UACUGG | | | |
| | | | | | | UUGAG | | | |
| | | | | | | UUA | | | |
| CD58_ | 129 | UAAUUU | 365 | CCAU | 366 | UAAUU | Chr1: | 367 | TTTTCC |
| Exon | | CUACUC | | CUUA | | UCUACU | 116536061- | | ATCTTA |
| 3_gRNA | | UUGUAG | | AAAU | | CUUGUA | 116536081 | | AAATAT |
| 16 | | AU | | AUAU | | GAUCCA | | | ATACTG |
| | | | | ACUG | | UCUUAA | | | G |
| | | | | G | | AAUAU | | | |
| | | | | | | AUACUG | | | |
| | | | | | | G | | | |
| CD58_ | 129 | UAAUUU | 368 | GGAU | 369 | UAAUU | Chr1: | 370 | CTTGGG |
| Exon | | CUACUC | | ACAG | | UCUACU | 116535974- | | ATACAG |
| 3_gRNA | | UUGUAG | | GUUG | | CUUGUA | 116535994 | | GTTGTC |
| 17 | | AU | | UCAA | | GAUGG | | | AAAATG |
| | | | | AAUG | | AUACAG | | | A |
| | | | | A | | GUUGUC | | | |
| | | | | | | AAAAU | | | |
| | | | | | | GA | | | |
| CD58_ | 129 | UAAUUU | 371 | UCCA | 372 | UAAUU | Chr1: | 373 | TTTTTC |
| Exon | | CUACUC | | GAGU | | UCUACU | 116536213- | | CAGAGT |
| 3_gRNA | | UUGUAG | | CUCU | | CUUGUA | 116536233 | | CTCTTC |
| 18 | | AU | | UCCA | | GAUUCC | | | CATCTC |
| | | | | UCUC | | AGAGUC | | | C |
| | | | | C | | UCUUCC | | | |
| | | | | | | AUCUCC | | | |
| CD58_ | 129 | UAAUUU | 374 | UUUU | 375 | UAAUU | Chr1: | 376 | TTTTTTT |
| Exon | | CUACUC | | CCAG | | UCUACU | 116536216- | | TCCAGA |
| 3_gRNA | | UUGUAG | | AGUC | | CUUGUA | 116536236 | | GTCTCT |
| 19 | | AU | | UCUU | | GAUUU | | | TCCATC |
| | | | | CCAU | | UUCCAG | | | |
| | | | | C | | AGUCUC | | | |
| | | | | | | UUCCAU | | | |
| | | | | | | C | | | |

TABLE 15
Exemplary CD58 gRNAs having >5% Editing Efficiency.

| | | | | | SEQ | | | | |
|---|---|---|---|---|---|---|---|---|---|
| | | | | | ID NO: | | | | |
| | SEQ | | SEQ | | for | | | SEQ ID | |
| | ID NO: | | ID NO: | | Repeat + | Repeat + | | NO: | |
| | for | | for | | Spacer | Spacer | | for | |
| | Repeat | Repeat | Spacer | Spacer | Complete | Complete | Chromosome | Genomic | Genomic |
| | Sequence | Sequence | Sequence | Sequence | Sequence | Sequence | location | Target | Target |
| ID | (5′-3′) | (5′-3′) | (5′-3′) | (5′-3′) | (5′-3′) | (5′-3′) | (hg38) | Region | Region |
| CD58_ | 129 | UAAU | 254 | ACUC | 255 | UAAUUUCU | Chr1: | 256 | CTTCA |
| Exon | | UUCU | | ACCA | | ACUCUUGU | 116570897- | | CTCAC |
| 1_gRNA | | ACUC | | AAGC | | AGAUACUC | 116570917 | | CAAA |
| 1 | | UUGU | | AGUG | | ACCAAAGC | | | GCAG |
| | | AGAU | | CAGC | | AGUGCAGC | | | TGCA |
| | | | | A | | A | | | GCA |
| CD58_ | 129 | UAAU | 269 | UUUA | 270 | UAAUUUCU | Chr1: | 271 | TTTAT |
| Exon | | UUCU | | GACA | | ACUCUUGU | 116544409- | | TTAGA |
| 2_gRNA | | ACUC | | CUGU | | AGAUUUUA | 116544429 | | CACTG |
| 5 | | UUGU | | GUCA | | GACACUGU | | | TGTCA |
| | | AGAU | | GGUA | | GUCAGGUA | | | GGTA |
| | | | | G | | G | | | G |
| CD58_ | 129 | UAAU | 272 | GACA | 273 | UAAUUUCU | Chr1: | 274 | TTTAG |
| Exon | | UUCU | | CUGU | | ACUCUUGU | 116544405- | | ACACT |
| 2_gRNA | | ACUC | | GUCA | | AGAUGACA | 116544425 | | GTGTC |
| 6 | | UUGU | | GGUA | | CUGUGUCA | | | AGGT |
| | | AGAU | | GCCU | | GGUAGCCU | | | AGCCT |
| | | | | C | | C | | | C |
| CD58_ | 129 | UAAU | 278 | AAGG | 279 | UAAUUUCU | Chr1: | 280 | TTTAA |
| Exon | | UUCU | | CACA | | ACUCUUGU | 116544524- | | AGGC |
| 2_gRNA | | ACUC | | UUGC | | AGAUAAGG | 116544544 | | ACATT |
| 9 | | UUGU | | UUGG | | CACAUUGC | | | GCTTG |
| | | AGAU | | UACA | | UUGGUACA | | | GTAC |
| | | | | U | | U | | | AT |
| CD58_ | 129 | UAAU | 302 | UUGG | 303 | UAAUUUCU | Chr1: | 304 | TTTGT |
| Exon | | UUCU | | GAAA | | ACUCUUGU | 116544582- | | TGGG |
| 2_gRNA | | ACUC | | AACA | | AGAUUUGG | 116544602 | | AAAA |
| 18 | | UUGU | | GCUG | | GAAAAACA | | | ACAG |
| | | AGAU | | AUGA | | GCUGAUGA | | | CTGAT |
| | | | | A | | A | | | GAA |
| CD58_ | 129 | UAAU | 326 | CUCA | 327 | UAAUUUCU | Chr1: | 328 | TTTAC |
| Exon | | UUCU | | CCGC | | ACUCUUGU | 116535960- | | TCACC |
| 3_gRNA | | ACUC | | UGCU | | AGAUCUCA | 116535980 | | GCTGC |
| 2 | | UUGU | | UGGG | | CCGCUGCU | | | TTGGG |
| | | AGAU | | AUAC | | UGGGAUAC | | | ATAC |
| | | | | A | | A | | | A |

### 9.6 Example 6: Exemplary Optimizing Pulse Codes for Nucleofection of RFX gRNAs

Preparation of RNP complexes. RFX5_Exon9_gRNA2 20 bp Alt-R crRNA was synthesized (IDT) and dissolved in nuclease free duplex buffer (IDT #11-05-01-12) at 200 μM. RNP complexes were prepared fresh on the day of nucleofection by mixing a ratio of 63 pmol (1 μl of 10 μg/μl) WT MAD7 (Aldevron) with 200 pmol (1 μl of 200 μM) crRNA and 100 μg of poly-L-glutamic acid sodium salt (PGA, MW15-50 kD, Sigma p4761). The RNP complex was incubated at room temperature for 30 minutes. P3 Buffer (Lonza #V4SP-3096) with 3 μM electroporation enhancer (IDT #1076301) was added for a total volume of 10 μL.

Generation of Knockout human iPSCs. Three human iPSC clones were pretreated with CEPT cocktail (chroman 1 (MedChem Express #HY-15392, emricasan (SelleckChem S7775), polyamine supplement (Sigma-Aldrich P8483), and trans-ISRIB (R&D Systems 5284)). The final concentrations of the CEPT cocktail: 50 nM chroman 1, 5 μM emricasan, polyamine supplement 1:1,000, and 0.7 μM trans-ISRIB were diluted in Stemfit Basic 04 Complete Type Medium (Ajinomoto Basic04CT). The iPSCs were collected (0.5e6 cells per reaction) and resuspended in 10 μL of P3 buffer with 3 μM electroporation enhancer. The cells were combined with 10 μL of RNP complex and nucleofected in 96 well cuvettes (Lonza #V4SP-3096) using one of the six different programs on the Lonza 4D system. The iPSCs were then transferred to one well of a 24 well plate coated with 0.5 μg/cm² of iMatrix-511 (Takara #T304) containing Stemfit Basic 04 Complete Type Medium with CEPT cocktail. The iPSCs were collected 48 hours post electroporation, the DNA was extracted, and the region around the gRNA target-site was amplified and Sanger sequenced. Editing efficiency was measured by the ICE tool (Synthego) to analyze sanger sequencing results (GENEWIZ).

Pulse code optimization was performed to determine the best pulse codes for nucleofection of RFX5 gRNAs in multiple γδT-iPSC clones. High editing efficiency was achieved with several pulse codes (CA-137, CA-118, CE-118, CM-113, DC-100, and DN-100) on the Lonza Nucleofector in three γδT-iPSC clones with gRNA RFX5_Exon9_gRNA 2 20 bp. This gRNA can efficiently edit RFX5 in several different iPSC clones.

Preparation of RNP complexes. RFX5_Exon9_gRNA2 20 bp Alt-R crRNA was synthesized (IDT) and dissolved in nuclease free duplex buffer (IDT #11-05-01-12) at 200 μM. RNP complexes were prepared fresh on the day of nucleofection by mixing a ratio of 63 pmol (1 μl of 10 μg/μl) WT MAD7 (Aldevron) with 200 pmol (1 μl of 200 μM) crRNA and 100 μg of poly-L-glutamic acid sodium salt (PGA, MW15-50 kD, Sigma p4761). The RNP complex was incubated at room temperature for 30 minutes. P3 Buffer (Lonza #V4SP-3096) with 3 μM electroporation enhancer (IDT #1076301) was added for a total volume of 10 μL.

Generation of knockout human iPSCs. Three human iPSC clones were pretreated with CEPT cocktail (chroman 1 (MedChem Express #HY-15392, emricasan (SelleckChem S7775), polyamine supplement (Sigma-Aldrich P8483), and trans-ISRIB (R&D Systems 5284)). The final concentrations of the CEPT cocktail: 50 nM chroman 1, 5 μM emricasan, polyamine supplement 1:1,000, and 0.7 μM trans-ISRIB were diluted in Stemfit Basic 04 Complete Type Medium (Ajinomoto Basic04CT). The iPSCs were collected (0.5e6 cells per reaction) and resuspended in 10 μL of P3 buffer with 3 μM electroporation enhancer. The cells were combined with 10 μL of RNP complex and nucleofected in 96 well cuvettes (Lonza #V4SP-3096) using one of the six different programs on the Lonza 4D system. The iPSCs were then transferred to one well of a 24 well plate coated with 0.5 μg/cm² of iMatrix-511 (Takara #T304) containing Stemfit Basic 04 Complete Type Medium with CEPT cocktail. The iPSCs were collected 48 hours post electroporation, the DNA was extracted, and the region around the gRNA target-site was amplified and Sanger sequenced. Editing efficiency was measured by the ICE tool (Synthego) to analyze sanger sequencing results (GENEWIZ).

### 9.7 Example 7: Exemplary CAR Knock-In

Preparation of RNP complexes. RFX5_Exon9_gRNA2 20 bp and RFX5_Exon10_gRNA1 20 bp Alt-R crRNA were synthesized (IDT) and dissolved in nuclease free duplex buffer (IDT #11-05-01-12) at 200 PM. RNP complexes were prepared fresh on the day of nucleofection by mixing a ratio of 63 pmol (1 μl of 10 μg/μl) WT MAD7 (Aldevron) with 200 pmol (1 μl of 200 μM) crRNA and 100 μg of poly-L-glutamic acid sodium salt (PGA, MW15-50 kD, Sigma p4761). The RNP complexes were incubated at room temperature for 30 minutes. P3 Buffer (Lonza #V4SP-3096) with 3 μM electroporation enhancer (IDT #1076301) was added for a total volume of 10 μL. 3 μg of appropriate donor DNA templates containing promoter and CAR sequence were added to the RNP and incubated at room temperature for 1 minute.

Generation of CAR Knock-In human iPSCs. iPSCs were pretreated with CEPT cocktail (chroman 1 (MedChem Express #HY-15392, emricasan (SelleckChem S7775), polyamine supplement (Sigma-Aldrich P8483), and trans-ISRIB (R&D Systems 5284)). The final concentrations of the CEPT cocktail: 50 nM chroman 1, 5 μM emricasan, polyamine supplement 1:1,000, and 0.7 μM trans-ISRIB were diluted in Stemfit Basic 04 Complete Type Medium (Ajinomoto Basic04CT). The iPSCs were collected (0.5e6 cells per reaction) and resuspended in 10 μL of P3 buffer with 3 μM electroporation enhancer. The cells were combined with 10 μL of RNP complex and nucleofected in 96 well cuvettes (Lonza #V4SP-3096) using the CA-137 program on the Lonza 4D system. The iPSCs were then transferred to one well of 6 well plate coated with 0.5 μg/cm² of iMatrix-511 (Takara #T304) containing Stemfit Basic 04 Complete Type Medium with CEPT cocktail and with and without 0.5 μM M3814 (SelleckChem 1637542-33-6). The media was changed daily for 6 days, at which surface expression of CAR was measured by flow cytometry.

The gRNAs RFX5_Exon10_gRNA1 20 bp and RFX5_Exon9_gRNA 2 20 bp, discussed above, were used to knock in a CAR into the RFX5 gene.

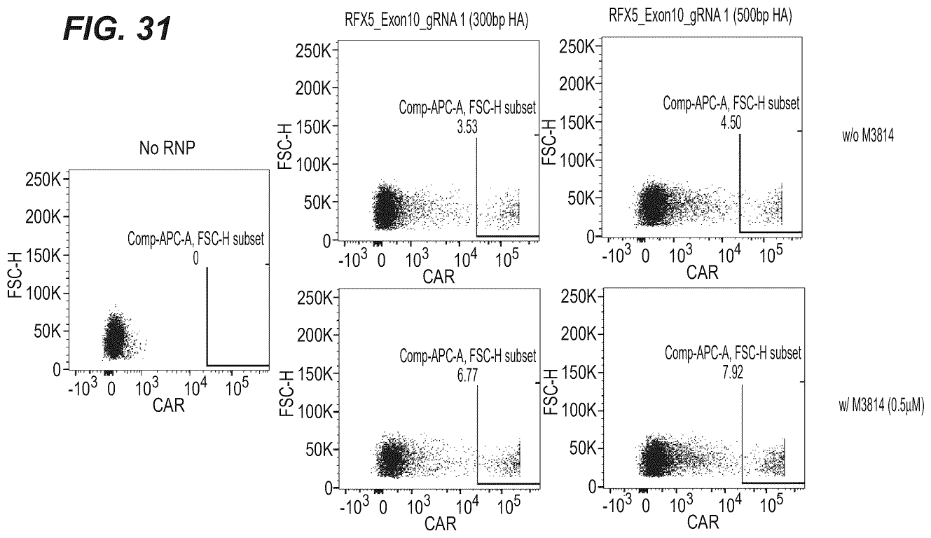

FIG. 31 shows the editing efficiency of CAR knock-in into RFX5 with gRNA RFX5_Exon00_gRNA1 20 bp. Four separate reactions were performed with either 300 bp or 500 bp homology arms in the DNA donor template and with and without M3814, a DNA-dependent protein kinase (DNA-PK) inhibitor that enhances DNA donor template repair through the HDR pathway. The flow plots shown are gated on live, single cells, and the CAR positive cells were determined by comparing the edited samples to the no RNP negative control. These results show RFX5_Exon10_gRNA1 20 bp can be used to knock-in a transgene containing a promoter and CAR into RFX5 resulting in CAR expression on the cell surface detected by flow cytometry. The knock-in efficiency increased with a longer homology arm length (500 bp versus 300 bp) and with the addition of M3814.

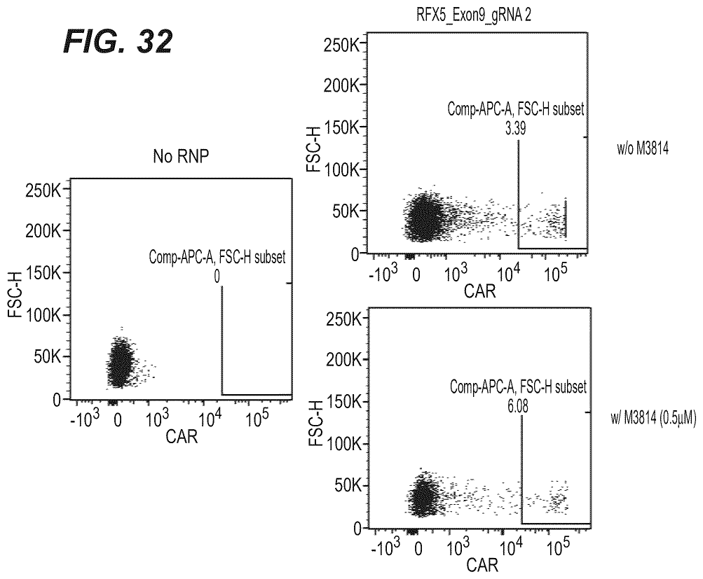

FIG. 32 shows CAR knock-in into RFX5 with gRNA RFX5_Exon9_gRNA 2 20 bp. The editing efficiency of CAR knock-in is shown with gRNA RFX5_Exon9_gRNA 2 20 bp with 500 bp homology arms in the DNA donor template and with and without M3814. The flow plots shown are gated on live, single cells, and the CAR positive cells were determined by comparing the edited samples to the no RNP negative control. This gRNA can be used to knock-in a transgene containing a promoter and CAR into RFX5 resulting in CAR expression on the cell surface detected by flow cytometry. The knock-in efficiency is increased with the addition of M3814.

### 9.8 Example 8: Exemplary Pulse Code Optimization of CAR Knock-In

Preparation of RNP complexes. RFX5_Exon9_gRNA2 20 bp Alt-R crRNA was synthesized (IDT) and dissolved in nuclease free duplex buffer (IDT #11-05-01-12) at 200 μM. RNP complexes were prepared fresh on the day of nucleofection by mixing a ratio of 63 pmol (1 μl of 10 μg/μl) WT MAD7 (Aldevron) with 200 pmol (1 μl of 200 μM) crRNA and 100 μg of poly-L-glutamic acid sodium salt (PGA, MW15-50 kD, Sigma p4761). The RNP complexes were incubated at room temperature for 30 minutes. P3 Buffer (Lonza #V4SP-3096) with 3 μM electroporation enhancer (IDT #1076301) was added for a total volume of 10 μL. 3 μg of donor DNA templates containing promoter and CAR sequence were added to the RNP and incubated at room temperature for 1 minute.

Generation of knockout human iPSCs. iPSCs were pretreated with CEPT cocktail (chroman 1 (MedChem Express #HY-15392, emricasan (SelleckChem S7775), polyamine supplement (Sigma-Aldrich P8483), and trans-ISRIB (R&D Systems 5284)). The final concentrations of the CEPT cocktail: 50 nM chroman 1, 5 μM emricasan, polyamine supplement 1:1,000, and 0.7 μM trans-ISRIB were diluted in Stemfit Basic 04 Complete Type Medium (Ajinomoto Basic04CT). The iPSCs were collected (0.5e6 cells per reaction) and resuspended in 10 μL of P3 buffer with 3 μM electroporation enhancer. The cells were combined with 10 μL of RNP complex and nucleofected in 96 well cuvettes (Lonza #V4SP-3096) using the CA-137 or DN100 programs on the Lonza 4D system. The iPSCs were then transferred to one well of 6 well plate coated with 0.5 μg/cm² of iMatrix-511 (Takara #T304) containing Stemfit Basic 04 Complete Type Medium with CEPT cocktail and with and without 0.5 μM M3814 (SelleckChem 1637542-33-6). The media was changed daily for 6 days, at which surface expression of CAR was measured by flow cytometry.

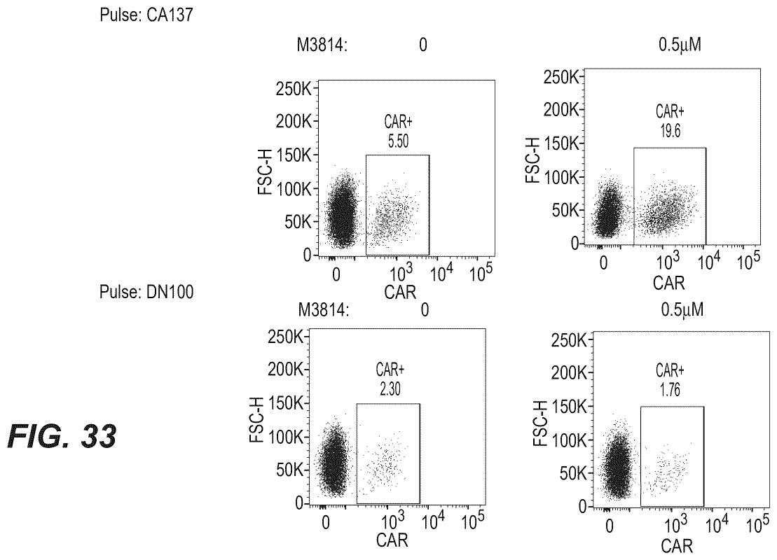

Pulse code optimization was performed to find the best pulse codes for nucleofection of gRNAs for performing a CAR knock-in into RFX5. FIG. 33 shows the editing efficiency of CAR knock-in was achieved with two pulse codes (CA-137, DN-100) on the Lonza Nucleofector with gRNA RFX5_Exon9_gRNA 2 20 bp with and without M3814. The flow plots shown are gated on live, single cells. This gRNA can be used to knock-in a transgene containing a promoter and CAR into RFX5 resulting in CAR expression on the cell surface detected by flow cytometry. The knock-in efficiency is increased with identifying the optimal electroporation condition and the addition of M3814.

### 9.9 Example 9: Surface Molecule Expression on RFX5 Knockout and CD58 Knockout iPSCs

Preparation of RNP complexes. RFX5_Exon9_gRNA2 20 bp and CD58_Exon2_gRNA 9 Alt-R crRNA were synthesized (IDT) and dissolved in nuclease free duplex buffer (IDT #11-05-01-12) at 200 μM. RNP complexes were prepared fresh on the day of nucleofection by mixing a ratio of 63 pmol (1 μl of 10 μg/μl) WT MAD7 (Aldevron) with 200 pmol (1 μl of 200 μM) crRNA and 100 μg of poly-L-glutamic acid sodium salt (PGA, MW15-50 kD, Sigma p4761). The RNP complexes were incubated at room temperature for 30 minutes. P3 Buffer (Lonza #V4SP-3096) with 3 μM electroporation enhancer (IDT #1076301) was added for a total volume of 10 μL.

Generation of Knockout human iPSCs. iPSCs were pretreated with CEPT cocktail (chroman 1 (MedChem Express #HY-15392, emricasan (SelleckChem S7775), polyamine supplement (Sigma-Aldrich P8483), and trans-ISRIB (R&D Systems 5284)). The final concentrations of the CEPT cocktail: 50 nM chroman 1, 5 μM emricasan, polyamine supplement 1:1,000, and 0.7 μM trans-ISRIB were diluted in Stemfit Basic 04 Complete Type Medium (Ajinomoto Basic04CT). The iPSCs were collected (0.5e6 cells per reaction) and resuspended in 10 μL of P3 buffer with 3 μM electroporation enhancer. The cells were combined with 10 μL of RNP complex and nucleofected in 96 well cuvettes (Lonza #V4SP-3096) using the CA-137 program on the Lonza 4D system. The iPSCs were then transferred to one well of 24 well plate coated with 0.5 μg/cm² of iMatrix-511 (Takara #T304) containing Stemfit Basic 04 Complete Type Medium (Ajinomoto Basic04CT) with CEPT cocktail. The iPSCs were expanded to 6 well plates two days post nucleofection and the media was changed daily for 7 days, at which surface expression of HLA class I and CD58 were measured by flow cytometry.

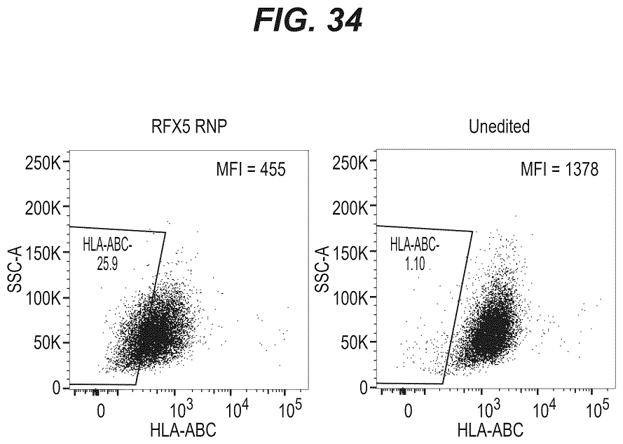

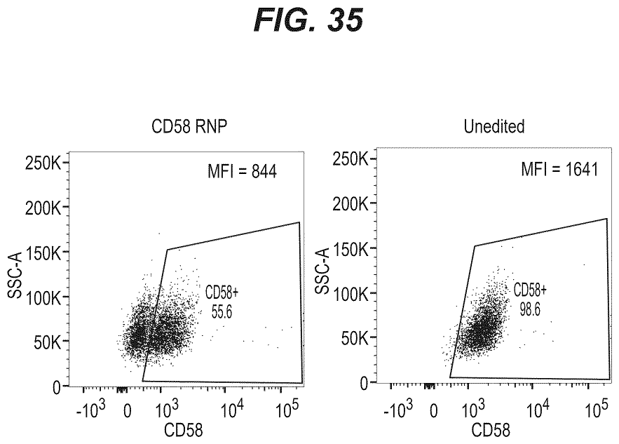

Expression of surface molecules including HLA class I molecules and CD58 were measured on iPSCs with a knockout in RFX5. FIG. 34 shows that cells edited with MAD7 and gRNA RFX5_Exon9_gRNA 2 20 bp (left panel) had decreased expression of HLA class I compared to the unedited cells (right panel). The flow plots shown are gated on live, single cells. The mean fluorescence intensity (MFI) of HLA class I of the edited cells decreased compared to the unedited sample and a quarter of the cells had no expression of HLA-ABC. This shows that editing with gRNA RFX5_Exon9_gRNA 2 20 bp leads to a reduction in HLA class I expression on the surface of iPSCs. FIG. 35 shows that cells edited with MAD7 and gRNA CD58_Exon2_gRNA 9 (left panel) had decreased expression of CD58 compared to the unedited cells (right panel). The flow plots shown are gated on live, single cells. The mean fluorescence intensity (MFI) of CD58 of the edited cells decreased compared to the unedited sample. Almost half of the edited cells were negative for CD58. This shows that editing with gRNA CD58_Exon2_gRNA 9 leads to a reduction in CD58 expression on the surface of iPSCs.

### 9.10 Example 10: Generation of iPSCs with CAR Knock-Ins

Generation of CAR⁺ Clones. Bulk edited BCMA knock-in into RFX5 cells were single cell sorted. The BCMA positive knock-in clones were expanded for 16 days and the pluripotency and CAR expression were measured by flow cytometry.

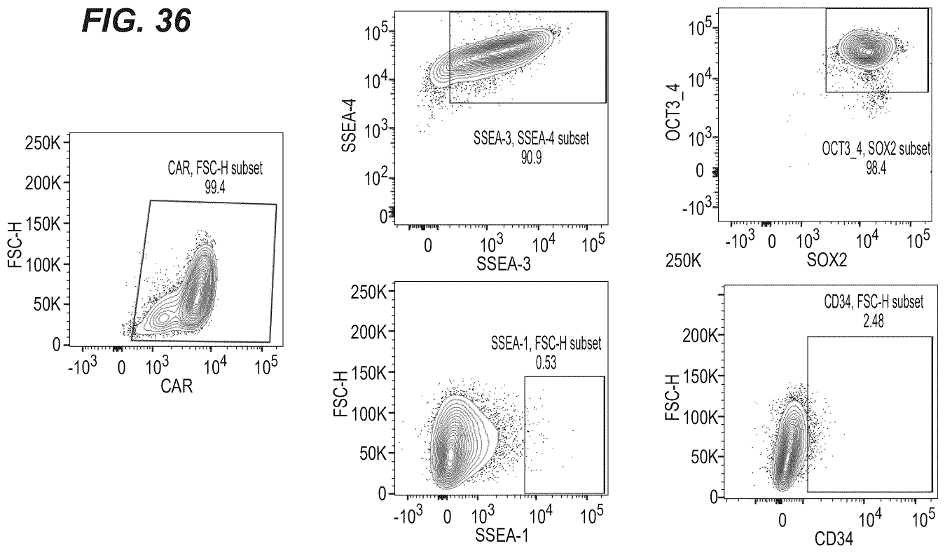

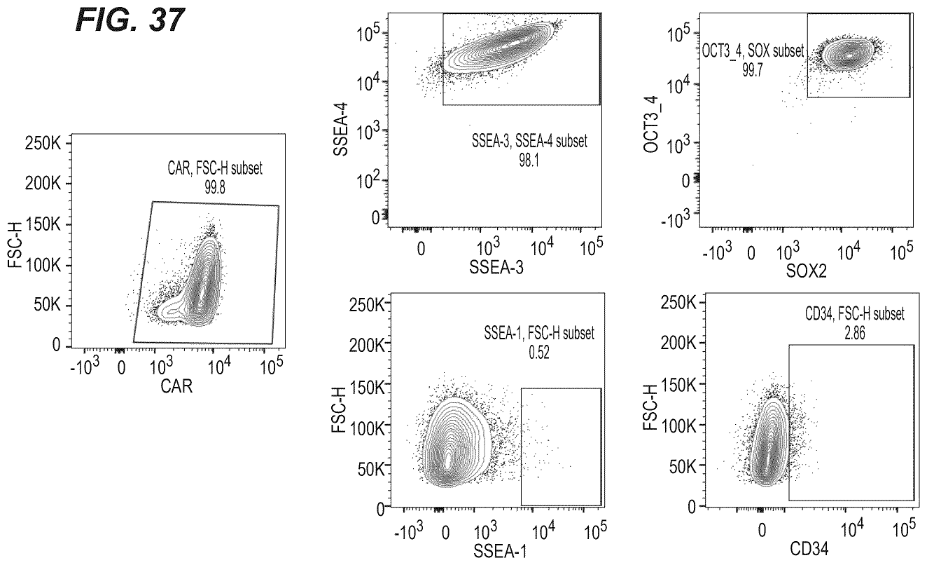

The gRNA RFX5_Exon9_gRNA 2 20 bp was successfully used to knock in a CAR into RFX5. FIG. 36 shows that the bulk edited cells were single-cell sorted to produce clonal CAR positive cells. The flow plots shown are gated on live, single cells. A representative clone, Clone D5, has nearly 100% CAR expression determined by flow cytometry. This clone was edited with a 12 bp deletion. The pluripotency markers SSEA-3, SSEA-4, OCT3/4, and SOX2 have high expression and the surface markers SSEA-1 and CD34 that are not expressed in iPSCs remain low after editing and cloning. This shows that the process of editing and cloning results in BCMA CAR positive clones without disrupting iPSC pluripotency. FIG. 37 shows that bulk edited cells were single-cell sorted to produce clonal CAR positive cells. The flow plots shown are gated on live, single cells. A representative clone, Clone C3, has nearly 100% CAR expression determined by flow cytometry. This clone was edited with a 15 bp deletion. The pluripotency markers SSEA-3, SSEA-4, OCT3/4, and SOX2 have high expression and the surface markers SSEA-1 and CD34 that are not expressed in iPSCs remain low after editing and cloning. These results show that the process of editing and cloning results in BCMA CAR positive clones without disrupting iPSC pluripotency.

### 9.11 Example 11: Editing Efficiencies of Exemplary Engineered gRNAs (Split, crRNA, and tracrRNA)

Preparation of RNP complexes. The crRNA, split crRNA, and split tracrRNA were synthesized (IDT) and dissolved in nuclease free duplex buffer (IDT #11-05-01-12) at 200 μM. Prior to RNP formation, split crRNA and corresponding split tracrRNA were added in equimolar mixture and incubated for 15 minutes at room temperature. RNP complexes were prepared fresh on the day of nucleofection by mixing a ratio of 63 pmol (1 μl of 10 μg/μl) WT MAD7 (Aldevron) with 200 pmol (1 μl of 200 μM) crRNA and 100 μg of poly-L-glutamic acid sodium salt (PGA, MW15-50 kD, Sigma p4761). The RNP complex was incubated at room temperature for 30 minutes. P3 Buffer (Lonza #V4SP-3096) with 3 μM electroporation enhancer (IDT #1076301) was added for a total volume of 10 μL.

Generation of CAR Knockout human iPSCs with Split gRNAs. iPSC were pretreated with CEPT cocktail (chroman 1 (MedChem Express #HY-15392, emricasan (SelleckChem S7775), polyamine supplement (Sigma-Aldrich P8483), and trans-ISRIB (R&D Systems 5284)). The final concentrations of the CEPT cocktail: 50 nM chroman 1, 5 μM emricasan, polyamine supplement 1:1,000, and 0.7 μM trans-ISRIB were diluted in Stemfit Basic 04 Complete Type Medium (Ajinomoto Basic04CT). The iPSCs were collected (0.5e6 cells per reaction) and resuspended in 10 μL of P3 buffer with 3 μM electroporation enhancer. The cells were combined with 10 μL of RNP complex and nucleofected in 96 well cuvettes (Lonza #V4SP-3096) CA-137 program on the Lonza 4D system. The iPSCs were then transferred to one well of 24 well plate coated with 0.5 μg/cm² of iMatrix-511 (Takara #T304) containing Stemfit Basic 04 Complete Type Medium with CEPT cocktail. The iPSCs were collected 48 hours post electroporation, the DNA was extracted, and the region around the gRNA target-site was amplified and Sanger sequenced. Editing efficiency was measured by the ICE tool (Synthego) to analyze sanger sequencing results (GENEWIZ).

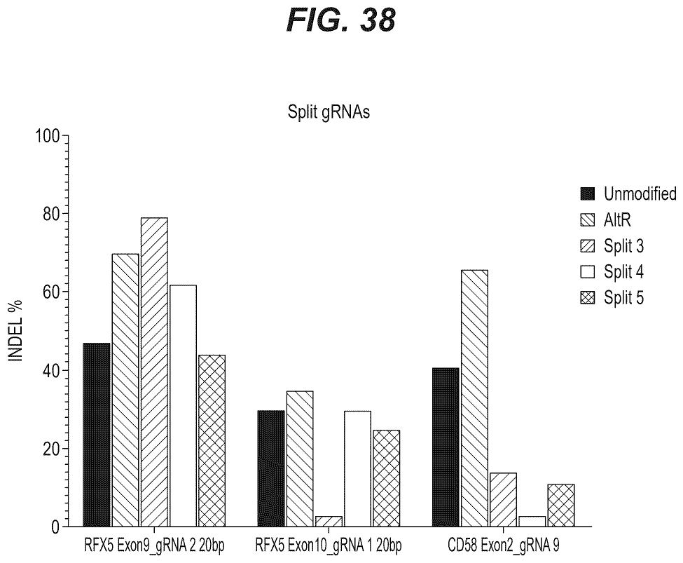

Split RFX5 and CD58 gRNAs were generated, and the editing efficiency of the split gRNAs was determined. The split gRNAs were formed by adding equimolar mixture of the split tracrRNA with relevant crRNA and incubating for 15 minutes at room temperature prior to RNP formation (Table 16). FIG. 38 shows Indel frequency of MAD7 with unmodified crRNA, AltR modified crRNA, and split gRNAs 3, 4, and 5 targeting the two RFX5 and CD58 loci. For RFX5 Exon9_gRNA 2 20 bp, Split 3 gave higher editing efficiency and Split 4 and Split 5 gave relatively similar editing efficiencies compared to the single crRNA format. For RFX5 Exon10_gRNA 1 20 bp, Split format 4 and 5 for RFX5 gave similar editing efficiencies to single crRNA format. The split format decreased editing efficiency for CD58 Exon2_gRNA 9, but still resulted in >10% indel formation for split 3 and 5. These results show that it is possible to retain high editing efficiency with a split gRNA format by identifying the optimal split design. Also, MAD7 is not solely reliant on the single crRNA format.

TABLE 16
Split gRNAs.

| ID | Sequence (5′-3′) | SEQ ID NO |
|---|---|---|
| Split 3 tracrRNA | UAAUUUCUACUC | 377 |
| Split 4 tracrRNA | UAAUUUCUACUCU | 378 |
| Split 5 tracrRNA | UAAUUUCUACUCUU | 379 |
| RFX5_Exon9_gRNA 2 crRNA 3 | UUGUAGAUAGGAUCCGCUCUGCCCAGUC | 380 |
| RFX5_Exon9_gRNA 2 crRNA 4 | UGUAGAUAGGAUCCGCUCUGCCCAGUC | 381 |
| RFX5_Exon9_gRNA 2 crRNA 5 | GUAGAUAGGAUCCGCUCUGCCCAGUC | 382 |
| RFX5_Exon10_gRNA 1 crRNA 3 | UUGUAGAUGAUGACCGUUCCCGAGGUGC | 383 |
| RFX5_Exon10_gRNA 1 crRNA 4 | UGUAGAUGAUGACCGUUCCCGAGGUGC | 384 |
| RFX5_Exon10_gRNA 1 crRNA 5 | GUAGAUGAUGACCGUUCCCGAGGUGC | 385 |
| CD58_Exon2_gRNA 9 crRNA 3 | UUGUAGAUAAGGCACAUUGCUUGGUACA | 386 |
| CD58_Exon2_gRNA 9 crRNA 4 | UGUAGAUAAGGCACAUUGCUUGGUACA | 387 |
| CD58_Exon2_gRNA 9 crRNA 5 | GUAGAUAAGGCACAUUGCUUGGUACA | 388 |

### 9.12 Example 12. Exemplary Evasion of Alloreactive T Cells and Primary NK Cells

Generation of gene edited human T cells using CRISPR. Human T cells were isolated from peripheral blood mononuclear cells (PBMCs) by negative selection (StemCell #17951) and rested overnight in TexMACS (Miltenyi #170-076-307), 30 IU/ml hIL-21S (Miltenyi #130-097-748), herein referred to as “media,” at 1×10⁶ cells/ml. The following day, T cells were collected, washed once with PBS, and nucleofected with 6 μl of RNP complexes specific for the indicated genes in 20 μl of P3 Buffer (Lonza #V4SP-3096) with 4 μM electroporation enhancer (IDT #1075916) in 96 well cuvettes (Lonza #V4SP-3096) using the EH-115 program on the Lonza 4D system. Immediately after nucleofection, T cells were recovered in 200 μl warm media for 2 hours at 37 C. T cells were then activated with a 1:17.5 dilution of TransAct (Miltenyi #130-019-011) in media at approximately 1×10⁶ cells/ml. T cells were expanded in culture by addition of fresh media every 2-3 days for an additional 14 days, at which point surface expression of relevant molecules was measured by flow cytometry before cryopreserving cells in Cryostor CS10 (Sigma #C2874-100ML).

Generation of allogeneic effector T cells. PBMCs from a non-HLA matched human donor were stimulated with irradiated (40 Gy) PBMCs from the human donor used to make HLA class I and II negative T cells at a 1:1 ratio in media [TexMACS (Miltenyi #170-076-307) and 100 IU/ml Penicillin+100 μg/ml Streptomycin (Gibco #15140-122)], without IL-2 at 2×10⁶ cells/ml. After 2 days, an equal volume of media containing 60 IU/ml hIL-21S (Miltenyi #130-097-748) was added to achieve a final concentration of 30 IU/ml IL-21S. After 5 additional days of culture, cells were washed, resuspended into media with 30 IU/ml IL-21S and restimulated with another round of irradiated (40 Gy) PBMCs from the human donor used to make HLA class I and II negative T cells at a 1:1 ratio. Following another 2 days of culture, an equal volume of media was added and IL-21S was supplied to 200 IU/ml. Two days later, fresh media and 200 IU/ml IL-21S was added to dilute the cells to 0.5×10⁶ cells/ml, and cells were cultured for another 3 days before being cryopreserved in CS10. In some experiments, alloreactive effector cells were separated into purified T cells (mixture of CD4⁺ and CD8⁺) and purified NK cells (CD56+CD3−) with an EasySep CD56+ isolation kit (STEMCELL Technologies #17855).

Isolation of primary NK cells. Human NK cells were isolated from leukapheresis (StemExpress and HemaCare) using the NK cell Isolation kit (Miltenyi #130-092-657) and program on a CliniMACS Prodigy (Miltenyi Biotec). NK cells were cryopreserved at 10⁶ cells/mL in Cryostor CS10 (Sigma #C2874-100ML).

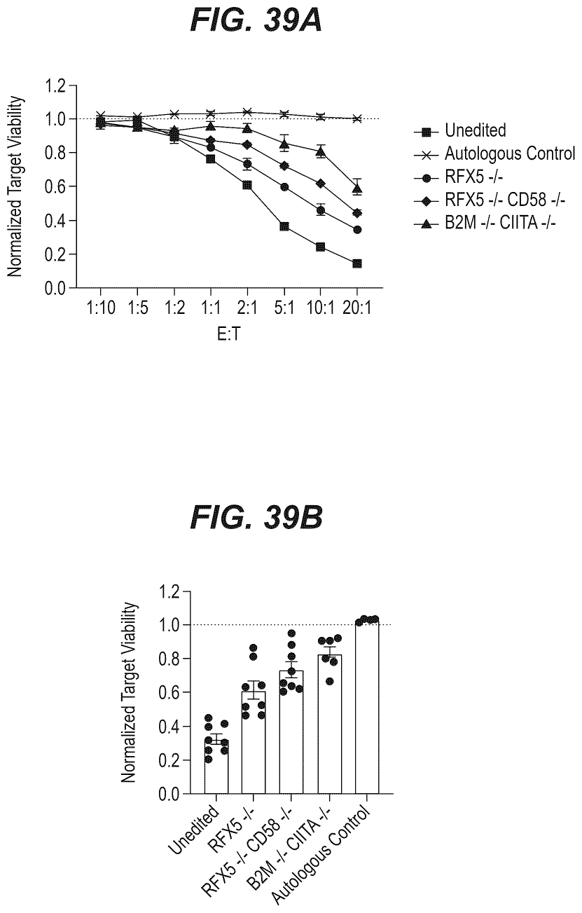

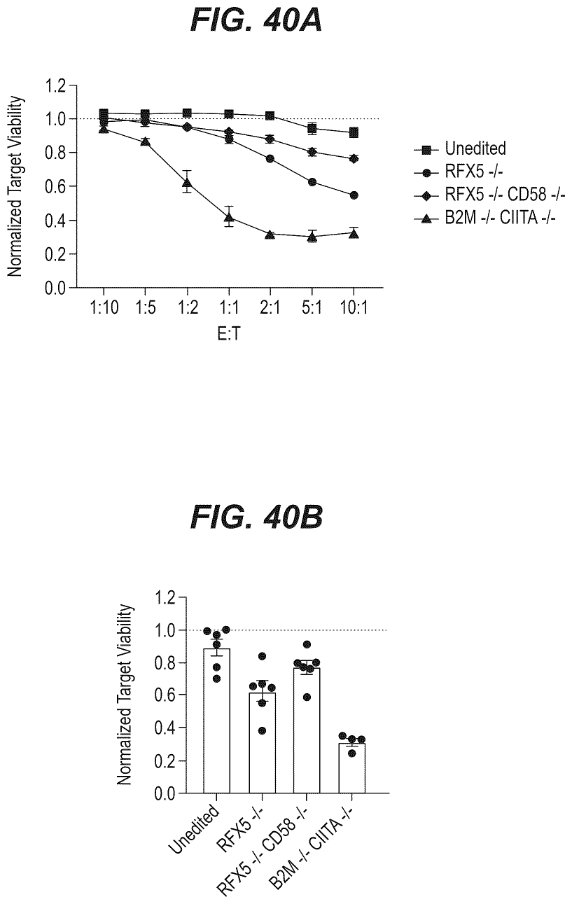

Allogeneic response assays. Cryopreserved gene edited T cells (“targets”) were thawed and rested overnight in RPMI+L-glutamine (Gibco #11875-093), 10% FBS (Gibco #16140-071), 100 IU/ml Penicillin+100 μg/ml Streptomycin (Gibco #15140-122), 1 mM Sodium Pyruvate (Gibco #11360-070), 10 mM HEPES (Gibco #15630-080), and 55 μM 2-mercaptoethanol (Gibco #21985-023), herein referred to as “assay media,” supplemented with 30 IU/ml hIL-21S (Miltenyi #130-097-748), at 1×10⁶ cells/ml. The next day, target T cells were washed to remove IL-2 and seeded in 96 well U-bottom plates in assay media at 10,000 cells/well. Allogeneic effector T cells were also thawed and rested one day prior to experiment setup in assay media supplemented with 30 IU/ml hIL-21S. Primary NK cells were also thawed and rested one day prior to experiment setup in assay media supplemented with 0.2 ng/mL IL-2 (Gibco #PHC0026). The next day, allogeneic effector T cells or NK cells were labelled with 1 μM Cell Trace Violet (Thermo #C34557) in PBS for 20 mins at 37° C., washed twice with assay media, and seeded with targets for cytotoxicity assays (various E:Ts). Cells were analyzed by flow cytometry 18-24 hours after plating. Normalized target viability was calculated as: % live targets at E:T/% live targets alone. Gene edited or control T cells (targets) were co-cultured with alloreactive effector T cells at the indicated E:Ts in an overnight cytotoxicity assay. Normalized target viability was calculated as: % live targets at E:T/% live targets alone, where a value of 1.0 indicates complete evasion of cytotoxicity. Top panel (FIG. 39A) shows data from one representative experiment with a single human donor. Bottom panel (FIG. 39B) shows aggregate data at E:T=10 from multiple experiments with several target and effector human donors. Top panel (FIG. 40A) shows data from one representative experiment with a single human donor. Bottom panel (FIG. 40B) shows aggregate data at E:T=10 from multiple experiments with several target and effector human donors.

Pan T cells with knockouts in RFX5, RFX5 plus CD58 (RFX5/CD58), and B2M plus CIITA (B2M/CIITA) were generated and evaluated for their ability to evade cytotoxicity from allogeneic T cells and NK cells from multiple human donors. A representative experiment from a single human donor is shown in FIGS. 39A and 40A, and combined data from multiple human donors at E:T=10 is shown in FIGS. 39B and 40B. As compared to unedited T cells, RFX5 knockout T cells had an improved ability to survive challenge with allogeneic effector T cells. RFX5/CD58 dual knockout T cells had a further enhanced ability to survive compared to RFX5 knockout T cells. B2M/CIITA dual knockout T cells also showed a strong ability to survive compared to unedited T cells (FIGS. 39A and 39B).

When challenged with primary NK cells, B2M/CIITA dual knockout T cells showed strong susceptibility to lysis. Relative to B2M/CIITA dual knockout T cells, RFX5 knockout T cells had an improved ability and RFX5/CD58 dual knockout had an even further improved ability to survive challenge with primary NK cells, to the point that RFX5/CD58 dual knockouts survived nearly as well as unedited T cells (FIGS. 40A and 40B).

### 9.13 Example 13: Exemplary Dual CAR and CD58 miR-shRNA Expression System for Expression of a CAR and Knockdown of Endogenous CD58 from a Single Vector

Generation of gene edited human T cells using CRISPR. Human T cells were isolated from peripheral blood mononuclear cells (PBMCs) by negative selection (StemCell #17951) and rested overnight in TexMACS (Miltenyi #170-076-307), 30 IU/ml hIL-21S (Miltenyi #130-097-748), herein referred to as “media,” at 1×10⁶ cells/ml. The following day, T cells were collected, washed once with PBS, and nucleofected with 6 μl of RNP complexes specific for the indicated genes in 20 μl of P3 Buffer (Lonza #V4SP-3096) with 4 μM electroporation enhancer (IDT #1075916) in 96 well cuvettes (Lonza #V4SP-3096) using the EH-115 program on the Lonza 4D system. Immediately after nucleofection, T cells were recovered in 200 μl warm media for 2 hours at 37 C. T cells were then activated with a 1:17.5 dilution of TransAct (Miltenyi #130-019-011) in media at approximately 1×10⁶ cells/ml. T cells were expanded in culture by addition of fresh media every 2-3 days for an additional 14 days, at which point surface expression of relevant molecules was measured by flow cytometry before cryopreserving cells in Cryostor CS10 (Sigma #C2874-100ML). In some experimental conditions, expanded T cells were transduced with lentivirus 1 day after TransAct stimulation.

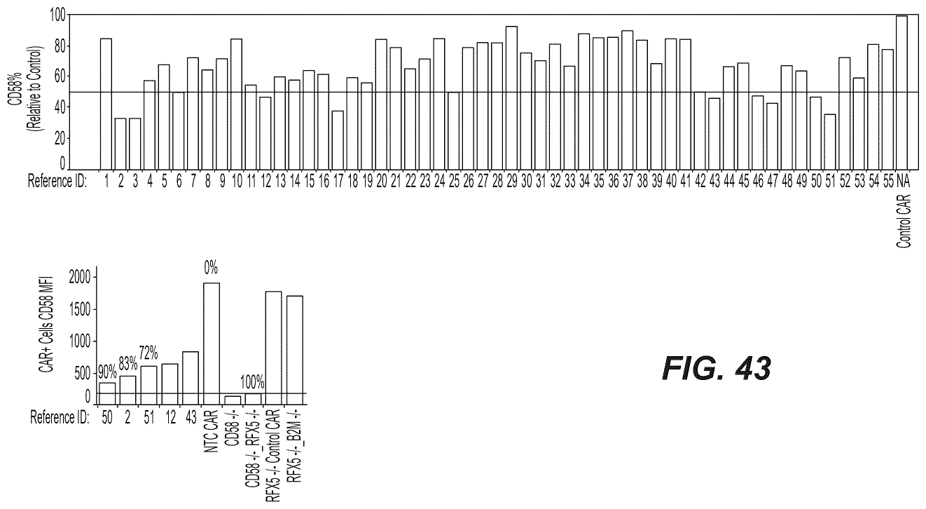

CD58 targeting miR-shRNAs in a lentiviral vector were transduced into 24-hour activated (TransAct, Miltenyi #130-019-011) primary human T cells at an MOI=5. CD58 expression was measured 14 days after transduction. The top panel of FIG. 43 depicts initial round screening of 55 different miR-shRNA constructs and a control CAR (without a miR-shRNA). CD58% is the MFI of CD58 for each construct/MFI of CD58 for the control CAR. The bottom panel of FIG. 43 depicts follow up screen of top 5 miR-shRNAs transduced into RFX5 knockout primary T cells along with 5 controls. Percentages above bars are the (CAR⁺ CD58 MFI of each construct/(CAR⁺ CD58 MFI NTC CAR −CAR⁺ CD58 MFI CD58 knockout_RFX5 knockout)). The sequences of CD58 miR-shRNAs, reference ID #s, mIR backbone, shRNA sequence, and orientation are listed in Table 17.

Generation of allogeneic effector T cells. PBMCs from a non-HLA matched human donor were stimulated with irradiated (40 Gy) PBMCs from the human donor used to make HLA class I and II negative T cells at a 1:1 ratio in media [TexMACS (Miltenyi #170-076-307) and 100 IU/ml Penicillin+100 μg/ml Streptomycin (Gibco #15140-122)], without IL-2 at 2×10⁶ cells/ml. After 2 days, an equal volume of media containing 60 IU/ml hIL-21S (Miltenyi #130-097-748) was added to achieve a final concentration of 30 IU/ml IL-21S. After 5 additional days of culture, cells were washed, resuspended into media with 30 IU/ml IL-21S and restimulated with another round of irradiated (40 Gy) PBMCs from the human donor used to make HLA class I and II negative T cells at a 1:1 ratio. Following another 2 days of culture, an equal volume of media was added and IL-21S was supplied to 200 IU/ml. Two days later, fresh media and 200 IU/ml IL-21S was added to dilute the cells to 0.5×10⁶ cells/ml, and cells were cultured for another 3 days before being cryopreserved in CS10. In some experiments, alloreactive effector cells were separated into purified T cells (mixture of CD4⁺ and CD8⁺) and purified NK cells (CD56+CD3−) with an EasySep CD56+ isolation kit (STEMCELL Technologies #17855).

Isolation of primary NK cells. Human NK cells were isolated from leukapheresis (StemExpress and HemaCare) using the NK cell Isolation kit (Miltenyi #130-092-657) and program on a CliniMACS Prodigy (Miltenyi Biotec). NK cells were cryopreserved at 10⁶ cells/mL in Cryostor CS10 (Sigma #C2874-100ML).

Allogeneic response assays. Cryopreserved gene edited T cells (“targets”) were thawed, enriched for CAR⁺ cells by magnetic isolation with anti-CAR AF647 and anti-AF647 microbeads using the Miltenyi AutoMACS, and rested overnight in RPMI+L-glutamine (Gibco #11875-093), 10% FBS (Gibco #16140-071), 100 IU/ml Penicillin+100 μg/ml Streptomycin (Gibco #15140-122), 1 mM Sodium Pyruvate (Gibco #11360-070), 10 mM HEPES (Gibco #15630-080), and 55 μM 2-mercaptoethanol (Gibco #21985-023), herein referred to as “assay media,” supplemented with 30 IU/ml hIL-21S (Miltenyi #130-097-748), at 1×10⁶ cells/ml. The next day, target T cells were washed to remove IL-2 and seeded in 96 well U-bottom plates in assay media at 10,000 cells/well. Allogeneic effector T cells were also thawed and rested one day prior to experiment setup in assay media supplemented with 30 IU/ml hIL-21S. Primary NK cells were also thawed and rested one day prior to experiment setup in assay media supplemented with 0.2 ng/mL IL-2 (Gibco #PHC0026). The next day, allogeneic effector T cells or NK cells were labelled with 1 μM Cell Trace Violet (Thermo #C34557) in PBS for 20 mins at 37° C., washed twice with assay media, and seeded with targets for cytotoxicity assays (various E:Ts). Cells were analyzed by flow cytometry 18-24 hours after plating. Normalized target cell viability was calculated as: 100*(% live targets at E:T/% live targets alone), where 100% indicates complete survival. Survival (Area under the Curve) Calculation: Normalized target viability was calculated as: % live targets at E:T/% live targets alone. Area under the curve (AUC) was calculated for each edit. Normalized AUC for NK cells was calculated by: AUC per edit/AUC for NTC. Normalized AUC for T cells was calculated by: AUC per edit/AUC of RFX5 knockout/B2M knockout.

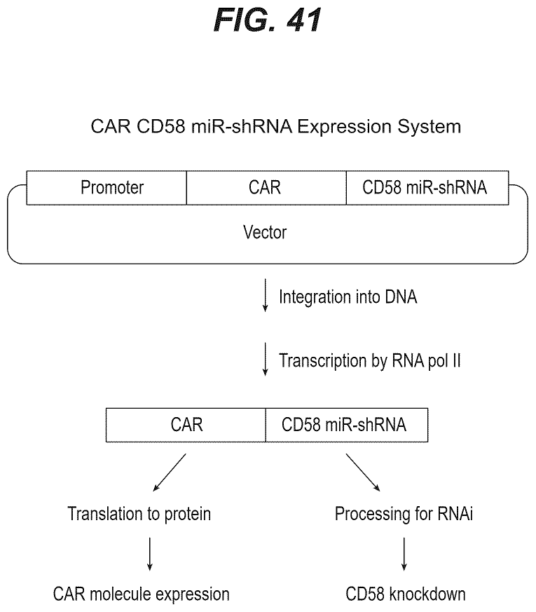

FIG. 41 shows a diagram of the dual CAR and CD58 miR-shRNA Expression System, where a single pol II promoter drives expression of a transcript encoding both the CAR and CD58 miR-shRNA. The CD58 miR-shRNA will be processed for RNAi by Drosha and Dicer and then loaded into RISC (RNA-induced silencing complex) for silencing of the endogenous CD58 gene. The CAR portion will be translated to protein for CAR molecule expression.

Fifty-five dual CAR and CD58 miR-shRNA constructs were designed and evaluated using a lentiviral transduction system in primary human T cells (Table 17).

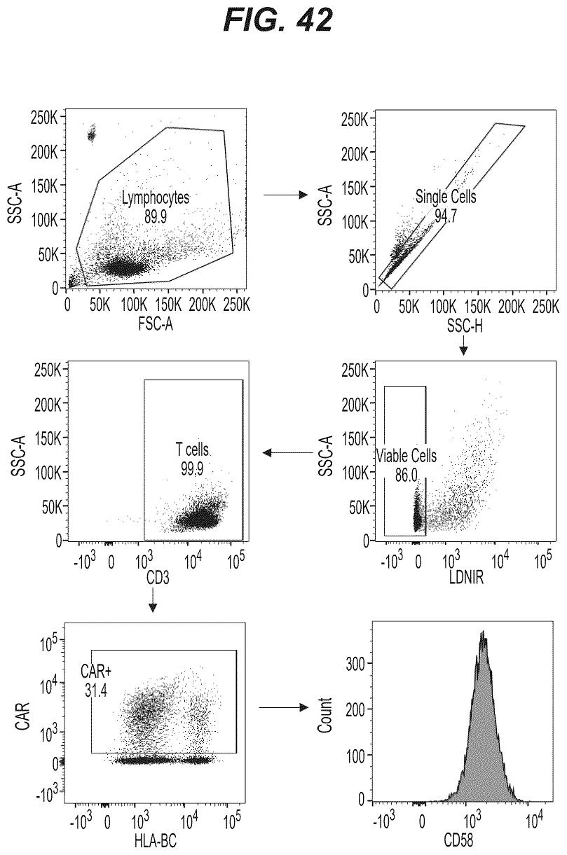

FIG. 42 shows the gating strategy for evaluating CAR expression and knockdown of endogenous CD58. FIG. 43 shows the results from screening all 55 constructs, with knockdown evaluated on CAR⁺ cells. All constructs showed some degree of knockdown of CD58, and five high performing constructs were validated in a follow up experiment where they were transduced into RFX5 knockout T cells and were directly compared to dual RFX5/CD58 knockout. The best three constructs demonstrated a reduction in CD58 expression of 90%, 83%, and 72%.

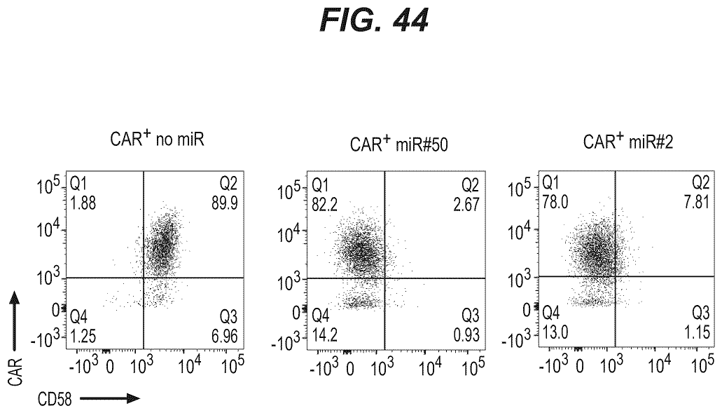

Constructs #50 and #2 were further evaluated for their ability to confer functional immune-evasion properties to RFX5 knockout primary T cells. T cells expressing the CAR were enriched with magnetic beads, and the expression of the CAR and endogenous CD58 are shown in FIG. 44.

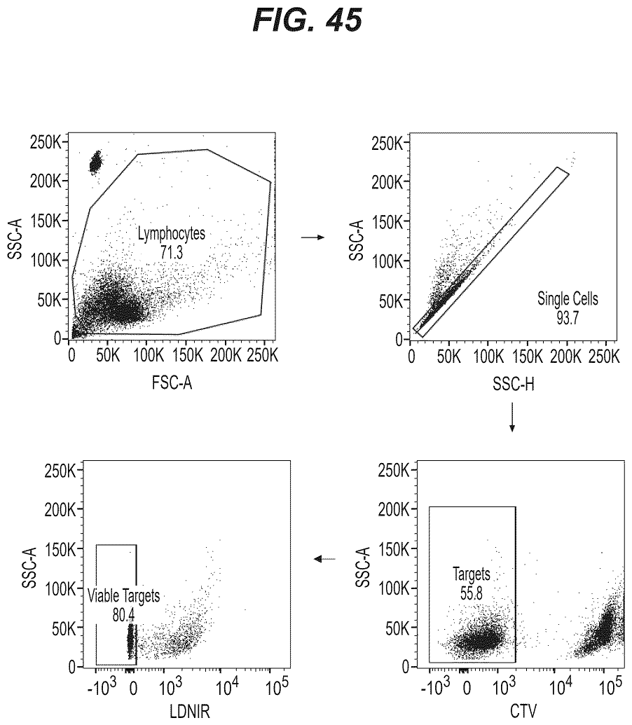

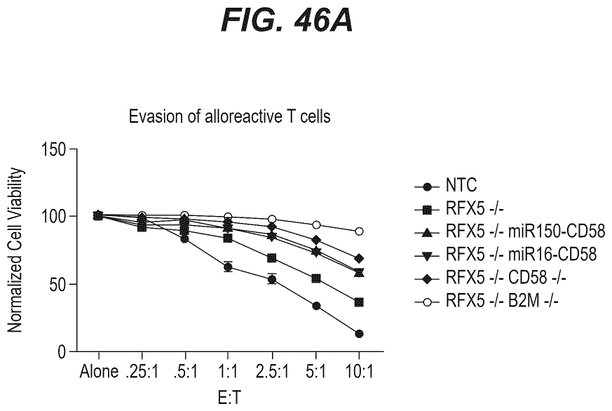

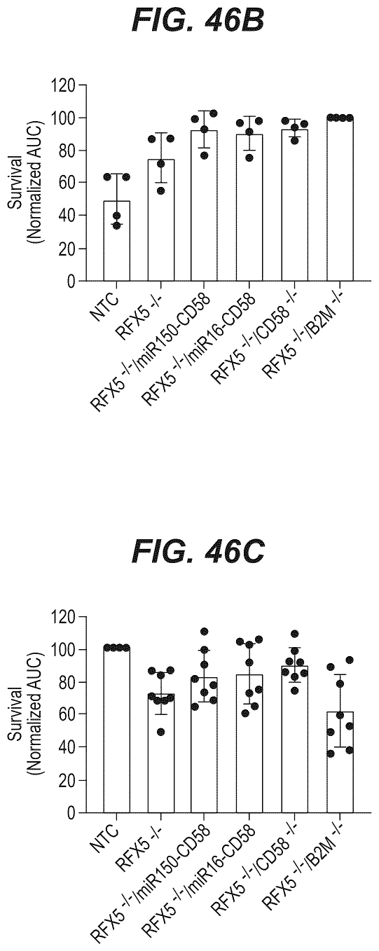

FIGS. 45 and 46A-46C show that CD58 knockdown improves the ability of RFX5 knockout cells to evade alloreactive effector T cells and NK cells. CAR-enriched gene edited or control T cells were co-cultured with alloreactive effector T cells or primary NK cells in an overnight cytotoxicity assay. The gating strategy for analysis of the co-culture experiments is shown in FIG. 45.

FIG. 46A shows data from one representative experiment with a single target human donor co-cultured with a single effector human donor. FIG. 46B-46C show aggregate data with an Area under the Curve (AUC) calculation from multiple experiments with several target and effector human donors.

FIGS. 46A and 46B show that when co-cultured with alloreactive T cells, the CD58 miR-shRNAs introduced to RFX5 knockout T cells from two different human donors improved evasion relative to RFX5 knockout alone. The level of evasion of alloreactive T cells for the two CD58-miR-shRNAs was equivalent to that achieved with a full CD58 knockout.

FIG. 46C shows that when co-cultured with primary NK cells from four different human donors, the CD58 miR-shRNAs introduced to RFX5 knockout T cells from two different human donors improved evasion relative to RFX5 knockout alone. The level of evasion of NK cells for the two CD58-miR-shRNAs was intermediate compared to that achieved with a full CD58 knockout.

Overall, the data demonstrate that the dual CAR and CD58 miR-shRNA Expression System can lead to both expression of a CAR and knockdown of endogenous CD58, and that CD58 miR-shRNAs were identified that lead to efficient knockdown of CD58 and functional immune-evasion of alloreactive T cells and NK cells.

TABLE 17
List of CD58 miR-shRNAs.

| SEQ | | | | | |
|---|---|---|---|---|---|
| ID | | miR | | Seq. Listing | Sequences |
| NO | ID | backbones | shRNA | Orientation | (written 5′ to 3′) |
| 74 | 1 | miR-30a | CD58#8 | 3′ | CAGAAGGCTCGAGAAGGTATATTGCTGTTGA |
| | | | | | CAGTGAGCGACGCTCACTATGTACAACTTAAC |
| | | | | | TGTGAAGCCACAGATGGGTTAAGTTGTAGAT |
| | | | | | AGTGAGGGCTGCCTACTGCCTCGGACTTCAAG |
| | | | | | GGGCTTGCGGCCGC |
| 75 | 2 | miR-16-1 | CD58#8 | 5′ | AACTTATGATAGCAATGTCAGCAGTGCCTTTA |
| | | | | | AGTTGTAGATAGTGAGGCTTTAAGATTCTAAA |
| | | | | | ATTATATCCTCACTATCCACAACTTACAAGTA |
| | | | | | AGGTTGACCATACTCTACAGTTGTT |
| 76 | 3 | miR-16-1 | CD58#8 | 3′ | AACTTATGATAGCAATGTCAGCAGTGCCTACT |
| | | | | | CACTATGTACAACTTAACGTTAAGATTCTAAA |
| | | | | | ATTATCCTTAAGTIGTAGATAGTGAGGCAGTA |
| | | | | | AGGTTGACCATACTCTACAGTTGTT |
| 77 | 4 | miR-16-1 | CD58#5 | 5 | AACTTATGATAGCAATGTCAGCAGTGCCTTAT |
| | | | | | ATACTGGTTGAGTTACGTTTTAAGATTCTAAA |
| | | | | | ATTATATCGTAACTCAAGCAGTATATCAAGTA |
| | | | | | AGGTTGACCATACTCTACAGTTGTT |
| 78 | 5 | miR-16-1 | CD58#4 | 5′ | AACTTATGATAGCAATGTCAGCAGTGCCTAAT |
| | | | | | AATGGATTGCTAAGAGTACTTAAGATTCTAAA |
| | | | | | ATTATGCACTCTTAGCATTCCATTATCAAGTA |
| | | | | | AGGTTGACCATACTCTACAGTTGTT |
| 79 | 6 | miR-122 | CD58#8 | 5′ | TTCGTGGCTACAGAGTTTCCTTAGCAGAGCTG |
| | | | | | TTAAGTIGTAGATAGTGAGGCTTGTCTAAACT |
| | | | | | ATAGCCTCACTATCAACAACTTACTAGCTACT |
| | | | | | GCTAGGCAATCCTTCCCTCGATAAATG |
| 80 | 7 | miR-122 | CD58#8 | 3′ | TTCGTGGCTACAGAGTTTCCTTAGCAGAGCAG |
| | | | | | ACTCACTATGTACAACTTAATGTGTCTAAACT |
| | | | | | ATCATTAAGTIGTAGATAGTGAGGCTGCTACT |
| | | | | | GCTAGGCAATCCTTCCCTCGATAAATG |
| 81 | 8 | miR-122 | CD58#5 | 5 | TTCGTGGCTACAGAGTTTCCTTAGCAGAGCTG |
| | | | | | TATATACTGGTTGAGTTACGTTTGTCTAAACT |
| | | | | | ATAACGTAACTCAAGCAGTATATCTAGCTACT |
| | | | | | GCTAGGCAATCCTTCCCTCGATAAATG |
| 82 | 9 | miR-122 | CD58#4 | 5′ | TTCGTGGCTACAGAGTTTCCTTAGCAGAGCTG |
| | | | | | AATAATGGATTGCTAAGAGTACTGTCTAAACT |
| | | | | | ATGTACTCTTAGCATTCCATTATCTAGCTACT |
| | | | | | GCTAGGCAATCCTTCCCTCGATAAATG |
| 83 | 10 | miR-E | CD58#8 | 5 | CGACTTCTTAACCCAACAGAAGGCTCGAGAA |
| | | | | | GGTATATTGCTGTTGACAGTGAGCGCGCCTCA |
| | | | | | CTATCTACAACTTAATAGTGAAGCCACAGATG |
| | | | | | TATTAAGTIGTAGATAGTGAGGCTTGCCTACT |
| | | | | | GCCTCGGACTTCAAGGGGCTAGAATTCGCGG |
| | | | | | CCGCAACCAGCGGCGGCTCCTCTCCCCATGGC |
| | | | | | CCTGCACTTGCGCTGGGTGATCTTGCTGGGCT |
| | | | | | CAGACCAGATCACCCTGCGCAAGTCCAGGGA |
| | | | | | CCTGGGGACCCCGGCACCGGCAGGCCGGATC |
| | | | | | C |
| 84 | 11 | miR-21 | CD58#8 | 5′ | CATCTCCATGGCTGTACCACCTTGTCGGTTAA |
| | | | | | GTTGTAGATAGTGAGGCCTGTTGAATCTCATG |
| | | | | | GCCTCACTATCCACAACTTATTCTGACATTTT |
| | | | | | GGTATCTTTCATCTGACCA |
| 85 | 12 | miR- | CD58#8 | 5′ | CATCTCCATGGCTGTACCACCTTGTCGGTTAA |
| | | 21_ | | | GTTGTAGATAGTGAGGCACGTTGAATCTCATG |
| | | modified | | | GCCTCACTATCCACAACTTATTCTGACATTTT |
| | | | | | GGTATCTTTCATCTGACCA |
| 86 | 13 | miR-21 | CD58#8 | 3 | CATCTCCATGGCTGTACCACCTTGTCGGGGTC |
| | | | | | ACTATGTACAACTTAAACTGTTGAATCTCATG |
| | | | | | GTTAAGTTGTAGATAGTGAGGCCTGACATTTT |
| | | | | | GGTATCTTTCATCTGACCA |
| 87 | 14 | miR-21 | CD58#5 | 5 | CATCTCCATGGCTGTACCACCTTGTCGGTATA |
| | | | | | TACTGGTTGAGTTACGTCTGTTGAATCTCATG |
| | | | | | GCGTAACTCAAGCAGTATATTTCTGACATTTT |
| | | | | | GGTATCTTTCATCTGACCA |
| 88 | 15 | miR- | CD58#5 | 5 | CATCTCCATGGCTGTACCACCTTGTCGGTATA |
| | | 21_ | | | TACTGGTTGAGTTACGTCCGTTGAATCTCATG |
| | | modified | | | GCGTAACTCAAGCAGTATATTTCTGACATTTT |
| | | | | | GGTATCTTTCATCTGACCA |
| 89 | 16 | miR-21 | CD58#4 | 5 | CATCTCCATGGCTGTACCACCTTGTCGGAATA |
| | | | | | ATGGATTGCTAAGAGTACTGTTGAATCTCATG |
| | | | | | GACTCTTAGCACTCCATTATAACTGACATTTT |
| | | | | | GGTATCTTTCATCTGACCA |
| 90 | 17 | miR-185 | CD58#8 | in | GGGCCTGGCTCGAGCAGGGGGCGAGGGATTT |
| | | | | | AAGTTGTAGATAGTGAGGCTTGGTCCCCTCCC |
| | | | | | CCCTCACTATCAACAACTTACGTCCTTCCCTC |
| | | | | | CCAATGACCGCGTCTTCGTC |
| 91 | 18 | miR- | CD58#8 | 5 | GGGCCTGGCTCGAGCAGGGGGCGAGGGATTT |
| | | 185_ | | | AAGTTGTAGATAGTGAGGCTTGGTCCCCTCCC |
| | | modified | | | GCCTCACTATCAACAACTTACGTCCTTCCCTC |
| | | | | | CCAATGACCGCGTCTTCGTC |
| 92 | 19 | miR-185 | CD58#8 | 3′ | GGGCCTGGCTCGAGCAGGGGGCGAGGGATAC |
| | | | | | TCACTATGTACAACTTAAGATGGTCCCCTCCC |
| | | | | | CTTAAGTTGTAGATAGTGAGGCTCCTTCCCTC |
| | | | | | CCAATGACCGCGTCTTCGTC |
| 93 | 20 | miR-185 | CD58#5 | 5 | GGGCCTGGCTCGAGCAGGGGGCGAGGGATTA |
| | | | | | TATACTGGTTGAGTTACGTTTGGTCCCCTCCC |
| | | | | | CCGTAACTCAAGCAGTATATCGTCCTTCCCTC |
| | | | | | CCAATGACCGCGTCTTCGTC |
| 94 | 21 | miR- | CD58#5 | 5 | GGGCCTGGCTCGAGCAGGGGGCGAGGGATTA |
| | | 185_ | | | TATACTGGTTGAGTTACGTTTGGTCCCCTCCC |
| | | modified | | | GCGTAACTCAAGCAGTATATCGTCCTTCCCTC |
| | | | | | CCAATGACCGCGTCTTCGTC |
| 95 | 22 | miR-185 | CD58#4 | 5 | GGGCCTGGCTCGAGCAGGGGGCGAGGGATAA |
| | | | | | TAATGGATTGCTAAGAGTACTGGTCCCCTCCC |
| | | | | | CACTCTTAGCATTCCATTATCGTCCTTCCCTCC |
| | | | | | CAATGACCGCGTCTTCGTC |
| 96 | 23 | miR- | CD58#4 | 5′ | GGGCCTGGCTCGAGCAGGGGGCGAGGGATAA |
| | | 185_ | | | TAATGGATTGCTAAGAGTACTGGTCCCCTCCC |
| | | modified | | | TACTCTTAGCATTCCATTATCGTCCTTCCCTCC |
| | | | | | CAATGACCGCGTCTTCGTC |
| 97 | 24 | miR-106b | CD58#8 | 5′ | GCTCGAGCCCTGCCGGGGCTTAAGTTGTAGAT |
| | | | | | AGTGAGGCAGTGGTCCTCTCCGTGCTATGCAC |
| | | | | | TATCAACAACTTCGTGCTCCAGCAGGGCACGC |
| | | | | | AAAGCTT |
| 98 | 25 | miR- | CD58#8 | 5′ | GCTCGAGCCCTGCCGGGGCTTAAGTTGTAGAT |
| | | 106b_ | | | AGTGAGGCAGTGGTCCTCTCCGTGCTGTGCAC |
| | | modified | | | TATCAACAACTTCGTGCTCCAGCAGGGCACGC |
| | | | | | AAAGCTT |
| 99 | 26 | miR-106b | CD58#8 | 3′ | GCTCGAGCCCTGCCGGGGGAGCACTATCAAC |
| | | | | | AACTTCCCTAGTGGTCCTCTCCGTGCTATTAA |
| | | | | | GTTGTAGATAGTGAGGCCTCCAGCAGGGCAC |
| | | | | | GCAAAGCTT |
| 100 | 27 | miR-106b | CD58#5 | 5 | GCTCGAGCCCTGCCGGGGCTATATACTGGTTG |
| | | | | | AGTTACGTAGTGGTCCTCTCCGTGCTAGAAAC |
| | | | | | TCAAGCAGTATAGCTGCTCCAGCAGGGCACG |
| | | | | | CAAAGCTT |
| 101 | 28 | miR-106b | CD58#4 | 5 | GCTCGAGCCCTGCCGGGGCAATAATGGATTG |
| | | | | | CTAAGAGTAAGTGGTCCTCTCCGTGCTATCCT |
| | | | | | TAGCATTCCATTAGCTGCTCCAGCAGGGCACG |
| | | | | | CAAAGCTT |
| 102 | 29 | miR- | CD58#4 | 5 | GCTCGAGCCCTGCCGGGGCAATAATGGATTG |
| | | 106b_ | | | CTAAGAGTAAGTGGTCCTCTCCGTGCTTTCCT |
| | | modified | | | TAGCATTCCATTAGCTGCTCCAGCAGGGCACG |
| | | | | | CAAAGCTT |
| 103 | 30 | miR-20 | CD58#8 | 3′ | ATGTAGAATCTGCCTGGTCTATCTGATGTGAC |
| | | dr_t5-19 | | | AGCTTCTGTAGCACTCTCACTATCTACAACTT |
| | | | | | AAGTAGTGTTTAGTTATCTATTAAGTTGTAGA |
| | | | | | TAGTGAGTTGTACTGCTAGCTGTAGAACTCCA |
| | | | | | GCTT |
| 104 | 31 | miR-20 | CD58#8 | 5′ | ATGTAGAATCTGCCTGGTCTATCTGATGTGAC |
| | | dr_t5-19 | | | AGCTTCTGTAGCACTTAAGTIGTAGATAGTGA |
| | | | | | GGTAGTGTTTAGTTATCTACTCACTATCTACA |
| | | | | | ACTTTTAGTACTGCTAGCTGTAGAACTCCAGC |
| | | | | | TT |
| 105 | 32 | miR-20 | CD58#5 | 5′ | ATGTAGAATCTGCCTGGTCTATCTGATGTGAC |
| | | dr_t5-19 | | | AGCTTCTGTAGCACTATATACTGGTTGAGTTA |
| | | | | | CGTAGTGTTTAGTTATCTAGTAACTCAACCAG |
| | | | | | TATAAAAGTACTGCTAGCTGTAGAACTCCAGC |
| | | | | | TT |
| 106 | 33 | miR-20 | CD58#4 | 5′ | ATGTAGAATCTGCCTGGTCTATCTGATGTGAC |
| | | dr_t5-19 | | | AGCTTCTGTAGCACAATAATGGATTGCTAAGA |
| | | | | | GGTAGTGTTTAGTTATCTACTCTTAGCAATCC |
| | | | | | ATTAAATGTACTGCTAGCTGTAGAACTCCAGC |
| | | | | | TT |
| 107 | 34 | miR-25 | CD58#8 | 3′ | CAGCGGCGGCTCCTGGCCAGTGTTGTCCCTCT |
| | | | | | CTATCATCAACTTATACTGGACGCTGCCCTGG |
| | | | | | GTTAAGTTGTAGATAGTGAGGCTCAGTGCCG |
| | | | | | GCCCCCGGCACCGGCAGGCC |
| 108 | 35 | miR-25 | CD58#8 | 5′ | CAGCGGCGGCTCCTGGCCAGTGTTGAGTTAA |
| | | | | | GTTGTAGATAGTGAGGCCTGGACGCTGCCCTG |
| | | | | | GGGGTCACTAATTACAAGTTAAGACAGTGCC |
| | | | | | GGCCCCCGGCACCGGCAGGCC |
| 109 | 36 | miR-25 | CD58#5 | 5′ | CAGCGGCGGCTCCTGGCCAGTGTTGAGTATAT |
| | | | | | ACTGGTTGAGTTACGTCTGGACGCTGCCCTGG |
| | | | | | GAATAACTTTTCCAGTTTATAGACAGTGCCGG |
| | | | | | CCCCCGGCACCGGCAGGCC |
| 110 | 37 | miR-25 | CD58#4 | 5′ | CAGCGGCGGCTCCTGGCCAGTGTTGAGAATA |
| | | | | | ATGGATTGCTAAGAGTACTGGACGCTGCCCTG |
| | | | | | GGTCTCTTACCGATCCAATATTGACAGTGCCG |
| | | | | | GCCCCCGGCACCGGCAGGCC |
| 111 | 38 | miR-26 | CD58#8 | 5 | CAGCGGCGGCTCCTGTGGCCTCGTTAAGTTGT |
| | | | | | AGATAGTGAGGCTGTGCAGGTCCCAATGGGC |
| | | | | | CTCACTGATTGCAACTTCACGGGGACGCCCCG |
| | | | | | GCACCGGCAGGCC |
| 112 | 39 | miR-26 | CD58#8 | 3 | CAGCGGCGGCTCCTGTGGCCTAGCGTCACTAT |
| | | | | | CTCCAACTTAACTGTGCAGGTCCCAATGGGTT |
| | | | | | AAGTTGTAGATAGTGAGGCTGGGACGCCCCG |
| | | | | | GCACCGGCAGGCC |
| 113 | 40 | miR-26 | CD58#5 | 5 | CAGCGGCGGCTCCTGTGGCCTCGTATATACTG |
| | | | | | GTTGAGTTACGTTGTGCAGGTCCCAATGGGCG |
| | | | | | TAACTTTATCAGTATAAACGGGGACGCCCCG |
| | | | | | GCACCGGCAGGCC |
| 114 | 41 | miR-26 | CD58#4 | 5 | CAGCGGCGGCTCCTGTGGCCTCGTATAATGGA |
| | | | | | TTGCTAAGAGTACGTGCAGGTCCCAATGGTAC |
| | | | | | TCTTGGAGATCCATTAATCGGGGACGCCCCGG |
| | | | | | CACCGGCAGGCC |
| 115 | 42 | miR-204 | CD58#8 | 5′ | GGCTACAGTCTTTCTTCATGTGACTCGTGGAC |
| | | | | | TTAAGTTGTAGATAGTGAGGCTGAGAATATAT |
| | | | | | GAAGGAGCCCACTAACTACAACTTACGTTCA |
| | | | | | ATTGTCATCACTGGC |
| 116 | 43 | miR-204 | CD58#8 | 3′ | GGCTACAGTCTTTCTTCATGTGACTCGTGGGC |
| | | | | | ATCACTATCTAGAACTTCAACTGAGAATATAT |
| | | | | | GAAGGAGTTAAGTTGTAGATAGTGAGGCTCA |
| | | | | | ATTGTCATCACTGGC |
| 117 | 44 | miR-204 | CD58#5 | 5′ | GGCTACAGTCTTTCTTCATGTGACTCGTGGAC |
| | | | | | TATATACTGGTTGAGTTACGTTGAGAATATAT |
| | | | | | GAAGGAACGAACTCTACCAGTATATCGTTCA |
| | | | | | ATTGTCATCACTGGC |
| 118 | 45 | miR-204 | CD58#4 | 5 | GGCTACAGTCTTTCTTCATGTGACTCGTGGAC |
| | | | | | AATAATGGATTGCTAAGAGTACGAGAATATA |
| | | | | | TGAAGGGTACCTTAGGAATCCATTATAGTTCA |
| | | | | | ATTGTCATCACTGGC |
| 119 | 46 | miR-204 | CD58#8 | 5′ | TTCATGTGACTCGTGGACTTAAGTTGTAGATA |
| | | short | | | GTGAGGCTGAGAATATATGAAGGAGCCCACT |
| | | | | | AACTACAACTTACGTTCAATTGTCATCACTGG |
| | | | | | C |
| 120 | 47 | miR-204 | CD58#8 | 3′ | TTCATGTGACTCGTGGGCATCACTATCTAGAA |
| | | short | | | CTTCAACTGAGAATATATGAAGGAGTTAAGTT |
| | | | | | GTAGATAGTGAGGCTCAATTGTCATCACTGGC |
| 121 | 48 | miR-204 | CD58#5 | 5′ | TTCATGTGACTCGTGGACTATATACTGGTTGA |
| | | short | | | GTTACGTTGAGAATATATGAAGGAACGAACT |
| | | | | | CTACCAGTATATCGTTCAATTGTCATCACTGG |
| | | | | | C |
| 122 | 49 | miR-204 | CD58#4 | 5 | TTCATGTGACTCGTGGACAATAATGGATTGCT |
| | | short | | | AAGAGTACGAGAATATATGAAGGGTACCTTA |
| | | | | | GGAATCCATTATAGTTCAATTGTCATCACTGG |
| | | | | | C |
| 123 | 50 | miR-150 | CD58#8 | 5′ | GAATTCCAGCGGCGGCTCCTCTCCCCATGGCC |
| | | | | | CTGTTAAGTTGTAGATAGTGAGGTGCTGGGCT |
| | | | | | CAGACCCCTCACTATCAACAACTTACCAGGG |
| | | | | | ACCTGGGGACCCCGGCACCGGCAGGCCGGAT |
| | | | | | CC |
| 124 | 51 | miR-150 | CD58#8 | 3′ | GAATTCCAGCGGCGGCTCCTCTCCCCATGGCC |
| | | | | | CTGGCTCACTATGTACAACTTAATGCTGGGCT |
| | | | | | CAGACCTTAAGTTGTAGATAGTGAGGCAGGG |
| | | | | | ACCTGGGGACCCCGGCACCGGCAGGCCGGAT |
| | | | | | CC |
| 125 | 52 | miR-93 | CD58#8 | 5 | GAATTCAGTCCTGGGGGCTCCTTAAGTTGTAG |
| | | | | | ATAGTGAGGCTTGTGATTACCCAACAGCCCAC |
| | | | | | ATCTATCAACTTACCCGAGCCCCCGGGATCCG |
| | | | | | TTC |
| 126 | 53 | miR-93 | CD58#8 | 3′ | GAATTCAGTCCTGGGGGCTCCACTCACTTCTA |
| | | | | | CAAACTTAAAGTGTGATTACCCAACCTTTAAG |
| | | | | | TTGTAGATAGTGAGGCTCGAGCCCCCGGGAT |
| | | | | | CCGTTC |
| 127 | 54 | miR-93 | CD58#5 | 5 | GAATTCAGTCCTGGGGGCTCCTATATACTGGT |
| | | | | | TGAGTTACGTTTGTGATTACCCAACAACGAAC |
| | | | | | CAACCTAGTATATCCCGAGCCCCCGGGATCCG |
| | | | | | TTC |
| 128 | 55 | miR-93 | CD58#4 | 5 | GAATTCAGTCCTGGGGGCTCCAATAATGGATT |
| | | | | | GCTAAGAGTACTGTGATTACCCAACGTACCTT |
| | | | | | GCAATACCATTATCCCGAGCCCCCGGGATCCG |
| | | | | | TTC |

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1. A method of hypoimmunogenicity, such as engineering hypoimmunogenicity, comprising:
a) reprogramming an immunogenic human cell to produce an induced pluripotent (iPS) human cell, wherein the immunogenic human cell comprises a heterodimeric T-cell receptor comprising a γ chain and a δ chain;
b) genetically modifying a beta-2-microglobulin (B2M) gene of the iPS human cell, wherein genetically modifying the B2M gene reduces expression of the B2M protein by the iPS human cell;
c) forming at least one embryoid body or multicellular body from the cell of step b) to produce at least one hypoimmunogenic cell, such as at least one engineered hypoimmunogenic cell;
d) subjecting the hypoimmunogenic cell, such as the engineered hypoimmunogenic cell, to an immune system; and
e) determining immunogenicity of the hypoimmunogenic cell, such as the engineered hypoimmunogenic cell, wherein the immunogenicity is altered as compared to an iPS human cell where the B2M gene is not genetically modified,
optionally wherein step b) further comprises genetically modifying one or more of a class II major histocompatibility complex transactivator (CIITA) gene, a regulatory factor X (RFX) gene, and a CD58 gene of the iPS human cell.

2-3. (canceled)

4. A method of producing an hypoimmunogenic cell, such as an engineered hypoimmunogenic cell, from an immunogenic cell, comprising:
(i) genetically modifying a beta-2-microglobulin (B2M) gene in the immunogenic cell, wherein genetically modifying the B2M gene reduces expression of the B2M protein in said cell, and
(ii) optionally further genetically modifying one or more genes selected from a class II major histocompatibility complex transactivator (CIITA) gene, a CD58 gene, and a regulatory factor X (RFX) gene in said immunogenic cell, wherein genetically modifying the one or more genes reduces expression of the corresponding one or more proteins in said immunogenic cell, wherein said method results in production of the hypoimmunogenic cell, such as the engineered hypoimmunogenic cell, which has one or more of the following properties:
a) having a reduced immunogenicity upon the hypoimmunogenic cell's, such as the engineered hypoimmunogenic cell's, presence in an allogeneic or non-MHC matched subject, as compared to a corresponding immunogenic cell, but without the genetic modification(s) of (i) and (ii);
b) causing a reduced immune response to said hypoimmunogenic cell, such as the engineered hypoimmunogenic cell, upon its presence in an allogeneic or non-MHC matched subject, as compared to a corresponding immunogenic cell, but without the genetic modification(s) of (i) and (ii); and
c) causing a reduced alloreactive T cell cytotoxicity to said hypoimmunogenic cell, such as the engineered hypoimmunogenic cell, upon its presence in an allogeneic or non-MHC matched subject, as compared to a corresponding immunogenic cell, but without the genetic modification(s) of (i) and (ii).

5. (canceled)

6. The method of claim 1, wherein the hypoimmunogenic cell, such as the engineered hypoimmunogenic cell, comprises a T-cell receptor (TCR) comprising a γ chain and a δ chain.

7. The method of claim 1, wherein the immunogenic human cell or immunogenic cell is an immune cell, optionally selected from T cells, natural killer (NK) cells, B cells, and hematopoietic stem cells (HSCs).

8. The method of claim 1, wherein the reduced immunogenicity of the hypoimmunogenic cell, such as the engineered hypoimmunogenic cell, comprises one or more of the following:
i) a reduced or ablated myeloid cell response to the hypoimmunogenic cell, such as the engineered hypoimmunogenic cell, upon the cell's presence in an allogeneic or non-MHC matched subject, as compared to a cell corresponding to the cell that was modified but without said genetic modification(s);
ii) a reduced or ablated T cell response to the hypoimmunogenic cell, such as the engineered hypoimmunogenic cell, upon the cell's presence in an allogeneic or non-MHC matched subject, as compared to a cell corresponding to the cell that was modified but without said genetic modification(s);
iii) a reduced or ablated natural killer (NK) cell response to the hypoimmunogenic cell, such as the engineered hypoimmunogenic cell, upon the cell's presence in an allogeneic or non-MHC matched subject, as compared to a cell corresponding to the cell that was modified but without said genetic modification(s);
iv) a reduced or ablated neutralizing antibody response to the hypoimmunogenic cell, such as the engineered hypoimmunogenic cell, upon the cell's presence in an allogeneic or non-MHC matched subject, as compared to a cell corresponding to the cell that was modified but without said genetic modification(s);
v) a reduced or ablated MHC class II mediated response to the hypoimmunogenic cell, such as the engineered hypoimmunogenic cell, upon the cell's presence in an allogeneic or non-MHC matched subject, as compared to a cell corresponding to the cell that was modified but without said genetic modification(s);
vi) a reduced or ablated neutralizing MHC class I mediated response to the hypoimmunogenic cell, such as the engineered hypoimmunogenic cell, upon the cell's presence in an allogeneic or non-MHC matched subject, as compared to a cell corresponding to the cell that was modified but without said genetic modification(s); and
vii) a reduced or ablated allogeneic host versus graft rejection of the hypoimmunogenic cell, such as the engineered hypoimmunogenic cell, upon the cell's presence in an allogeneic subject, as compared to a cell corresponding to the cell that was modified but without said genetic modification(s).

9. The method of claim 4, wherein the immunogenic cell is a human cell.

10. The method of claim 9, wherein in the hypoimmunogenic cell the:
i) expression of HLA class II molecules is reduced or ablated;
ii) expression of HLA-A, HLA-B, and/or HLA-C is reduced; and
iii) expression of HLA-E is reduced but remains detectable.

11. The method of claim 4, wherein the method comprises forming at least one embryoid body or multicellular body from the genetically modified cell to produce the hypoimmunogenic cell, such as the engineered hypoimmunogenic cell.

12. The method of claim 4, further comprising determining immunogenicity of the hypoimmunogenic cell, such as the engineered hypoimmunogenic cell.

13. The method of claim 1, further comprising administering the hypoimmunogenic cell, such as the engineered hypoimmunogenic cell to an allogeneic or non-MHC matched subject.

14. The method of claim 1, wherein the immunogenicity of the hypoimmunogenic cell, such as the engineered hypoimmunogenic cell, is altered as compared to an immunogenic cell or an immunogenic human cell or an iPS human cell or an iPS cell, where the only difference between the hypoimmunogenic cell, such as the engineered hypoimmunogenic cell, and the immunogenic cell or the immunogenic human cell or the iPSC human cell or the iPS cell is that the B2M gene and optionally one or more of the RFX gene, the CIITA gene, and the CD58 gene is not genetically modified in the immunogenic cell or the immunogenic human cell or the iPSC human cell.

15. The method of claim 1, wherein the immunogenic human cell or the immunogenic cell is allogeneic or non-HLA matched or non-MHC matched to cells, receptors, or polypeptides of the immune system of a recipient subject.

16-22. (canceled)

23. The method of claim 1, further comprising genetically modifying a RFX gene, wherein the RFX gene is RFX5, RFXANK or RFXAP.

24-26. (canceled)

27. The method of claim 23, wherein genetically modifying the RFX gene results in reducing or ablating MHC class II mediated response to the hypoimmunogenic cell, such as the engineered hypoimmunogenic cell.

28. The method of claim 23, wherein genetically modifying the RFX gene results in reducing or neutralizing MHC class I mediated response to the hypoimmunogenic cell, such as the engineered hypoimmunogenic cell.

29. The method of claim 1, further comprising genetically modifying a CIITA gene, wherein genetically modifying the CIITA gene results in reducing or ablating expression of HLA class II molecules on the hypoimmunogenic cell, such as the engineered hypoimmunogenic cell.

30. The method of claim 1, further comprising genetically modifying a CD58 gene, wherein genetically modifying the CD58 gene eliminates or reduces the CD58 expression.

31. (canceled)

32. The method of claim 1, wherein genetically modifying the B2M gene comprises:
(i) modifying the DNA sequence of the B2M gene, optionally through a CRISPR-Cas system;
(ii) repressing transcription or translation of the B2M mRNA through a RNAi system, optionally the RNAi system comprises shRNA, siRNA, or miR-adapted shRNA; or
(iii) reducing or ablating transcription of the B2M gene, optionally through recruiting or directing transcriptional repressors to the B2M gene.

33. (canceled)

34. The method of claim 1, wherein the method further comprises genetically modifying at least one of a TNFRSF14 gene, a TNFRSF1A gene, a TNFRSF1B gene, an ICAM1 gene, and a herpesvirus entry mediator (HVEM) gene.

35-43. (canceled)

44. A γδT cell-derived induced pluripotent stem (iPS) human cell, comprising a means for reducing expression of an B2M protein through a genetically modified B2M gene, and/or a means for altering immunogenicity of an immune system to the iPS human cell as compared to a iPS human cell where the B2M gene is not genetically modified; optionally wherein the iPS human cell further comprises a means for reducing expression of a RFX protein, a CD58 protein, and/or a CIITA protein through a genetically modified RFX gene, a genetically modified CD58 gene, and/or a genetically modified CIITA gene.


SEQUENCE LISTING (391 sequences)

SEQ ID NO: 1 - DNA, 20 bp, unidentified
  source 1..20: mol_type = unassigned DNA
cgtgagtaaacctgaatctt

SEQ ID NO: 2 - DNA, 20 bp, unidentified
  source 1..20: mol_type = unassigned DNA
aagtcaacttcaatgtcgga

SEQ ID NO: 3 - DNA, 20 bp, unidentified
  source 1..20: mol_type = unassigned DNA
agtcgctcactggtcccact

SEQ ID NO: 4 - DNA, 20 bp, unidentified
  source 1..20: mol_type = unassigned DNA
ccgtggacagtgaatccact

SEQ ID NO: 5 - DNA, 20 bp, unidentified
  source 1..20: mol_type = unassigned DNA
ctctcaccgatcacttcatc

SEQ ID NO: 6 - DNA, 20 bp, unidentified
  source 1..20: mol_type = unassigned DNA
gtggttgcggatccacctat

SEQ ID NO: 7 - DNA, 20 bp, unidentified
  source 1..20: mol_type = unassigned DNA
acctcgagatgaactggtgg

SEQ ID NO: 8 - DNA, 20 bp, unidentified
  source 1..20: mol_type = unassigned DNA
gaaccctttaggtcaagtcc

SEQ ID NO: 9 - DNA, 20 bp, unidentified
  source 1..20: mol_type = unassigned DNA
ctctcaccaaccggcagcga

SEQ ID NO: 10 - DNA, 20 bp, unidentified
  source 1..20: mol_type = unassigned DNA
aacggtctcaatctctccaa

SEQ ID NO: 11 - DNA, 20 bp, unidentified
  source 1..20: mol_type = unassigned DNA
gtctggatgaggtcttctgc

SEQ ID NO: 12 - DNA, 20 bp, unidentified
  source 1..20: mol_type = unassigned DNA
gttagacacttcggaccctc

SEQ ID NO: 13 - DNA, 20 bp, unidentified
  source 1..20: mol_type = unassigned DNA
cgttaggtacctgtgcgaag

SEQ ID NO: 14 - DNA, 20 bp, unidentified
  source 1..20: mol_type = unassigned DNA
gacgaggagactcactcggg

SEQ ID NO: 15 - DNA, 21 bp, unidentified
  source 1..21: mol_type = unassigned DNA
agtgggggtgaattcagtgta

SEQ ID NO: 16 - DNA, 24 bp, unidentified
  source 1..24: mol_type = unassigned DNA
ccgatattcctcaggtactccaaa

SEQ ID NO: 17 - DNA, 24 bp, unidentified
  source 1..24: mol_type = unassigned DNA
atccatccgacattgaagttgact

SEQ ID NO: 18 - DNA, 24 bp, unidentified
  source 1..24: mol_type = unassigned DNA
ctcacgtcatccagcagagaatgg

SEQ ID NO: 19 - DNA, 24 bp, unidentified
  source 1..24: mol_type = unassigned DNA
aattctctctccattcttcagtaa

SEQ ID NO: 20 - DNA, 24 bp, unidentified
  source 1..24: mol_type = unassigned DNA
actttccattctctgctggatgac

SEQ ID NO: 21 - DNA, 24 bp, unidentified
  source 1..24: mol_type = unassigned DNA
agcaaggactggtctttctatctc

SEQ ID NO: 22 - DNA, 24 bp, unidentified
  source 1..24: mol_type = unassigned DNA
cattctctgctggatgacgtgagt

SEQ ID NO: 23 - DNA, 24 bp, unidentified
  source 1..24: mol_type = unassigned DNA
tggcctggaggctatccagcgtga

SEQ ID NO: 24 - DNA, 24 bp, unidentified
  source 1..24: mol_type = unassigned DNA
ctgaattgctatgtgtctgggttt

SEQ ID NO: 25 - DNA, 24 bp, unidentified
  source 1..24: mol_type = unassigned DNA
tatctcttgtactacactgaattc

SEQ ID NO: 26 - DNA, 24 bp, unidentified
  source 1..24: mol_type = unassigned DNA
tcacagcccaagatagttaagtgg

SEQ ID NO: 27 - DNA, 24 bp, unidentified
  source 1..24: mol_type = unassigned DNA
tccactgtctttttcatagatcga

SEQ ID NO: 28 - DNA, 20 bp, unidentified
  source 1..20: mol_type = unassigned DNA
ccttggggctctgacaggta

SEQ ID NO: 29 - DNA, 20 bp, unidentified
  source 1..20: mol_type = unassigned DNA
tctgcagccttcccagagga

SEQ ID NO: 30 - DNA, 20 bp, unidentified
  source 1..20: mol_type = unassigned DNA
ccaggtcttccacatccttc

SEQ ID NO: 31 - DNA, 21 bp, unidentified
  source 1..21: mol_type = unassigned DNA
gggaaagcctgggggcctgag

SEQ ID NO: 32 - DNA, 20 bp, unidentified
  source 1..20: mol_type = unassigned DNA
caaggacttcagctggggga

SEQ ID NO: 33 - DNA, 20 bp, unidentified
  source 1..20: mol_type = unassigned DNA
ctttggcaaagggagaggta

SEQ ID NO: 34 - DNA, 20 bp, unidentified
  source 1..20: mol_type = unassigned DNA
gctggtggagcctgcccact

SEQ ID NO: 35 - DNA, 21 bp, unidentified
  source 1..21: mol_type = unassigned DNA
aggggaccaagggaattttat

SEQ ID NO: 36 - DNA, 21 bp, unidentified
  source 1..21: mol_type = unassigned DNA
agggcacctgaagaaagcctg

SEQ ID NO: 37 - DNA, 20 bp, unidentified
  source 1..20: mol_type = unassigned DNA
cctggacctggacctgggcc

SEQ ID NO: 38 - DNA, 24 bp, unidentified
  source 1..24: mol_type = unassigned DNA
gcatcacttgctgtatcctctata

SEQ ID NO: 39 - DNA, 24 bp, unidentified
  source 1..24: mol_type = unassigned DNA
tgacaatgacaagctgtatctcta

SEQ ID NO: 40 - DNA, 24 bp, unidentified
  source 1..24: mol_type = unassigned DNA
agggcacctgaagaaagcctgggg

SEQ ID NO: 41 - DNA, 24 bp, unidentified
  source 1..24: mol_type = unassigned DNA
gctggtggagcctgcccactggcc

SEQ ID NO: 42 - DNA, 24 bp, unidentified
  source 1..24: mol_type = unassigned DNA
cctgcacccctgagcctgtgaatc

SEQ ID NO: 43 - DNA, 24 bp, unidentified
  source 1..24: mol_type = unassigned DNA
cctgccccatctcagtgcaacagg

SEQ ID NO: 44 - DNA, 24 bp, unidentified
  source 1..24: mol_type = unassigned DNA
cccatggagcttacccagcctgca

SEQ ID NO: 45 - DNA, 24 bp, unidentified
  source 1..24: mol_type = unassigned DNA
gagcaaagacaacagcagtttcca

SEQ ID NO: 46 - DNA, 24 bp, unidentified
  source 1..24: mol_type = unassigned DNA
gaacaagtgttaaatcaaaaaaga

SEQ ID NO: 47 - DNA, 24 bp, unidentified
  source 1..24: mol_type = unassigned DNA
acaatggagagtatgttatctgca

SEQ ID NO: 48 - DNA, 24 bp, unidentified
  source 1..24: mol_type = unassigned DNA
ctgggataaatgataaaaatgcaa

SEQ ID NO: 49 - DNA, 24 bp, unidentified
  source 1..24: mol_type = unassigned DNA
tacttgtccttgtacatcttgttg

SEQ ID NO: 50 - DNA, 24 bp, unidentified
  source 1..24: mol_type = unassigned DNA
ccgcgctgccagtcgaggcagtcc

SEQ ID NO: 51 - DNA, 24 bp, unidentified
  source 1..24: mol_type = unassigned DNA
acgtttcccgcgctgccagtcgag

SEQ ID NO: 52 - DNA, 24 bp, unidentified
  source 1..24: mol_type = unassigned DNA
ttgcacatccacggtttgcgctgc

SEQ ID NO: 53 - DNA, 21 bp, unidentified
  source 1..21: mol_type = unassigned DNA
gaccacgctgaggacccccag

SEQ ID NO: 54 - DNA, 20 bp, unidentified
  source 1..20: mol_type = unassigned DNA
gtcaatgcacaagttagtgt

SEQ ID NO: 55 - DNA, 20 bp, unidentified
  source 1..20: mol_type = unassigned DNA
aaggaggacgagtacccagt

SEQ ID NO: 56 - DNA, 20 bp, unidentified
  source 1..20: mol_type = unassigned DNA
acacagggtgtcctgactct

SEQ ID NO: 57 - DNA, 20 bp, unidentified
  source 1..20: mol_type = unassigned DNA
gtttaatgtatcgctaccaa

SEQ ID NO: 58 - DNA, 20 bp, unidentified
  source 1..20: mol_type = unassigned DNA
ggaaactcaagcctgcactc

SEQ ID NO: 59 - DNA, 20 bp, unidentified
  source 1..20: mol_type = unassigned DNA
tgacgtgtgcagtaatactg

SEQ ID NO: 60 - DNA, 21 bp, unidentified
  source 1..21: mol_type = unassigned DNA
gcctcactatctacaacttaa

SEQ ID NO: 61 - DNA, 21 bp, unidentified
  source 1..21: mol_type = unassigned DNA
gtgttgtgtatgggaatgtaa

SEQ ID NO: 62 - DNA, 21 bp, unidentified
  source 1..21: mol_type = unassigned DNA
gaagacaacagcataactaaa

SEQ ID NO: 63 - DNA, 21 bp, unidentified
  source 1..21: mol_type = unassigned DNA
acgtaactcaaccagtatata

SEQ ID NO: 64 - DNA, 21 bp, unidentified
  source 1..21: mol_type = unassigned DNA
tactcttagcaatccattatt

SEQ ID NO: 65 - DNA, 21 bp, unidentified
  source 1..21: mol_type = unassigned DNA
gcattgactaatggaagcatt

SEQ ID NO: 66 - DNA, 21 bp, unidentified
  source 1..21: mol_type = unassigned DNA
gtgctgtatatgaatggtatt

SEQ ID NO: 67 - DNA, 21 bp, unidentified
  source 1..21: mol_type = unassigned DNA
gcggtcattcaagacacagat

SEQ ID NO: 68 - DNA, 21 bp, unidentified
  source 1..21: mol_type = unassigned DNA
aggatccgctctgcccagtca

SEQ ID NO: 69 - DNA, 21 bp, unidentified
  source 1..21: mol_type = unassigned DNA
gatgaccgttcccgaggtgca

SEQ ID NO: 70 - DNA, 21 bp, unidentified
  source 1..21: mol_type = unassigned DNA
gagaacccagagggtggagcc

SEQ ID NO: 71 - DNA, 21 bp, unidentified
  source 1..21: mol_type = unassigned DNA
gtacctctgcagaagaggacg

SEQ ID NO: 72 - DNA, 20 bp, unidentified
  source 1..20: mol_type = unassigned DNA
aggatccgctctgcccagtc

SEQ ID NO: 73 - DNA, 20 bp, unidentified
  source 1..20: mol_type = unassigned DNA
gatgaccgttcccgaggtgc

SEQ ID NO: 74 - DNA, 140 bp, unidentified
  source 1..140: mol_type = unassigned DNA
cgccggcgttcggggaacttcaggctccgtcatccgtcgggagtgatagatgttgaattgggtagacaccgaagtgtcaattcaacatgtatcactcgcagcgagtgacagttgtcgttatatggaagagctcggaagac

SEQ ID NO: 75 - DNA, 121 bp, unidentified
  source 1..121: mol_type = unassigned DNA
aacttatgatagcaatgtcagcagtgcctttaagttgtagatagtgaggctttaagattctaaaattatatcctcactatccacaacttacaagtaaggttgaccatactctacagttgtt

SEQ ID NO: 76 - DNA, 121 bp, unidentified
  source 1..121: mol_type = unassigned DNA
ttgttgacatctcataccagttggaatgacggagtgatagatgttgaattcctattaaaatcttagaattgcaattcaacatgtatcactcatccgtgacgactgtaacgatagtattcaa

SEQ ID NO: 77 - DNA, 121 bp, unidentified
  source 1..121: mol_type = unassigned DNA
aacttatgatagcaatgtcagcagtgccttatatactggttgagttacgttttaagattctaaaattatatcgtaactcaagcagtatatcaagtaaggttgaccatactctacagttgtt

SEQ ID NO: 78 - DNA, 121 bp, unidentified
  source 1..121: mol_type = unassigned DNA
aacttatgatagcaatgtcagcagtgcctaataatggattgctaagagtacttaagattctaaaattatgcactcttagcattccattatcaagtaaggttgaccatactctacagttgtt

SEQ ID NO: 79 - DNA, 123 bp, unidentified
  source 1..123: mol_type = unassigned DNA
ttcgtggctacagagtttccttagcagagctgttaagttgtagatagtgaggcttgtctaaactatagcctcactatcaacaacttactagctactgctaggcaatccttccctcgataaatg

SEQ ID NO: 80 - DNA, 123 bp, unidentified
  source 1..123: mol_type = unassigned DNA
gtaaatagctcccttcctaacggatcgtcatcgtcggagtgatagatgttgaattactatcaaatctgtgtaattcaacatgtatcactcagacgagacgattcctttgagacatcggtgctt

SEQ ID NO: 81 - DNA, 123 bp, unidentified
  source 1..123: mol_type = unassigned DNA
ttcgtggctacagagtttccttagcagagctgtatatactggttgagttacgtttgtctaaactataacgtaactcaagcagtatatctagctactgctaggcaatccttccctcgataaatg

SEQ ID NO: 82 - DNA, 123 bp, unidentified
  source 1..123: mol_type = unassigned DNA
ttcgtggctacagagtttccttagcagagctgaataatggattgctaagagtactgtctaaactatgtactcttagcattccattatctagctactgctaggcaatccttccctcgataaatg

SEQ ID NO: 83 - DNA, 285 bp, unidentified
  source 1..285: mol_type = unassigned DNA
cgacttcttaacccaacagaaggctcgagaaggtatattgctgttgacagtgagcgcgcctcactatctacaacttaatagtgaagccacagatgtattaagttgtagatagtgaggcttgcctactgcctcggacttcaaggggctagaattcgcggccgcaaccagcggcggctcctctccccatggccctgcacttgcgctgggtgatcttgctgggctcagaccagatcaccctgcgcaagtccagggacctggggaccccggcaccggcaggccggatcc

SEQ ID NO: 84 - DNA, 115 bp, unidentified
  source 1..115: mol_type = unassigned DNA
catctccatggctgtaccaccttgtcggttaagttgtagatagtgaggcctgttgaatctcatggcctcactatccacaacttattctgacattttggtatctttcatctgacca

SEQ ID NO: 85 - DNA, 115 bp, unidentified
  source 1..115: mol_type = unassigned DNA
catctccatggctgtaccaccttgtcggttaagttgtagatagtgaggcacgttgaatctcatggcctcactatccacaacttattctgacattttggtatctttcatctgacca

SEQ ID NO: 86 - DNA, 115 bp, unidentified
  source 1..115: mol_type = unassigned DNA
accagtctactttctatggttttacagtccggagtgatagatgttgaattggtactctaagttgtcaaattcaacatgtatcactggggctgttccaccatgtcggtacctctac

SEQ ID NO: 87 - DNA, 115 bp, unidentified
  source 1..115: mol_type = unassigned DNA
catctccatggctgtaccaccttgtcggtatatactggttgagttacgtctgttgaatctcatggcgtaactcaagcagtatatttctgacattttggtatctttcatctgacca

SEQ ID NO: 88 - DNA, 115 bp, unidentified
  source 1..115: mol_type = unassigned DNA
catctccatggctgtaccaccttgtcggtatatactggttgagttacgtccgttgaatctcatggcgtaactcaagcagtatatttctgacattttggtatctttcatctgacca

SEQ ID NO: 89 - DNA, 115 bp, unidentified
  source 1..115: mol_type = unassigned DNA
catctccatggctgtaccaccttgtcggaataatggattgctaagagtactgttgaatctcatggactcttagcactccattataactgacattttggtatctttcatctgacca

SEQ ID NO: 90 - DNA, 115 bp, unidentified
  source 1..115: mol_type = unassigned DNA
gggcctggctcgagcagggggcgagggatttaagttgtagatagtgaggcttggtcccctcccccctcactatcaacaacttacgtccttccctcccaatgaccgcgtcttcgtc

SEQ ID NO: 91 - DNA, 115 bp, unidentified
  source 1..115: mol_type = unassigned DNA
gggcctggctcgagcagggggcgagggatttaagttgtagatagtgaggcttggtcccctcccgcctcactatcaacaacttacgtccttccctcccaatgaccgcgtcttcgtc

SEQ ID NO: 92 - DNA, 115 bp, unidentified
  source 1..115: mol_type = unassigned DNA
ctgcttctgcgccagtaaccctcccttcctcggagtgatagatgttgaattcccctcccctggtagaattcaacatgtatcactcatagggagcgggggacgagctcggtccggg

SEQ ID NO: 93 - DNA, 115 bp, unidentified
  source 1..115: mol_type = unassigned DNA
gggcctggctcgagcagggggcgagggattatatactggttgagttacgtttggtcccctcccccgtaactcaagcagtatatcgtccttccctcccaatgaccgcgtcttcgtc

SEQ ID NO: 94 - DNA, 115 bp, unidentified
  source 1..115: mol_type = unassigned DNA
gggcctggctcgagcagggggcgagggattatatactggttgagttacgtttggtcccctcccgcgtaactcaagcagtatatcgtccttccctcccaatgaccgcgtcttcgtc

SEQ ID NO: 95 - DNA, 115 bp, unidentified
  source 1..115: mol_type = unassigned DNA
gggcctggctcgagcagggggcgagggataataatggattgctaagagtactggtcccctccccactcttagcattccattatcgtccttccctcccaatgaccgcgtcttcgtc

SEQ ID NO: 96 - DNA, 115 bp, unidentified
  source 1..115: mol_type = unassigned DNA
gggcctggctcgagcagggggcgagggataataatggattgctaagagtactggtcccctccctactcttagcattccattatcgtccttccctcccaatgaccgcgtcttcgtc

SEQ ID NO: 97 - DNA, 103 bp, unidentified
  source 1..103: mol_type = unassigned DNA
gctcgagccctgccggggcttaagttgtagatagtgaggcagtggtcctctccgtgctatgcactatcaacaacttcgtgctccagcagggcacgcaaagctt

SEQ ID NO: 98 - DNA, 103 bp, unidentified
  source 1..103: mol_type = unassigned DNA
gctcgagccctgccggggcttaagttgtagatagtgaggcagtggtcctctccgtgctgtgcactatcaacaacttcgtgctccagcagggcacgcaaagctt

SEQ ID NO: 99 - DNA, 103 bp, unidentified
  source 1..103: mol_type = unassigned DNA
ttcgaaacgcacgggacgacctccggagtgatagatgttgaattatcgtgcctctcctggtgatcccttcaacaactatcacgagggggccgtcccgagctcg

SEQ ID NO: 100 - DNA, 103 bp, unidentified
  source 1..103: mol_type = unassigned DNA
gctcgagccctgccggggctatatactggttgagttacgtagtggtcctctccgtgctagaaactcaagcagtatagctgctccagcagggcacgcaaagctt

SEQ ID NO: 101 - DNA, 103 bp, unidentified
  source 1..103: mol_type = unassigned DNA
gctcgagccctgccggggcaataatggattgctaagagtaagtggtcctctccgtgctatccttagcattccattagctgctccagcagggcacgcaaagctt

SEQ ID NO: 102 - DNA, 103 bp, unidentified
  source 1..103: mol_type = unassigned DNA
gctcgagccctgccggggcaataatggattgctaagagtaagtggtcctctccgtgctttccttagcattccattagctgctccagcagggcacgcaaagctt

SEQ ID NO: 103 - DNA, 132 bp, unidentified
  source 1..132: mol_type = unassigned DNA
ttcgacctcaagatgtcgatcgtcatgttgagtgatagatgttgaattatctattgatttgtgatgaattcaacatctatcactctcacgatgtcttcgacagtgtagtctatctggtccgtctaagatgta

SEQ ID NO: 104 - DNA, 130 bp, unidentified
  source 1..130: mol_type = unassigned DNA
atgtagaatctgcctggtctatctgatgtgacagcttctgtagcacttaagttgtagatagtgaggtagtgtttagttatctactcactatctacaacttttagtactgctagctgtagaactccagctt

SEQ ID NO: 105 - DNA, 130 bp, unidentified
  source 1..130: mol_type = unassigned DNA
atgtagaatctgcctggtctatctgatgtgacagcttctgtagcactatatactggttgagttacgtagtgtttagttatctagtaactcaaccagtataaaagtactgctagctgtagaactccagctt

SEQ ID NO: 106 - DNA, 130 bp, unidentified
  source 1..130: mol_type = unassigned DNA
atgtagaatctgcctggtctatctgatgtgacagcttctgtagcacaataatggattgctaagaggtagtgtttagttatctactcttagcaatccattaaatgtactgctagctgtagaactccagctt

SEQ ID NO: 107 - DNA, 115 bp, unidentified
  source 1..115: mol_type = unassigned DNA
ccggacggccacggcccccggccgtgactcggagtgatagatgttgaattgggtcccgtcgcaggtcatattcaactactatctctccctgttgtgaccggtcctcggcggcgac

SEQ ID NO: 108 - DNA, 115 bp, unidentified
  source 1..115: mol_type = unassigned DNA
cagcggcggctcctggccagtgttgagttaagttgtagatagtgaggcctggacgctgccctgggggtcactaattacaagttaagacagtgccggcccccggcaccggcaggcc

SEQ ID NO: 109 - DNA, 115 bp, unidentified
  source 1..115: mol_type = unassigned DNA
cagcggcggctcctggccagtgttgagtatatactggttgagttacgtctggacgctgccctgggaataacttttccagtttatagacagtgccggcccccggcaccggcaggcc

SEQ ID NO: 110 - DNA, 115 bp, unidentified
  source 1..115: mol_type = unassigned DNA
cagcggcggctcctggccagtgttgagaataatggattgctaagagtactggacgctgccctgggtctcttaccgatccaatattgacagtgccggcccccggcaccggcaggcc

SEQ ID NO: 111 - DNA, 108 bp, unidentified
  source 1..108: mol_type = unassigned DNA
cagcggcggctcctgtggcctcgttaagttgtagatagtgaggctgtgcaggtcccaatgggcctcactgattgcaacttcacggggacgccccggcaccggcaggcc

SEQ ID NO: 112 - DNA, 108 bp, unidentified
  source 1..108: mol_type = unassigned DNA
ccggacggccacggccccgcagggtcggagtgatagatgttgaattgggtaaccctggacgtgtcaattcaacctctatcactgcgatccggtgtcctcggcggcgac

SEQ ID NO: 113 - DNA, 108 bp, unidentified
  source 1..108: mol_type = unassigned DNA
cagcggcggctcctgtggcctcgtatatactggttgagttacgttgtgcaggtcccaatgggcgtaactttatcagtataaacggggacgccccggcaccggcaggcc

SEQ ID NO: 114 - DNA, 108 bp, unidentified
  source 1..108: mol_type = unassigned DNA
cagcggcggctcctgtggcctcgtataatggattgctaagagtacgtgcaggtcccaatggtactcttggagatccattaatcggggacgccccggcaccggcaggcc

SEQ ID NO: 115 - DNA, 110 bp, unidentified
  source 1..110: mol_type = unassigned DNA
ggctacagtctttcttcatgtgactcgtggacttaagttgtagatagtgaggctgagaatatatgaaggagcccactaactacaacttacgttcaattgtcatcactggc

SEQ ID NO: 116 - DNA, 110 bp, unidentified
  source 1..110: mol_type = unassigned DNA
cggtcactactgttaactcggagtgatagatgttgaattgaggaagtatataagagtcaacttcaagatctatcactacgggtgctcagtgtacttctttctgacatcgg

SEQ ID NO: 117 - DNA, 110 bp, unidentified
  source 1..110: mol_type = unassigned DNA
ggctacagtctttcttcatgtgactcgtggactatatactggttgagttacgttgagaatatatgaaggaacgaactctaccagtatatcgttcaattgtcatcactggc

SEQ ID NO: 118 - DNA, 110 bp, unidentified
  source 1..110: mol_type = unassigned DNA
ggctacagtctttcttcatgtgactcgtggacaataatggattgctaagagtacgagaatatatgaagggtaccttaggaatccattatagttcaattgtcatcactggc

SEQ ID NO: 119 - DNA, 96 bp, unidentified
  source 1..96: mol_type = unassigned DNA
ttcatgtgactcgtggacttaagttgtagatagtgaggctgagaatatatgaaggagcccactaactacaacttacgttcaattgtcatcactggc

SEQ ID NO: 120 - DNA, 96 bp, unidentified
  source 1..96: mol_type = unassigned DNA
cggtcactactgttaactcggagtgatagatgttgaattgaggaagtatataagagtcaacttcaagatctatcactacgggtgctcagtgtactt

SEQ ID NO: 121 - DNA, 96 bp, unidentified
  source 1..96: mol_type = unassigned DNA
ttcatgtgactcgtggactatatactggttgagttacgttgagaatatatgaaggaacgaactctaccagtatatcgttcaattgtcatcactggc

SEQ ID NO: 122 - DNA, 96 bp, unidentified
  source 1..96: mol_type = unassigned DNA
ttcatgtgactcgtggacaataatggattgctaagagtacgagaatatatgaagggtaccttaggaatccattatagttcaattgtcatcactggc

SEQ ID NO: 123 - DNA, 128 bp, unidentified
  source 1..128: mol_type = unassigned DNA
gaattccagcggcggctcctctccccatggccctgttaagttgtagatagtgaggtgctgggctcagacccctcactatcaacaacttaccagggacctggggaccccggcaccggcaggccggatcc

SEQ ID NO: 124 - DNA, 128 bp, unidentified
  source 1..128: mol_type = unassigned DNA
cctaggccggacggccacggccccaggggtccagggacggagtgatagatgttgaattccagactcgggtcgtaattcaacatgtatcactcggtcccggtacccctctcctcggcggcgaccttaag

SEQ ID NO: 125 - DNA, 99 bp, unidentified
  source 1..99: mol_type = unassigned DNA
gaattcagtcctgggggctccttaagttgtagatagtgaggcttgtgattacccaacagcccacatctatcaacttacccgagcccccgggatccgttc

SEQ ID NO: 126 - DNA, 101 bp, unidentified
  source 1..101: mol_type = unassigned DNA
cttgcctagggcccccgagctcggagtgatagatgttgaatttccaacccattagtgtgaaattcaaacatcttcactcacctcgggggtcctgacttaag

SEQ ID NO: 127 - DNA, 99 bp, unidentified
  source 1..99: mol_type = unassigned DNA
gaattcagtcctgggggctcctatatactggttgagttacgtttgtgattacccaacaacgaaccaacctagtatatcccgagcccccgggatccgttc

SEQ ID NO: 128 - DNA, 99 bp, unidentified
  source 1..99: mol_type = unassigned DNA
gaattcagtcctgggggctccaataatggattgctaagagtactgtgattacccaacgtaccttgcaataccattatcccgagcccccgggatccgttc

SEQ ID NO: 129 - RNA, 20 nt, unidentified
  source 1..20: mol_type = unassigned RNA
taatttctactcttgtagat

SEQ ID NO: 130 - RNA, 21 nt, unidentified
  source 1..21: mol_type = unassigned RNA
cgaaatggtacctcggagcct

SEQ ID NO: 131 - RNA, 41 nt, unidentified
  source 1..41: mol_type = unassigned RNA
taatttctactcttgtagatcgaaatggtacctcggagcct

SEQ ID NO: 132 - DNA, 25 bp, unidentified
  source 1..25: mol_type = unassigned DNA
cttacgaaatggtacctcggagcct

SEQ ID NO: 133 - RNA, 21 nt, unidentified
  source 1..21: mol_type = unassigned RNA
ttcagaggctccgaggtacca

SEQ ID NO: 134 - RNA, 41 nt, unidentified
  source 1..41: mol_type = unassigned RNA
taatttctactcttgtagatttcagaggctccgaggtacca

SEQ ID NO: 135 - DNA, 25 bp, unidentified
  source 1..25: mol_type = unassigned DNA
cttcttcagaggctccgaggtacca

SEQ ID NO: 136 - RNA, 21 nt, unidentified
  source 1..21: mol_type = unassigned RNA
gagatgtgatgagtacttacg

SEQ ID NO: 137 - RNA, 41 nt, unidentified
  source 1..41: mol_type = unassigned RNA
taatttctactcttgtagatgagatgtgatgagtacttacg

SEQ ID NO: 138 - DNA, 25 bp, unidentified
  source 1..25: mol_type = unassigned DNA
cttggagatgtgatgagtacttacg

SEQ ID NO: 139 - RNA, 21 nt, unidentified
  source 1..21: mol_type = unassigned RNA
gggctcttagcatcaggctca

SEQ ID NO: 140 - RNA, 41 nt, unidentified
  source 1..41: mol_type = unassigned RNA
taatttctactcttgtagatgggctcttagcatcaggctca

SEQ ID NO: 141 - DNA, 25 bp, unidentified
  source 1..25: mol_type = unassigned DNA
cttggggctcttagcatcaggctca

SEQ ID NO: 142 - RNA, 21 nt, unidentified
  source 1..21: mol_type = unassigned RNA
agaggctccgaggtaccattt

SEQ ID NO: 143 - RNA, 41 nt, unidentified
  source 1..41: mol_type = unassigned RNA
taatttctactcttgtagatagaggctccgaggtaccattt

SEQ ID NO: 144 - DNA, 25 bp, unidentified
  source 1..25: mol_type = unassigned DNA
cttcagaggctccgaggtaccattt

SEQ ID NO: 145 - RNA, 21 nt, unidentified
  source 1..21: mol_type = unassigned RNA
ttctgcacggccttgctgtgg

SEQ ID NO: 146 - RNA, 41 nt, unidentified
  source 1..41: mol_type = unassigned RNA
taatttctactcttgtagatttctgcacggccttgctgtgg

SEQ ID NO: 147 - DNA, 25 bp, unidentified
  source 1..25: mol_type = unassigned DNA
tttgttctgcacggccttgctgtgg

SEQ ID NO: 148 - RNA, 21 nt, unidentified
  source 1..21: mol_type = unassigned RNA
cccacagcaaggccgtgcaga

SEQ ID NO: 149 - RNA, 41 nt, unidentified
  source 1..41: mol_type = unassigned RNA
taatttctactcttgtagatcccacagcaaggccgtgcaga

SEQ ID NO: 150 - DNA, 25 bp, unidentified
  source 1..25: mol_type = unassigned DNA
cttccccacagcaaggccgtgcaga

SEQ ID NO: 151 - RNA, 21 nt, unidentified
  source 1..21: mol_type = unassigned RNA
gttctgcacggccttgctgtg

SEQ ID NO: 152 - RNA, 41 nt, unidentified
  source 1..41: mol_type = unassigned RNA
taatttctactcttgtagatgttctgcacggccttgctgtg

SEQ ID NO: 153 - DNA, 25 bp, unidentified
  source 1..25: mol_type = unassigned DNA
ctttgttctgcacggccttgctgtg

SEQ ID NO: 154 - RNA, 21 nt, unidentified
  source 1..21: mol_type = unassigned RNA
gtctccagtggtgggtcctga

SEQ ID NO: 155 - RNA, 41 nt, unidentified
  source 1..41: mol_type = unassigned RNA
taatttctactcttgtagatgtctccagtggtgggtcctga

SEQ ID NO: 156 - DNA, 25 bp, unidentified
  source 1..25: mol_type = unassigned DNA
ttttgtctccagtggtgggtcctga

SEQ ID NO: 157 - RNA, 21 nt, unidentified
  source 1..21: mol_type = unassigned RNA
ctgacaatgacaagctgtatc

SEQ ID NO: 158 - RNA, 41 nt, unidentified
  source 1..41: mol_type = unassigned RNA
taatttctactcttgtagatctgacaatgacaagctgtatc

SEQ ID NO: 159 - DNA, 25 bp, unidentified
  source 1..25: mol_type = unassigned DNA
ttttctgacaatgacaagctgtatc

SEQ ID NO: 160 - RNA, 21 nt, unidentified
  source 1..21: mol_type = unassigned RNA
gactggccccggccaccaagc

SEQ ID NO: 161 - RNA, 41 nt, unidentified
  source 1..41: mol_type = unassigned RNA
taatttctactcttgtagatgactggccccggccaccaagc

SEQ ID NO: 162 - DNA, 25 bp, unidentified
  source 1..25: mol_type = unassigned DNA
cttggactggccccggccaccaagc

SEQ ID NO: 163 - RNA, 21 nt, unidentified
  source 1..21: mol_type = unassigned RNA
gtggccggggccagtccaagt

SEQ ID NO: 164 - RNA, 41 nt, unidentified
  source 1..41: mol_type = unassigned RNA
taatttctactcttgtagatgtggccggggccagtccaagt

SEQ ID NO: 165 - DNA, 25 bp, unidentified
  source 1..25: mol_type = unassigned DNA
cttggtggccggggccagtccaagt

SEQ ID NO: 166 - RNA, 21 nt, unidentified
  source 1..21: mol_type = unassigned RNA
cctgttgccgcccactcagca

SEQ ID NO: 167 - RNA, 41 nt, unidentified
  source 1..41: mol_type = unassigned RNA
taatttctactcttgtagatcctgttgccgcccactcagca

SEQ ID NO: 168 - DNA, 25 bp, unidentified
  source 1..25: mol_type = unassigned DNA
cttgcctgttgccgcccactcagca

SEQ ID NO: 169 - RNA, 21 nt, unidentified
  source 1..21: mol_type = unassigned RNA
aggtcaagtccaggcaggggt

SEQ ID NO: 170 - RNA, 41 nt, unidentified
  source 1..41: mol_type = unassigned RNA
taatttctactcttgtagataggtcaagtccaggcaggggt

SEQ ID NO: 171 - DNA, 25 bp, unidentified
  source 1..25: mol_type = unassigned DNA
ctttaggtcaagtccaggcaggggt

SEQ ID NO: 172 - RNA, 21 nt, unidentified
  source 1..21: mol_type = unassigned RNA
acttgcatcagatattgctac

SEQ ID NO: 173 - RNA, 41 nt, unidentified
  source 1..41: mol_type = unassigned RNA
taatttctactcttgtagatacttgcatcagatattgctac

SEQ ID NO: 174 - DNA, 25 bp, unidentified
  source 1..25: mol_type = unassigned DNA
tttaacttgcatcagatattgctac

SEQ ID NO: 175 - RNA, 21 nt, unidentified
  source 1..21: mol_type = unassigned RNA
ctccttatgccactgtagcaa

SEQ ID NO: 176 - RNA, 41 nt, unidentified
  source 1..41: mol_type = unassigned RNA
taatttctactcttgtagatctccttatgccactgtagcaa

SEQ ID NO: 177 - DNA, 25 bp, unidentified
  source 1..25: mol_type = unassigned DNA
cttcctccttatgccactgtagcaa

SEQ ID NO: 178 - RNA, 21 nt, unidentified
  source 1..21: mol_type = unassigned RNA
cactctcagaaccctttaggt

SEQ ID NO: 179 - RNA, 41 nt, unidentified
  source 1..41: mol_type = unassigned RNA
taatttctactcttgtagatcactctcagaaccctttaggt

SEQ ID NO: 180 - DNA, 25 bp, unidentified
  source 1..25: mol_type = unassigned DNA
cttacactctcagaaccctttaggt

SEQ ID NO: 181 - RNA, 21 nt, unidentified
  source 1..21: mol_type = unassigned RNA
agttccatcgttgaggtcgcc

SEQ ID NO: 182 - RNA, 41 nt, unidentified
  source 1..41: mol_type = unassigned RNA
taatttctactcttgtagatagttccatcgttgaggtcgcc

SEQ ID NO: 183 - DNA, 25 bp, unidentified
  source 1..25: mol_type = unassigned DNA
cttcagttccatcgttgaggtcgcc

SEQ ID NO: 184 - RNA, 21 nt, unidentified
  source 1..21: mol_type = unassigned RNA
aggatccgctctgcccagtca

SEQ ID NO: 185 - RNA, 41 nt, unidentified
  source 1..41: mol_type = unassigned RNA
taatttctactcttgtagataggatccgctctgcccagtca

SEQ ID NO: 186 - DNA, 25 bp, unidentified
  source 1..25: mol_type = unassigned DNA
tttcaggatccgctctgcccagtca

SEQ ID NO: 187 - RNA, 21 nt, unidentified
  source 1..21: mol_type = unassigned RNA
aggccatggggctcgctggtg

SEQ ID NO: 188 - RNA, 41 nt, unidentified
  source 1..41: mol_type = unassigned RNA
taatttctactcttgtagataggccatggggctcgctggtg

SEQ ID NO: 189 - DNA, 25 bp, unidentified
  source 1..25: mol_type = unassigned DNA
cttaaggccatggggctcgctggtg

SEQ ID NO: 190 - RNA, 21 nt, unidentified
  source 1..21: mol_type = unassigned RNA
ctgctacagcagcatctcatc

SEQ ID NO: 191 - RNA, 41 nt, unidentified
  source 1..41: mol_type = unassigned RNA
taatttctactcttgtagatctgctacagcagcatctcatc

SEQ ID NO: 192 - DNA, 25 bp, unidentified
  source 1..25: mol_type = unassigned DNA
cttcctgctacagcagcatctcatc

SEQ ID NO: 193 - RNA, 21 nt, unidentified
  source 1..21: mol_type = unassigned RNA
gatgaccgttcccgaggtgca

SEQ ID NO: 194 - RNA, 41 nt, unidentified
  source 1..41: mol_type = unassigned RNA
taatttctactcttgtagatgatgaccgttcccgaggtgca

SEQ ID NO: 195 - DNA, 25 bp, unidentified
  source 1..25: mol_type = unassigned DNA
tttagatgaccgttcccgaggtgca

SEQ ID NO: 196 - RNA, 21 nt, unidentified
  source 1..21: mol_type = unassigned RNA
gtttagatgaccgttcccgag

SEQ ID NO: 197 - RNA, 41 nt, unidentified
  source 1..41: mol_type = unassigned RNA
taatttctactcttgtagatgtttagatgaccgttcccgag

SEQ ID NO: 198 - DNA, 25 bp, unidentified
  source 1..25: mol_type = unassigned DNA
tttggtttagatgaccgttcccgag

SEQ ID NO: 199 - RNA, 21 nt, unidentified
  source 1..21: mol_type = unassigned RNA
ggtttagatgaccgttcccga

SEQ ID NO: 200 - RNA, 41 nt, unidentified
  source 1..41: mol_type = unassigned RNA
taatttctactcttgtagatggtttagatgaccgttcccga

SEQ ID NO: 201 - DNA, 25 bp, unidentified
  source 1..25: mol_type = unassigned DNA
ctttggtttagatgaccgttcccga

SEQ ID NO: 202 - RNA, 21 nt, unidentified
  source 1..21: mol_type = unassigned RNA
gagaacccagagggtggagcc

SEQ ID NO: 203 - RNA, 41 nt, unidentified
  source 1..41: mol_type = unassigned RNA
taatttctactcttgtagatgagaacccagagggtggagcc

SEQ ID NO: 204 - DNA, 25 bp, unidentified
  source 1..25: mol_type = unassigned DNA
tttagagaacccagagggtggagcc

SEQ ID NO: 205 - RNA, 21 nt, unidentified
  source 1..21: mol_type = unassigned RNA
gtacctctgcagaagaggacg

SEQ ID NO: 206 - RNA, 41 nt, unidentified
  source 1..41: mol_type = unassigned RNA
taatttctactcttgtagatgtacctctgcagaagaggacg

SEQ ID NO: 207 - DNA, 25 bp, unidentified
  source 1..25: mol_type = unassigned DNA
tttggtacctctgcagaagaggacg

SEQ ID NO: 208 - RNA, 21 nt, unidentified
  source 1..21: mol_type = unassigned RNA
ggtacctctgcagaagaggac

SEQ ID NO: 209 - RNA, 41 nt, unidentified
  source 1..41: mol_type = unassigned RNA
taatttctactcttgtagatggtacctctgcagaagaggac

SEQ ID NO: 210 - DNA, 25 bp, unidentified
  source 1..25: mol_type = unassigned DNA
ttttggtacctctgcagaagaggac

SEQ ID NO: 211 - RNA, 21 nt, unidentified
  source 1..21: mol_type = unassigned RNA
cttacctgggccagtctctct

SEQ ID NO: 212 - RNA, 41 nt, unidentified
  source 1..41: mol_type = unassigned RNA
taatttctactcttgtagatcttacctgggccagtctctct

SEQ ID NO: 213 - DNA, 25 bp, unidentified
  source 1..25: mol_type = unassigned DNA
cttccttacctgggccagtctctct

SEQ ID NO: 214 - RNA, 21 nt, unidentified
  source 1..21: mol_type = unassigned RNA
agctgtcctcttgacaccctt

SEQ ID NO: 215 - RNA, 41 nt, unidentified
  source 1..41: mol_type = unassigned RNA
taatttctactcttgtagatagctgtcctcttgacaccctt

SEQ ID NO: 216 - DNA, 25 bp, unidentified
  source 1..25: mol_type = unassigned DNA
cttcagctgtcctcttgacaccctt

SEQ ID NO: 217 - RNA, 21 nt, unidentified
  source 1..21: mol_type = unassigned RNA
agctggtggagcctgcccact

SEQ ID NO: 218 - RNA, 41 nt, unidentified
  source 1..41: mol_type = unassigned RNA
taatttctactcttgtagatagctggtggagcctgcccact

SEQ ID NO: 219 - DNA, 25 bp, unidentified
  source 1..25: mol_type = unassigned DNA
ctttagctggtggagcctgcccact

SEQ ID NO: 220 - RNA, 21 nt, unidentified
  source 1..21: mol_type = unassigned RNA
cgctctccacgtgcgagagga

SEQ ID NO: 221 - RNA, 41 nt, unidentified
  source 1..41: mol_type = unassigned RNA
taatttctactcttgtagatcgctctccacgtgcgagagga

SEQ ID NO: 222 - DNA, 25 bp, unidentified
  source 1..25: mol_type = unassigned DNA
cttccgctctccacgtgcgagagga

SEQ ID NO: 223 - RNA, 21 nt, unidentified
  source 1..21: mol_type = unassigned RNA
agggcacctgaagaaagcctg

SEQ ID NO: 224 - RNA, 41 nt, unidentified
  source 1..41: mol_type = unassigned RNA
taatttctactcttgtagatagggcacctgaagaaagcctg

SEQ ID NO: 225 - DNA, 25 bp, unidentified
  source 1..25: mol_type = unassigned DNA
tttcagggcacctgaagaaagcctg

SEQ ID NO: 226 - RNA, 21 nt, unidentified
  source 1..21: mol_type = unassigned RNA
acacccttgtcatgtggtcct

SEQ ID NO: 227 - RNA, 41 nt, unidentified
  source 1..41: mol_type = unassigned RNA
taatttctactcttgtagatacacccttgtcatgtggtcct

SEQ ID NO: 228 - DNA, 25 bp, unidentified
  source 1..25: mol_type = unassigned DNA
cttgacacccttgtcatgtggtcct

SEQ ID NO: 229 - RNA, 21 nt, unidentified
  source 1..21: mol_type = unassigned RNA
gctggtggagcctgcccactg

SEQ ID NO: 230 - RNA, 41 nt, unidentified
  source 1..41: mol_type = unassigned RNA
taatttctactcttgtagatgctggtggagcctgcccactg

SEQ ID NO: 231 - DNA, 25 bp, unidentified
  source 1..25: mol_type = unassigned DNA
tttagctggtggagcctgcccactg

SEQ ID NO: 232 - RNA, 21 nt, unidentified
  source 1..21: mol_type = unassigned RNA
aggtgccctgaaagtggctac

SEQ ID NO: 233 - RNA, 41 nt, unidentified
  source 1..41: mol_type = unassigned RNA
taatttctactcttgtagataggtgccctgaaagtggctac

SEQ ID NO: 234 - DNA, 25 bp, unidentified
  source 1..25: mol_type = unassigned DNA
cttcaggtgccctgaaagtggctac

SEQ ID NO: 235 - RNA, 21 nt, unidentified
  source 1..21: mol_type = unassigned RNA
ggaatttctactcttgtagat

SEQ ID NO: 236 - RNA, 42 nt, unidentified
  source 1..42: mol_type = unassigned RNA
ggaatttctactcttgtagataggatccgctctgcccagtca

SEQ ID NO: 237 - RNA, 35 nt, unidentified
  source 1..35: mol_type = unassigned RNA
gtcaaaagacctttggaatttctactcttgtagat

SEQ ID NO: 238 - RNA, 56 nt, unidentified
  source 1..56: mol_type = unassigned RNA
gtcaaaagacctttggaatttctactcttgtagataggatccgctctgcccagtca

SEQ ID NO: 239 - RNA, 20 nt, unidentified
  source 1..20: mol_type = unassigned RNA
aggatccgctctgcccagtc

SEQ ID NO: 240 - RNA, 40 nt, unidentified
  source 1..40: mol_type = unassigned RNA
taatttctactcttgtagataggatccgctctgcccagtc

SEQ ID NO: 241 - DNA, 24 bp, unidentified
  source 1..24: mol_type = unassigned DNA
tttcaggatccgctctgcccagtc

SEQ ID NO: 242 - RNA, 41 nt, unidentified
  source 1..41: mol_type = unassigned RNA
ggaatttctactcttgtagataggatccgctctgcccagtc

SEQ ID NO: 243 - RNA, 55 nt, unidentified
  source 1..55: mol_type = unassigned RNA
gtcaaaagacctttggaatttctactcttgtagataggatccgctctgcccagtc

SEQ ID NO: 244 - RNA, 42 nt, unidentified
  source 1..42: mol_type = unassigned RNA
ggaatttctactcttgtagatgatgaccgttcccgaggtgca

SEQ ID NO: 245 - RNA, 56 nt, unidentified
  source 1..56: mol_type = unassigned RNA
gtcaaaagacctttggaatttctactcttgtagatgatgaccgttcccgaggtgca

SEQ ID NO: 246 - RNA, 20 nt, unidentified
  source 1..20: mol_type = unassigned RNA
gatgaccgttcccgaggtgc

SEQ ID NO: 247 - RNA, 40 nt, unidentified
  source 1..40: mol_type = unassigned RNA
taatttctactcttgtagatgatgaccgttcccgaggtgc

SEQ ID NO: 248 - DNA, 24 bp, unidentified
  source 1..24: mol_type = unassigned DNA
tttagatgaccgttcccgaggtgc

SEQ ID NO: 249 - RNA, 41 nt, unidentified
  source 1..41: mol_type = unassigned RNA
ggaatttctactcttgtagatgatgaccgttcccgaggtgc

SEQ ID NO: 250 - RNA, 55 nt, unidentified
  source 1..55: mol_type = unassigned RNA
gtcaaaagacctttggaatttctactcttgtagatgatgaccgttcccgaggtgc

SEQ ID NO: 251 - RNA, 21 nt, unidentified
  source 1..21: mol_type = unassigned RNA
agtgggggtgaattcagtgta

SEQ ID NO: 252 - RNA, 41 nt, unidentified
  source 1..41: mol_type = unassigned RNA
taatttctactcttgtagatagtgggggtgaattcagtgta

SEQ ID NO: 253 - DNA, 25 bp, unidentified
  source 1..25: mol_type = unassigned DNA
tttcagtgggggtgaattcagtgta

SEQ ID NO: 254 - RNA, 21 nt, unidentified
  source 1..21: mol_type = unassigned RNA
actcaccaaagcagtgcagca

SEQ ID NO: 255 - RNA, 41 nt, unidentified
  source 1..41: mol_type = unassigned RNA
taatttctactcttgtagatactcaccaaagcagtgcagca

SEQ ID NO: 256 - DNA, 25 bp, unidentified
  source 1..25: mol_type = unassigned DNA
cttcactcaccaaagcagtgcagca

SEQ ID NO: 257 - RNA, 21 nt, unidentified
  source 1..21: mol_type = unassigned RNA
atggtatcagtaatatttggc

SEQ ID NO: 258 - RNA, 41 nt, unidentified
  source 1..41: mol_type = unassigned RNA
taatttctactcttgtagatatggtatcagtaatatttggc

SEQ ID NO: 259 - DNA, 25 bp, unidentified
  source 1..25: mol_type = unassigned DNA
cttcatggtatcagtaatatttggc

SEQ ID NO: 260 - RNA, 21 nt, unidentified
  source 1..21: mol_type = unassigned RNA
gcgattccatttcatactcat

SEQ ID NO: 261 - RNA, 41 nt, unidentified
  source 1..41: mol_type = unassigned RNA
taatttctactcttgtagatgcgattccatttcatactcat

SEQ ID NO: 262 - DNA, 25 bp, unidentified
  source 1..25: mol_type = unassigned DNA
tttggcgattccatttcatactcat

SEQ ID NO: 263 - RNA, 21 nt, unidentified
  source 1..21: mol_type = unassigned RNA
taaaggcacattgcttggtac

SEQ ID NO: 264 - RNA, 41 nt, unidentified
  source 1..41: mol_type = unassigned RNA
taatttctactcttgtagattaaaggcacattgcttggtac

SEQ ID NO: 265 - DNA, 25 bp, unidentified
  source 1..25: mol_type = unassigned DNA
cttttaaaggcacattgcttggtac

SEQ ID NO: 266 - RNA, 21 nt, unidentified
  source 1..21: mol_type = unassigned RNA
cataggacctcttttaaaggc

SEQ ID NO: 267 - RNA, 41 nt, unidentified
  source 1..41: mol_type = unassigned RNA
taatttctactcttgtagatcataggacctcttttaaaggc

SEQ ID NO: 268 - DNA, 25 bp, unidentified
  source 1..25: mol_type = unassigned DNA
tttccataggacctcttttaaaggc

SEQ ID NO: 269 - RNA, 21 nt, unidentified
  source 1..21: mol_type = unassigned RNA
tttagacactgtgtcaggtag

SEQ ID NO: 270 - RNA, 41 nt, unidentified
  source 1..41: mol_type = unassigned RNA
taatttctactcttgtagattttagacactgtgtcaggtag

SEQ ID NO: 271 - DNA, 25 bp, unidentified
  source 1..25: mol_type = unassigned DNA
tttatttagacactgtgtcaggtag

SEQ ID NO: 272 - RNA, 21 nt, unidentified
  source 1..21: mol_type = unassigned RNA
gacactgtgtcaggtagcctc

SEQ ID NO: 273 - RNA, 41 nt, unidentified
  source 1..41: mol_type = unassigned RNA
taatttctactcttgtagatgacactgtgtcaggtagcctc

SEQ ID NO: 274 - DNA, 25 bp, unidentified
  source 1..25: mol_type = unassigned DNA
tttagacactgtgtcaggtagcctc

SEQ ID NO: 275 - RNA, 21 nt, unidentified
  source 1..21: mol_type = unassigned RNA
catgtaccaagcaatgtgcct

SEQ ID NO: 276 - RNA, 41 nt, unidentified
  source 1..41: mol_type = unassigned RNA
taatttctactcttgtagatcatgtaccaagcaatgtgcct

SEQ ID NO: 277 - DNA, 25 bp, unidentified
  source 1..25: mol_type = unassigned DNA
tttccatgtaccaagcaatgtgcct

SEQ ID NO: 278 - RNA, 21 nt, unidentified
  source 1..21: mol_type = unassigned RNA
aaggcacattgcttggtacat

SEQ ID NO: 279 - RNA, 41 nt, unidentified
  source 1..41: mol_type = unassigned RNA
taatttctactcttgtagataaggcacattgcttggtacat

SEQ ID NO: 280 - DNA, 25 bp, unidentified
  source 1..25: mol_type = unassigned DNA
tttaaaggcacattgcttggtacat

SEQ ID NO: 281 - RNA, 21 nt, unidentified
  source 1..21: mol_type = unassigned RNA
cccaacaaatatatggtgttg

SEQ ID NO: 282 - RNA, 41 nt, unidentified
  source 1..41: mol_type = unassigned RNA
taatttctactcttgtagatcccaacaaatatatggtgttg

SEQ ID NO: 283 - DNA, 25 bp, unidentified
  source 1..25: mol_type = unassigned DNA
ttttcccaacaaatatatggtgttg

SEQ ID NO: 284 - RNA, 21 nt, unidentified
  source 1..21: mol_type = unassigned RNA
ctctcctaggtttcatcagct

SEQ ID NO: 285 - RNA, 41 nt, unidentified
  source 1..41: mol_type = unassigned RNA
taatttctactcttgtagatctctcctaggtttcatcagct

SEQ ID NO: 286 - DNA, 25 bp, unidentified
  source 1..25: mol_type = unassigned DNA
ttttctctcctaggtttcatcagct

SEQ ID NO: 287 - RNA, 21 nt, unidentified
  source 1..21: mol_type = unassigned RNA
ccataggacctcttttaaagg

SEQ ID NO: 288 - RNA, 41 nt, unidentified
  source 1..41: mol_type = unassigned RNA
taatttctactcttgtagatccataggacctcttttaaagg

SEQ ID NO: 289 - DNA, 25 bp, unidentified
  source 1..25: mol_type = unassigned DNA
ttttccataggacctcttttaaagg

SEQ ID NO: 290 - RNA, 21 nt, unidentified
  source 1..21: mol_type = unassigned RNA
tggaatactcaccaagcacat

SEQ ID NO: 291 - RNA, 41 nt, unidentified
  source 1..41: mol_type = unassigned RNA
taatttctactcttgtagattggaatactcaccaagcacat

SEQ ID NO: 292 - DNA, 25 bp, unidentified
  source 1..25: mol_type = unassigned DNA
cttatggaatactcaccaagcacat

SEQ ID NO: 293 - RNA, 21 nt, unidentified
  source 1..21: mol_type = unassigned RNA
gtacatggaaagttacattcc

SEQ ID NO: 294 - RNA, 41 nt, unidentified
  source 1..41: mol_type = unassigned RNA
taatttctactcttgtagatgtacatggaaagttacattcc

SEQ ID NO: 295 - DNA, 25 bp, unidentified
  source 1..25: mol_type = unassigned DNA
cttggtacatggaaagttacattcc

SEQ ID NO: 296 - RNA, 21 nt, unidentified
  source 1..21: mol_type = unassigned RNA
tcccaacaaatatatggtgtt

SEQ ID NO: 297 - RNA, 41 nt, unidentified
  source 1..41: mol_type = unassigned RNA
taatttctactcttgtagattcccaacaaatatatggtgtt

SEQ ID NO: 298 - DNA, 25 bp, unidentified
  source 1..25: mol_type = unassigned DNA
tttttcccaacaaatatatggtgtt

SEQ ID NO: 299 - RNA, 21 nt, unidentified
  source 1..21: mol_type = unassigned RNA
acatcatcagatgaagatgag

SEQ ID NO: 300 - RNA, 41 nt, unidentified
  source 1..41: mol_type = unassigned RNA
taatttctactcttgtagatacatcatcagatgaagatgag

SEQ ID NO: 301 - DNA, 25 bp, unidentified
  source 1..25: mol_type = unassigned DNA
cttaacatcatcagatgaagatgag

SEQ ID NO: 302 - RNA, 21 nt, unidentified
  source 1..21: mol_type = unassigned RNA
ttgggaaaaacagctgatgaa

SEQ ID NO: 303 - RNA, 41 nt, unidentified
  source 1..41: mol_type = unassigned RNA
taatttctactcttgtagatttgggaaaaacagctgatgaa

SEQ ID NO: 304 - DNA, 25 bp, unidentified
  source 1..25: mol_type = unassigned DNA
tttgttgggaaaaacagctgatgaa

SEQ ID NO: 305 - RNA, 21 nt, unidentified
  source 1..21: mol_type = unassigned RNA
ttctctcctaggtttcatcag

SEQ ID NO: 306 - RNA, 41 nt, unidentified
  source 1..41: mol_type = unassigned RNA
taatttctactcttgtagatttctctcctaggtttcatcag

SEQ ID NO: 307 - DNA, 25 bp, unidentified
  source 1..25: mol_type = unassigned DNA
ttttttctctcctaggtttcatcag

SEQ ID NO: 308 - RNA, 21 nt, unidentified
  source 1..21: mol_type = unassigned RNA
tctctcctaggtttcatcagc

SEQ ID NO: 309 - RNA, 41 nt, unidentified
  source 1..41: mol_type = unassigned RNA
taatttctactcttgtagattctctcctaggtttcatcagc

SEQ ID NO: 310 - DNA, 25 bp, unidentified
  source 1..25: mol_type = unassigned DNA
tttttctctcctaggtttcatcagc

SEQ ID NO: 311 - RNA, 21 nt, unidentified
  source 1..21: mol_type = unassigned RNA
atcagctgtttttcccaacaa

SEQ ID NO: 312 - RNA, 41 nt, unidentified
  source 1..41: mol_type = unassigned RNA
taatttctactcttgtagatatcagctgtttttcccaacaa

SEQ ID NO: 313 - DNA, 25 bp, unidentified
  source 1..25: mol_type = unassigned DNA
tttcatcagctgtttttcccaacaa

SEQ ID NO: 314 - RNA, 21 nt, unidentified
  source 1..21: mol_type = unassigned RNA
atactcatcttcatctgatga

SEQ ID NO: 315 - RNA, 41 nt, unidentified
  source 1..41: mol_type = unassigned RNA
taatttctactcttgtagatatactcatcttcatctgatga

SEQ ID NO: 316 - DNA, 25 bp, unidentified
  source 1..25: mol_type = unassigned DNA
tttcatactcatcttcatctgatga

SEQ ID NO: 317 - RNA, 21 nt, unidentified
  source 1..21: mol_type = unassigned RNA
tctcctaggtttcatcagctg

SEQ ID NO: 318 - RNA, 41 nt, unidentified
  source 1..41: mol_type = unassigned RNA
taatttctactcttgtagattctcctaggtttcatcagctg

SEQ ID NO: 319 - DNA, 25 bp, unidentified
  source 1..25: mol_type = unassigned DNA
tttctctcctaggtttcatcagctg

SEQ ID NO: 320 - RNA, 21 nt, unidentified
  source 1..21: mol_type = unassigned RNA
aaaagaggtcctatggaaaaa

SEQ ID NO: 321 - RNA, 41 nt, unidentified
  source 1..41: mol_type = unassigned RNA
taatttctactcttgtagataaaagaggtcctatggaaaaa

SEQ ID NO: 322 - DNA, 25 bp, unidentified
  source 1..25: mol_type = unassigned DNA
ctttaaaagaggtcctatggaaaaa

SEQ ID NO: 323 - RNA, 21 nt, unidentified
  source 1..21: mol_type = unassigned RNA
cattgctccataggacaatcc

SEQ ID NO: 324 - RNA, 41 nt, unidentified
  source 1..41: mol_type = unassigned RNA
taatttctactcttgtagatcattgctccataggacaatcc

SEQ ID NO: 325 - DNA, 25 bp, unidentified
  source 1..25: mol_type = unassigned DNA
tttacattgctccataggacaatcc

SEQ ID NO: 326 - RNA, 21 nt, unidentified
  source 1..21: mol_type = unassigned RNA
ctcaccgctgcttgggataca

SEQ ID NO: 327 - RNA, 41 nt, unidentified
  source 1..41: mol_type = unassigned RNA
taatttctactcttgtagatctcaccgctgcttgggataca

SEQ ID NO: 328 - DNA, 25 bp, unidentified
  source 1..25: mol_type = unassigned DNA
tttactcaccgctgcttgggataca

SEQ ID NO: 329 - RNA, 21 nt, unidentified
  source 1..21: mol_type = unassigned RNA
catctcccacactaacttgtg

SEQ ID NO: 330 - RNA, 41 nt, unidentified
  source 1..41: mol_type = unassigned RNA
taatttctactcttgtagatcatctcccacactaacttgtg

SEQ ID NO: 331 - DNA, 25 bp, unidentified
  source 1..25: mol_type = unassigned DNA
cttccatctcccacactaacttgtg

SEQ ID NO: 332 - RNA, 21 nt, unidentified
  source 1..21: mol_type = unassigned RNA
gacaacctgtatcccaagcag

SEQ ID NO: 333 - RNA, 41 nt, unidentified
  source 1..41: mol_type = unassigned RNA
taatttctactcttgtagatgacaacctgtatcccaagcag

SEQ ID NO: 334 - DNA, 25 bp, unidentified
  source 1..25: mol_type = unassigned DNA
ttttgacaacctgtatcccaagcag

SEQ ID NO: 335 - RNA, 21 nt, unidentified
  source 1..21: mol_type = unassigned RNA
acaacctgtatcccaagcagc

SEQ ID NO: 336 - RNA, 41 nt, unidentified
  source 1..41: mol_type = unassigned RNA
taatttctactcttgtagatacaacctgtatcccaagcagc

SEQ ID NO: 337 - DNA, 25 bp, unidentified
  source 1..25: mol_type = unassigned DNA
tttgacaacctgtatcccaagcagc

SEQ ID NO: 338 - RNA, 21 nt, unidentified
  source 1..21: mol_type = unassigned RNA
gttatttactcaccgctgctt

SEQ ID NO: 339 - RNA, 41 nt, unidentified
  source 1..41: mol_type = unassigned RNA
taatttctactcttgtagatgttatttactcaccgctgctt

SEQ ID NO: 340 - DNA, 25 bp, unidentified
  source 1..25: mol_type = unassigned DNA
tttagttatttactcaccgctgctt

SEQ ID NO: 341 - RNA, 21 nt, unidentified
  source 1..21: mol_type = unassigned RNA
cattagtcaatgcacaagtta

SEQ ID NO: 342 - RNA, 41 nt, unidentified
  source 1..41: mol_type = unassigned RNA
taatttctactcttgtagatcattagtcaatgcacaagtta

SEQ ID NO: 343 - DNA, 25 bp, unidentified
  source 1..25: mol_type = unassigned DNA
cttccattagtcaatgcacaagtta

SEQ ID NO: 344 - RNA, 21 nt, unidentified
  source 1..21: mol_type = unassigned RNA
taatgtactcatgggattgtc

SEQ ID NO: 345 - RNA, 41 nt, unidentified
  source 1..41: mol_type = unassigned RNA
taatttctactcttgtagattaatgtactcatgggattgtc

SEQ ID NO: 346 - DNA, 25 bp, unidentified
  source 1..25: mol_type = unassigned DNA
cttataatgtactcatgggattgtc

SEQ ID NO: 347 - RNA, 21 nt, unidentified
  source 1..21: mol_type = unassigned RNA
tgcattgactaatggaagcat

SEQ ID NO: 348 - RNA, 41 nt, unidentified
  source 1..41: mol_type = unassigned RNA
taatttctactcttgtagattgcattgactaatggaagcat

SEQ ID NO: 349 - DNA, 25 bp, unidentified
  source 1..25: mol_type = unassigned DNA
cttgtgcattgactaatggaagcat

SEQ ID NO: 350 - RNA, 21 nt, unidentified
  source 1..21: mol_type = unassigned RNA
aatgcttccattagtcaatgc

SEQ ID NO: 351 - RNA, 41 nt, unidentified
  source 1..41: mol_type = unassigned RNA
taatttctactcttgtagataatgcttccattagtcaatgc

SEQ ID NO: 352 - DNA, 25 bp, unidentified
  source 1..25: mol_type = unassigned DNA
cttcaatgcttccattagtcaatgc

SEQ ID NO: 353 - RNA, 21 nt, unidentified
  source 1..21: mol_type = unassigned RNA
agatggaaaatgatcttccac

SEQ ID NO: 354 - RNA, 41 nt, unidentified
  source 1..41: mol_type = unassigned RNA
taatttctactcttgtagatagatggaaaatgatcttccac

SEQ ID NO: 355 - DNA, 25 bp, unidentified
  source 1..25: mol_type = unassigned DNA
tttaagatggaaaatgatcttccac

SEQ ID NO: 356 - RNA, 21 nt, unidentified
  source 1..21: mol_type = unassigned RNA
gcaatccattatttaatacaa

SEQ ID NO: 357 - RNA, 41 nt, unidentified
  source 1..41: mol_type = unassigned RNA
taatttctactcttgtagatgcaatccattatttaatacaa

SEQ ID NO: 358 - DNA, 25 bp, unidentified
  source 1..25: mol_type = unassigned DNA
cttagcaatccattatttaatacaa

SEQ ID NO: 359 - RNA, 21 nt, unidentified
  source 1..21: mol_type = unassigned RNA
ttccagagtctcttccatctc

SEQ ID NO: 360 - RNA, 41 nt, unidentified
  source 1..41: mol_type = unassigned RNA
taatttctactcttgtagatttccagagtctcttccatctc

SEQ ID NO: 361 - DNA, 25 bp, unidentified
  source 1..25: mol_type = unassigned DNA
ttttttccagagtctcttccatctc

SEQ ID NO: 362 - RNA, 21 nt, unidentified
  source 1..21: mol_type = unassigned RNA
aaatatatactggttgagtta

SEQ ID NO: 363 - RNA, 41 nt, unidentified
  source 1..41: mol_type = unassigned RNA
taatttctactcttgtagataaatatatactggttgagtta

SEQ ID NO: 364 - DNA, 25 bp, unidentified
  source 1..25: mol_type = unassigned DNA
cttaaaatatatactggttgagtta

SEQ ID NO: 365 - RNA, 21 nt, unidentified
  source 1..21: mol_type = unassigned RNA
ccatcttaaaatatatactgg

SEQ ID NO: 366 - RNA, 41 nt, unidentified
  source 1..41: mol_type = unassigned RNA
taatttctactcttgtagatccatcttaaaatatatactgg

SEQ ID NO: 367 - DNA, 25 bp, unidentified
  source 1..25: mol_type = unassigned DNA
ttttccatcttaaaatatatactgg

SEQ ID NO: 368 - RNA, 21 nt, unidentified
  source 1..21: mol_type = unassigned RNA
ggatacaggttgtcaaaatga

SEQ ID NO: 369 - RNA, 41 nt, unidentified
  source 1..41: mol_type = unassigned RNA
taatttctactcttgtagatggatacaggttgtcaaaatga

SEQ ID NO: 370 - DNA, 25 bp, unidentified
  source 1..25: mol_type = unassigned DNA
cttgggatacaggttgtcaaaatga

SEQ ID NO: 371 - RNA, 21 nt, unidentified
  source 1..21: mol_type = unassigned RNA
tccagagtctcttccatctcc

SEQ ID NO: 372 - RNA, 41 nt, unidentified
  source 1..41: mol_type = unassigned RNA
taatttctactcttgtagattccagagtctcttccatctcc

SEQ ID NO: 373 - DNA, 25 bp, unidentified
  source 1..25: mol_type = unassigned DNA
tttttccagagtctcttccatctcc

SEQ ID NO: 374 - RNA, 21 nt, unidentified
  source 1..21: mol_type = unassigned RNA
ttttccagagtctcttccatc

SEQ ID NO: 375 - RNA, 41 nt, unidentified
  source 1..41: mol_type = unassigned RNA
taatttctactcttgtagatttttccagagtctcttccatc

SEQ ID NO: 376 - DNA, 25 bp, unidentified
  source 1..25: mol_type = unassigned DNA
ttttttttccagagtctcttccatc

SEQ ID NO: 377 - RNA, 12 nt, unidentified
  source 1..12: mol_type = unassigned RNA
taatttctactc

SEQ ID NO: 378 - RNA, 13 nt, unidentified
  source 1..13: mol_type = unassigned RNA
taatttctactct

SEQ ID NO: 379 - RNA, 14 nt, unidentified
  source 1..14: mol_type = unassigned RNA
taatttctactctt

SEQ ID NO: 380 - RNA, 28 nt, unidentified
  source 1..28: mol_type = unassigned RNA
ttgtagataggatccgctctgcccagtc

SEQ ID NO: 381 - RNA, 27 nt, unidentified
  source 1..27: mol_type = unassigned RNA
tgtagataggatccgctctgcccagtc

SEQ ID NO: 382 - RNA, 26 nt, unidentified
  source 1..26: mol_type = unassigned RNA
gtagataggatccgctctgcccagtc

SEQ ID NO: 383 - RNA, 28 nt, unidentified
  source 1..28: mol_type = unassigned RNA
ttgtagatgatgaccgttcccgaggtgc

SEQ ID NO: 384 - RNA, 27 nt, unidentified
  source 1..27: mol_type = unassigned RNA
tgtagatgatgaccgttcccgaggtgc

SEQ ID NO: 385 - RNA, 26 nt, unidentified
  source 1..26: mol_type = unassigned RNA
gtagatgatgaccgttcccgaggtgc

SEQ ID NO: 386 - RNA, 28 nt, unidentified
  source 1..28: mol_type = unassigned RNA
ttgtagataaggcacattgcttggtaca

SEQ ID NO: 387 - RNA, 27 nt, unidentified
  source 1..27: mol_type = unassigned RNA
tgtagataaggcacattgcttggtaca

SEQ ID NO: 388 - RNA, 26 nt, unidentified
  source 1..26: mol_type = unassigned RNA
gtagataaggcacattgcttggtaca

SEQ ID NO: 389 - DNA, 40 bp, unidentified
  source 1..40: mol_type = unassigned DNA
tgccctgacctgtgactgggcagagcggatcctgaaacgg

SEQ ID NO: 390 - DNA, 28 bp, synthetic construct
  source 1..28: mol_type = other DNA
tgccctgacagagcggatcctgaaacgg

SEQ ID NO: 391 - DNA, 25 bp, synthetic construct
  source 1..25: mol_type = other DNA
tgccgcagagcggatcctgaaacgg